SEQUENCE LISTING (14 sequences)

SEQ ID NO: 1 - protein, 77 aa, synthetic construct
LQCYNCPNPTADCKTAVNCSSDFDACLITKAGLQVYNKCWKFEHCNFNDVTTRLRENELTYYCCKKDLCNFNEQLEN

SEQ ID NO: 2 - DNA, 40469 bp, Homo sapiens
  source 1..40469: mol_type = genomic DNA
tttgtaattaggctaatccatttagtgattcctaatattttgtactcacagagaactaattgactaaacaacttgaacgctagtggtttgtccttagacaatctgtctttgaatttaaagtctttatcgctaagaccttgactttaaatttttcatcactacaaccttgaatttaatttcaggtcttcaacatgatgaccttggatttaatttaaagtcttcaacactatgcgctttatcatattattcacagatgcatttttgaaatgtagtatgtaaaagtatgtaacgtgctgtttattaacaaaagattgttcacaacatctcatgtagtttaaatttgtaaatactgcttctgttttgtttctcctttatacacttgactgtctttgtgataagtgacatgaattttatgttaggattaagtatgttttcctgaaacttggattttttttgtaattatataattgagagttaagaatgaaatccttcaagtgttaaaaactcacattttaaaagcaaattttggttccaattctgtattttgtataattgcttattttccataaactaatattagtcagcatttgcttttaaggtagaagatgggatggattcttgctgacctgctgcaaaagtagcaggtccactcaaagtcatctcctagctttgactgtactgtcttgtgtgccccaccatccattttcccccaagaacagttattttatagagaaaactgtaagaaaacaagtggcattcaaggctaagcagctgccattagggaacatgaagacaaacttatcctttcattttcacacatgtcaaggtgttctgaaatagactaagtcatttttcatgtttacttttcatttaatagagtataatcaggctatcaaatcctattatggctatccttttttttttttttttttgagacggagtctcactctgtggcccaggctagagtgcggtagtgtgatctcgacttactgcaagctccacctcccgggttcacgccattctcctgccacagcctcccaagtagctgggattacaggtgcccaccatcacggccggctaagtttttgtatttttagtagagacagggtttcaccatgttagccaggatggtctcgatctcctgacctcatgatctgcccaccttggcctcccaaagtgctgggattacaggcgtgagccaccgcccccggcggctatcctttttagaaaaaaagtcaattgtctttatttagtttgttataacaaaataccttagactgagtcatttacaaacaatagagatttattgctcacagttctggagggtgggaagtccaagatcaaggcatcagcaaatttggtgtctggtaagtacccactctctgcttcaaagatggcaccttcttgctgtgttttatgtggtggaaagagcaaacaaacttcctcaggcctcttttataagggcactaatccccttcatgagggcagagcctaaaggccctacctcttaatactattgctttagggattggatttcaacaatttgggggaacaaaaacattcagaccatagcgtcaaaccacttaattttttcaaaaccgaacttttccaataaagcagtaatcaaaaactagtaaatcttggcaattaaggctggccctggaatcccttgccattttttctttctcttttctgatgacctctctctctatttccagagtacttatgatgccccaaatctcccttccaaccctgtctaacctttgctcctcagaatgctttgatttgctttatcctctcatcagaattcacattctcttagtatgttgaatgagtgacctctagttcatactgaggtgtagttttttgtttgttttttcagacagtctcgctgtgtctcccagactggagtgcagtggcatgatctcggctgactccaacctccgcctcccaggttcaagtgattcttatgcctcggcctcgtgggtagctgggatcacaggtgtgcactaccatgcccttttttgcatttttagtagagccagggtttcactatattgaccaggctggtctcaaactcctgtctttaagtgatcctcctgcctctggctcccaaagtgttgggattacaggtgtgagccaccgcgcccggcctaacgtgaagtttgttggtaacaagcttctgtattttctgctattggcaagaatgcttattcacacgtaacaggaatttaacagttggcattaatgttgcccaaaccagactctacttttggtggtgaaaaggccatttctggctctgaggacccttgaaaacacaactgattttttttactaatttctctgaggcttgatatatcactagaataagacaaatacctaaattaacagtagcttaaatagtttgggggtggttattcttgcgaatagtcagaggtagctagttcaaggctgacatgatggcacagtggtgttatcaacagtccagattggctccatctttctgacctgccatccttagcacaagacttttcctctcaaagacgatcatggttgcatactggctgctgcaggcatattgtattcacaaaaggatgatgataacaaggtaaaatgaagtggaaaatgaaaagtgaggcatgaggctggagaggtaaactagccaggtctctagcagccgtgtggagggcaagcagagaaggggcaggggatgcaggaattgacatgggcaacatggggacctggtattaaggaattgtaatggctcaagcaacgcaaactacagatctgaattagcacagtagcaatgtgaaaaaggaatgaaattgagatattgaaatggtaaaataggcaggaatggctgagtagatgtgagggagtggaagaggtcaacgttgattcctaggtttctgtcttcatttatgaaaaaaattcaggaaaagcagtcactctttaaagatgaattcagatttgtgcacattaagttcaagatgtctgtggaacatataagtggagatgtccagcagtcagctggatatgaatatgaagatagggggagtggtcagggatggagacagacttttgaatcatctgcaaacatacatcccaaggtaaaagagtagatacactatagtctcccagggagcacacacagccatggttctcagtctaatcctgggacaccaaattattagtgggtaaaggaaaagaatctcaagaccgaatggtcaagaaaccattctagggaaccaaactaggagagagtggcatctcagaagccaagcgtgtcaaggaaggaatagctaacaggatgtgtgtaaggtatgtacacaaaagagctttggaatccatcttgggaaatatacagtattagctgttttaaggagacatgctttaaatatcaaagcaagtcatcctgatttaaaaaactcccacttgtatagaggaataccaggatttgagcaccaccaaatggaactcattccctgacatcccaaagccatcagaataacagtagaagcgtcaagacagttactatatgattttccctgcaagacattcagtaatctatgattccccctgcttaaattttatctcatgccttcttccctgaccagttcaacacaagcagccctgagatgtaatatcttctcaccctgaccctcaaagtgtgtctcatgctttctaacagaggccatcctcttggaggaagacttgtacttttggcctgtagccccaccctccctcctatggcgttcttacagtcagttctgcagtgagtgggacactttccaaatataatagcaaatggaaacttctaattacatctgcacataaaactaatgttccaaatggtggttgcggttctggtgagtgcttataattttttgttacctcagcatccatttttgaatatgtttaacttttaataacagaagcaagtctcagtcaccattgacagtttccagtactataccccacgcaaatggctctagctggtggccagagataaacatgcagtgatgtgataacagggcactactgcagcttcaaactctggttcaagttcctcccacctctgccttttcacctgcttgcttttaaatccaccagttagagctctccagaggaaacctaactggataaagttgctggcccaaaggtccgttcctcccttccctctccctgttctccctgatctccgtgtgtgtgtgtgtgtgtgtgtgtgtgtgtgtgtgtgtgtcctctaggcaagctgtgtaccacccagggtccgtaagtactaaaaactcttaaactttcacaccgctgtgttattgaaaccatgcctacaatccagtccctgaggcgaggccaccctgaagggacagactcatgcaggcggatcccctggtcttgctcttctgtccctgcctctggtggctgctggggaacagtagctaccagctaagttgatagagacactcgaagaggttcattcaaaacactggtccccgagcagcccagtaagcttttaaacgcataacattttgtttgggtctcaaacaagtcacaaggcacggatacggatgcggctgtggctgatggctatttggatgctgcccaagcctcgcagtaggaagcagcttcagactgcagcagggccgcatagcccgatagccaggccctggggcacccagggcaccagcaagacgcacagaaacgttttgaaaaacattttaatatacttaaaaaaaaagtaaaagggaaaatcagaagtctttggaagtttcctactgtttttatgtcccatagcaaatccgaggaggacgccccaaactttcagttccctggggttttgaaggtgctcattgggtcctggccacccggccttctcagaacctgggccaggaggctgagctccgcgcgggggtggagggagaggaggaggttcctgccgaggtgcggctgcgcggtgcgggagccgggagcctgggagggcaagggcatcctgaggggcggggccggggggcggagccttgcgggctggagcgaaagaatgcgggggctgagcgcagaagcggctcgaggctggaagaggatcttgggcgccgccaggtaagaaggcccgaaggcctgctggggtttgggtgagccgagccgaggtggcggcggcagtctgggcccgggcggagggtctgtgggggctacccccgtcctcgctttccactcgacccctgccgggcgagcctcccgaggcctggcccttccaacccgtcaccgcggcccggtgtcctagccgaacgctggcttcattttatctacgaggaaggagaaacggggagcgcgccctctcccagaagcagccccgttacccccgtcgcgccccccattccctcgggggcagcctcgcacccgggcccctgccatcgctttcgtctcaagggaagctctagcggtccagagaaactgcgccgggggaaaggccagccctccgtggtcacggcagttcctctctcccctcgcggtctaaatgtgtaattttgaaaagcaggtgtcgggcgaaacggacttttttttttttttgagatggagtttcgctgttgcccaggctggagtgcagtggcgcgatctgggttcaccgcaacctccgtgtaccgggttcaagcgattctcctgccacagcctcctgagtagctgggattacaggcatgcgccaccatgcctggctaaattttgtatttttagtagaaacggggttttcccatgttggtcaggctgatcttgaattcctgacctcgtgatccgcccgcctccgcctcccaaagtgttgggattacaggtgtgagccaccgcgcccggccgaaacggacgttttaagtacaacgtgatgttttgttttcttgggtcggctcgggccgcacccttcttagagtctgactgctgacttttcaaaaaacaacattaacccgggaggctgaggtagcggtgagccgagatcgcgccattgccctactgcctgggcaataagagcgagactccgcctcaaaaacgaacaaaacccacaaagaacaaacaaaaaaaaccaacattaagcagtgtgccttttatggccgtttagtgatacccaggctcttcactggtcatttctggaagtatttaacagcacttgatgggtaaaacaataggcttcacttcacagggtggttgagatgattgacggaaagcgctcagcacaaacgcggcggctgctaattatttcaggtaccctgcgatccctgagcagttgttcaagttctccactcctggcggtttactcccagttccctgaagaaggcacagagccccgaatgttgagggagatggcaacgtttgccaaaatgtggctatttacgtggcagtttatgaaggagaggcagaaagggaggctgcctcccctacaagccaaacagtcctaaaacttgtggcctctgtattttcacagtggaaactagaaactgtttgctgaacttagctgtaagaaaaacatcccattagctgagtggcttaggagataaaagcctaaataaaatggatcttagggtgagagagtggggtgcctaatggcattcatctttgacagctgctggtagaagctcttgaaaagtagagcatggaaaattctgggccacctctttaagaacctcagccaaaccgccacagacattgccaagcccagaagtgcttgaggctttctaaaggcaaagacttctttttacttatgtcctatatcttaggaaacatattcactaaaccttggttttgcatgtgtacttaacacgggaggggccgtgagtgcggagataggactctttggagagttagggattggcattccggaacaaactgcaggaaaggcccttgtaggggaagctgcagatttgtttggtccaaagagttctggcctaataagtgggagctgaccaatgagaacacttggacacagggaggggaacaacacacactggcgcctgtcggcaggtggggtgggtggggatagagcattaggaaaaacagctaatgcatgctgggcttaatacctaggtgatgggttgataggtgcagcaaaccaccatggtacacgtttacctatgaaacaaatctgcacatcctgcacatgtaccctagaatttatatgtatatcacacaaagagttttggagtggttattcctggcatgagaaccttggctggattggaaagccttttccgttatgatggttgttttctctttgtctcttcctgctgatgtaatttgttccgtgaactgagtgatggatgtttgtctgggcgcttcaacaggcatggctagagcaggtattgtgtatgtctccaaggtgacacgcagacctggggttcaaaagctctagttctctgacattgctgacactaggtatcttgtgagttgccagcccctggcgaatagaaacaagatccatccagggtggttttgcgactaacatgttgccttgggacaggtaatttcatcccactgggcctccactaatttcctataaaataaaaacattgggttttattttgaggacacttctagccttaaaattcttcagtctgtaaggcctcaggctcctaggaggagtaaccatactcatgggcaactgtgaatcttgtggaaacaccactgatgtcaactctcttctctaatgacctcagcttaactaatgtatttcagaagctgaatttataagaagtctcccgtctccccactagagtttaactatggttcgttcactcagagcttagacctgggtttaaatgctgttctaccagtttccagttcagaggttgctataataattttaataatttttttttttaattttgagacagtctctctctgttgcccaagctggagtgcagtggcacagtcttggctcactgcaacctccacctcctgggtgcaagcgattctcgtgtctcagcctctcaagtagctgggattacaggtgagtgccaccacatctggctaatttttgtattttttttagtagagacagggtttcaccatgttggccaggctggtctggaactcctggcctcaagtgatccacccgcctcagcctcccaaagtgctgggattataggcatgagccaccacactcggccttataataattttctactgctgctgaaaccaccacaaatttagctgtttcaaatgccacaagtttattaatctttccattctgtaaatcagaagtctgacacaggtctcacagggctgtaatcaaggtggcagggctctgtgctgctctggaggctctaggggagagtctgtttctgtgctcattcaggttcctggtagaatatagtccttgtgactggaggactgaggttctgtttctttgctgagtgtcagttgaggaccattcttagcttctaaaggccgcctgtgtgccttggcttagggaacccttcgtgtcttcatctcattcttcaaatttcttgcctctttttctgttgtcggtcatgagtctttgacagactctgctaccttcttcctctttcaatgacccctgggattacattgggcccattgaataatccaaaataatctccttaatctcatctacaaagtcacttgccatgcaaggtctcctgttttcacaggttcctggagttaggatacggacatttttggggaagagaggcattattctacctaccacataagttagttgcataaattagttctcttcctcaatcttgtttcctccccctgtaattggaggcaaaggttcagtttaccttatggtattattgtgaggattaactccagttactatgtcaagagcatctatcttagcaactggcccttgatatttgctcaccaaatggaagttaacacaggaacaaggtgtattagtccattctcacactgctataaagaagtacctgagactgggtaatttataaagaaaagaggtttaattggctcgcagttctttgggctgtataggcttctgcttctgggaaggccttaggaaacttagaatcatggtggagaaggggaagcaagaatgccttacttggcgggagcaggaggaagacagcaaagggggaagtgctacacactttcaaacaaccagatttcctaagctctgccacaagacagcactagggggatggtgctgaactgttagaaaccaccccataatccagtcacctcccaccaggtccctcctccgacactggggattaaaattcaacatgagattttggaggtgtattagtccattttcatgctgctgataaagacataccgaagactgggtaatttgtaaagaaatagaggtttaatggactcacagttccacgtggctagggatgcctcacaatcatggcagagggtgaaaggcacgtcttactggcagcagacaagacagaaatatgagagccaagtgaaaggggaaaccccttataaaaccatcagctcttgtgtgacttattcactaccacaagaacagtatgggggaaaccgtccccagtgattcaattatctcccaccaggtccctcccacaacacgtgggaattatgggagttacaattcaaaatgagatttgggtgggaacacaaccaaaccatatcacaaggtcttgcctaatggcttgcattcaatgtcataatcatatgggcccttaaaagtagaagcggctcctaaatatttaagaactaacagcttcttttataaccctgttggaagaaacatttccaacccaatctaaagtaacctggcccttatgccaggtcaccctctaacccagtggttcttagtgtgatccctgaaccagcagaaacgcagcacctgagtgctggctcaaaatgcacattcttgagccttacactagcccacctgaattaaaacctctagggctggagtctaatcatctacttaagagccctccaggtgattctgatgctccctaaagtttgagaaccactgttatgaccctcttttattttcgtcctagtctttagcaccagttggtgtaggagttgagacctacttcacagtagtaagtatactgatgagtgaattccttaaatgttattaaataattatttgatgtgttaaatgttttatgttcaatttctttggattgcatatatatatgtgcatatatatgtgtatttgtgtgtgtatatataatgtatgtatatgtgtgtttttacgtatatatgtaatatatattatatatgtttatatacagttgaccctaaaacaatgcagggttagggacactgaccacctacacggttgaagattcaagtttaacttttgactcccccaaaacttaattactaatagcctactgttgaccagaaactttaccgataacataaacagttgattaacacacatttggtgtgttatatgtattgcctactgtattcttatgataaagtaagctagagaaaagaaaatgttacttagaaaatcgtaaggaagagaaaatccattacagttctgtactatagttattgataccatcagtttatgtcgtctgtttacaagatgaattacttgtctgaaatggcaggcggccacaactgcagacctcaatctgtagtacatatcaagtagttcaactgttttttggtaacatcatgacttttttctgcttcttgggagcgtggccagcatcacttgtggcaccctgtaagggtctcatgtgtcattcaagttttacactattgcgcttaacacgataaagaaaatactcaagaactgcaagagatcaccttttactgtgatgtgcaatttcttaaagaggcaaacagctcacagataattagcatcacatgatactaatcatatccctgctgataagtagatccttgcaacacttgagctcactgcaatagcaacaggaggtggctatggcattattaacagtatatactatagttgattttatgcagtatgatttaatactgcatctttacatttgtttacatttctctttactttggtgccatgtctggtctgtgtttgtgtgtgtaagttttaataatttatttttttgagacagggtcttactctgtcgcccaggtgggagtgcagtggtgtgtcttggcccactgcagcctcaacctcccaggctcaagcaatccttccacctcagcctcctgagtagttggaactacaggcacacgccaccacacctggctatttttttttttgtagagatggggttttgccagtctcgtctcaaactcttggctcaagcaatccacccacctcggcccctcaaagtgctgaaattacaggcaagatccaccatgcctggctgacacattttaactttataatagatttgtgtatgtttttgggtagtgataaaatagactagtatctatatacattttatacattcatgacatacctaaatttttcttaattttaaaaaatatttctaggctatgcagtttatctaacagttttttcaattgcaaatctccaaaatattttccaatatattttcagaaaaaaaattgcatataaatggacccctgcagttcaaacctgtgttgttcaagggtcaaatgtgtgtgggtgtatatatatatttaattgatatttggggggccggttggggttttggatgaacagtaacacgttttcctacttaaaatagtggaaatagactcattacattttctacccaaatacccagaacatttgctatctaaaacattttcaggaacttgataagaagtgcctgtattttttttttttctcggagatacggggtttaagttcttttttaaaaattatactttacgttctggggtacatgtgcacaatgtgcaggtttgttacataggtatacatgtgctatgttggtgtgctgcacccatcaacttgtcgtttacattaggtatttctcctaatgctatccctcccccaccccccaactcccgacaggccctggtgtctgatgttcccctccctgtgtccatgtgttctcattgtttaactcccactgacaagtgagaacatgtggtgtttggttttctcttcttgtgttactttgctgagaatgatggtttccagtttcatccatgtccctgcaaaggacatgaactcatcctttttcctgcaaagaactataactcatagtattccatggtgtatatgtgccacattttctttatccagtctatcattgatgggtatttgggttggttccaagaatttgctattgtgaacagtgctgcaataagcatacgtgtgcatgtgtctttatagtagaatgatttataatcctttggatatatacccagtaatgggattgctgggtcagatgttctagatccttgaggagtcaccataccgtcttccacaatggttgaactaatttacactcccaccaacagtgtaaaagcattcctgtttctccacatcctctccagcatctgttgtttcctgactttttaatgatcgccattctaactggtgtgagatggtatctcattgtggttttgatttgcatttctctaatgaccagtgatggtgagcattttttcataagtttgttggctgcataaatgtcttcttttgagaagtgtctgtttatatccttcacccactttttgatgaaattgtttgttttttcttgtaaatttgtttatttgtagattctggatattagccctttgtcagatggatagattgcaaacattttctcccattctttaggttgccttttcactctgctgacagtttcttttgctgtgcagaagctctttagtttaattagatcccatttgtctattttggcttttgttgccattgcttttggtgttttagtcatgaagtctttgtccatgcctatgtcctgaatggtattgcctaggttttcttctagggtttttatggttttaggtcttacatttaagtctttaatccatcttaagttaacttttgtgtaaggtgtaaggaaaggatccagtttcagctttctacatatggctagccagttttctcagaaccatttattaaatagggaatcctttccctgtagcttgtttttgtcaggtttgtcaaagatcagatggttgtagatgtgtggtgttatttctgaggcctttgttctgttccattggtctatatatctgtttcggtaccagtaccatgctgttttggttactgtagccttgtagtatagtttgaagtcaggtagcgtgatgcctccagctttgttcttttggcttaggattgtcttggctatgtgggctcttttttggttccatatgaaatttaaagtagttttttccaattctgtgaagaaagtcagtggtagcttgatgggaatagcattgaatctataaactactttgggcagtgtggccattttcatgatattgattcttcctatccatgaacatggaattttcttccatttgtttatgtcctcttttatttccttgagcagtggtttgtagttctccctgaagaggtccttcacatcccttgtgagttagattcctaggtattttattctttttatagcaattgtgaatgggagttcactcatgatttggctctctgtttgtctgttattggtgtataggaatgcttgtgatttttgcacattgattttgtatcctgagactttgctgaagttgcttatcagctgaaggagattttgtgctgagacgatgggattttctaaatacacagtcatgtcatctgcaaacagacaatttgacttccttttttccaattgaataccctttatttctttctgttgcctaattgccctggccagaacttccaatactgtgttgaataggaatggtgagagagggcatccttgtcttgtgccagttttcaaggggaatgcttccagtttttgcccattcagtatgatattggctgtgggtttgtcataaatagctcttattattttgagatacattccatcaatatctagtttattgagagtttttagcatgaagcgctgttgaattttgttgaaggcgttttctgtgtctattgagataatcatgtggtttttgtcgttggttctgtttatgtgatggattatgtttattgatttgcatatgttgaaccagccttgcatcccagggatgaagccgacttgattgtggtgaatgagctttttgatgtgctgctggattcagtttgccagtattttattgaggattttcacatagatgttcatcagggatattggcctaaaattttgttgttgttgtgtctctgccaggtttcggtgtcaagatgatgctggcctcataaaatgagttagggaggattccctctttttctattgattggaatagtttcagaaggaatggtaccagctcctctttgtacctctggtagaattcagctattaatccatctggtcctggactttttttggttggtaggctattaattattgccttaatttcagaacctgttattggtctgttcagagattcgacttcttcctggtttagtcttgggagggtgtatgtgcccaggaatttatccatttcttctagattttctagtttatttgcgtagaagtgtttacagtattctctgatggtagtttgtatttctgtgggattggtagtgatatcccctttatcattttttattgcgtctatttgattcttctctctttccttctttattagtctggctaacagtctattctgttgatcttttcaaaaaaccagctcctggattcattgattttttgaagggtcttttgtatctctatctccttcacatctgctctgatcttagttatttcttgtcgtctgttagcttttgaatttgtttgctcttgcttctctagttcttttaattgtgatgttagggtgtcaattttagatctttcctgctttctcttgtgggcatttagtgctataaatttccctctacacactgctttaaatgtgtctcagagattctggtacgtcgtgtctttgttctcattggtttcaaagaacatctttatgtctgccttcatttcgttatttacccagtaattcaggagcaggttgttcagtttccatgtagttgtgcggttttgagtgggtttcttaatcctgagttctaatttgattgcactgtgttctgagagacagtttgttgtgatttctgttcttttacatttgctgaggagtgttttacttccaattatgtggtcaattttagaataattaccatgcggtgctaagaagaatgtatattctgttgatttgggatggagagttctgtagatgtctgttaggtccacttggtccagagctgagttcaagtcctggatatccttgttaatttcctgtcttttgatctgtctaatattgacagtggggtgttaaagtctcccactattattgtgtggaaatctaagtctctttgtaggtctctaagaacttgctttatgcgtctaggtgctcctgtgttgggtgcatatatatttaggatagttagctcttcttgttgcattgatctccttaccattatgtaatgcccttctttatctcttttgatctttgttggtttaaagtcttttatcagagactaggattgcaacccctgctttttttgctttccatttgcttggcagatcttaattcattgtgagtagccttccacgcccattacttttcctggctatgcggttccccaccatggaagtgtaccatagtatactcatttccttattgttggatgtgtgtatcactaaatttttgctattttaaagaatgtttgtatgcccttgactatttcttattcctgtgatgaaaggcctacttggagctcccattacggaaatgactttagagtcaacttacgcaccacccaggaaagcctgccgttccttcatcatgggggaagacttgttcctacatatcttctttgagggatttttcagttgtgcattctttagaatgattaaaaatcattcagaacaatagcagtattattagaacagtcgttagggacaaacccaccgttattaatgaatcctgaaatgtttggaaaggagttgagtgttgatgctttctgagtgtatcttgcatttaagtacttggtttgggtggaatggcaacacttttggttcatttaccatcttatctacacttaacttatgggcatcttcttttttttttttttttttccttaaaatggagggtgttttagtctagggagtagttttaccaaaagatagatgatctatgactactagaaaaggaacattgtcatgtgtgaattttcctgatgtttctgattgtaaaagtaatacttggttaattatagaatatttggggaaaggatggcaaaaagtaaaggatcaggaagaaaaataaaaagccataaatgatcttgcttagtgttagttaccatctacattgtagggcatttataaaatagctttaaaacaatcagctgtttctttttctgcctcccgccctcctccgccccccatacacatacacatactccatatgaacgttttgctcttggaaattcatattttggcagtggttcataaaggcctggtcttattttgatggaacagcccgaccttggcatctacacatagctacttcctttctctgctgtcttggcttcccctgcagcatgtcatgggatcagctatagcaacaaccagccagtcatgtggaactgaggctaagacttgacactccccaaccatcccagaaaactgagggcttccttaggtttgatgcctgaggtctcccctgctcattcaacccagggtgtttctctggcttcctcacctcacatcaccctctgtctgccttcccaccgttcaccccccaacactccctccccacacaatttcttcctcaagtaacctcattactcctgagtagcactaacagtggccacagctttttgagtgccatctttggccaggcattgtgctatgcagtacatgtaactatctccatgcttactagccaaagcgtgatcagaggttaacacatagcaaagaggtgacagagccagaactatctggccttgttcagaggcctccagattggactggaatgcctgtgctctgaaccctttcattgtgttcttcctgctttcttagcaggccacttctttctaaggtacttcaaagaacagctccccatcaggggccctgtgtgttgtggcagtgtgatttatgtcaaggaccattccaagggaagtgtcctgaaccggcagctcctgccagcctgacacctttccagtgcctaacccgagtcaggagattccttccttcctccttccgtggggatgcctgacattgtagctacattgggtttttgtttgtttgtttggagagaggaccttgctgtgtcacccaggctggagtgcagtggcacagtcacagctcactgtagcctttacctcctgggctcaagcgatcctcccacctcagcctcccaagtagctgggactacagttatgtgccaccacaccagtctaatttttttttaatgtttttatttttgtagagacagggtctcactataatacccaggctccgatcatgctatgcttaacttttaggaagtgtctggagctctctgttcggggacctcagaccaataccttcctctccctgctcacttgtagcagttccttgtatactggagccctgtctaacctgagtctataaatcacccttcctactcagaaacccctgaaaatggggaaccaagcaagccgctggcatgtgctggagcccaggtctcctgggaaggtccacatgggggatggtgtcccgtagcaggctgtaggtttcagagcccagcgggtggtggggagccctcaatttgtaaggaaaggccagatgacaattgtctttcttcctggttcctcacatatcataaaaccacctctttctcatctccaccacccccatctccaggcttgactcactgattgttttcttttttggacaatgtcagaccctgcccttttcctggaacttctcagtcctccactggcctgcgtcagcaaattcctaagtagcagatgaccttcaagatgtgtgagaaggaagggacaggaatgattgtcaggattgtcagttccccagggaactctgcatttctgacttagggagggtagatgactcacccttccaagaacccatctgtctgtccgggcctgtggggctctggagaatagaatatggagtaccctggtttacaggcactgccacctccttatttcaggatgggttcagggatgtaggattggagttgggagatttcttagttctgctatggatgctttgtacttaggcaagttcaaaacctccctaagctttggcttctttactgggtaaagtagggttggaggcatttcaggctttccttcttctcacctgatgctatcactacacatctaggcattcacgtaatggaaacttacaaagttgctcaatacctctcttcatgtccccaaagagagcaagcaagtgtccccattatacttcttacagtgaggagccagagcttccaggtatgctaagccattgctccctgttccactgctgaatgcatgctggtgccttccagagtcacccctttggtctgattcacttagtgctgtggctccgagaagtggccctctctttcagccagcagcttggggtggggccagtagagtgggaaccacagtggctatatgagtcaatttgctcatattgtcatttgactcactgaccctgatggggaagatagtgttattcccacagtgtggtagtacacactggggcttatagggactgagcctactcaagggtatatggtgctgtgggtcagagctggggcatggcccagggattcagtgtgccttgactccccctgtaaatgttcctctcagaagccttcttggccttccagcccttggtttttgagacaaccagcagtcatttgttcgttcctgacattccttcctgtcccttccttccaggttctgtggacaatcacaatgggaatccaaggagggtctgtcctgttcgggctgctgctcgtcctggctgtcttctgccattcaggtgagtgctccagtctcaggacatggatctaggctgccttggccatgaactcccttcttaagcctcagtttcagccccagctgctcctccaggcctggctttggttcttttgccttccagtgttacaggtctggggttgtatgacagctccttcctatgttatgaccttgtcctcagtggggcatcgtcctgagttacagcccgtcctgagttacagcgtggcttttggcaggggtgggtcaacaatcggcggctgaagtttcactcctgtcaccataagctgcttggggacaccaatggataccattcttagagtgctgtgcaagtccctttggggtccactctgcttagagccatctgagctcagtccttctgctctgaggctcctcctgtgttttaatgatgatgatggtcatagtgacagcagcagcagctaactaaccctgaagcatttatgtgccagtcactactctaagcattttacgtgtatttgcttatttaatcctcatgccaacctgatggagttggtggtatttttatccttgttttacaagcaacaagattgaggcctccccaaaattaataacttgtcaaataacacacagaagtggtacacgggaatgtgaacacacagtctggcaacagagtccctgctcttccctcctgctccaggccatctgtcactgggccaccccctctcctcagccacccctcccctggttgctggaggaaattgtgtgcttctgagtcaacttctataacagtggaaacacacatgcttcattgtaaagatcaagtgtggttgtctgcctgtgtcatgttggtataatatcttggccactgctggttttttgttttttataaatatggttggtggggggagcagaaaagaaagtggcaatacagtgagcaatttaaaaaaagatttccacaccactgtagcatgtcttagaatattaacagcaaccatatattcactgtttattgtttactggggactgtgccagagcctgtctcatttaaatccgtccagccattccgggaggtcggtactgttacctcccatattacagctgaggaaatgaaggcaaagtagtaacttggcgccaggacacactttaatatgttctccctttctgagcagaaagagaagtcatggcaccccttcttccccaccttccgtgattctagccagggtgcactgtaatgtggttcacctgtgttaaaacactatattttttctataattattttttatttattcatttttcttggttccttctttttttctgtctctgctgcttaagttggaatattttctagaattttaatagcccagtgacatcctacttccagcagtctgcatttgtttatggagcaagcttgggaccatcagcttctctgtgacaagtcttggggtgcacttcagccctttctcagagaattgagatgtgaagctcacatgcccacagatgagaggccttctttgctgctgcccaaaagggtctcaggaccctgcaggggaactttcctctgagctctgcctcttatggcccaggggaaacctcgtctaccctggcttagtggctaaaatatcaaaccattgtccactgacctattgcttgtttttttttgtttgtttttttcagggggatggagtctcactctgtcgcccaggctggagcgcagtggcgcgatctcggctcactgcaacctccgtgtcccaggttccagcgattctcctgcctcagtcttctgagtagctgggattacaggagcatgccactatgcctggctaatttttgtatttttttttatttactttttaatttttgtatttttagtagaggcgggctttcaccatgttggccaggctggtctggaattcctgacctcaaatgatccacctacctcagcctgccaaagtgttgggattacaggcgtgagccattgcacctggccctgttgcttgtttttgacctcagtttccaccataatctagagtttccttctcttcccactcaggtcaattagaagaacatcatggttaaccaagcttggattttagaactgacagcctgagtttgagctcagctcggtcacttagtagctgtgtggctttgaacaatttagttaacatcttagtcttagttttctcttgcaaggtggggatgaagataattgagcctagttctcttaacaatcctatgaagagcttagcacatagatgcacatatatttgaacactgaatgaacatgtttgccatgaatatttttgcccacagacgttccaaccatgggttttagcctttccttctaaagctttcttaagaggccaaatgagcccgggatctagccaccacaaaaatccatgtctacaggtaaccttttaaaatggccctcaaacatggccagtctcaagaacaacacccttcatcttagactcttttcatttcatgcgtgtgaatacatgtctggcacaaggaagggaccaaagaaaaattgcatctttgcgccttctccttgtggcttccctctcacacgggcatgacagccacaggctgacagtgggggagaacaaacaaagcctggtggatggggccctgcccacaggccccagggctgccgccagctctgctgctttgtagccaggtgtccttgtgtgagtcacatcacctctccagacatctgtcttgtcatctgcaaaaaagttggggaggaggttggtttttattttattttattttatttattttttagacatggtctcgctctgtctcccaggctggagtgcagtggtgcaagcttggctcactgcaccctccacctcccaagctcaaatgattctcatgccttagcctcctgaatagctgggattacaggcatgtaccaccatgcccggctaatctttgtatattttgtagaggcgaggtttcaccatgttggccagggtggtctcaaactcctggctacaagtgatccacccacctcggcctcccaaagtgctgggattgtaggtgtgagccgccacacctggcctagtttgttgttgttttttaaaactaatatttcaaggcattaatctgtaaccttcacaagtcagaggatcctctattgggtaggatttcacacctgtgagggcagacatggccatttcagatgctctagagaactgggttcctgtgtgtgctcagaaagtgaggacccaccttgcttccactggactttccactgaaaaatctatttccatggtgataaacctagccttcagccttagaactagaagtcagctgttgggctctaaaacccacacttggtttaaccatgacattcttttgtgagattttacgtgtttcatctttgaaaatgtcttttcacagaggtaggtacttccaaatactgattacatggtagaagtcatatggcatgaagatagtttatctctgatgatggaaaagggggtatccatacaaaccaggaagatttccttccaactcttgcttcctcagtctctccccatcacacgtgtggactgtaactcatctttttgttgtcactgttgttacaaaaatggagaaggaaaaataacccagcaaaaactgacagctagcaaggatgagtcttaagaattgactgggagcaataattgtttttcaaactagggaactggaggcaatttgcttctgatgtaagatgaccctcacgttgtgtctgctggtacccagctgtcctatgcccatgggtttaaagtgcacagctgttgtcttgtctttgacagtgtgtactcctggcattcggtcagtcccttagaagggagtagttcagtaatgataccctgtcccagaggttaccaaactttttctgtacaggaccagatagcaatttttttttttttttaaacagagttttgctctgtcacccaagctggagtgcagtggtgcaattgtggctcactgcaacctctgcctcctgggttcaagcgattctcctgcctcagcctcccaagtagctgggattataggtgtgcaccaccatgcctggctaatttttttatttttagtagagatggggtttcgctatgttggccaggctggtgtcaaactcctgacctcaggtgatccaccctcctcggcttccctaagtgctgggattacaggcgtgagccactgcgcctggcccagatagtaaatattttaagctttgcagaccaagaggcaaaatcaagggtattatgagacttacctcaataataagaaacaaaattttcacagatttttttattgacaaaattcataacataattattgagtccagcataataatacacatctactaatgaggggaatagaattctttttagggggataaccatttgcttaattggggttcaaaattagtgttccctcgcatcaaatcaattgcagatgttcatctattaatactaatctgtgatgacattttacatatttcattttgaaaatgtattttcacgcagataggtattgccagacattgatcatcaacccacgaggatgtgatttaattgggcatattcattgcttggaagacacttatagaattctgtttgtttctcggtaatttcttcttaacacatcattacattgcagactaatctccttccagttgcaggttaggaggaagcttctcaattgcacagttagatggatattcaaatgtggatatttcctttgtgcttgcatcagggtccaaaaaatgttgctggaactgtagtttgaatttggaaagatataactgctatacatttgtgcgggagtggaagtataccacaagttgctgactttgggcccatattaatggagatggtgggctgccaggggacaagtcagtgctgctttaagagatcctgactttcttcctgattctaggtcatagcctgcagtgctacaactgtcctaacccaactgctgactgcaaaacagccgtcaattgttcatctgattttgatgcgtgtctcattaccaaagctggtaagagcctcccctgtctgtctcctaaatgtaatggggtaataagtgcctgggaaaaaaattgtgccactgtaatcctcattaggcttgcatcaagtaatagctaccatttattagcgtcaggtgtgtattgggctaagcactttcttcatacattttaaatttaatgttttgcacttctaagggtgatcttttatttccatttgataaatgtggaaatggaggttcagattaagcaagaaattatggttaagtaaccgactcaggaattgaacatggaagtccatctgattccaaagcttgtgctcttttacagcagccctgtgctgcctcccagaccttggagaacctactgtgtgcccagcagagggttaggagctagggatgcaaagataaagaaaggacacatacaattaaaattatgacaaaggcctgtttggctgtcctaaatgtcattatatatgcctctgtctcattccatggggtatctggtatattcagagtcctgaggagactcaagctgagctcaaaggacacgtaggaggtaagagggctaagaagggaagggtgtgcctgggaggggcaacagcaggtgccagggctcggtagcctcagagcaaggtgctcatgaatttggggatgggcaatgctaacatgcacaagtcctgtccagtctgcattttccaaatttgttagactacagaatattcacctccatcaccctgttaaaatagagtttaggaagtggtagacagaggaaagaaaccagcaaagacatgccctctgttcttgctggggtgtggttctggatagaatcgatgtgtgcaaatgatgccagcgaggaatggcagtttcagaatgttcttagtgtccaaggtaaaaccctcaggcttagatttctaagagattaataataaaaacactccacaaggttatttgtactcttagaccttctacaccagctccccccagtgtcaacatcaagaaatggtgaacatgtcaggtaaactcagaaaagccttgggacacccacatgttcagaaaaacctgagataaagatgattaacttccccagaaaggagaaatccctcaataactaaagacaaattatagttaccctgtctaaaaaaacaggggcatctgataattttgatatatttagctggatttgataaatttgatatagtttagctggattgcagctaaaactccaaaccttccattcagagctcttgattctgtagggcaccagtccctctgcccccactggaagaccatattcattcactgttaatgtaggaagcgtagtaaggcccctagcgagaacatggtcattatgtacgagcacagaagacttggctctttagcttgatcaactgtgtctcttggactcctcactactaagtataattaaactaagtatagtttccaaatgagacgataagggcaattactatgtgttgaatgaatattacagcagcaattagagctctgacttctgtcctgccaccagctagctctctccacctctgtttttcatttatgaaatgtatgtgttggacagcagaggtagggaggagaggaaaggaggtcaaagccaagatctttttccagctcttaaatcccaaaatttcttataaatggttaatgtaaacaaaaatgagacacacctattaaaatggccaaactccaaaactctgacaactccagatgctggtgaggatgtggaacaatgggaactttgtttttctggttttttttttcttttttgagacaaggtcttgctctgtcacctaggctagagggcagtggtgcaatcttggctcactgcaacctctgcctccagggttcaagtgattctcatgcctcagcctcctgagtaattgggattacaggcataagccaccatgcccagctaatttttgtatttttagtagagacggggtttcactattttgcccaggctcatctcaaactcctggcctcaagtgatctgcctgcctcggcctcccaaagtgctgggattacaggcgtgagctaccacgcccagcagcagtgggaactttgattcattcctagtgggaatgcaaaccagtatagctactgtggatgacagtttggtagtttattacaaaactaaaatataacacaatatataatacttgatattttgataattatatacagtatataatattttataataaatgtgttactggtttgtgtatttactatattttttatcatgttagcatgtagtccttctacttactaaaaaaattaactataaaacagcctctcgggaggctgaggcagaagaattgcttgaacccaggaggcagaggttgcagtgagccaaggtcgtgccattgtactctagcctgggtgacagaacaagactctgtctctacaaaagaaaaaaaaaaaaaaaaacagcctcagcaggcaggtccttcaggaggtattccagaagaaggcattgctctcctaggacaggacagccccatgagtattcttacccctaagaccttccagtgggacaagatgaagaggtggaaaacagtgatactgatgatccagaccctatgtaggactaggctaatgtgtgtgcttgtgtcttcatttttaacaaaaacaagaaaagtttttaaaaatttttttaaataaaaaagcttataaaataagaatagaaagaaaatatttttgtgccactgtataacatgtttgtgttttaccctgttttattacaaaggtaaaaacatttacaattaaaaaatttctaaagtaaagtggcagtaaactagggttgattttttaaaataagcttagtgtggactaaatgtacaatatttaaaacatctacagtagtgtacagcaatgtcctaggccttcacattcgcccaccactcattcactgacttccaatctcacaagctccattcacagtaagtgccctgtacaggtgtaccatttttaaaatcttttgtgtcatatttttactgtatttttctatgtttagatatggtcagatacacaaatatttaccactgcattacagttgcctacagtattcagtatagtaacttgctgtacaggtttgtagcctaagagcaataggacataccacatagcctgagtgtgtactaggctattccatgtaagtttgtgtaactacacgctatgacgtttgcacagagacagatatgatggtgctatacaaagtaagtctagaagagagacaggttggctgctaattcaaccaatatttactggcatcctagcagtataaaccatggaagtgtaagcaaggttaaatgtacttagtcttgaaggagcctctgatgacagtgtcaagtggtcactgctgtaatgacgttctgaacaagggccacatttaagggtgcctaggacagctgaagactgttgactttagatagacttgagccgaagtcttcaaaaagagtgaggaggagaaaacctgtaacactccagggattctgatataagccacttttatgtggataattccaaggctggctttggtgagattattaccatcgttaaggctgttgtttgaatatgataattcaagtaaagcactttggtacgctgcacgacacatagtaaatgattaattggtagcaattattcataaattactgtctgttctccccaagcttctaggcttccctaattaaaattctgatagctttaaagcttttcaagttccaatgcgtaattactgttacaaagcagtaacttgtctgaatcaccagcagcctctagtacaatattagaagcttccagttatttagttaagacttcagcaactagagccatttctggaatactctatgtggactctgccacttgactaaacttcattaaattccaatccagatacccatcccctcagaggaggaggacaggacagttatgcctgcagttataaactgagaagtgttgtgttccaagctctgcatggaattgggtaaattagctacaagcctgaaataaggaagtatgcatctgcgtttttttctttccagcacagaagataagaaaccactttcagggcaaggacaaataccttcttataccccagtatctagcacacaggcactcagttatttttggagaagaaaatctaccataataccactctctcaactaatacatttttcacctaacatgctttttctgaatattaaaaaaactgtgataagatacttggagtatatacttctggcctttattttcctgtgtgcccaaggctatttttccaaaaatgggaccatgatggcgtgttttttgtagaaacttttccagtattactgaataatctaatgatttttaaaatgaagtttcaaatattttaaaaataaatcatccatgtggcaaattaaaaaaactcaatagaaacataaacgaagtgtattcttccatgatgataacactattaaattacttgggaatccttccagagaaaaataaatgcacttacctcaatgtatatgtgctttatttccacaaatgggtcacattctatacactgttctgcatctagggtttttattgagatataattcacagaccagaacgtttacattttaaagtgtataattcagtggctcttagtatatttacaaggttgtatgacttatcgcaactgtctaattccaggatctttctgtcaccttgaaaagaaacctgttagcagtcagtcctcattcctccctcctgcatgccccagcagtgactgatgtactttctgtcttcatagatttggctctcctgggcacttcatatgcaaggaatcatacagtattgtggcactttgcgactagcttttttttcactaaacataatgtttccaaggttcactcatgctgtggcatgtatcagtacttcattctgttgtggccacatagtattctattgtgtggatatatcacattttgtttatccattcaccagtaatggacatatgggttgtttccactttttagcaattttgaatcatggtgccatgaacattcatgtggaaacttgtgtgtgaacgtagttttcaattcaaactacgataagatacttggagtgtatacatctggcctttattttcctgtgtgcccaaggctatttttccaaaaatgagaccatgatagcatgttcttatagaaacttttcctgtattactgaataatctaatgatttttaaaatgaagtttcaaatattttaaaaattatccatgtggcaaatttgaaaaactcaatagaaacataaacgaagtgtattcttccatgatgataacagtattaaattacttgggaatccttccagagaatggcagaagtatatacacttgtgagtggaattgctcagtcaaatggataactctatctttaactttttgcagaaatgccaaatttgtccagagtatctacaccattttacattcccaccagcaatgtttgagggttccaattttttccacttccttgcaacatttgctatttaccattttcttgatcttagccatcctagtgagtataaagtggtatctcactgtgattttcatttgcatgttcctaatgactaatgatatcaagcatcttttcatgtacttattggccatttgagaatcttctttggagaaatgtttattcaaatcctttgcccatttttttttaaagaggcaaggtcttgctttgttgcctaggctggaatgcagcagtacaatcatagctcactgcagccttaaagtcctgagtccaagtgatcctcctgcctcagcctcccaagtagctaagactaaaggcgcaacaccctgcctggctaatttttttattttttatttttgtagagttgggatatcactatgttgcccaggctggccgcaggcaatcctctcacttcagcctcctaaagtgctgggattacaggtgtgagccatcacactggcctcattgtccatattttaattgggttagtctttttattgtagagtttaagaatgttttctatatcctggatactatgctcttacatatgactttaaaaaagaaatagagagggggtcttgccatgttgaccaggctggtctgaaacttcccgcctcaaacaattctcccatctcagcctcccaaagtgttgggattacaggtgtgagccaccgcacctggtggtttacatgtgatttgcaaacattttctccctttctgtgagttatctttttattttttgataatgtcctttgaagcacaaaagttttaaattttgatggagtccagtttacctatttttctgccccccccccacctttatttttttttttttaaatcaagctccttccatgttagcacttgaaggcccacgtcatttcttatctgcatggaacttgcattgtaaggaattctattgtaatttctttaacactttttaaaaattaatggactgggtttttcaggtttttttctgaccattcttgagcatatatcttatacatagctttggctgggcatggtggctcacccctataattctatcactttgggaggctgaagcaggaggaccacttgagcccaggagttcaagaccagatgaccgggcaagaaagtgagaccttatctctctaaaaaaaaaaaagaaaagaaaaggaaaaaatccccaagtaacttcatagcgtaaattctaaaagtggaattgctggatcaaagggggcatgtgtttaaaatcttggcagatattaccaaatttccctcttaacagtataaatgcctgttcctcctccgtcccacccccataactatactggctctgatgagaccttggttttctgtaaaagctctatttagaggtgtatcattatttacttaattgttctcctttacaacccacctgggatgagcatcttgcctagaagtctctacttgcacaggatacatacgaaatagaattgaggattcaaaagcagatacagaactcttcccacttactttcttacccttgtgtgtgtctccccacagggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaagggcctttaatatgtaataacaagagctaatgttgaacacaagccattgccaggtacttaaactgtctcacttagtcttgccaacctaagcaagtagatgccattactattcccattatattgatgaaaaaatgaggttgagaaaggttcagccgcacagccaaatacatagtggatccagggtttggtcccaagtcttgagtccaaaaccctggttcttaccattactgtggttctgtaggggtgaaaaaatttttcttctaatctcttaggttcagagcctggggcctgagaattaaactgacaaagacagattaacagaagacaaagtttattacgcaggtacatggggttcttacagaaacaaagtgaaaacccaaagaagtggcttgagagcttatatacaattttaacaatgggcgattgtagaaaagtgataagacaaaggaaaagaggtttgagcttcttgggcagtaaattatgggaagttaaatatgtgggggaaactaatggaagataagggtattttttattttattttattttatttattttgagacagggtcctgccttgtcacccaggctggagtgcagtgctatgattgtggctcactgcagcctcaacctcagcctccttggttcaagaaatcctcccacttcagctcctgagtggctccaggcaaatgccaccacacctggttaattttttaaattattttatagagacagaatctcgctatgttgcccctggctgttctcaaactcttggcctcaagagatcccacctcagactcccagagtgctgggattacaggctgggtgctgagctatcgcacatggcctgtattagccatagactgaggggaaatacattttcttgagagcctcagcctagtgatcacttgtatgaataaacaatgatgggtggctgccaaagtcagagagcaaatactgcgtgtgacaactaagtgtgggaagctcagtgtgggaagaaaaagcttcccagtttcagacatgccccttagtagggacagctgcctcttgtgtttggacaaagtgtccaagttcaagtgtttcacacacacactgttccgtcttctgtttgatcacaaccctatgaggcagcctggttttcttatttctattttatggaaaacctacttaaaagtcaagcaacttgtccaaggctacccagcttgaatatctgactctctccactttgctgctacacacacacacacacacacacacacactttgcactgtgtgtgcagatacatatgcatatatatatttaacttgtgaagctgatactgactctttaactgccctccacccatcactagtcataccagtgatcattaggcagaggctctcaagaaaatgtattagttatttcccctcagtctatggctaatacaggttactcacataaatgagctgtcatgtcctttggacaaaatgtccaaatcgctcataagcaatgggtgaagtatttgaggtagggcggacaggatttccttttggtggttagctcgttgcctgttagctcctggtgatatctgtgtttggaagagaactaaactgcatttcactgaaaacactccttcaggtctattacaaaccagatgaggatccccttttctagaatcttcctcaacactgctaacgtttggcagatacataggttggctctaacagccattcccaatcaatcctcttctcctttgtctgcctcccacagtagtggatgaaaaatcagacactgattttctcagacttctttgctgtgggcgcggccagtgaggcaatgaggtaagtctgctggtgcaggggccaggtttctaggaaatacttttctccctcatgggagaaagagaggaagcctgaagtaagctttcttctgcagccctggcccgctgcctgttctggatgagggtatgctgggatatgacaacggaagccagcggggtcgcagagcaagagcgaggcggtgggcgg

SEQ ID NO: 3 - DNA, 7563 bp, Homo sapiens
  source 1..7563: mol_type = genomic DNA
agaagcggctcgaggctggaagaggatcttgggcgccgccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaa

SEQ ID NO: 4 - DNA, 7608 bp, Homo sapiens
  source 1..7608: mol_type = genomic DNA
agaagcggctcgaggctggaagaggatcttgggcgccgccagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaa

SEQ ID NO: 5 - DNA, 7796 bp, Homo sapiens
  source 1..7796: mol_type = genomic DNA
ggggccggggggcggagccttgcgggctggagcgaaagaatgcgggggctgagcgcagaagcggctcgaggctggaagaggatcttgggcgccgccagtctctctctgttgcccaagctggagtgcagtggcacagtcttggctcactgcaacctccacctcctgggtgcaagcgattctcgtgtctcagcctctcaagtagctgggattacagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 6 - DNA, 7679 bp, Homo sapiens
  source 1..7679: mol_type = genomic DNA
agaagcggctcgaggctggaagaggatcttgggcgccgccagtctctctctgttgcccaagctggagtgcagtggcacagtcttggctcactgcaacctccacctcctgggtgcaagcgattctcgtgtctcagcctctcaagtagctgggattacaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaa

SEQ ID NO: 7 - DNA, 7801 bp, Homo sapiens
  source 1..7801: mol_type = genomic DNA
atttgactcactgaccctgatggggaagatagtgttattcccacagtgtggtagtacacactggggcttatagggactgagcctactcaagggtatatggtgctgtgggtcagagctggggcatggcccagggattcagtgtgccttgactccccctgtaaatgttcctctcagaagccttcttggccttccagcccttggtttttgagacaaccagcagtcatttgttcgttcctgacattccttcctgtcccttccttccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 8 - DNA, 7678 bp, Homo sapiens
  source 1..7678: mol_type = genomic DNA
atttgactcactgaccctgatggggaagatagtgttattcccacagtgtggtagtacacactggggcttatagggactgagcctactcaagggtatatggtgctgtgggtcagagctggggcatggcccagggattcagtgtgccttgactccccctgttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaa

SEQ ID NO: 9 - DNA, 7613 bp, Homo sapiens
  source 1..7613: mol_type = genomic DNA
atttgactcactgaccctgatggggaagatagtgttattcccacagtgtggtagtacacactggggcttatagggactgagcctactcaagggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaa

SEQ ID NO: 10 - DNA, 7554 bp, Homo sapiens
  source 1..7554: mol_type = genomic DNA
atacttcttacagtgaggagccagagcttccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaa

SEQ ID NO: 11 - DNA, 387 bp, synthetic construct
  source 1..387: mol_type = other DNA
atgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaa

SEQ ID NO: 12 - DNA, 314 bp, synthetic construct
  source 1..314: mol_type = other DNA
atagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccc

SEQ ID NO: 13 - RNA, 21 nt, synthetic construct
  source 1..21: mol_type = other RNA
ggacctgtgtaactttaactt

SEQ ID NO: 14 - RNA, 21 nt, synthetic construct
  source 1..21: mol_type = other RNA
gttaaagttacacaggtcctt

(19) United States
(12) Patent Application Publication
(10) Pub. No.: US 2026/0234637 A1
(43) Pub. Date: Aug. 13, 2026

(54) TARGETED REDUCTION OF CD59 FOR USE IN TREATING DISEASE

(71) Applicant: HADASIT MEDICAL RESEARCH SERVICES AND DEVELOPMENT LTD., Jerusalem (IL)

(72) Inventors: Dror MEVORACH, Jerusalem (IL); Marian ZEIBAK, Nazareth (IL)

(73) Assignee: CUMBARA BIOTECHNOLOGIES LTD, Oshrat (IL)

(21) Appl. No.: 19/155,286

(22) PCT Filed: Feb. 8, 2024
(86) PCT No.: PCT/IL2024/050149

§ 371 Date: Aug. 11, 2025

Related U.S. Application Data
(60) Provisional application No. 63/444,954, filed on Feb. 12, 2023.

(51) Int. Cl. C12N 15/113; C12N 9/22; C12N 15/11

(52) U.S. Cl. CPC C12N 15/1138; C12N 9/226; C12N 15/111; C12N 2310/11; C12N 2310/20; C12N 2310/351

(57) ABSTRACT

The present disclosure provides methods of using a compound effective in reducing GPI-anchored CD59 expression or activity in the liver for treatment of a liver disease or condition. Examples of compounds useful for the methods include oligonucleotides, for example but not limited to antisense oligonucleotides, interfering RNA compounds (RNAi), siRNA, miRNA, or guide RNA.

### SEQUENCE LISTING STATEMENT

The instant application contains a Sequence Listing which has been submitted electronically in XML format and is hereby incorporated by reference in its entirety. The XML format copy, created on Jan. 17, 2024, is named P-619391-PC-SQL.xml and is 112.8 kilobytes in size.

### FIELD OF INVENTION

The present application is related in general to methods of treating liver diseases, glucose intolerance, diabetes, obesity, and peripheral insulin resistance, and combinations thereof. In one embodiment, the methods described herein comprise targeted reduction of CD59 function.

### BACKGROUND

The complement system is a part of the immune system enhancing the ability of antibodies and phagocytic cells (1) to clear microbes and damaged cells from an organism, (2) to promote inflammation, and (3) to attack a pathogen's cell membrane. The complement system is part of the innate immune system and is a functional bridge between innate and adaptive immune responses.

Complement activation is known to occur through three different pathways: a classical pathway, an alternate pathway, and a lectin pathway. Complement activation can be divided into four main steps: initiation of complement activation, C3 convertase activation and amplification, C5 convertase activation, and assembly of the terminal complement complex (TCC), also known as the membrane attack complex (MAC). All the pathways converge at C3, resulting in formation of the activation products C3a and C5a, which promote inflammation, C3b, which clears microbial intruders, and MAC, which lyses susceptible pathogens.

Progression of the complement cascade and the action of effector molecules of the complement system are strictly controlled at each level by multiple complement regulators and inhibitors. These regulators discriminate between self- and non-self surfaces, such as cells tissues. The complement regulatory proteins are present in the plasma and on cell membranes. One example of a complement control protein is CD59 glycoprotein, which inhibits the formation of MAC. CD59 is a membrane regulator located on the surface of a host cell.

The CD59 gene encodes a CD59 glycoprotein preproprotein comprising a 5′ signal sequence and 3′ terminal sequences that are cleaved to produce a mature 77-amino acid glycosylphosphatidylinositol (GPI)-anchored cell surface glycoprotein. (NCBI Reference Sequence: NP_000602.1; UniProt #P13987) The mature CD59 glycoprotein is thus initially synthesized as a 128-amino acid protein that includes the signal sequence and the sequence for a GPI anchor replacement. Multiple alternatively spliced transcript variants, which encode the same protein, have been identified for this gene. The soluble form of CD59 retains its specific complement binding activity, but exhibits greatly reduced ability to inhibit MAC assembly on cell membranes.

Screening of CD59 mutants in a clonal rat pancreatic beta (0) cell line showed that a non-GPI-linked isoform of CD59 is transported into the cytosol in an N-linked glycosylation-dependent manner, in which it interacts with pancreatic 3-cell intracellular exocytotic machinery, consistent with CD59s requirement for glucose-stimulated insulin secretion (GSIS). These observations were restricted to 3-cells.

A noncanonical role of CD59 in beta-cell secretion of insulin has been suggested, wherein CD59 plays a role in insulin secretion (Krus et al., (2014) “The complement inhibitor CD59 regulates insulin secretion by modulating exocytotic events.” Cell Metab 19(5): 883-890.) The main finding from this study concerned the GPI anchored CD59. A version of CD59 lacking the C-terminal GPI signal peptide and attachment site rescued insulin secretion in cells lacking endogenous CD59, showing that non-GPI-anchored CD59 isoforms were involved in insulin secretion (Golec et al. (2019) A cryptic non-GPI-anchored cytosolic isoform of CD59 controls insulin exocytosis in pancreatic beta-cells by interaction with SNARE proteins. FASEB J 33:12425-34.)

There remains an unmet need for methods for treating liver diseases, including fatty liver, and associated conditions such as glucose intolerance and peripheral insulin resistance. It is essential to obtain therapeutics that treat and/or ameliorate liver diseases and conditions. The work presented herein provides CD59 as novel target for such therapeutics, wherein the examples demonstrate that elimination of GPI-anchored CD59 activity protects mice from developing a fatty liver and diabetes that occur in a high fat diet mouse model.

### SUMMARY

In certain aspects, described herein is a method of treating a liver disease or condition in a subject in need thereof, said method comprising administering to the subject a compound effective in reducing GPI-anchored CD59 expression or activity in the liver, skeletal muscle, or adipose tissue, or any combination thereof, thereby treating a liver disease or condition in said subject.

In another aspect, described herein is a method of modulating weight loss in a subject in need, said method comprising administering to the subject a compound effective in reducing GPI-anchored CD59 expression or activity in the liver, skeletal muscle, or adipose tissue, or any combination thereof, said subject suffering from a liver disease or condition, said modulating weight loss comprising maintaining weight, reducing weight, or reducing increased weight gain compared with a subject not administered said compound.

In another aspect, described herein is a method of modulating weight loss in a subject in need who is not suffering from a liver disease or condition, said method comprising administering to the subject a compound effective in reducing GPI-anchored CD59 expression or activity in the liver, skeletal muscle, or adipose tissue, or any combination thereof, said modulating weight loss comprising maintaining weight, reducing weight, or reducing increased weight gain compared with a subject not administered said compound.

In another aspect, described herein is a method of treating diabetes in a subject in need, said method comprising administering to the subject a compound effective in reducing GPI-anchored CD59 expression or activity in the liver, skeletal muscle, or adipose tissue, or any combination thereof, wherein said diabetes may comprise Type II diabetes, Type I diabetes, diabetes associated with weight gain, diabetes associated with insulin resistance, prediabetes, and wherein said subject may be further suffering from a liver disease or condition or obesity, said treating diabetes comprising reducing or inhibit the occurrence of the disease, reduce the severity of the disease, reducing glucose intolerance, or reducing peripheral insulin resistance, or any combination thereof, compared with a subject not administered said compound.

In another aspect, described herein is a method of reducing insulin resistance in a subject in need, said method comprising administering to the subject a compound effective in reducing GPI-anchored CD59 expression or activity in the liver, skeletal muscle, or adipose tissue, or any combination thereof, said reducing insulin resistance comprising reducing insulin resistance compared with a subject not administered said compound. In a related aspect, wherein said subject may be further suffering from a liver disease or condition, obesity, or diabetes, or a combination thereof.

In a related aspect, the reduction of GPI-anchored CD59 expression or activity comprises reducing CD59 expression, reducing the quantity of GPI-anchored CD59, or inhibiting functional activities of GPI-anchored CD59. In a further related aspect, an effective compound comprises an oligonucleotide, antibody or a binding fragment thereof, a polypeptide, a peptide, or small molecule.

In still a further aspect, an oligonucleotide comprises an antisense oligonucleotide, interfering RNA compounds (RNAi), a siRNA, a miRNA, or guide RNA. In yet a further related aspect, an oligonucleotide comprises a conjugate group attached at the 5′ or 3′ end of the oligonucleotide. In still a further related aspect, a conjugate group comprises at least one GalNAc moiety. In a further related aspect, an antisense oligonucleotide comprises a contiguous nucleotide sequence at least 80%, 85%, 90%, 95%, or 100% complementary to an equal length portion of a target CD59 mRNA transcript or CD59 mRNA precursor.

In another further related aspect, a target CD59 mRNA comprises the nucleotide sequence set forth in any one of SEQ ID NOs:3-10. In still another further related aspect, a siRNA or miRNA comprises a contiguous nucleotide sequence at least 80%, 85%, 90%, 95%, or 100% complementary to an equal length portion of a target nucleic acid sequence encoding the CD59 protein, said target nucleic acid sequence comprises the sequence of one of SEQ ID NOs:3-12, or is at least 80%, 85%, 90%, 95%, or 100% complementary to an equal length portion of a nucleic acid sequence upstream or downstream of the sequence encoding the CD59 protein.

In a related aspect, administration of an effective compound reduces expression of CD59 in the liver, skeletal muscle, or adipose tissue, or any combination thereof, reduces the quantity of GPI-anchored CD59 in the liver, skeletal muscle, or adipose tissue, or any combination thereof, or inhibits functional activities of GPI-anchored CD59 in the liver, skeletal muscle, or adipose tissue, or any combination thereof, or a combination thereof.

In another related aspect, an oligonucleotide comprises a guide RNA, and the method further comprises administering a polynucleotide encoding a CRISPR-Cas9 endonuclease operatively linked to a liver promoter, the guide RNA comprising a contiguous nucleotide sequence complementary to an equal length portion of a target nucleic acid sequence encoding the CD59 protein, said target nucleic acid sequence comprising the sequence of any one of SEQ ID NOs:3-12, or a complementary sequence of equal length portion of a nucleic acid sequence upstream or downstream of the sequence encoding the CD59 protein.

In a related aspect, a liver disease or condition comprises fatty liver disease, NASH, or peripheral insulin resistance. In a further related aspect, a fatty liver disease comprises non-alcoholic fatty liver disease (NAFLD), alcoholic fatty liver disease (AFLD), alcoholic steatohepatitis (ASH) (cirrhotic or non-cirrhotic ASH), non-alcoholic steatohepatitis (NASH) (cirrhotic or non-cirrhotic NASH), hepatocellular carcinoma (HCC), or liver fibrosis, or any combination thereof. In another further related aspect, a subject suffering from the fatty liver disease has liver damage, steatosis, liver inflammation, liver scarring or cirrhosis, liver failure, or peripheral insulin resistance, or any combination thereof. In yet another further related aspect, a subject is suffering from diabetes or obesity or a combination thereof. In another aspect, a subject suffering from fatty liver disease, NASH, or peripheral insulin resistance is also suffering from diabetes or obesity or a combination thereof. In a related aspect, a subject suffering from diabetes is not suffering from a liver disease or condition. In a related aspect, a subject suffering from insulin resistance is not suffering from a liver disease or condition. In a related aspect, a subject suffering from obesity is not suffering from a liver disease or condition. In a related aspect, a subject in need of modulation of weight loss is not suffering from a liver disease or condition.

In a related aspect of the methods described herein, treating a subject further treats glucose intolerance or insulin resistance associated with said liver disease or condition, or a combination thereof.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

The subject matter regarded as the methods of treating diseases, for example but not limited to liver diseases, is particularly pointed out and distinctly claimed in the concluding portion of the specification. The methods, however, both as to organization and method of operation, together with objects, features, and advantages thereof, may best be understood by reference to the following detailed description when read with the accompanying drawings in which:

FIGS. 1A and 1B present weekly measurements of body weight in wild-type (CD59⁺/CD59⁺) mice and Knockout (CD59⁻/CD59⁻) mice on lean (LD) and high fat diet (HFD). FIG. 1A presents body weight over time, while FIG. 1B presents body weight as the percent of initial weight over time. HFD—High Fat Diet; LD—Lean Diet.

FIGS. 2A-2L present metabolic cage results measuring the respirometry and other parameters of mice during light and dark periods. Comparisons between wild-type (CD59⁺/CD59⁺) mice and Knockout (CD59⁻/CD59⁻) mice are shown for Respiratory quotient (FIGS. 2A-2B), VO₂ l/d/Kg (liter per day pe kg) eff. mass (FIGS. 2C-2D), VCO₂ l/d/Kg eff. mass (FIGS. 2E-2F), total energy expenditure (TEE) Kcal/h/Kg eff. mass (FIGS. 2G-2H), Fat Oxidation g/d/Kg eff. mass (FIGS. 2I-2J), and CH Oxidation g/d/Kg eff. mass (FIGS. 2K-2L). Wild-type—WT, Knock-out KO, HFD—High Fat Diet; LD—Lean Diet.

FIGS. 3A-3H present Intraperitoneal Glucose Tolerance Test (IPGTT) results of HFD for WT and KO CD59 mice. WT and CD59 KO mice (males) were randomly chosen (n=6). Mice fasted for 16 hours (overnight fast) before the experiment. Following overnight fasting, glucose solution (0.225 g/ml) was injected intraperitoneally (1.5 g glucose per 1 kg mouse). Glucose blood levels were measured at time 0, 15-, 30-, 60-, and 120-min post injection. This glucose tolerance test measured the clearance of an intraperitoneally injected glucose load from wild-type (CD59⁺/CD59⁺) mice and Knockout (CD59⁻/CD59⁻) mice over time after 1-month (FIGS. 3A-3B), 2-months (FIGS. 3C-3D), 3-months (FIGS. 3E-3F) and 4-months (FIGS. 3G-3H). HFD—High Fat Diet; LD—Lean Diet. FIGS. 3A, 3C, 3E, and 3G show blood glucose levels (IPGTT) at different ages of WT and KO CD59 mice. FIGS. 3B, 3D, 3F, and 3H present calculated AUC. Values are mean±s.e.m.

FIGS. 4A-4F present Insulin Tolerance Test (ITT) results of HFD. These experiments determined the whole-body sensitivity of insulin receptors in wild-type (CD59⁺/CD59⁺) mice and Knockout (CD59⁻/CD59⁻) mice at different stages of high-fat feeding by measuring blood glucose levels changes before and after insulin administration. (FIGS. 4A, 4C, and 4E) ITT at different time progression of diets. (FIGS. 4B, 4D, and 4F) corresponding AUC. Values are mean±s.e.m.). WT and CD59 KO mice (males) were randomly chosen (n=6). Mice fasted for 16 hours (overnight fast) before the experiment. Following overnight fasting, insulin solution (1 U/kg) was injected intraperitoneally. Glucose blood levels were measured at time 0, 15-, 30-, 60-, and 120-min post injection. Glucose blood levels over time are shown after 1-month (FIGS. 4A-4B), 2-months (FIGS. 4C-4D), and 3-months (FIGS. 4E-4F). HFD—High Fat Diet; LD—Lean Diet. Values are mean±s.e.m.

FIGS. 5A and 5B present insulin and glucagon plasma levels of four hours fast mice. FIG. 5A shows insulin plasma levels in wild-type (CD59⁺/CD59⁺) mice and Knockout (CD59⁻/CD59⁻) mice on HFD and LD. FIG. 5B shows glucagon plasma levels in wild-type (CD59⁺/CD59⁺) mice and Knockout (CD59⁻/CD59⁻) mice on HFD and LD. As shown, there were significantly higher levels of insulin in CD59 WT mice on a HFD compared to all other groups. Glucagon levels were significantly increased in WT mice on a HFD compared to a LD but did not reach significance when compared to levels in CD59 KO mice. Representative of 3 experiments. ELISA was used for measurement.

FIGS. 6A-6C present liver and fat changes in LD and HFD of WT and CD59 KO. FIG. 6A: Fat content and appearance in an MRI imaging. FIG. 6B shows macroscopic appearance of livers under different conditions. FIG. 6C: H&E histology staining of the liver showing extensive fat distribution in wild type HFD as compared to CD59KO.

FIG. 7A-7E show AKT and phosphorylated AKT (pAKT) signaling in liver and muscle following injection of insulin. Western Blot results are presented examining insulin resistance by measuring AKT and phosphorylated AKT (pAKT) in wild-type (CD59⁺/CD59⁺) mice and Knockout (CD59⁻/CD59⁻) mice on HFD and LD. FIGS. 7A and 7B show the staining pattern and intensity of liver samples with insulin injection (FIG. 7A) or without insulin injection (FIG. 7B). FIG. 7A: Anti-pAKT is shown, with no appearance in WT mice on an HFD. FIG. 7C presents the results in muscle, which were similar to the results to liver, but anti-AKT was weak. Tissues were taken 5 min following injection of insulin. Representative of 2 samples. FIGS. 6D and 7E show the band volume intensity of the Pakt/AKT ratio of wild-type (CD59⁺/CD59⁺) mice and Knockout (CD59⁻/CD59⁻) mice on HFD and LD, in liver samples with and without inulin injection, respectfully. Liver tissue was extracted 5 minutes after insulin injection.

FIG. 8 presents the percent liver weight per total body weight in wild-type (CD59⁺/CD59⁺) mice and Knockout (CD59⁻/CD59⁻) mice on HFD and LD.

FIG. 9 presents confirmatory data using MRI showing higher insulin resistance in livers of CD59 wild-type mice on HFD. Measurements of ¹⁸F-fluorodeoxyglucose (FDG) are shown for wild-type (CD59⁺/CD59⁺) mice and Knockout (CD59⁻/CD59⁻) mice on HFD and LD. The labeled sure allows analysis of sugar metabolism.

FIG. 10 presents p110 and pS6 signaling in the liver following injection of insulin. Anti-p110 and anti-pS6 are shown. Weak-to-no appearance is seen with a HFD but not in CD59 KO mice. Representative of 2 experiments. Tissues were taken 5 min following injection of insulin. Representative of 2 samples.

### DETAILED DESCRIPTION

In the following detailed description, numerous specific details are set forth in order to provide a thorough understanding of the methods of treating liver disease in a subject by effectively reducing GPI-anchored CD59 expression or activity or both. However, it will be understood by those skilled in the art that the methods described may be practiced without these specific details. In other instances, well-known methods, procedures, and components have not been described in detail so as not to obscure the present invention.

### CD59 Polypeptide and Gene

Human CD59 is a cell surface glycoprotein that functions as a potent inhibitor of the complement membrane attack complex (MAC) action. The CD59 glycoprotein acts by binding to the C8 and/or C9 complements of the assembling MAC, thereby preventing incorporation of the multiple copies of C9 required for complete formation of the osmolytic pore. This inhibitor activity appears to be species-specific. Further, CD59 is involved in signal transduction for T cell activation complexed to a protein tyrosine kinase. The soluble form from urine retains its specific complement binding activity, but exhibits greatly reduced ability to inhibit MAC assembly on cell membranes. (P13987 CD59_HUMAN1 The UniProt Consortium, UniProt: the universal protein knowledgebase in 2021, Nucleic Acids Research, Volume 49, Issue D1, 8 Jan. 2021, Pages D480-D489; https://www.uniprot.org/uniprotkb/P13987/entry#function; NCBI Reference Sequence: NP_000602.1)

As used herein, the term complement “membrane attack complex” (MAC) may be used interchangeably with “terminal complement complex” (TCC), having all the same meanings and qualities.

The findings presented herein point to a critical non-canonical role for CD59 glycoprotein in the regulation of pancreatic beta-cell secretion of insulin, wherein reduced or eliminated CD59 cell surface expression and or activity may in some embodiments treat glucose intolerance, peripheral insulin resistance, or fatty liver, or a combination thereof.

The findings provided in the Examples below present results showing the absence of CD59 function played a protective role in preventing the development of insulin resistance and fatty liver. Thus, while the presence of CD59 glycoprotein plays a protective role in complement regulation of canonical activation, reduction of CD59 appears to provide non-canonical beneficial effects in the prevention of liver disease and peripheral insulin resistance.

In some embodiments, insulin resistance comprises peripheral insulin resistance.

Two isoforms of CD59 preproprotein have been identified to date and are produced by alternative splicing. Eight variants of the CD59 preproprotein have been identified, wherein the amino acid sequence of the mature protein is the same for each of them. The amino acid sequence of the mature protein CD59 glycoprotein is set forth in SEQ ID NO:1: LQCYNCPNPTADCKTAVNCSSDFDACLITKAGLQVYNKCWKFEHCNFNDVTT RLRENELTYYCCKKDLCNFNEQLEN. Glycosylphosphatidylinositol or glycophosphatidylinositol (GPI) is a phosphoglyceride that can be attached to the C-terminus of a protein during posttranslational modification. The mature CD59 glycoprotein comprises a GPI anchor attached to Asparagine 77 (Asn77; N77).

In some embodiments, a compound used in a method disclosed herein comprises an antibody that disrupts a non-canonical function of CD59. In some embodiments, a compound used herein binds to the mature CD59 glycoprotein as set forth in SEQ ID NO: 1 or a polypeptide homolog having at least 80% identity with SEQ ID NO: 1, and the antibody disrupts a non-canonical function of CD59. In some embodiments, a compound used herein binds to the mature CD59 glycoprotein as set forth in SEQ ID NO: 1 or binds a polypeptide produce by an alternative splice form of the mRNA encoding CD59. In some embodiments, a compound used herein binds to the mature CD59 glycoprotein and disrupts a non-canonical function of CD59 in liver and or muscle tissue. In some embodiments, a compound used in a method disclosed herein comprises an antibody that disrupts the improper secretion of insulin from pancreatic beta-cells. In some embodiments, the antibody disrupts a non-canonical function of CD59 but not a canonical function of CD59 in the complement cascade.

In some embodiments, a mature CD59 glycoprotein is a GPI-linked cell-surface glycoprotein (Golec et al., (2019) A cryptic non-GPI-anchored cytosolic isoform of CD59 controls insulin exocytosis in pancreatic beta-cells by interaction with SNARE proteins. FASEB J; 33:12425-34. In some embodiments, the amino acid sequence of a mature CD59 glycoprotein is set forth in SEQ ID NO: 1. In other embodiments, alternative splicing appears to encode functional intracellular CD59 isoform that mediate insulin secretion and are downregulated in diabetic islets.

The nucleotide sequence of the gene encoding CD59 is set forth in SEQ ID NO:2:

TTTGTAATTAGGCTAATCCATTTAGTGATTCCTAATATTTTGTACTCACAGAGAACTAATTGACTAAACA
ACTTGAACGCTAGTGGITTGTCCTTAGACAATCTGTCTTTGAATTTAAAGTCTTTATCGCTAAGACCTTG
ACTTTAAATTTTTCATCACTACAACCTTGAATTTAATTTCAGGICTTCAACATGATGACCTTGGATITAA
ITTAAAGTCTTCAACACTATGCGCTTTATCATATTATTCACAGATGCATTTTTGAAATGTAGTATGTAAA
AGTATGTAACGIGCTGITTATTAACAAAAGATTGTTCACAACATCTCATGTAGTTTAAATTTGTAAATAC
TGCTTCTGTTTTGITTCTCCITTATACACTTGACTGICITTGIGATAAGIGACATGAATTTTATGTTAGG
ATTAAGTATGITTTCCTGAAACTIGGATTTTTTTTGTAATTATATAATTGAGAGITAAGAATGAAATCCT
TCAAGTGTTAAAAACTCACATTTTAAAAGCAAATTTTGGTTCCAATTCTGTATTTTGTATAATTGCTTAT
TTTCCATAAACTAATATTAGTCAGCATTTGCTTTTAAGGTAGAAGATGGGATGGATTCTTGCTGACCTGC
TGCAAAAGTAGCAGGTCCACTCAAAGTCATCTCCTAGCTTTGACTGTACTGTCTTGTGTGCCCCACCATC
CATTTTCCCCCAAGAACAGTTATTTTATAGAGAAAACTGTAAGAAAACAAGTGGCATTCAAGGCTAAGCA
GCTGCCATTAGGGAACATGAAGACAAACTTATCCTTTCATTTTCACACATGTCAAGGIGTTCTGAAATAG
ACTAAGTCATTTTTCATGITTACTTTTCATTTAATAGAGTATAATCAGGCTATCAAATCCTATTATGGCT
ATCCTTTTTTTTTTTTTTTTTTGAGACGGAGTCTCACTCTGIGGCCCAGGCTAGAGTGCGGTAGTGTGAT
CTCGACTTACTGCAAGCTCCACCTCCCGGGTTCACGCCATTCTCCTGCCACAGCCTCCCAAGTAGCTGGG
ATTACAGGTGCCCACCATCACGGCCGGCTAAGTTTTTGTATTTTTAGTAGAGACAGGGITTCACCATGTT
AGCCAGGATGGTCTCGATCTCCTGACCTCATGATCTGCCCACCTTGGCCTCCCAAAGTGCTGGGATTACA
GGCGTGAGCCACCGCCCCCGGCGGCTATCCTTTTTAGAAAAAAAGTCAATTGTCTTTATTTAGTTTGITA
TAACAAAATACCTTAGACTGAGTCATTTACAAACAATAGAGATTTATTGCTCACAGTTCTGGAGGGTGGG
AAGTCCAAGATCAAGGCATCAGCAAATTTGGTGTCTGGTAAGTACCCACTCTCTGCTTCAAAGATGGCAC
CTTCTTGCTGTGTTTTATGTGGTGGAAAGAGCAAACAAACTTCCTCAGGCCTCTTTTATAAGGGCACTAA
TCCCCTTCATGAGGGCAGAGCCTAAAGGCCCTACCTCTTAATACTATTGCTTTAGGGATTGGATTTCAAC
AATTTGGGGGAACAAAAACATTCAGACCATAGCGTCAAACCACTTAATTTTTTCAAAACCGAACTTTTCC
AATAAAGCAGTAATCAAAAACTAGTAAATCTTGGCAATTAAGGCTGGCCCTGGAATCCCTTGCCATTTTT
TCTTTCTCTTTTCTGATGACCTCTCTCTCTATTTCCAGAGTACTTATGATGCCCCAAATCTCCCTTCCAA
CCCTGTCTAACCTTTGCTCCTCAGAATGCTTTGATTTGCTTTATCCTCTCATCAGAATTCACATTCTCTT
AGTATGTTGAATGAGTGACCTCTAGTTCATACTGAGGTGTAGTTTTTTGTTTGTTTTTTCAGACAGTCTC
GCTGTGTCTCCCAGACTGGAGTGCAGTGGCATGATCTCGGCTGACTCCAACCTCCGCCTCCCAGGTTCAA
GTGATTCTTATGCCTCGGCCTCGTGGGTAGCTGGGATCACAGGTGTGCACTACCATGCCCTTTTTTGCAT
TTTTAGTAGAGCCAGGGITTCACTATATTGACCAGGCTGGTCTCAAACTCCTGTCTTTAAGTGATCCTCC
TGCCTCTGGCTCCCAAAGTGTTGGGATTACAGGTGTGAGCCACCGCGCCCGGCCTAACGTGAAGTTTGTT
GGTAACAAGCTTCTGTATTTTCTGCTATTGGCAAGAATGCTTATTCACACGTAACAGGAATTTAACAGTT
GGCATTAATGTTGCCCAAACCAGACTCTACTTTTGGTGGTGAAAAGGCCATTTCTGGCTCTGAGGACCCT
TGAAAACACAACTGATTTTTTTTACTAATTTCTCTGAGGCTTGATATATCACTAGAATAAGACAAATACC
TAAATTAACAGTAGCTTAAATAGTTTGGGGGTGGTTATTCTTGCGAATAGTCAGAGGTAGCTAGTTCAAG
GCTGACATGATGGCACAGTGGTGTTATCAACAGTCCAGATTGGCTCCATCTTTCTGACCTGCCATCCTTA
GCACAAGACTTTTCCTCTCAAAGACGATCATGGTTGCATACTGGCTGCTGCAGGCATATTGTATTCACAA
AAGGATGATGATAACAAGGTAAAATGAAGTGGAAAATGAAAAGTGAGGCATGAGGCTGGAGAGGTAAACT
AGCCAGGTCTCTAGCAGCCGTGTGGAGGGCAAGCAGAGAAGGGGCAGGGGATGCAGGAATTGACATGGGC
AACATGGGGACCTGGTATTAAGGAATTGTAATGGCTCAAGCAACGCAAACTACAGATCTGAATTAGCACA
GTAGCAATGTGAAAAAGGAATGAAATTGAGATATTGAAATGGTAAAATAGGCAGGAATGGCTGAGTAGAT
GTGAGGGAGTGGAAGAGGTCAACGTTGATTCCTAGGTTTCTGTCTTCATTTATGAAAAAAATTCAGGAAA
AGCAGTCACTCTTTAAAGATGAATTCAGATTTGTGCACATTAAGTTCAAGATGTCTGTGGAACATATAAG
TGGAGATGTCCAGCAGTCAGCTGGATATGAATATGAAGATAGGGGGAGTGGTCAGGGATGGAGACAGACT
TTTGAATCATCTGCAAACATACATCCCAAGGTAAAAGAGTAGATACACTATAGTCTCCCAGGGAGCACAC
ACAGCCATGGTTCTCAGTCTAATCCTGGGACACCAAATTATTAGTGGGTAAAGGAAAAGAATCTCAAGAC
CGAATGGTCAAGAAACCATTCTAGGGAACCAAACTAGGAGAGAGTGGCATCTCAGAAGCCAAGCGTGTCA
AGGAAGGAATAGCTAACAGGATGTGTGTAAGGTATGTACACAAAAGAGCTTTGGAATCCATCTTGGGAAA
TATACAGTATTAGCTGTTTTAAGGAGACATGCTTTAAATATCAAAGCAAGTCATCCTGATTTAAAAAACT
CCCACTTGTATAGAGGAATACCAGGATTTGAGCACCACCAAATGGAACTCATTCCCTGACATCCCAAAGC
CATCAGAATAACAGTAGAAGCGTCAAGACAGTTACTATATGATTTTCCCTGCAAGACATTCAGTAATCTA
TGATTCCCCCTGCTTAAATTTTATCTCATGCCTTCTTCCCTGACCAGITCAACACAAGCAGCCCTGAGAT
GTAATATCTTCTCACCCTGACCCTCAAAGTGTGTCTCATGCTTTCTAACAGAGGCCATCCTCTTGGAGGA
AGACTTGTACTTTTGGCCTGTAGCCCCACCCTCCCTCCTATGGCGTTCTTACAGTCAGITCTGCAGTGAG
TGGGACACTTTCCAAATATAATAGCAAATGGAAACTTCTAATTACATCTGCACATAAAACTAATGTTCCA
AATGGTGGTTGCGGTTCTGGTGAGTGCTTATAATTTTTTGITACCTCAGCATCCATTTTTGAATATGTTT
AACTTTTAATAACAGAAGCAAGTCTCAGTCACCATTGACAGTTTCCAGTACTATACCCCACGCAAATGGC
TCTAGCTGGTGGCCAGAGATAAACATGCAGTGATGTGATAACAGGGCACTACTGCAGCTTCAAACTCTGG
TTCAAGTTCCTCCCACCTCTGCCTTTTCACCTGCTTGCTTTTAAATCCACCAGTTAGAGCTCTCCAGAGG
AAACCTAACTGGATAAAGTTGCTGGCCCAAAGGTCCGTTCCTCCCTTCCCTCTCCCTGTTCTCCCTGATC
TCCGTGTGTGTGTGTGTGTGTGTGTGTGTGTGTGTGTGTGTGTCCTCTAGGCAAGCTGTGTACCACCCAG
GGTCCGTAAGTACTAAAAACTCTTAAACTTTCACACCGCTGTGTTATTGAAACCATGCCTACAATCCAGT
CCCTGAGGCGAGGCCACCCTGAAGGGACAGACTCATGCAGGCGGATCCCCTGGTCTTGCTCTTCTGTCCC
TGCCTCTGGTGGCTGCTGGGGAACAGTAGCTACCAGCTAAGTTGATAGAGACACTCGAAGAGGTTCATTC
AAAACACTGGTCCCCGAGCAGCCCAGTAAGCTTTTAAACGCATAACATTTTGTTTGGGTCTCAAACAAGT
CACAAGGCACGGATACGGATGCGGCTGTGGCTGATGGCTATTTGGATGCTGCCCAAGCCTCGCAGTAGGA
AGCAGCTTCAGACTGCAGCAGGGCCGCATAGCCCGATAGCCAGGCCCTGGGGCACCCAGGGCACCAGCAA
GACGCACAGAAACGTTTTGAAAAACATTTTAATATACTTAAAAAAAAAGTAAAAGGGAAAATCAGAAGTC
TTTGGAAGTTTCCTACTGTTTTTATGTCCCATAGCAAATCCGAGGAGGACGCCCCAAACTTTCAGITCCC
TGGGGTTTTGAAGGTGCTCATTGGGTCCTGGCCACCCGGCCTTCTCAGAACCTGGGCCAGGAGGCTGAGC
TCCGCGCGGGGGTGGAGGGAGAGGAGGAGGTTCCTGCCGAGGTGCGGCTGCGCGGTGCGGGAGCCGGGAG
CCTGGGAGGGCAAGGGCATCCTGAGGGGGGGGGCCGGGGGGCGGAGCCTTGCGGGCTGGAGCGAAAGAAT
GCGGGGGCTGAGCGCAGAAGCGGCTCGAGGCTGGAAGAGGATCTTGGGCGCCGCCAGGTAAGAAGGCCCG
AAGGCCTGCTGGGGTTTGGGTGAGCCGAGCCGAGGTGGCGGCGGCAGTCTGGGCCCGGGCGGAGGGTCTG
TGGGGGCTACCCCCGTCCTCGCTTTCCACTCGACCCCTGCCGGGCGAGCCTCCCGAGGCCTGGCCCTTCC
AACCCGTCACCGCGGCCCGGTGTCCTAGCCGAACGCTGGCTTCATTTTATCTACGAGGAAGGAGAAACGG
GGAGCGCGCCCTCTCCCAGAAGCAGCCCCGTTACCCCCGTCGCGCCCCCCATTCCCTCGGGGGCAGCCTC
GCACCCGGGCCCCTGCCATCGCTTTCGTCTCAAGGGAAGCTCTAGCGGTCCAGAGAAACTGCGCCGGGGG
AAAGGCCAGCCCTCCGTGGTCACGGCAGTTCCTCTCTCCCCTCGCGGTCTAAATGTGTAATTTTGAAAAG
CAGGTGTCGGGCGAAACGGACTTTTTTTTTTTTTTGAGATGGAGTTTCGCTGTTGCCCAGGCTGGAGTGC
AGTGGCGCGATCTGGGTTCACCGCAACCTCCGTGTACCGGGTTCAAGCGATTCTCCTGCCACAGCCTCCT
GAGTAGCTGGGATTACAGGCATGCGCCACCATGCCTGGCTAAATTTTGTATTTTTAGTAGAAACGGGGIT
TTCCCATGTTGGTCAGGCTGATCTTGAATTCCTGACCTCGTGATCCGCCCGCCTCCGCCTCCCAAAGTGT
TGGGATTACAGGTGTGAGCCACCGCGCCCGGCCGAAACGGACGITTTAAGTACAACGTGATGTTTTGTTT
TCTTGGGTCGGCTCGGGCCGCACCCTTCTTAGAGTCTGACTGCTGACTTTTCAAAAAACAACATTAACCC
GGGAGGCTGAGGTAGCGGTGAGCCGAGATCGCGCCATTGCCCTACTGCCTGGGCAATAAGAGCGAGACTC
CGCCTCAAAAACGAACAAAACCCACAAAGAACAAACAAAAAAAACCAACATTAAGCAGTGTGCCTITTAT
GGCCGTTTAGTGATACCCAGGCTCTTCACTGGTCATTTCTGGAAGTATTTAACAGCACTTGATGGGTAAA
ACAATAGGCTTCACTTCACAGGGTGGTTGAGATGATTGACGGAAAGCGCTCAGCACAAACGCGGCGGCTG
CTAATTATTTCAGGTACCCTGCGATCCCTGAGCAGTIGTTCAAGITCTCCACTCCTGGCGGITTACTCCC
AGTTCCCTGAAGAAGGCACAGAGCCCCGAATGTTGAGGGAGATGGCAACGTTTGCCAAAATGTGGCTATT
TACGTGGCAGTTTATGAAGGAGAGGCAGAAAGGGAGGCTGCCTCCCCTACAAGCCAAACAGTCCTAAAAC
TTGTGGCCTCTGTATTTTCACAGTGGAAACTAGAAACTGTTTGCTGAACTTAGCTGTAAGAAAAACATCC
CATTAGCTGAGTGGCTTAGGAGATAAAAGCCTAAATAAAATGGATCTTAGGGTGAGAGAGTGGGGTGCCT
AATGGCATTCATCTTTGACAGCTGCTGGTAGAAGCTCTTGAAAAGTAGAGCATGGAAAATTCTGGGCCAC
CTCTTTAAGAACCTCAGCCAAACCGCCACAGACATTGCCAAGCCCAGAAGTGCTTGAGGCTTTCTAAAGG
CAAAGACTTCTTTTTACTTATGTCCTATATCTTAGGAAACATATTCACTAAACCTTGGTTTTGCATGTGT
ACTTAACACGGGAGGGGCCGTGAGTGCGGAGATAGGACTCTTTGGAGAGTTAGGGATTGGCATTCCGGAA
CAAACTGCAGGAAAGGCCCTTGTAGGGGAAGCTGCAGATTTGTTTGGTCCAAAGAGTTCTGGCCTAATAA
GTGGGAGCTGACCAATGAGAACACTTGGACACAGGGAGGGGAACAACACACACTGGCGCCTGTCGGCAGG
TGGGGTGGGTGGGGATAGAGCATTAGGAAAAACAGCTAATGCATGCTGGGCTTAATACCTAGGTGATGGG
TTGATAGGTGCAGCAAACCACCATGGTACACGTTTACCTATGAAACAAATCTGCACATCCTGCACATGTA
CCCTAGAATTTATATGTATATCACACAAAGAGTTTTGGAGTGGTTATTCCTGGCATGAGAACCTTGGCTG
GATTGGAAAGCCTTTTCCGTTATGATGGTTGTTTTCTCTTTGTCTCTTCCTGCTGATGTAATTTGTTCCG
TGAACTGAGTGATGGATGTTTGTCTGGGCGCTTCAACAGGCATGGCTAGAGCAGGTATTGTGTATGTCTC
CAAGGTGACACGCAGACCTGGGGTTCAAAAGCTCTAGTTCTCTGACATTGCTGACACTAGGTATCTTGTG
AGTTGCCAGCCCCTGGCGAATAGAAACAAGATCCATCCAGGGTGGTTTTGCGACTAACATGTTGCCTTGG
GACAGGTAATTTCATCCCACTGGGCCTCCACTAATTTCCTATAAAATAAAAACATTGGGTTTTATTTTGA
GGACACTTCTAGCCTTAAAATTCTTCAGTCTGTAAGGCCTCAGGCTCCTAGGAGGAGTAACCATACTCAT
GGGCAACTGTGAATCTTGTGGAAACACCACTGATGTCAACTCTCTTCTCTAATGACCTCAGCTTAACTAA
TGTATTICAGAAGCTGAATTTATAAGAAGTCTCCCGTCTCCCCACTAGAGTTTAACTATGGTTCGTTCAC
TCAGAGCTTAGACCTGGGTTTAAATGCTGTTCTACCAGITTCCAGITCAGAGGTTGCTATAATAATTTTA
ATAATTTTTTTTTTTAATTTTGAGACAGTCTCTCTCTGTTGCCCAAGCTGGAGTGCAGTGGCACAGTCTT
GGCTCACTGCAACCTCCACCTCCTGGGTGCAAGCGATTCTCGTGTCTCAGCCTCTCAAGTAGCTGGGATT
ACAGGTGAGTGCCACCACATCTGGCTAATTTTTGTATTTTTTTTAGTAGAGACAGGGTTTCACCATGTTG
GCCAGGCTGGTCTGGAACTCCTGGCCTCAAGTGATCCACCCGCCTCAGCCTCCCAAAGTGCTGGGATTAT
AGGCATGAGCCACCACACTCGGCCTTATAATAATTTTCTACTGCTGCTGAAACCACCACAAATTTAGCTG
TTTCAAATGCCACAAGTTTATTAATCTTTCCATTCTGTAAATCAGAAGTCTGACACAGGTCTCACAGGGC
TGTAATCAAGGTGGCAGGGCTCTGTGCTGCTCTGGAGGCTCTAGGGGAGAGTCTGITTCTGTGCTCATTC
AGGTTCCTGGTAGAATATAGTCCTTGTGACTGGAGGACTGAGGITCTGITTCTTTGCTGAGTGTCAGTTG
AGGACCATTCTTAGCTTCTAAAGGCCGCCTGTGTGCCTTGGCTTAGGGAACCCTTCGTGTCTTCATCTCA
TTCTTCAAATTTCTTGCCTCTTTTTCTGTTGTCGGTCATGAGTCTTTGACAGACTCTGCTACCTTCTTCC
TCTTTCAATGACCCCTGGGATTACATTGGGCCCATTGAATAATCCAAAATAATCTCCTTAATCTCATCTA
CAAAGTCACTTGCCATGCAAGGTCTCCTGTTTTCACAGGTTCCTGGAGTTAGGATACGGACATTTTTGGG
GAAGAGAGGCATTATTCTACCTACCACATAAGTTAGTTGCATAAATTAGTTCTCTTCCTCAATCTTGTTT
CCTCCCCCTGTAATTGGAGGCAAAGGTTCAGTTTACCTTATGGTATTATTGTGAGGATTAACTCCAGTTA
CTATGTCAAGAGCATCTATCTTAGCAACTGGCCCTTGATATTTGCTCACCAAATGGAAGTTAACACAGGA
ACAAGGTGTATTAGTCCATTCTCACACTGCTATAAAGAAGTACCTGAGACTGGGTAATTTATAAAGAAAA
GAGGTTTAATTGGCTCGCAGTTCTTTGGGCTGTATAGGCTTCTGCTTCTGGGAAGGCCTTAGGAAACTTA
GAATCATGGTGGAGAAGGGGAAGCAAGAATGCCTTACTTGGCGGGAGCAGGAGGAAGACAGCAAAGGGGG
AAGTGCTACACACTTTCAAACAACCAGATTTCCTAAGCTCTGCCACAAGACAGCACTAGGGGGATGGTGC
TGAACTGTTAGAAACCACCCCATAATCCAGTCACCTCCCACCAGGTCCCTCCTCCGACACTGGGGATTAA
AATTCAACATGAGATTTTGGAGGTGTATTAGTCCATTTTCATGCTGCTGATAAAGACATACCGAAGACTG
GGTAATTTGTAAAGAAATAGAGGTTTAATGGACTCACAGTTCCACGTGGCTAGGGATGCCTCACAATCAT
GGCAGAGGGTGAAAGGCACGTCTTACTGGCAGCAGACAAGACAGAAATATGAGAGCCAAGTGAAAGGGGA
AACCCCTTATAAAACCATCAGCTCTTGTGTGACTTATTCACTACCACAAGAACAGTATGGGGGAAACCGT
CCCCAGTGATTCAATTATCTCCCACCAGGTCCCTCCCACAACACGTGGGAATTATGGGAGTTACAATTCA
AAATGAGATTTGGGTGGGAACACAACCAAACCATATCACAAGGTCTTGCCTAATGGCTTGCATTCAATGT
CATAATCATATGGGCCCTTAAAAGTAGAAGCGGCTCCTAAATATTTAAGAACTAACAGCTTCTTTTATAA
CCCTGTTGGAAGAAACATTTCCAACCCAATCTAAAGTAACCTGGCCCTTATGCCAGGTCACCCTCTAACC
CAGTGGTTCTTAGTGTGATCCCTGAACCAGCAGAAACGCAGCACCTGAGTGCTGGCTCAAAATGCACATT
CTTGAGCCTTACACTAGCCCACCTGAATTAAAACCTCTAGGGCTGGAGTCTAATCATCTACTTAAGAGCC
CTCCAGGTGATTCTGATGCTCCCTAAAGTTTGAGAACCACTGTTATGACCCTCTTTTATTTTCGTCCTAG
TCTTTAGCACCAGTTGGTGTAGGAGTTGAGACCTACTTCACAGTAGTAAGTATACTGATGAGTGAATTCC
TTAAATGTTATTAAATAATTATTTGATGTGTTAAATGTTTTATGTTCAATTTCTTTGGATTGCATATATA
TATGTGCATATATATGTGTATTTGTGTGTGTATATATAATGTATGTATATGTGTGTTTTTACGTATATAT
GTAATATATATTATATATGTTTATATACAGTTGACCCTAAAACAATGCAGGGTTAGGGACACTGACCACC
TACACGGTTGAAGATTCAAGTTTAACTTTTGACTCCCCCAAAACTTAATTACTAATAGCCTACTGTTGAC
CAGAAACTTTACCGATAACATAAACAGTTGATTAACACACATTTGGTGTGTTATATGTATTGCCTACTGT
ATTCTTATGATAAAGTAAGCTAGAGAAAAGAAAATGTTACTTAGAAAATCGTAAGGAAGAGAAAATCCAT
TACAGTTCTGTACTATAGTTATTGATACCATCAGTTTATGTCGTCTGTTTACAAGATGAATTACTTGTCT
GAAATGGCAGGCGGCCACAACTGCAGACCTCAATCTGTAGTACATATCAAGTAGITCAACTGTTTTTTGG
TAACATCATGACTTTTTTCTGCTTCTTGGGAGCGTGGCCAGCATCACTTGTGGCACCCTGTAAGGGTCTC
ATGTGTCATTCAAGTTTTACACTATTGCGCTTAACACGATAAAGAAAATACTCAAGAACTGCAAGAGATC
ACCTTTTACTGTGATGTGCAATTTCTTAAAGAGGCAAACAGCTCACAGATAATTAGCATCACATGATACT
AATCATATCCCTGCTGATAAGTAGATCCTTGCAACACTTGAGCTCACTGCAATAGCAACAGGAGGTGGCT
ATGGCATTATTAACAGTATATACTATAGTTGATTTTATGCAGTATGATTTAATACTGCATCTTTACATTT
GITTACATTTCTCTTTACTTTGGTGCCATGTCTGGTCTGTGTTTGTGTGTGTAAGTTTTAATAATTTATT
TTTTTGAGACAGGGTCTTACTCTGTCGCCCAGGIGGGAGTGCAGTGGTGTGTCTTGGCCCACTGCAGCCT
CAACCTCCCAGGCTCAAGCAATCCTTCCACCTCAGCCTCCTGAGTAGTTGGAACTACAGGCACACGCCAC
CACACCTGGCTATTTTTTTTTTTGTAGAGATGGGGTTTTGCCAGTCTCGTCTCAAACTCTTGGCTCAAGC
AATCCACCCACCTCGGCCCCTCAAAGTGCTGAAATTACAGGCAAGATCCACCATGCCTGGCTGACACATT
TTAACTTTATAATAGATTTGTGTATGTTTTTGGGTAGTGATAAAATAGACTAGTATCTATATACATTTTA
TACATTCATGACATACCTAAATTTTTCTTAATTTTAAAAAATATTTCTAGGCTATGCAGTTTATCTAACA
GTTTTTTCAATTGCAAATCTCCAAAATATTTTCCAATATATTTTCAGAAAAAAAATTGCATATAAATGGA
CCCCTGCAGTTCAAACCTGTGTTGTTCAAGGGTCAAATGTGTGTGGGTGTATATATATATTTAATTGATA
TTTGGGGGGCCGGTTGGGGTTTTGGATGAACAGTAACACGTTTTCCTACTTAAAATAGTGGAAATAGACT
CATTACATTTTCTACCCAAATACCCAGAACATTTGCTATCTAAAACATTTTCAGGAACTTGATAAGAAGT
GCCTGTATTTTTTTTTTTTCTCGGAGATACGGGGTTTAAGTTCTTTTTTAAAAATTATACTTTACGTTCT
GGGGTACATGTGCACAATGTGCAGGTTTGTTACATAGGTATACATGTGCTATGTTGGTGTGCTGCACCCA
TCAACTTGTCGTTTACATTAGGTATTTCTCCTAATGCTATCCCTCCCCCACCCCCCAACTCCCGACAGGC
CCTGGTGTCTGATGTTCCCCTCCCTGTGTCCATGTGTTCTCATTGTTTAACTCCCACTGACAAGTGAGAA
CATGTGGTGTTTGGTTTTCTCTTCTTGTGTTACTTTGCTGAGAATGATGGTTTCCAGTTTCATCCATGTC
CCTGCAAAGGACATGAACTCATCCTTTTTCCTGCAAAGAACTATAACTCATAGTATTCCATGGTGTATAT
GTGCCACATTTTCTTTATCCAGTCTATCATTGATGGGTATTTGGGTTGGTTCCAAGAATTTGCTATTGTG
AACAGTGCTGCAATAAGCATACGTGTGCATGTGTCTTTATAGTAGAATGATTTATAATCCTTTGGATATA
TACCCAGTAATGGGATTGCTGGGTCAGATGTTCTAGATCCTTGAGGAGTCACCATACCGTCTTCCACAAT
GGTTGAACTAATTTACACTCCCACCAACAGTGTAAAAGCATTCCTGTTTCTCCACATCCTCTCCAGCATC
TGTTGITTCCTGACTTTTTAATGATCGCCATTCTAACTGGTGTGAGATGGTATCTCATTGTGGTTTTGAT
TTGCATTTCTCTAATGACCAGTGATGGTGAGCATTTTTTCATAAGTTTGTTGGCTGCATAAATGTCTTCT
TTTGAGAAGTGTCTGITTATATCCTTCACCCACTTTTTGATGAAATTGTTTGTTTTTTCTTGTAAATTTG
TTTATTTGTAGATTCTGGATATTAGCCCTTTGTCAGATGGATAGATTGCAAACATTTTCTCCCATTCITT
AGGTTGCCTTTTCACTCTGCTGACAGTTTCTTTTGCTGTGCAGAAGCTCTTTAGITTAATTAGATCCCAT
TTGTCTATTTTGGCTTTTGTTGCCATTGCTTTTGGTGTTTTAGTCATGAAGTCTTTGTCCATGCCTATGT
CCTGAATGGTATTGCCTAGGTTTTCTTCTAGGGTTTTTATGGTTTTAGGTCTTACATTTAAGTCTTTAAT
CCATCTTAAGTTAACTTTTGTGTAAGGTGTAAGGAAAGGATCCAGTTTCAGCTTTCTACATATGGCTAGC
CAGTTTTCTCAGAACCATTTATTAAATAGGGAATCCTTTCCCTGTAGCTTGTTTTTGTCAGGTTTGTCAA
AGATCAGATGGTTGTAGATGTGTGGTGTTATTTCTGAGGCCTTTGTTCTGTTCCATTGGTCTATATATCT
GTTTCGGTACCAGTACCATGCTGTTTTGGTTACTGTAGCCTTGTAGTATAGTTTGAAGTCAGGTAGCGTG
ATGCCTCCAGCTTTGTTCTTTTGGCTTAGGATTGTCTTGGCTATGTGGGCTCTTTTTTGGTTCCATATGA
AATTTAAAGTAGTTTTTTCCAATTCTGTGAAGAAAGTCAGTGGTAGCTTGATGGGAATAGCATTGAATCT
ATAAACTACTTTGGGCAGTGTGGCCATTTTCATGATATTGATTCTTCCTATCCATGAACATGGAATTTTC
TTCCATTTGTTTATGTCCTCTTTTATTTCCTTGAGCAGTGGTTTGTAGTTCTCCCTGAAGAGGTCCTTCA
CATCCCTTGTGAGTTAGATTCCTAGGTATTTTATTCTTTTTATAGCAATTGTGAATGGGAGTTCACTCAT
GATTTGGCTCTCTGITTGTCTGTTATTGGTGTATAGGAATGCTTGTGATTTTTGCACATTGATTTTGTAT
CCTGAGACTTTGCTGAAGTTGCTTATCAGCTGAAGGAGATTTTGTGCTGAGACGATGGGATTTTCTAAAT
ACACAGTCATGTCATCTGCAAACAGACAATTTGACTTCCTTTTTTCCAATTGAATACCCTTTATTTCTTT
CTGTTGCCTAATTGCCCTGGCCAGAACTTCCAATACTGTGTTGAATAGGAATGGTGAGAGAGGGCATCCT
TGTCTTGTGCCAGTTTTCAAGGGGAATGCTTCCAGTTTTTGCCCATTCAGTATGATATTGGCTGTGGGIT
TGTCATAAATAGCTCTTATTATTTTGAGATACATTCCATCAATATCTAGTTTATTGAGAGTTTTTAGCAT
GAAGCGCTGTTGAATTTTGTTGAAGGCGTTTTCTGTGTCTATTGAGATAATCATGTGGTTTTTGTCGTTG
GTTCTGTTTATGTGATGGATTATGTTTATTGATTTGCATATGTTGAACCAGCCTTGCATCCCAGGGATGA
AGCCGACTTGATTGTGGTGAATGAGCTTTTTGATGTGCTGCTGGATTCAGTTTGCCAGTATTTTATTGAG
GATTTTCACATAGATGTTCATCAGGGATATTGGCCTAAAATTTTGTTGTTGTTGTGTCTCTGCCAGGITT
CGGTGTCAAGATGATGCTGGCCTCATAAAATGAGTTAGGGAGGATTCCCTCTTTTTCTATTGATTGGAAT
AGTTTCAGAAGGAATGGTACCAGCTCCTCTTTGTACCTCTGGTAGAATTCAGCTATTAATCCATCTGGTC
CTGGACTTTTTTTGGTTGGTAGGCTATTAATTATTGCCTTAATTTCAGAACCTGTTATTGGTCTGTTCAG
AGATTCGACTTCTTCCTGGTTTAGTCTTGGGAGGGTGTATGTGCCCAGGAATTTATCCATTTCTTCTAGA
TTTTCTAGTTTATTTGCGTAGAAGTGTTTACAGTATTCTCTGATGGTAGTTTGTATTTCTGTGGGATTGG
TAGTGATATCCCCTTTATCATTTTTTATTGCGTCTATTTGATTCTTCTCTCTTTCCTTCTTTATTAGTCT
GGCTAACAGTCTATTCTGTTGATCTTTTCAAAAAACCAGCTCCTGGATTCATTGATTTTTTGAAGGGTCT
TTTGTATCTCTATCTCCTTCACATCTGCTCTGATCTTAGTTATTTCTTGTCGTCTGTTAGCTTTTGAATT
TGTTTGCTCTTGCTTCTCTAGITCTTTTAATTGTGATGTTAGGGTGTCAATTTTAGATCTTTCCTGCITT
CTCTTGTGGGCATTTAGTGCTATAAATTTCCCTCTACACACTGCTTTAAATGTGTCTCAGAGATTCTGGT
ACGTCGTGTCTTTGTTCTCATTGGTTTCAAAGAACATCTTTATGTCTGCCTTCATTTCGTTATTTACCCA
GTAATTCAGGAGCAGGTTGTTCAGITTCCATGTAGTIGTGCGGTTTTGAGTGGGTTTCITAATCCTGAGT
TCTAATTTGATTGCACTGTGTTCTGAGAGACAGTTTGTTGTGATTTCTGTTCTTTTACATTTGCTGAGGA
GTGTTTTACTTCCAATTATGTGGTCAATTTTAGAATAATTACCATGCGGTGCTAAGAAGAATGTATATTC
TGTTGATTTGGGATGGAGAGTTCTGTAGATGTCTGTTAGGTCCACTTGGTCCAGAGCTGAGTTCAAGTCC
TGGATATCCTTGTTAATTTCCTGTCTTTTGATCTGTCTAATATTGACAGTGGGGTGTTAAAGTCTCCCAC
TATTATTGTGTGGAAATCTAAGTCTCTTTGTAGGTCTCTAAGAACTTGCTTTATGCGTCTAGGTGCTCCT
GTGTTGGGTGCATATATATTTAGGATAGTTAGCTCTTCTTGTTGCATTGATCTCCTTACCATTATGTAAT
GCCCTTCTTTATCTCTTTTGATCTTTGTTGGTTTAAAGTCTTTTATCAGAGACTAGGATTGCAACCCCTG
CTTTTTTTGCTTTCCATTTGCTTGGCAGATCTTAATTCATTGTGAGTAGCCTTCCACGCCCATTACTTTT
CCTGGCTATGCGGITCCCCACCATGGAAGTGTACCATAGTATACTCATTTCCTTATTGTTGGATGTGTGT
ATCACTAAATTTTTGCTATTTTAAAGAATGITTGTATGCCCTTGACTATTTCTTATTCCTGTGATGAAAG
GCCTACTTGGAGCTCCCATTACGGAAATGACTTTAGAGTCAACTTACGCACCACCCAGGAAAGCCTGCCG
TTCCTTCATCATGGGGGAAGACTTGTTCCTACATATCTTCTTTGAGGGATTTTTCAGTTGTGCATTCITT
AGAATGATTAAAAATCATTCAGAACAATAGCAGTATTATTAGAACAGTCGTTAGGGACAAACCCACCGTT
ATTAATGAATCCTGAAATGTTTGGAAAGGAGTTGAGTGTTGATGCTTTCTGAGTGTATCTTGCATTTAAG
TACTTGGTTTGGGTGGAATGGCAACACTTTTGGTTCATTTACCATCTTATCTACACTTAACTTATGGGCA
TCTTCTTTTTTTTTTTTTTTTTTCCTTAAAATGGAGGGTGTTTTAGTCTAGGGAGTAGTTTTACCAAAAG
ATAGATGATCTATGACTACTAGAAAAGGAACATTGTCATGTGTGAATTTTCCTGATGTTTCTGATTGTAA
AAGTAATACTTGGTTAATTATAGAATATTTGGGGAAAGGATGGCAAAAAGTAAAGGATCAGGAAGAAAAA
TAAAAAGCCATAAATGATCTTGCTTAGTGTTAGTTACCATCTACATTGTAGGGCATTTATAAAATAGCTT
TAAAACAATCAGCTGTTTCTTTTTCTGCCTCCCGCCCTCCTCCGCCCCCCATACACATACACATACTCCA
TATGAACGTTTTGCTCTTGGAAATTCATATTTTGGCAGTGGTTCATAAAGGCCTGGTCTTATTTTGATGG
AACAGCCCGACCTTGGCATCTACACATAGCTACTTCCTTTCTCTGCTGTCTTGGCTTCCCCTGCAGCATG
TCATGGGATCAGCTATAGCAACAACCAGCCAGTCATGTGGAACTGAGGCTAAGACTTGACACTCCCCAAC
CATCCCAGAAAACTGAGGGCTTCCTTAGGTTTGATGCCTGAGGTCTCCCCTGCTCATTCAACCCAGGGTG
TTTCTCTGGCTTCCTCACCTCACATCACCCTCTGTCTGCCTTCCCACCGTTCACCCCCCAACACTCCCTC
CCCACACAATTTCTTCCTCAAGTAACCTCATTACTCCTGAGTAGCACTAACAGTGGCCACAGCTTTTTGA
GTGCCATCTTTGGCCAGGCATTGTGCTATGCAGTACATGTAACTATCTCCATGCTTACTAGCCAAAGCGT
GATCAGAGGTTAACACATAGCAAAGAGGTGACAGAGCCAGAACTATCTGGCCTTGTTCAGAGGCCTCCAG
ATTGGACTGGAATGCCTGTGCTCTGAACCCTTTCATTGTGTTCTTCCTGCTTTCTTAGCAGGCCACTTCT
TTCTAAGGTACTTCAAAGAACAGCTCCCCATCAGGGGCCCTGTGTGTTGTGGCAGTGTGATTTATGTCAA
GGACCATTCCAAGGGAAGTGTCCTGAACCGGCAGCTCCTGCCAGCCTGACACCTTTCCAGTGCCTAACCC
GAGTCAGGAGATTCCTTCCTTCCTCCTTCCGTGGGGATGCCTGACATTGTAGCTACATTGGGTTTTTGTT
TGTTTGITTGGAGAGAGGACCTTGCTGTGTCACCCAGGCTGGAGTGCAGTGGCACAGTCACAGCTCACTG
TAGCCTTTACCTCCTGGGCTCAAGCGATCCTCCCACCTCAGCCTCCCAAGTAGCTGGGACTACAGTTATG
TGCCACCACACCAGTCTAATTTTTTTTTAATGTTITTATTTTTGTAGAGACAGGGTCTCACTATAATACC
CAGGCTCCGATCATGCTATGCTTAACTTTTAGGAAGTGTCTGGAGCTCTCTGTTCGGGGACCTCAGACCA
ATACCTTCCTCTCCCTGCTCACTTGTAGCAGTTCCTTGTATACTGGAGCCCTGTCTAACCTGAGTCTATA
AATCACCCTTCCTACTCAGAAACCCCTGAAAATGGGGAACCAAGCAAGCCGCTGGCATGTGCTGGAGCCC
AGGTCTCCTGGGAAGGTCCACATGGGGGATGGTGTCCCGTAGCAGGCTGTAGGTTTCAGAGCCCAGCGGG
TGGTGGGGAGCCCTCAATTTGTAAGGAAAGGCCAGATGACAATTGTCTTTCTTCCTGGTTCCTCACATAT
CATAAAACCACCTCTTTCTCATCTCCACCACCCCCATCTCCAGGCTTGACTCACTGATTGTTTTCTTTTT
TGGACAATGTCAGACCCTGCCCTTTTCCTGGAACTTCTCAGTCCTCCACIGGCCTGCGTCAGCAAATTCC
TAAGTAGCAGATGACCTTCAAGATGTGTGAGAAGGAAGGGACAGGAATGATTGTCAGGATTGICAGTTCC
CCAGGGAACTCTGCATTTCTGACTTAGGGAGGGTAGATGACTCACCCTTCCAAGAACCCATCTGTCTGTC
CGGGCCTGTGGGGCTCTGGAGAATAGAATATGGAGTACCCTGGTTTACAGGCACTGCCACCTCCTTATTT
CAGGATGGGTTCAGGGATGTAGGATTGGAGTTGGGAGATTTCTTAGTTCTGCTATGGATGCTTTGTACTT
AGGCAAGTTCAAAACCTCCCTAAGCTTTGGCTTCTTTACTGGGTAAAGTAGGGTTGGAGGCATTTCAGGC
TTTCCTTCTTCTCACCTGATGCTATCACTACACATCTAGGCATTCACGTAATGGAAACTTACAAAGTTGC
TCAATACCTCTCTTCATGTCCCCAAAGAGAGCAAGCAAGTGTCCCCATTATACTTCTTACAGTGAGGAGC
CAGAGCTTCCAGGTATGCTAAGCCATTGCTCCCTGTTCCACTGCTGAATGCATGCTGGTGCCTTCCAGAG
TCACCCCTTTGGTCTGATTCACTTAGTGCTGTGGCTCCGAGAAGTGGCCCTCTCTTTCAGCCAGCAGCTT
GGGGTGGGGCCAGTAGAGTGGGAACCACAGTGGCTATATGAGTCAATTTGCTCATATTGTCATTTGACTC
ACTGACCCTGATGGGGAAGATAGTGTTATTCCCACAGTGTGGTAGTACACACTGGGGCTTATAGGGACTG
AGCCTACTCAAGGGTATATGGTGCTGTGGGTCAGAGCTGGGGCATGGCCCAGGGATTCAGTGTGCCTTGA
CTCCCCCTGTAAATGTTCCTCTCAGAAGCCTTCTTGGCCTTCCAGCCCTTGGTTTTTGAGACAACCAGCA
GTCATTTGTTCGTTCCTGACATTCCTTCCTGTCCCTTCCTTCCAGGTTCTGTGGACAATCACAATGGGAA
TCCAAGGAGGGTCTGTCCTGTTCGGGCTGCTGCTCGTCCTGGCTGTCTTCTGCCATTCAGGTGAGTGCTC
CAGTCTCAGGACATGGATCTAGGCTGCCTTGGCCATGAACTCCCTTCTTAAGCCTCAGTTTCAGCCCCAG
CTGCTCCTCCAGGCCTGGCITTGGTTCTTTTGCCTTCCAGTGTTACAGGTCTGGGGITGTATGACAGCTC
CTTCCTATGTTATGACCTTGTCCTCAGTGGGGCATCGTCCTGAGTTACAGCCCGTCCTGAGITACAGCGT
GGCTTTTGGCAGGGGTGGGTCAACAATCGGCGGCTGAAGTTTCACTCCTGTCACCATAAGCTGCTTGGGG
ACACCAATGGATACCATTCTTAGAGTGCTGTGCAAGTCCCTTTGGGGTCCACTCTGCTTAGAGCCATCTG
AGCTCAGTCCTTCTGCTCTGAGGCTCCTCCTGTGTTTTAATGATGATGATGGTCATAGTGACAGCAGCAG
CAGCTAACTAACCCTGAAGCATTTATGTGCCAGTCACTACTCTAAGCATTTTACGTGTATTTGCTTATTT
AATCCTCATGCCAACCTGATGGAGTTGGTGGTATTTTTATCCTTGTTTTACAAGCAACAAGATTGAGGCC
TCCCCAAAATTAATAACTTGTCAAATAACACACAGAAGTGGTACACGGGAATGTGAACACACAGTCIGGC
AACAGAGTCCCTGCTCTTCCCTCCTGCTCCAGGCCATCTGTCACTGGGCCACCCCCTCTCCTCAGCCACC
CCTCCCCTGGTTGCTGGAGGAAATTGTGTGCTTCTGAGTCAACTTCTATAACAGTGGAAACACACATGCT
TCATTGTAAAGATCAAGTGTGGTTGTCTGCCTGTGTCATGTTGGTATAATATCTTGGCCACTGCTGGITT
TTTGTTTTTTATAAATATGGTTGGTGGGGGGAGCAGAAAAGAAAGTGGCAATACAGTGAGCAATTTAAAA
AAAGATTTCCACACCACTGTAGCATGTCTTAGAATATTAACAGCAACCATATATTCACTGTTTATTGTTT
ACTGGGGACTGTGCCAGAGCCTGTCTCATTTAAATCCGTCCAGCCATTCCGGGAGGTCGGTACTGTTACC
TCCCATATTACAGCTGAGGAAATGAAGGCAAAGTAGTAACTTGGCGCCAGGACACACTTTAATATGTTCT
CCCTTTCTGAGCAGAAAGAGAAGTCATGGCACCCCTTCTTCCCCACCTTCCGTGATTCTAGCCAGGGTGC
ACTGTAATGTGGTTCACCTGTGTTAAAACACTATATTTTTTCTATAATTATTTTTTATTTATTCATTTTT
CTTGGTTCCTTCTTTTTTTCTGTCTCTGCTGCTTAAGTTGGAATATTTTCTAGAATTTTAATAGCCCAGT
GACATCCTACTTCCAGCAGTCTGCATTTGTTTATGGAGCAAGCTTGGGACCATCAGCTTCTCTGTGACAA
GTCTTGGGGTGCACTTCAGCCCTTTCTCAGAGAATTGAGATGTGAAGCTCACATGCCCACAGATGAGAGG
CCTTCTTTGCTGCTGCCCAAAAGGGTCTCAGGACCCTGCAGGGGAACTTTCCTCTGAGCTCTGCCTCTTA
TGGCCCAGGGGAAACCTCGTCTACCCTGGCTTAGTGGCTAAAATATCAAACCATTGTCCACTGACCTATT
GCTTGTTTTTTTTTGITTGTTTTTTTCAGGGGGATGGAGTCTCACTCTGTCGCCCAGGCTGGAGCGCAGT
GGCGCGATCTCGGCTCACTGCAACCTCCGTGTCCCAGGTTCCAGCGATTCTCCTGCCTCAGTCTTCTGAG
TAGCTGGGATTACAGGAGCATGCCACTATGCCTGGCTAATTTTTGTATTTTTTTTTATTTACTTTTTAAT
TTTTGTATTITTAGTAGAGGCGGGCITTCACCATGTTGGCCAGGCTGGTCTGGAATTCCTGACCTCAAAT
GATCCACCTACCTCAGCCTGCCAAAGTGTTGGGATTACAGGCGTGAGCCATTGCACCTGGCCCTGTTGCT
TGTTTTTGACCTCAGITTCCACCATAATCTAGAGTTTCCTTCTCTTCCCACTCAGGTCAATTAGAAGAAC
ATCATGGTTAACCAAGCTTGGATTTTAGAACTGACAGCCTGAGTTTGAGCTCAGCTCGGTCACTTAGTAG
CTGTGTGGCTTTGAACAATTTAGTTAACATCTTAGTCTTAGTTTTCTCTTGCAAGGTGGGGATGAAGATA
ATTGAGCCTAGITCTCTTAACAATCCTATGAAGAGCTTAGCACATAGATGCACATATATTTGAACACTGA
ATGAACATGTTTGCCATGAATATTTTTGCCCACAGACGTTCCAACCAIGGGITTTAGCCTTTCCTTCTAA
AGCTTTCTTAAGAGGCCAAATGAGCCCGGGATCTAGCCACCACAAAAATCCATGTCTACAGGTAACCTTT
TAAAATGGCCCTCAAACATGGCCAGTCTCAAGAACAACACCCTTCATCTTAGACTCTTTTCATTTCATGC
GTGTGAATACATGTCTGGCACAAGGAAGGGACCAAAGAAAAATTGCATCTTTGCGCCTTCTCCTTGTGGC
TTCCCTCTCACACGGGCATGACAGCCACAGGCTGACAGTGGGGGAGAACAAACAAAGCCTGGTGGATGGG
GCCCTGCCCACAGGCCCCAGGGCTGCCGCCAGCTCTGCTGCTTTGTAGCCAGGTGTCCTTGTGTGAGTCA
CATCACCTCTCCAGACATCTGTCTTGTCATCTGCAAAAAAGTTGGGGAGGAGGTTGGTTTTTATTTTATT
TTATTTTATTTATTTTTTAGACATGGTCTCGCTCTGTCTCCCAGGCTGGAGTGCAGTGGTGCAAGCTTGG
CTCACTGCACCCTCCACCTCCCAAGCTCAAATGATTCTCATGCCTTAGCCTCCTGAATAGCTGGGATTAC
AGGCATGTACCACCATGCCCGGCTAATCTTTGTATATTTTGTAGAGGCGAGGITTCACCATGTTGGCCAG
GGTGGTCTCAAACTCCTGGCTACAAGTGATCCACCCACCTCGGCCTCCCAAAGTGCTGGGATTGTAGGTG
TGAGCCGCCACACCTGGCCTAGTTTGTTGTTGTTTTTTAAAACTAATATTTCAAGGCATTAATCTGTAAC
CTTCACAAGTCAGAGGATCCTCTATTGGGTAGGATTTCACACCTGTGAGGGCAGACATGGCCATTTCAGA
TGCTCTAGAGAACTGGGTTCCTGTGTGTGCTCAGAAAGTGAGGACCCACCTTGCTTCCACIGGACTTTCC
ACTGAAAAATCTATTTCCATGGTGATAAACCTAGCCTTCAGCCTTAGAACTAGAAGTCAGCTGTTGGGCT
CTAAAACCCACACTTGGTTTAACCATGACATTCTTTTGTGAGATTTTACGTGTTTCATCTTTGAAAATGT
CTTTTCACAGAGGTAGGTACTTCCAAATACTGATTACATGGTAGAAGTCATATGGCATGAAGATAGTTTA
TCTCTGATGATGGAAAAGGGGGTATCCATACAAACCAGGAAGATTTCCTTCCAACTCTTGCTTCCTCAGT
CTCTCCCCATCACACGTGTGGACTGTAACTCATCTTTTTGTTGTCACTGTTGTTACAAAAATGGAGAAGG
AAAAATAACCCAGCAAAAACTGACAGCTAGCAAGGATGAGTCTTAAGAATTGACTGGGAGCAATAATTGT
TTTTCAAACTAGGGAACTGGAGGCAATTTGCTTCTGATGTAAGATGACCCTCACGTTGTGTCTGCTGGTA
CCCAGCTGTCCTATGCCCATGGGTTTAAAGTGCACAGCTGTTGTCTTGTCTTTGACAGTGTGTACTCCTG
GCATTCGGTCAGTCCCTTAGAAGGGAGTAGTTCAGTAATGATACCCTGTCCCAGAGGTTACCAAACTTTT
TCTGTACAGGACCAGATAGCAATTTTTTTTTTTTTTTAAACAGAGTTTTGCTCTGTCACCCAAGCTGGAG
TGCAGTGGTGCAATTGTGGCTCACTGCAACCTCTGCCTCCTGGGTTCAAGCGATTCTCCTGCCTCAGCCT
CCCAAGTAGCIGGGATTATAGGTGTGCACCACCATGCCTGGCTAATTTTTTTATTTTTAGTAGAGATGGG
GITTCGCTATGTTGGCCAGGCTGGTGTCAAACTCCTGACCTCAGGTGATCCACCCTCCTCGGCTTCCCTA
AGTGCTGGGATTACAGGCGTGAGCCACTGCGCCTGGCCCAGATAGTAAATATTTTAAGCTTTGCAGACCA
AGAGGCAAAATCAAGGGTATTATGAGACTTACCTCAATAATAAGAAACAAAATTTTCACAGATTTTTTTA
TTGACAAAATTCATAACATAATTATTGAGTCCAGCATAATAATACACATCTACTAATGAGGGGAATAGAA
TTCTTTTTAGGGGGATAACCATTIGCTTAATTGGGGTTCAAAATTAGTGTTCCCTCGCATCAAATCAATT
GCAGATGTTCATCTATTAATACTAATCTGTGATGACATTTTACATATTTCATTTTGAAAATGTATTTTCA
CGCAGATAGGTATTGCCAGACATTGATCATCAACCCACGAGGATGTGATTTAATTGGGCATATTCATTGC
TTGGAAGACACTTATAGAATTCTGTTTGTTTCTCGGTAATTTCTTCTTAACACATCATTACATTGCAGAC
TAATCTCCTTCCAGTTGCAGGTTAGGAGGAAGCTTCTCAATTGCACAGTTAGATGGATATTCAAATGTGG
ATATTTCCTTTGTGCTTGCATCAGGGTCCAAAAAATGTTGCTGGAACTGTAGTTTGAATTTGGAAAGATA
TAACTGCTATACATTTGTGCGGGAGTGGAAGTATACCACAAGTTGCTGACTTTGGGCCCATATTAATGGA
GATGGTGGGCTGCCAGGGGACAAGTCAGTGCTGCTTTAAGAGATCCTGACTTTCTTCCTGATTCTAGGTC
ATAGCCTGCAGTGCTACAACTGTCCTAACCCAACTGCTGACTGCAAAACAGCCGTCAATTGTTCATCTGA
TTTTGATGCGTGTCTCATTACCAAAGCTGGTAAGAGCCTCCCCTGTCTGTCTCCTAAATGTAATGGGGTA
ATAAGTGCCTGGGAAAAAAATTGTGCCACTGTAATCCTCATTAGGCTTGCATCAAGTAATAGCTACCATT
TATTAGCGTCAGGTGTGTATTGGGCTAAGCACTTTCTTCATACATTTTAAATTTAATGTTTTGCACTTCT
AAGGGTGATCTTTTATTTCCATTTGATAAATGTGGAAATGGAGGTTCAGATTAAGCAAGAAATTATGGTT
AAGTAACCGACTCAGGAATTGAACATGGAAGTCCATCTGATTCCAAAGCTTGTGCTCTTTTACAGCAGCC
CTGTGCTGCCTCCCAGACCTTGGAGAACCTACTGTGTGCCCAGCAGAGGGTTAGGAGCTAGGGATGCAAA
GATAAAGAAAGGACACATACAATTAAAATTATGACAAAGGCCTGTTTGGCTGTCCTAAATGTCATTATAT
ATGCCTCTGTCTCATTCCATGGGGTATCTGGTATATTCAGAGTCCTGAGGAGACTCAAGCTGAGCTCAAA
GGACACGTAGGAGGTAAGAGGGCTAAGAAGGGAAGGGTGTGCCTGGGAGGGGCAACAGCAGGTGCCAGGG
CTCGGTAGCCTCAGAGCAAGGTGCTCATGAATTTGGGGATGGGCAATGCTAACATGCACAAGTCCTGTCC
AGTCTGCATTTTCCAAATTTGTTAGACTACAGAATATTCACCTCCATCACCCTGTTAAAATAGAGTTTAG
GAAGTGGTAGACAGAGGAAAGAAACCAGCAAAGACATGCCCTCTGTTCTTGCTGGGGTGTGGTTCTGGAT
AGAATCGATGTGTGCAAATGATGCCAGCGAGGAATGGCAGTTTCAGAATGTTCTTAGTGTCCAAGGTAAA
ACCCTCAGGCTTAGATTTCTAAGAGATTAATAATAAAAACACTCCACAAGGTTATTTGTACTCTTAGACC
TTCTACACCAGCTCCCCCCAGTGTCAACATCAAGAAATGGTGAACATGTCAGGTAAACTCAGAAAAGCCT
TGGGACACCCACATGTTCAGAAAAACCTGAGATAAAGATGATTAACTTCCCCAGAAAGGAGAAATCCCTC
AATAACTAAAGACAAATTATAGTTACCCTGTCTAAAAAAACAGGGGCATCTGATAATTTTGATATATTTA
GCTGGATTTGATAAATTTGATATAGTTTAGCTGGATTGCAGCTAAAACTCCAAACCTTCCATTCAGAGCT
CTTGATTCTGTAGGGCACCAGTCCCTCTGCCCCCACTGGAAGACCATATTCATTCACTGTTAATGTAGGA
AGCGTAGTAAGGCCCCTAGCGAGAACATGGTCATTATGTACGAGCACAGAAGACTTGGCTCTTTAGCTTG
ATCAACTGTGTCTCTTGGACTCCTCACTACTAAGTATAATTAAACTAAGTATAGTTTCCAAATGAGACGA
TAAGGGCAATTACTATGTGTTGAATGAATATTACAGCAGCAATTAGAGCTCTGACTTCTGTCCTGCCACC
AGCTAGCTCTCTCCACCTCTGTTTTTCATTTATGAAATGTATGTGTTGGACAGCAGAGGTAGGGAGGAGA
GGAAAGGAGGTCAAAGCCAAGATCTTTTTCCAGCTCTTAAATCCCAAAATTTCTTATAAATGGTTAATGT
AAACAAAAATGAGACACACCTATTAAAATGGCCAAACTCCAAAACTCTGACAACTCCAGATGCTGGTGAG
GATGTGGAACAATGGGAACTTTGTTTTTCTGGTTTTTTTTTTCTTTTTTGAGACAAGGICTTGCTCTGTC
ACCTAGGCTAGAGGGCAGTGGTGCAATCTTGGCTCACTGCAACCTCTGCCTCCAGGGTTCAAGTGATTCT
CATGCCTCAGCCTCCTGAGTAATTGGGATTACAGGCATAAGCCACCATGCCCAGCTAATTTTTGTATTTT
TAGTAGAGACGGGGTTTCACTATTTTGCCCAGGCTCATCTCAAACTCCTGGCCTCAAGTGATCTGCCTGC
CTCGGCCTCCCAAAGTGCTGGGATTACAGGCGTGAGCTACCACGCCCAGCAGCAGTGGGAACTTTGATTC
ATTCCTAGTGGGAATGCAAACCAGTATAGCTACTGTGGATGACAGTTTGGTAGTTTATTACAAAACTAAA
ATATAACACAATATATAATACTTGATATTTTGATAATTATATACAGTATATAATATTTTATAATAAATGT
GTTACTGGTTTGTGTATTTACTATATTTTTTATCATGTTAGCATGTAGTCCTTCTACTTACTAAAAAAAT
TAACTATAAAACAGCCTCTCGGGAGGCTGAGGCAGAAGAATTGCTTGAACCCAGGAGGCAGAGGTTGCAG
TGAGCCAAGGTCGTGCCATTGTACTCTAGCCTGGGTGACAGAACAAGACTCTGTCTCTACAAAAGAAAAA
AAAAAAAAAAAACAGCCTCAGCAGGCAGGTCCTTCAGGAGGTATTCCAGAAGAAGGCATTGCTCTCCTAG
GACAGGACAGCCCCATGAGTATTCTTACCCCTAAGACCTTCCAGTGGGACAAGATGAAGAGGTGGAAAAC
AGTGATACTGATGATCCAGACCCTATGTAGGACTAGGCTAATGTGTGTGCTTGTGTCTTCATTTTTAACA
AAAACAAGAAAAGTTTTTAAAAATTTTTTTAAATAAAAAAGCTTATAAAATAAGAATAGAAAGAAAATAT
TTTTGTGCCACTGTATAACATGTTTGTGTTTTACCCTGTTTTATTACAAAGGTAAAAACATTTACAATTA
AAAAATTTCTAAAGTAAAGTGGCAGTAAACTAGGGTTGATTTTTTAAAATAAGCTTAGTGTGGACTAAAT
GTACAATATTTAAAACATCTACAGTAGTGTACAGCAATGTCCTAGGCCTTCACATTCGCCCACCACTCAT
TCACTGACTTCCAATCTCACAAGCTCCATTCACAGTAAGTGCCCTGTACAGGTGTACCATTTTTAAAATC
TTTTGTGTCATATTTTTACTGTATTTTTCTATGTTTAGATATGGTCAGATACACAAATATTTACCACTGC
ATTACAGTTGCCTACAGTATTCAGTATAGTAACTTGCTGTACAGGTTTGTAGCCTAAGAGCAATAGGACA
TACCACATAGCCTGAGTGTGTACTAGGCTATTCCATGTAAGTTTGTGTAACTACACGCTATGACGITTGC
ACAGAGACAGATATGATGGTGCTATACAAAGTAAGTCTAGAAGAGAGACAGGTTGGCTGCTAATTCAACC
AATATTTACTGGCATCCTAGCAGTATAAACCATGGAAGTGTAAGCAAGGTTAAATGTACTTAGTCTTGAA
GGAGCCTCTGATGACAGTGTCAAGTGGTCACTGCTGTAATGACGTTCTGAACAAGGGCCACATTTAAGGG
TGCCTAGGACAGCTGAAGACTGTTGACTTTAGATAGACTTGAGCCGAAGTCTTCAAAAAGAGTGAGGAGG
AGAAAACCTGTAACACTCCAGGGATTCTGATATAAGCCACTTTTATGTGGATAATTCCAAGGCTGGCTTT
GGTGAGATTATTACCATCGTTAAGGCTGTTGTTTGAATATGATAATTCAAGTAAAGCACTTTGGTACGCT
GCACGACACATAGTAAATGATTAATTGGTAGCAATTATTCATAAATTACTGTCTGTTCTCCCCAAGCTTC
TAGGCTTCCCTAATTAAAATTCTGATAGCTTTAAAGCTTTTCAAGTTCCAATGCGTAATTACTGTTACAA
AGCAGTAACTTGTCTGAATCACCAGCAGCCTCTAGTACAATATTAGAAGCTTCCAGITATTTAGTTAAGA
CTTCAGCAACTAGAGCCATTTCTGGAATACTCTATGTGGACTCTGCCACTTGACTAAACTTCATTAAATT
CCAATCCAGATACCCATCCCCTCAGAGGAGGAGGACAGGACAGTTATGCCTGCAGTTATAAACTGAGAAG
TGTTGTGTTCCAAGCTCTGCATGGAATTGGGTAAATTAGCTACAAGCCTGAAATAAGGAAGTATGCATCT
GCGTTTTTTTCTTTCCAGCACAGAAGATAAGAAACCACTTTCAGGGCAAGGACAAATACCTTCTTATACC
CCAGTATCTAGCACACAGGCACTCAGTTATTTTTGGAGAAGAAAATCTACCATAATACCACTCTCTCAAC
TAATACATTTTTCACCTAACATGCTTTTTCTGAATATTAAAAAAACTGTGATAAGATACTTGGAGTATAT
ACTTCTGGCCTTTATTTTCCTGTGTGCCCAAGGCTATTTTTCCAAAAATGGGACCATGATGGCGTGTTTT
TTGTAGAAACTTTTCCAGTATTACTGAATAATCTAATGATTTTTAAAATGAAGTTTCAAATATTTTAAAA
ATAAATCATCCATGTGGCAAATTAAAAAAACTCAATAGAAACATAAACGAAGTGTATTCTTCCATGATGA
TAACACTATTAAATTACTTGGGAATCCTTCCAGAGAAAAATAAATGCACTTACCTCAATGTATATGTGCT
TTATTTCCACAAATGGGTCACATTCTATACACTGTTCTGCATCTAGGGTTTTTATTGAGATATAATTCAC
AGACCAGAACGTTTACATTTTAAAGTGTATAATTCAGTGGCTCTTAGTATATTTACAAGGTTGTATGACT
TATCGCAACTGTCTAATTCCAGGATCTTTCTGTCACCTTGAAAAGAAACCTGTTAGCAGTCAGTCCTCAT
TCCTCCCTCCTGCATGCCCCAGCAGTGACTGATGTACTTTCTGTCTTCATAGATTTGGCTCTCCTGGGCA
CTTCATATGCAAGGAATCATACAGTATTGTGGCACTTTGCGACTAGCTTTTTTTTCACTAAACATAATGT
TTCCAAGGTTCACTCATGCTGTGGCATGTATCAGTACTICATTCTGTTGTGGCCACATAGTATTCTATTG
TGTGGATATATCACATTTTGTTTATCCATTCACCAGTAATGGACATATGGGTIGTTTCCACTTTTTAGCA
ATTTTGAATCATGGTGCCATGAACATTCATGTGGAAACTTGTGTGTGAACGTAGITTTCAATTCAAACTA
CGATAAGATACTTGGAGTGTATACATCTGGCCTTTATTTTCCTGTGTGCCCAAGGCTATTTTTCCAAAAA
TGAGACCATGATAGCATGTTCTTATAGAAACTTTTCCTGTATTACTGAATAATCTAATGATTTTTAAAAT
GAAGTTTCAAATATTTTAAAAATTATCCATGTGGCAAATTTGAAAAACTCAATAGAAACATAAACGAAGT
GTATTCTTCCATGATGATAACAGTATTAAATTACTTGGGAATCCTTCCAGAGAATGGCAGAAGTATATAC
ACTTGTGAGTGGAATTGCTCAGTCAAATGGATAACTCTATCTTTAACTTTTTGCAGAAATGCCAAATTTG
TCCAGAGTATCTACACCATTTTACATTCCCACCAGCAATGTTTGAGGGTTCCAATTTTTTCCACTICCTT
GCAACATTTGCTATTTACCATTTTCTTGATCTTAGCCATCCTAGTGAGTATAAAGTGGTATCTCACTGTG
ATTTTCATTTGCATGTTCCTAATGACTAATGATATCAAGCATCTTTTCATGTACTTATTGGCCATTTGAG
AATCTTCTTTGGAGAAATGTTTATTCAAATCCTTTGCCCATTTTTTTTTAAAGAGGCAAGGTCTTGCTTT
GTTGCCTAGGCTGGAATGCAGCAGTACAATCATAGCTCACTGCAGCCTTAAAGTCCTGAGTCCAAGTGAT
CCTCCTGCCTCAGCCTCCCAAGTAGCTAAGACTAAAGGCGCAACACCCTGCCTGGCTAATTTTTTTATTT
TTTATTTTTGTAGAGTTGGGATATCACTATGTTGCCCAGGCTGGCCGCAGGCAATCCTCTCACTTCAGCC
TCCTAAAGTGCTGGGATTACAGGTGTGAGCCATCACACTGGCCTCATTGTCCATATTTTAATTGGGTTAG
TCTTTTTATTGTAGAGITTAAGAATGTTTTCTATATCCTGGATACTATGCTCTTACATATGACTTTAAAA
AAGAAATAGAGAGGGGGTCTTGCCATGTTGACCAGGCTGGTCTGAAACTTCCCGCCTCAAACAATTCTCC
CATCTCAGCCTCCCAAAGTGTTGGGATTACAGGTGTGAGCCACCGCACCTGGTGGTTTACATGTGATTTG
CAAACATTTTCTCCCTTTCTGTGAGTTATCTTTTTATTTTTTGATAATGTCCTTTGAAGCACAAAAGTTT
TAAATTTTGATGGAGTCCAGTTTACCTATTTTTCTGCCCCCCCCCCACCTTTATTTTTTTTTTTTTAAAT
CAAGCTCCTTCCATGTTAGCACTTGAAGGCCCACGTCATTTCTTATCTGCATGGAACTTGCATTGTAAGG
AATTCTATTGTAATTTCTTTAACACTTTTTAAAAATTAATGGACTGGGTTTTTCAGGTTTTTTTCTGACC
ATTCTTGAGCATATATCTTATACATAGCTTTGGCTGGGCATGGTGGCTCACCCCTATAATTCTATCACTT
TGGGAGGCTGAAGCAGGAGGACCACTTGAGCCCAGGAGTTCAAGACCAGATGACCGGGCAAGAAAGTGAG
ACCTTATCTCTCTAAAAAAAAAAAAGAAAAGAAAAGGAAAAAATCCCCAAGTAACTTCATAGCGTAAATT
CTAAAAGTGGAATTGCTGGATCAAAGGGGGCATGTGTTTAAAATCTTGGCAGATATTACCAAATTTCCCT
CTTAACAGTATAAATGCCTGTTCCTCCTCCGTCCCACCCCCATAACTATACTGGCTCTGATGAGACCTTG
GTTTTCTGTAAAAGCTCTATTTAGAGGTGTATCATTATTTACTTAATTGTTCTCCTTTACAACCCACCTG
GGATGAGCATCTTGCCTAGAAGTCTCTACTTGCACAGGATACATACGAAATAGAATTGAGGATTCAAAAG
CAGATACAGAACTCTTCCCACTTACTTTCTTACCCTTGTGTGTGTCTCCCCACAGGGTTACAAGTGTATA
ACAAGTGTTGGAAGTTTGAGCATTGCAATTTCAACGACGTCACAACCCGCTTGAGGGAAAATGAGCTAAC
GTACTACTGCTGCAAGAAGGACCTGTGTAACTTTAACGAACAGCTTGAAAATGGTGGGACATCCTTATCA
GAGAAAACAGTTCTTCTGCTGGTGACTCCATTTCTGGCAGCAGCCTGGAGCCTTCATCCCTAAGICAACA
CCAGGAGAGCTTCTCCCAAACTCCCCGTTCCTGCGTAGTCCGCTTTCTCTTGCTGCCACATTCTAAAGGC
TTGATATTTTCCAAATGGATCCTGTTGGGAAAGAATAAAATTAGCTTGAGCAACCTGGCTAAGATAGAGG
GGCTCTGGGAGACTTTGAAGACCAGTCCTGTTTGCAGGGAAGCCCCACTTGAAGGAAGAAGTCTAAGAGT
GAAGTAGGTGTGACTTGAACTAGATTGCATGCTTCCTCCTTTGCTCTTGGGAAGACCAGCTTTGCAGTGA
CAGCTTGAGTGGGTTCTCTGCAGCCCTCAGATTATTTTTCCTCTGGCTCCTTGGATGTAGTCAGTTAGCA
TCATTAGTACATCTTTGGAGGGTGGGGCAGGAGTATATGAGCATCCTCTCTCACATGGAACGCTTTCATA
AACTTCAGGGATCCCGTGTTGCCATGGAGGCATGCCAAATGTTCCATATGTGGGTGTCAGTCAGGGACAA
CAAGATCCTTAATGCAGAGCTAGAGGACTTCTGGCAGGGAAGTGGGGAAGTGTTCCAGATAGCAGGGCAT
GAAAACTTAGAGAGGTACAAGTGGCTGAAAATCGAGTTTTTCCTCTGTCTTTAAATTTTATATGGGCTTT
GTTATCTTCCACTGGAAAAGTGTAATAGCATACATCAATGGTGTGTTAAAGCTATTTCCTTGCCTTTTTT
TTATTGGAATGGTAGGATATCTTGGCTTTGCCACACACAGTTACAGAGTGAACACTCTACTACATGTGAC
TGGCAGTATTAAGTGTGCTTATTTTAAATGTTACTGGTAGAAAGGCAGTTCAGGTATGTGTGTATATAGT
ATGAATGCAGTGGGGACACCCTTTGTGGTTACAGTTTGAGACTTCCAAAGGTCATCCTTAATAACAACAG
ATCTGCAGGGGTATGTTTTACCATCTGCATCCAGCCTCCTGCTAACTCCTAGCTGACTCAGCATAGATTG
TATAAAATACCTTTGTAACGGCTCTTAGCACACTCACAGATGTTTGAGGCTTTCAGAAGCTCTTCTAAAA
AATGATACACACCTTTCACAAGGGCAAACTTTTTCCTTTTCCCTGTGTATTCTAGTGAATGAATCTCAAG
ATTCAGTAGACCTAATGACATTTGTATTTTATGATCTTGGCTGTATTTAATGGCATAGGCTGACTTTTGC
AGATGGAGGAATTTCTTGATTAATGTTGAAAAAAAACCCTTGATTATACTCTGTTGGACAAACCGAGTGC
AATGAATGATGCTTTTCTGAAAATGAAATATAACAAGTGGGTGAATGTGGTTATGGCCGAAAAGGATATG
CAGTATGCTTAATGGTAGCAACTGAAAGAAGACATCCTGAGCAGTGCCAGCTTTCTTCTGTTGATGCCGT
TCCCTGAACATAGGAAAATAGAAACTTGCTTATCAAAACTTAGCATTACCTTGGTGCTCTGTGTTCTCTG
TTAGCTCAGTGTCTTTCCTTACATCAATAGGTTTTTTTTTTTTTTTTTGGCCTGAGGAAGTACTGACCAT
GCCCACAGCCACCGGCTGAGCAAAGAAGCTCATTTCATGTGAGTTCTAAGGAATGAGAAACAATTTTGAT
GAATTTAAGCAGAAAATGAATTTCTGGGAACTTTTTTGGGGGGGGGGGGGTGGGGAATTCAGCCACACTC
CAGAAAGCCAGGAGTCGACAGITTTGGAAGCCTCTCTCAGGATTGAGATTCTAGGATGAGATTGGCTTAC
TGCTATCTTGTGTCATGTACCCACTTTTTGGCCAGACTACACTGGGAAGAAGGTAGTCCTCTAAAGCAAA
ATCTGAGTGCCACTAAATGGGGAGATGGGGCTGTTAAGCTGTCCAAATCAACAAGGGTCATATAAATGGC
CTTAAACTTTGGGGTTGCTTTCTGCAAAAAGTTGCTGTGACTCATGCCATAGACAAGGTTGAGTGCCTGG
ACCCAAAGGCAATACTGTAATGTAAAGACATTTATAGTACTAGGCAAACAGCACCCCAGGTACTCCAGGC
CCTCCTGGCTGGAGAGGGCTGTGGCAATAGAAAATTAGTGCCAACTGCAGTGAGTCAGCCTAGGTTAAAT
AGAGAGTGTAAGAGTGCTGGACAGGAACCTCCACCCTCATGTCACATTTCTTCAATGTGACCCTTCTGGC
CCCTCTCCTCCTGACAGCGGAACAATGACTGCCCCGATAGGTGAGGCTGGAGGAAGAATCAGTCCTGTCC
TTGGCAAGCTCTTCACTATGACAGTAAAGGCTCTCTGCCTGCTGCCAAGGCCTGTGACTTTCTAACCTGG
CCTCACGCTGGGTAAGCTTAAGGTAGAGGTGCAGGATTAGCAAGCCCACCTGGCTACCAGGCCGACAGCT
ACATCCTCCAACTGACCCTGATCAACGAAGAGGGATTCATGTGTCTGTCTCAGTTGGTTCCAAATGAAAC
CAGGGAGCAGGGGAGTTAGGAATCGAACACCAGTCATGCCTACTGGCTCTCTGCTCGAGAGCCAATACCC
TGTGCCCTCCACTCATCTGGATTTACAGGAACTGTCATAGTGTTCAGTATTGGGTGGTGATAAGCCCATT
GGATTGTCCCCTTGGGGGGATGAGCTAGGGGTGCAAGGAACACCTGATGAGTAGATAAGTGGAGCTCATG
GTATTTCCTGAAAGATGCTAATCTATTTGCCAAACTTGGTCTTGAATGTACTGGGGGCTTCAAGGTATGG
GTATATTTTTCTTGTGTCCTTGCAGTTAGCCCCCATGTCTTATGTGTGTCCTGAAAAAATAAGAGCCTGC
CCAAGACTTTGGGCCTCTTGACAGAATTAACCACTTTTATACATCTGAGTTCTCTTGGTAAGTTCTTTAG
CAGTGTTCAAAGTCTACTAGCTCGCATTAGTTTCTGTTGCTGCCAACAGATCTGAACTAATGCTAACAGA
TCCCCCTGAGGGATTCTTGATGGGCTGAGCAGCTGGCTGGAGCTAGTACTGACTGACATTCATTGTGATG
AGGGCAGCTTTCTGGTACAGGATTCTAAGCTCTATGTTTTATATACATTTTCATCTGTACTTGCACCTCA
CTTTACACAAGAGGAAACTATGCAAAGTTAGCTGGATCGCTCAAGGTCACTTAGGTAAGTTGGCAAGTCC
ATGCTTCCCACTCAGCTCCTCAGGTCAGCAAGTCTACTTCTCTGCCTATTTTGTATACTCTCTTTAATAT
GTGCCTAGCTTTGGAAAGTCTAGAATGGGTCCCTGGTGCCTTTTTACTTTGAAGAAATCAGTTTCTGCCT
CTTTTTGGAAAAGAAAACAAAGTGCAATTGTTTTTTACTGGAAAGTTACCCAATAGCATGAGGTGAACAG
GACGTAGTTAGGCCTTCCTGTAAACAGAAAATCATATCAAAACACTATCTTCCCATCTGTTTCTCAATGC
CTGCTACTTCTTGTAGATATTTCATTTCAGGAGAGCAGCAGTTAAACCCGTGGATTTTGTAGTTAGGAAC
CTGGGTTCAAACCCTCTTCCACTAATTGGCTATGTCTCTGGACAAGTTTTTTTTTTTTTTTTTTTTTAAA
CCCTTTCTGAACTTTCACTTTCTATGTCTACCTCAAAGAATTGTTGTGAGGCTTGAGATAATGCATTTGT
AAAGGGTCTGCCAGATAGGAAGATGCTAGTTATGGATTTACAAGGTTGTTAAGGCTGTAAGAGTCTAAAA
CCTACAGTGAATCACAATGCATTTACCCCCACTGACTTGGACATAAGTGAAAACTAGCCAGAAGTCTCTT
TTTCAAATTACTTACAGGTTATTCAATATAAAATTTTTGTAATGGATAATCTTATTTATCTAAACTAAAG
CTTCCTGTTTATACACACTCCTGTTATTCTGGGATAAGATAAATGACCACAGTACCTTAATTTCTAGGTG
GGTGCCTGTGATGGTTCATTGTAGGTAAGGACATTTTCTCTTTTTCAGCAGCTGTGTAGGTCCAGAGCCT
CTGGGAGAGGAGGGGGGTAGCATGCACCCAGCAGGGGACTGAACTGGGAAACTCAAGGTTCTTTTTACTG
TGGGGTAGTGAGCTGCCTTTCTGTGATCGGTTTCCCTAGGGATGTTGCTGTTCCCCTCCTTGCTATTCGC
AGCTACATACAACGTGGCCAACCCCAGTAGGCTGATCCTATATATGATCAGTGCTGGTGCTGACTCTCAA
TAGCCCCACCCAAGCTGGCTATAGGITTACAGATACATTAATTAGGCAACCTAAAATATTGATGCTGGTG
TTGGTGTGACATAATGCTATGGCCAGAACTGAAACTTAGAGITATAATTCATGTATTAGGGTTCTCCAGA
GGGACAGAATTAGTAGGATATATGTATATATGAAAGGGAGGTTATTAGGGAGAACTGGCTCCCACAGTTA
GAAGGCGAAGTCGCACAATAGGCCGTCTGCAAGCTGGGTTAGAGAGAAGCCAGTAGTGGCTCAGCCTGAG
TTCAAAAACCTCAAAACTGGGGAAGCTGACAGTGCAGCCAGCCTTCAGTCTGTGGCCAAAGGCCCAAGAG
CCCCTGGCAACCAACCCACTGGTGCAAGTCCTAGATTCCAAAGGCTGAAGAACCTGGAGTCTGATGTCCA
AGAGCAGGAAGAGTGGAAGAAAGCCAGAAGACTCAGCAAACAAGGTAGACAGTGTCTACCACCATAGTGG
CCATACCAAAGAGGCTACCGATTCCTTCCTGCTACCTGGATCCCTGAAGTTGCCCTGGTCTCTGCACCTT
CTAAACCTAGTTCTTAAGAGCTTTCCATTACATGAGCTGTCTCAAAGCCCTCCAATAAATTCTCAGTGTA
AGCTTCTGTTGCTTGTGGACAGAAAATTCTGACAGACCTACCCTATAAGTGTTACTGTCAGGATAACATG
AGAACGCACAACAGTAAGTGGTCACTAAGTGTTAGCTACGGTTATTTTGCCCAAGGTAGCATGGCTAGIT
GATGCCGGITGATGGGGCTTAAACCCAGCTCCCTCATCTTCCAGGCCTCTGTACTCCCTATTCCACTAAA
CTACCTCTCAGGITTATTTTTTTAAATTCTTACTCTGCAAGTACATAGGACCACATTTACCTGGGAAAAC
AAGAATAAAGGCTGCTCTGCATTTTTTAGAAACTTTTTTGAAAGGGAGATGGGAATGCCTGCACCCCCAA
GTCCAGACCAACACAATGGTTAATTGAGATGAATAATAAAGGAAAGACTGTTCTGGGCTTCCCAGAATAG
CTTGGTCCTTAAATTGTGGCACAAACAACCTCCTGTCAGAGCCAGCCTCCTGCCAGGAAGAGGGGTAGGA
GACTAGAGGCCGTGTGTGCAGCCTTGCCCTGAAGGCTAGGGTGACAATTTGGAGGCTGTCCAAACACCCT
GGCCTCTAGAGCTGGCCTGTCTATTTGAAATGCCGGCTCTGATGCTAATCGGCGACCCTCAGGCAAGTTA
CTTAACCTTACATGCCTCAGTTTTCTCATCTGGAAAATGAGAACCCTAGGTTTAGGGTTGTTAGAAAAGT
TAAATGAGTTAAGACAAGTGCCTGGGACACAGTAGCCTCTTGTGTGTGTTTATCATTATGTCCTCAGCAG
GTCGTAGAAGCAGCTTCTCAGGTGTGAGGCTGGCGCGATTATCTGGAGTGGGTTGGGTTTTCTAGGATGG
ACCCCCTGCTGCATTTTCCTCATTCATCCACCAGGGCTTAATGGGGAATCAAGGAATCCATGTGTAACTG
TATAATAACTGTAGCCACACTCCAATGACCACCTACTAGITGTCCCTGGCACTGCTTATACATATGTCCA
TCAAATCAATCCTATGAAGTAGATACTGTCTTCATTTTATAGATCAGAGACAATTGGGGITCAGAGAGCT
GATGTGATTTTCCCAGGGTCACAGAGAGTCCCAGATTCAGGCACAACTCTTGTATTCCAAGACACAACCA
CTACATGTCCAAAGGCTGCCCAGAGCCACCGGGCACGGCAAATTGTGACATATCCCTAAAGAGGCTGAGC
ACCTGGTCAGGATCTGATGGCTGACAGTGTGTCCAGATGCAGAGCTGGAGTGGGGGAGGGGAAGGGGGGC
TCCTTGGGACAGAGAAGGCTTTCTGTGCTTTCTCTGAAGGGAGCAGTCTGAGGACCAAGGGAACCCGGCA
AACAGCACCTCAGGTACTCCAGGCCCTCCTGGCTGGAGAGGGCTGTGGCAATGGAAAATTAGTGCCAACT
GCAATGAGTCAGCCTCGGTTAAATAGAGAGTGAAGAATGCTGGACAGGAACCTCCACCCTCATGTCACAT
TTCTTCAGTGTGACCCTTCTGGCCCCTCTCCTCCTGACAGCGGAACAATGACTGCCCCGATAGGTGAGGC
TGGAGGAAGAATCAGTCCTGTCCTTGGCAAGCTCTTCACTATGACAGTAAAGGCTCTCTGCCTGCTGCCA
AGGCCTGTGACTTTCTAACCTGGCCTCACGCTGGGTAAGCTTAAGGTAGAGGTGCAGGATTAGCAAGCCC
ACCTGGCTACCAGGCCGACAGCTACATCTTTCAACTGACCCTGATCAACGAAGAGGGACTTGTGTCTCTC
AGTTGGTTCCAAATGAAACCAGGGAGCAGGGGCGTTAGGAAGCTCCAACAGGATGGTACTTAATGGGGCA
TTTGAGTGGAGAGGTAGGTGACATAGTGCTTTGGAGCCCAGGGAGGGAAAGGTTCTGCTGAAGTTGAATT
CAAGACTGTTCTTTCATCACAAACTTGAGTTTCCTGGACATTTGITTGCAGAAACAACCGTAGGGTTTTG
CCTTAACCTCGTGGGTTTATTATTACCTCATAGGGACTTTGCCTCCTGACAGCAGTTTATGGGTGTTCAT
TGTGGCACTTGAGTTTTCTTGCATACTTGTTAGAGAAACCAAGTTTGTCATCAACTTCTTATTTAACCCC
CTGGCTATAACTTCATGGATTATGTTATAATTAAGCCATCCAGAGTAAAATCTGTTTAGATTATCTTGGA
GTAAGGGGGAAAAAATCTGTAATTTTTTCTCCTCAACTAGATATATACATAAAAAATGATTGTATTGCTT
CATTTAAAAAATATAACGCAAAATCTCTTTTCCTTCTAAGGGCCTTTAATATGTAATAACAAGAGCTAAT
GTTGAACACAAGCCATTGCCAGGTACTTAAACTGTCTCACTTAGTCTTGCCAACCTAAGCAAGTAGATGC
CATTACTATTCCCATTATATTGATGAAAAAATGAGGTTGAGAAAGGTTCAGCCGCACAGCCAAATACATA
GTGGATCCAGGGTTTGGTCCCAAGTCTTGAGTCCAAAACCCTGGTTCTTACCATTACTGTGGTTCTGTAG
GGGTGAAAAAATTTTTCTTCTAATCTCTTAGGTTCAGAGCCTGGGGCCTGAGAATTAAACTGACAAAGAC
AGATTAACAGAAGACAAAGTTTATTACGCAGGTACATGGGGTTCTTACAGAAACAAAGTGAAAACCCAAA
GAAGTGGCTTGAGAGCTTATATACAATTTTAACAATGGGCGATTGTAGAAAAGTGATAAGACAAAGGAAA
AGAGGTTTGAGCTTCTTGGGCAGTAAATTATGGGAAGTTAAATATGTGGGGGAAACTAATGGAAGATAAG
GGTATTTTTTATTTTATTTTATTTTATTTATTTTGAGACAGGGTCCTGCCTTGTCACCCAGGCTGGAGTG
CAGTGCTATGATTGTGGCTCACTGCAGCCTCAACCTCAGCCTCCTTGGITCAAGAAATCCTCCCACTTCA
GCTCCTGAGTGGCTCCAGGCAAATGCCACCACACCIGGTTAATTTTTTAAATTATTTTATAGAGACAGAA
TCTCGCTATGTTGCCCCTGGCTGTTCTCAAACTCTTGGCCTCAAGAGATCCCACCTCAGACTCCCAGAGT
GCTGGGATTACAGGCTGGGTGCTGAGCTATCGCACATGGCCTGTATTAGCCATAGACTGAGGGGAAATAC
ATTTTCTTGAGAGCCTCAGCCTAGTGATCACTTGTATGAATAAACAATGATGGGIGGCTGCCAAAGTCAG
AGAGCAAATACTGCGTGTGACAACTAAGTGTGGGAAGCTCAGTGTGGGAAGAAAAAGCTTCCCAGTTTCA
GACATGCCCCTTAGTAGGGACAGCTGCCTCTTGTGTTTGGACAAAGTGTCCAAGTTCAAGTGTTTCACAC
ACACACTGTTCCGTCTTCTGITTGATCACAACCCTATGAGGCAGCCTGGTTTTCTTATTTCTATTTTATG
GAAAACCTACTTAAAAGTCAAGCAACTTGTCCAAGGCTACCCAGCTTGAATATCTGACTCTCTCCACITT
GCTGCTACACACACACACACACACACACACACACTTTGCACTGTGTGTGCAGATACATATGCATATATAT
ATTTAACTTGTGAAGCTGATACTGACTCTTTAACTGCCCTCCACCCATCACTAGTCATACCAGTGATCAT
TAGGCAGAGGCTCTCAAGAAAATGTATTAGTTATTTCCCCTCAGTCTATGGCTAATACAGGTTACTCACA
TAAATGAGCTGTCATGTCCTTTGGACAAAATGTCCAAATCGCTCATAAGCAATGGGTGAAGTATTTGAGG
TAGGGCGGACAGGATTTCCTTTTGGTGGTTAGCTCGTTGCCTGTTAGCTCCTGGTGATATCTGTGTTTGG
AAGAGAACTAAACTGCATTTCACTGAAAACACTCCTTCAGGTCTATTACAAACCAGATGAGGATCCCCTT
TTCTAGAATCTTCCTCAACACTGCTAACGTTTGGCAGATACATAGGTTGGCTCTAACAGCCATTCCCAAT
CAATCCTCTTCTCCTTTGTCTGCCTCCCACAGTAGTGGATGAAAAATCAGACACTGATTTTCTCAGACTT
CTTTGCTGTGGGCGCGGCCAGTGAGGCAATGAGGTAAGTCTGCTGGTGCAGGGGCCAGGTTTCTAGGAAA
TACTTTTCTCCCTCATGGGAGAAAGAGAGGAAGCCTGAAGTAAGCTTTCTTCTGCAGCCCTGGCCCGCTG
CCTGTTCTGGATGAGGGTATGCTGGGATATGACAACGGAAGCCAGCGGGGTCGCAGAGCAAGAGCGAGGC
GGTGGGCGG
(NG_008057.1 Homo sapiens CD59 molecule (CD59 blood group)
(CD59), RefSeqGene (LRG_41) on chromosome 11)

In some embodiments, the nucleotide sequence of an mRNA encoding CD59 is set forth in SEQ ID NO:3:

AGAAGCGGCTCGAGGCTGGAAGAGGATCTTGGGCGCCGCCAGGTTCTGTGGACAATCACAATGGGAATCC
AAGGAGGGTCTGTCCTGTTCGGGCTGCTGCTCGTCCTGGCTGTCTTCTGCCATTCAGGTCATAGCCTGCA
GTGCTACAACTGTCCTAACCCAACTGCTGACTGCAAAACAGCCGTCAATTGTTCATCTGATTTTGATGCG
TGTCTCATTACCAAAGCTGGGTTACAAGTGTATAACAAGTGTTGGAAGTTTGAGCATTGCAATTTCAACG
ACGTCACAACCCGCTTGAGGGAAAATGAGCTAACGTACTACTGCTGCAAGAAGGACCTGTGTAACTTTAA
CGAACAGCTTGAAAATGGTGGGACATCCTTATCAGAGAAAACAGTTCTTCTGCTGGTGACTCCATTTCTG
GCAGCAGCCTGGAGCCTTCATCCCTAAGTCAACACCAGGAGAGCTTCTCCCAAACTCCCCGTTCCTGCGT
AGTCCGCTTTCTCTTGCTGCCACATTCTAAAGGCTTGATATTTTCCAAATGGATCCTGTTGGGAAAGAAT
AAAATTAGCTTGAGCAACCTGGCTAAGATAGAGGGGCTCTGGGAGACTTTGAAGACCAGTCCTGTTTGCA
GGGAAGCCCCACTTGAAGGAAGAAGTCTAAGAGTGAAGTAGGTGTGACTTGAACTAGATTGCATGCTTCC
TCCTTTGCTCTTGGGAAGACCAGCTTTGCAGTGACAGCTTGAGTGGGTTCTCTGCAGCCCTCAGATTATT
TTTCCTCTGGCTCCTTGGATGTAGTCAGTTAGCATCATTAGTACATCTTTGGAGGGTGGGGCAGGAGTAT
ATGAGCATCCTCTCTCACATGGAACGCTTTCATAAACTTCAGGGATCCCGTGTTGCCATGGAGGCATGCC
AAATGTTCCATATGTGGGTGTCAGTCAGGGACAACAAGATCCTTAATGCAGAGCTAGAGGACTTCTGGCA
GGGAAGTGGGGAAGTGTTCCAGATAGCAGGGCATGAAAACTTAGAGAGGTACAAGTGGCTGAAAATCGAG
TTTTTCCTCTGTCTTTAAATTTTATATGGGCTTTGTTATCTTCCACTGGAAAAGTGTAATAGCATACATC
AATGGTGTGTTAAAGCTATTTCCTTGCCTTTTTTTTATTGGAATGGTAGGATATCTTGGCTTTGCCACAC
ACAGTTACAGAGTGAACACTCTACTACATGTGACTGGCAGTATTAAGTGTGCTTATTTTAAATGTTACTG
GTAGAAAGGCAGTTCAGGTATGTGTGTATATAGTATGAATGCAGTGGGGACACCCTTTGTGGTTACAGTT
TGAGACTTCCAAAGGTCATCCTTAATAACAACAGATCTGCAGGGGTATGTTTTACCATCTGCATCCAGCC
TCCTGCTAACTCCTAGCTGACTCAGCATAGATTGTATAAAATACCTTTGTAACGGCTCTTAGCACACTCA
CAGATGTTTGAGGCTTTCAGAAGCTCTTCTAAAAAATGATACACACCTTTCACAAGGGCAAACTTTTTCC
TTTTCCCTGTGTATTCTAGTGAATGAATCTCAAGATTCAGTAGACCTAATGACATTTGTATTTTATGATC
TTGGCTGTATTTAATGGCATAGGCTGACTTTTGCAGATGGAGGAATTTCTTGATTAATGTTGAAAAAAAA
CCCTTGATTATACTCTGTTGGACAAACCGAGTGCAATGAATGATGCTTTTCTGAAAATGAAATATAACAA
GTGGGTGAATGTGGTTATGGCCGAAAAGGATATGCAGTATGCTTAATGGTAGCAACTGAAAGAAGACATC
CTGAGCAGTGCCAGCTTTCTTCTGTTGATGCCGTTCCCTGAACATAGGAAAATAGAAACTTGCTTATCAA
AACTTAGCATTACCTTGGTGCTCTGTGTTCTCTGTTAGCTCAGTGTCTTTCCTTACATCAATAGGTTTTT
TTTTTTTTTTTTGGCCTGAGGAAGTACTGACCATGCCCACAGCCACCGGCTGAGCAAAGAAGCTCATTTC
ATGTGAGTTCTAAGGAATGAGAAACAATTTTGATGAATTTAAGCAGAAAATGAATTTCTGGGAACTTTTT
TGGGGGGGGGGGGGTGGGGAATTCAGCCACACTCCAGAAAGCCAGGAGTCGACAGTTTTGGAAGCCTCTC
TCAGGATTGAGATTCTAGGATGAGATTGGCTTACTGCTATCTTGTGTCATGTACCCACTTTTTGGCCAGA
CTACACTGGGAAGAAGGTAGTCCTCTAAAGCAAAATCTGAGTGCCACTAAATGGGGAGATGGGGCTGTTA
AGCTGTCCAAATCAACAAGGGTCATATAAATGGCCTTAAACTTTGGGGTTGCTTTCTGCAAAAAGTTGCT
GTGACTCATGCCATAGACAAGGTTGAGTGCCTGGACCCAAAGGCAATACTGTAATGTAAAGACATTTATA
GTACTAGGCAAACAGCACCCCAGGTACTCCAGGCCCTCCTGGCTGGAGAGGGCTGTGGCAATAGAAAATT
AGTGCCAACTGCAGTGAGTCAGCCTAGGTTAAATAGAGAGTGTAAGAGTGCTGGACAGGAACCTCCACCC
TCATGTCACATTTCTTCAATGTGACCCTTCTGGCCCCTCTCCTCCTGACAGCGGAACAATGACTGCCCCG
ATAGGTGAGGCTGGAGGAAGAATCAGTCCTGTCCTTGGCAAGCTCTTCACTATGACAGTAAAGGCTCTCT
GCCTGCTGCCAAGGCCTGTGACTTTCTAACCTGGCCTCACGCTGGGTAAGCTTAAGGTAGAGGTGCAGGA
TTAGCAAGCCCACCTGGCTACCAGGCCGACAGCTACATCCTCCAACTGACCCTGATCAACGAAGAGGGAT
TCATGTGTCTGTCTCAGTTGGTTCCAAATGAAACCAGGGAGCAGGGGAGTTAGGAATCGAACACCAGTCA
TGCCTACTGGCTCTCTGCTCGAGAGCCAATACCCTGTGCCCTCCACTCATCTGGATTTACAGGAACTGTC
ATAGTGTTCAGTATTGGGTGGTGATAAGCCCATTGGATTGTCCCCTTGGGGGGATGAGCTAGGGGTGCAA
GGAACACCTGATGAGTAGATAAGTGGAGCTCATGGTATTTCCTGAAAGATGCTAATCTATTTGCCAAACT
TGGTCTTGAATGTACTGGGGGCTTCAAGGTATGGGTATATTTTTCTTGTGTCCTTGCAGTTAGCCCCCAT
GTCTTATGTGTGTCCTGAAAAAATAAGAGCCTGCCCAAGACTTTGGGCCTCTTGACAGAATTAACCACTT
TTATACATCTGAGTTCTCTTGGTAAGTTCTTTAGCAGTGTTCAAAGTCTACTAGCTCGCATTAGTTTCTG
TTGCTGCCAACAGATCTGAACTAATGCTAACAGATCCCCCTGAGGGATTCTTGATGGGCTGAGCAGCTGG
CTGGAGCTAGTACTGACTGACATTCATTGTGATGAGGGCAGCTTTCTGGTACAGGATTCTAAGCTCTATG
TTTTATATACATTTTCATCTGTACTTGCACCTCACTTTACACAAGAGGAAACTATGCAAAGTTAGCTGGA
TCGCTCAAGGTCACTTAGGTAAGTTGGCAAGTCCATGCTTCCCACTCAGCTCCTCAGGTCAGCAAGTCTA
CTTCTCTGCCTATTTTGTATACTCTCTTTAATATGTGCCTAGCTTTGGAAAGTCTAGAATGGGTCCCTGG
TGCCTTTTTACTTTGAAGAAATCAGTTTCTGCCTCTTTTTGGAAAAGAAAACAAAGTGCAATTGTTTTTT
ACTGGAAAGTTACCCAATAGCATGAGGTGAACAGGACGTAGTTAGGCCTTCCTGTAAACAGAAAATCATA
TCAAAACACTATCTTCCCATCTGTTTCTCAATGCCTGCTACTTCTTGTAGATATTTCATTTCAGGAGAGC
AGCAGTTAAACCCGTGGATTTTGTAGTTAGGAACCTGGGTTCAAACCCTCTTCCACTAATTGGCTATGTC
TCTGGACAAGTTTTTTTTTTTTTTTTTTTTTAAACCCTTTCTGAACTTTCACTTTCTATGTCTACCTCAA
AGAATTGTTGTGAGGCTTGAGATAATGCATTTGTAAAGGGTCTGCCAGATAGGAAGATGCTAGTTATGGA
TTTACAAGGTTGTTAAGGCTGTAAGAGTCTAAAACCTACAGTGAATCACAATGCATTTACCCCCACTGAC
TTGGACATAAGTGAAAACTAGCCAGAAGTCTCTTTTTCAAATTACTTACAGGTTATTCAATATAAAATTT
TTGTAATGGATAATCTTATTTATCTAAACTAAAGCTTCCTGTTTATACACACTCCTGTTATTCTGGGATA
AGATAAATGACCACAGTACCTTAATTTCTAGGTGGGTGCCTGTGATGGTTCATTGTAGGTAAGGACATTT
TCTCTTTTTCAGCAGCTGTGTAGGTCCAGAGCCTCTGGGAGAGGAGGGGGGTAGCATGCACCCAGCAGGG
GACTGAACTGGGAAACTCAAGGTTCTTTTTACTGTGGGGTAGTGAGCTGCCTTTCTGTGATCGGTTTCCC
TAGGGATGTTGCTGTTCCCCTCCTTGCTATTCGCAGCTACATACAACGTGGCCAACCCCAGTAGGCTGAT
CCTATATATGATCAGTGCTGGTGCTGACTCTCAATAGCCCCACCCAAGCTGGCTATAGGTTTACAGATAC
ATTAATTAGGCAACCTAAAATATTGATGCTGGTGTTGGTGTGACATAATGCTATGGCCAGAACTGAAACT
TAGAGTTATAATTCATGTATTAGGGTTCTCCAGAGGGACAGAATTAGTAGGATATATGTATATATGAAAG
GGAGGTTATTAGGGAGAACTGGCTCCCACAGTTAGAAGGCGAAGTCGCACAATAGGCCGTCTGCAAGCTG
GGTTAGAGAGAAGCCAGTAGTGGCTCAGCCTGAGTTCAAAAACCTCAAAACTGGGGAAGCTGACAGTGCA
GCCAGCCTTCAGTCTGTGGCCAAAGGCCCAAGAGCCCCTGGCAACCAACCCACTGGTGCAAGTCCTAGAT
TCCAAAGGCTGAAGAACCTGGAGTCTGATGTCCAAGAGCAGGAAGAGTGGAAGAAAGCCAGAAGACTCAG
CAAACAAGGTAGACAGTGTCTACCACCATAGTGGCCATACCAAAGAGGCTACCGATTCCTTCCTGCTACC
TGGATCCCTGAAGTTGCCCTGGTCTCTGCACCTTCTAAACCTAGTTCTTAAGAGCTTTCCATTACATGAG
CTGTCTCAAAGCCCTCCAATAAATTCTCAGTGTAAGCTTCTGTTGCTTGTGGACAGAAAATTCTGACAGA
CCTACCCTATAAGTGTTACTGTCAGGATAACATGAGAACGCACAACAGTAAGTGGTCACTAAGTGTTAGC
TACGGTTATTTTGCCCAAGGTAGCATGGCTAGTTGATGCCGGTTGATGGGGCTTAAACCCAGCTCCCTCA
TCTTCCAGGCCTCTGTACTCCCTATTCCACTAAACTACCTCTCAGGTTTATTTTTTTAAATTCTTACTCT
GCAAGTACATAGGACCACATTTACCTGGGAAAACAAGAATAAAGGCTGCTCTGCATTTTTTAGAAACTTT
TTTGAAAGGGAGATGGGAATGCCTGCACCCCCAAGTCCAGACCAACACAATGGTTAATTGAGATGAATAA
TAAAGGAAAGACTGTTCTGGGCTTCCCAGAATAGCTTGGTCCTTAAATTGTGGCACAAACAACCTCCTGT
CAGAGCCAGCCTCCTGCCAGGAAGAGGGGTAGGAGACTAGAGGCCGTGTGTGCAGCCTTGCCCTGAAGGC
TAGGGTGACAATTTGGAGGCTGTCCAAACACCCTGGCCTCTAGAGCTGGCCTGTCTATTTGAAATGCCGG
CTCTGATGCTAATCGGCGACCCTCAGGCAAGTTACTTAACCTTACATGCCTCAGTTTTCTCATCTGGAAA
ATGAGAACCCTAGGTTTAGGGTTGTTAGAAAAGTTAAATGAGTTAAGACAAGTGCCTGGGACACAGTAGC
CTCTTGTGTGTGTTTATCATTATGTCCTCAGCAGGTCGTAGAAGCAGCTTCTCAGGTGTGAGGCTGGCGC
GATTATCTGGAGTGGGTTGGGTTTTCTAGGATGGACCCCCTGCTGCATTTTCCTCATTCATCCACCAGGG
CTTAATGGGGAATCAAGGAATCCATGTGTAACTGTATAATAACTGTAGCCACACTCCAATGACCACCTAC
TAGTTGTCCCTGGCACTGCTTATACATATGTCCATCAAATCAATCCTATGAAGTAGATACTGTCTTCATT
TTATAGATCAGAGACAATTGGGGTTCAGAGAGCTGATGTGATTTTCCCAGGGTCACAGAGAGTCCCAGAT
TCAGGCACAACTCTTGTATTCCAAGACACAACCACTACATGTCCAAAGGCTGCCCAGAGCCACCGGGCAC
GGCAAATTGTGACATATCCCTAAAGAGGCTGAGCACCTGGTCAGGATCTGATGGCTGACAGTGTGTCCAG
ATGCAGAGCTGGAGTGGGGGAGGGGAAGGGGGGCTCCTTGGGACAGAGAAGGCTTTCTGTGCTTTCTCTG
AAGGGAGCAGTCTGAGGACCAAGGGAACCCGGCAAACAGCACCTCAGGTACTCCAGGCCCTCCTGGCTGG
AGAGGGCTGTGGCAATGGAAAATTAGTGCCAACTGCAATGAGTCAGCCTCGGTTAAATAGAGAGTGAAGA
ATGCTGGACAGGAACCTCCACCCTCATGTCACATTTCTTCAGTGTGACCCTTCTGGCCCCTCTCCTCCTG
ACAGCGGAACAATGACTGCCCCGATAGGTGAGGCTGGAGGAAGAATCAGTCCTGTCCTTGGCAAGCTCTT
CACTATGACAGTAAAGGCTCTCTGCCTGCTGCCAAGGCCTGTGACTTTCTAACCTGGCCTCACGCTGGGT
AAGCTTAAGGTAGAGGTGCAGGATTAGCAAGCCCACCTGGCTACCAGGCCGACAGCTACATCTTTCAACT
GACCCTGATCAACGAAGAGGGACTTGTGTCTCTCAGTTGGTTCCAAATGAAACCAGGGAGCAGGGGCGTT
AGGAAGCTCCAACAGGATGGTACTTAATGGGGCATTTGAGTGGAGAGGTAGGTGACATAGTGCTTTGGAG
CCCAGGGAGGGAAAGGTTCTGCTGAAGTTGAATTCAAGACTGTTCTTTCATCACAAACTTGAGTTTCCTG
GACATTTGTTTGCAGAAACAACCGTAGGGTTTTGCCTTAACCTCGTGGGTTTATTATTACCTCATAGGGA
CTTTGCCTCCTGACAGCAGTTTATGGGTGTTCATTGTGGCACTTGAGTTTTCTTGCATACTTGTTAGAGA
AACCAAGTTTGTCATCAACTTCTTATTTAACCCCCTGGCTATAACTTCATGGATTATGTTATAATTAAGC
CATCCAGAGTAAAATCTGTTTAGATTATCTTGGAGTAAGGGGGAAAAAATCTGTAATTTTTTCTCCTCAA
CTAGATATATACATAAAAAATGATTGTATTGCTTCATTTAAAAAATATAACGCAAAATCTCTTTTCCTTC
TAA (NM_000611.6 Homo sapiens CD59 molecule (CD59 blood group) (CD59) ,
transcript variant 2, mRNA.

In some embodiments, the nucleotide sequence of an mRNA encoding CD59 is set forth in SEQ ID NO:4:

AGAAGCGGCTCGAGGCTGGAAGAGGATCTTGGGCGCCGCCAGTCTTTAGCACCAGTTGGTGTAGGAGTTG
AGACCTACTTCACAGTAGTTCTGTGGACAATCACAATGGGAATCCAAGGAGGGTCTGTCCTGTTCGGGCT
GCTGCTCGTCCTGGCTGTCTTCTGCCATTCAGGTCATAGCCTGCAGTGCTACAACTGTCCTAACCCAACT
GCTGACTGCAAAACAGCCGTCAATTGTTCATCTGATTTTGATGCGTGTCTCATTACCAAAGCTGGGTTAC
AAGTGTATAACAAGTGTTGGAAGTTTGAGCATTGCAATTTCAACGACGTCACAACCCGCTTGAGGGAAAA
TGAGCTAACGTACTACTGCTGCAAGAAGGACCTGTGTAACTTTAACGAACAGCTTGAAAATGGTGGGACA
TCCTTATCAGAGAAAACAGTTCTTCTGCTGGTGACTCCATTTCTGGCAGCAGCCTGGAGCCTTCATCCCT
AAGTCAACACCAGGAGAGCTTCTCCCAAACTCCCCGTTCCTGCGTAGTCCGCTTTCTCTTGCTGCCACAT
TCTAAAGGCTTGATATTTTCCAAATGGATCCTGTTGGGAAAGAATAAAATTAGCTTGAGCAACCTGGCTA
AGATAGAGGGGCTCTGGGAGACTTTGAAGACCAGTCCTGTTTGCAGGGAAGCCCCACTTGAAGGAAGAAG
TCTAAGAGTGAAGTAGGTGTGACTTGAACTAGATTGCATGCTTCCTCCTTTGCTCTTGGGAAGACCAGCT
TTGCAGTGACAGCTTGAGTGGGTTCTCTGCAGCCCTCAGATTATTTTTCCTCTGGCTCCTTGGATGTAGT
CAGTTAGCATCATTAGTACATCTTTGGAGGGTGGGGCAGGAGTATATGAGCATCCTCTCTCACATGGAAC
GCTTTCATAAACTTCAGGGATCCCGTGTTGCCATGGAGGCATGCCAAATGTTCCATATGTGGGTGTCAGT
CAGGGACAACAAGATCCTTAATGCAGAGCTAGAGGACTTCTGGCAGGGAAGTGGGGAAGTGTTCCAGATA
GCAGGGCATGAAAACTTAGAGAGGTACAAGTGGCTGAAAATCGAGTTTTTCCTCTGTCTTTAAATTTTAT
ATGGGCTTTGTTATCTTCCACTGGAAAAGTGTAATAGCATACATCAATGGTGTGTTAAAGCTATTTCCTT
GCCTTTTTTTTATTGGAATGGTAGGATATCTTGGCTTTGCCACACACAGTTACAGAGTGAACACTCTACT
ACATGTGACTGGCAGTATTAAGTGTGCTTATTTTAAATGTTACTGGTAGAAAGGCAGTTCAGGTATGTGT
GTATATAGTATGAATGCAGTGGGGACACCCTTTGTGGTTACAGTTTGAGACTTCCAAAGGTCATCCTTAA
TAACAACAGATCTGCAGGGGTATGTTTTACCATCTGCATCCAGCCTCCTGCTAACTCCTAGCTGACTCAG
CATAGATTGTATAAAATACCTTTGTAACGGCTCTTAGCACACTCACAGATGTTTGAGGCTTTCAGAAGCT
CTTCTAAAAAATGATACACACCTTTCACAAGGGCAAACTTTTTCCTTTTCCCTGTGTATTCTAGTGAATG
AATCTCAAGATTCAGTAGACCTAATGACATTTGTATTTTATGATCTTGGCTGTATTTAATGGCATAGGCT
GACTTTTGCAGATGGAGGAATTTCTTGATTAATGTTGAAAAAAAACCCTTGATTATACTCTGTTGGACAA
ACCGAGTGCAATGAATGATGCTTTTCTGAAAATGAAATATAACAAGTGGGTGAATGTGGTTATGGCCGAA
AAGGATATGCAGTATGCTTAATGGTAGCAACTGAAAGAAGACATCCTGAGCAGTGCCAGCTTTCTTCTGT
TGATGCCGTTCCCTGAACATAGGAAAATAGAAACTTGCTTATCAAAACTTAGCATTACCTTGGTGCTCTG
TGTTCTCTGTTAGCTCAGTGTCTTTCCTTACATCAATAGGTTTTTTTTTTTTTTTTTGGCCTGAGGAAGT
ACTGACCATGCCCACAGCCACCGGCTGAGCAAAGAAGCTCATTTCATGTGAGTTCTAAGGAATGAGAAAC
AATTTTGATGAATTTAAGCAGAAAATGAATTTCTGGGAACTTTTTTGGGGGGGGGGGGGTGGGGAATTCA
GCCACACTCCAGAAAGCCAGGAGTCGACAGTTTTGGAAGCCTCTCTCAGGATTGAGATTCTAGGATGAGA
TTGGCTTACTGCTATCTTGTGTCATGTACCCACTTTTTGGCCAGACTACACTGGGAAGAAGGTAGTCCTC
TAAAGCAAAATCTGAGTGCCACTAAATGGGGAGATGGGGCTGTTAAGCTGTCCAAATCAACAAGGGTCAT
ATAAATGGCCTTAAACTTTGGGGTTGCTTTCTGCAAAAAGTTGCTGTGACTCATGCCATAGACAAGGTTG
AGTGCCTGGACCCAAAGGCAATACTGTAATGTAAAGACATTTATAGTACTAGGCAAACAGCACCCCAGGT
ACTCCAGGCCCTCCTGGCTGGAGAGGGCTGTGGCAATAGAAAATTAGTGCCAACTGCAGTGAGTCAGCCT
AGGTTAAATAGAGAGTGTAAGAGTGCTGGACAGGAACCTCCACCCTCATGTCACATTTCTTCAATGTGAC
CCTTCTGGCCCCTCTCCTCCTGACAGCGGAACAATGACTGCCCCGATAGGTGAGGCTGGAGGAAGAATCA
GTCCTGTCCTTGGCAAGCTCTTCACTATGACAGTAAAGGCTCTCTGCCTGCTGCCAAGGCCTGTGACTTT
CTAACCTGGCCTCACGCTGGGTAAGCTTAAGGTAGAGGTGCAGGATTAGCAAGCCCACCTGGCTACCAGG
CCGACAGCTACATCCTCCAACTGACCCTGATCAACGAAGAGGGATTCATGTGTCTGTCTCAGTTGGTTCC
AAATGAAACCAGGGAGCAGGGGAGTTAGGAATCGAACACCAGTCATGCCTACTGGCTCTCTGCTCGAGAG
CCAATACCCTGTGCCCTCCACTCATCTGGATTTACAGGAACTGTCATAGTGTTCAGTATTGGGTGGTGAT
AAGCCCATTGGATTGTCCCCTTGGGGGGATGAGCTAGGGGTGCAAGGAACACCTGATGAGTAGATAAGTG
GAGCTCATGGTATTTCCTGAAAGATGCTAATCTATTTGCCAAACTTGGTCTTGAATGTACTGGGGGCTTC
AAGGTATGGGTATATTTTTCTTGTGTCCTTGCAGTTAGCCCCCATGTCTTATGTGTGTCCTGAAAAAATA
AGAGCCTGCCCAAGACTTTGGGCCTCTTGACAGAATTAACCACTTTTATACATCTGAGTTCTCTTGGTAA
GTTCTTTAGCAGTGTTCAAAGTCTACTAGCTCGCATTAGTTTCTGTTGCTGCCAACAGATCTGAACTAAT
GCTAACAGATCCCCCTGAGGGATTCTTGATGGGCTGAGCAGCTGGCTGGAGCTAGTACTGACTGACATTC
ATTGTGATGAGGGCAGCTTTCTGGTACAGGATTCTAAGCTCTATGTTTTATATACATTTTCATCTGTACT
TGCACCTCACTTTACACAAGAGGAAACTATGCAAAGTTAGCTGGATCGCTCAAGGTCACTTAGGTAAGTT
GGCAAGTCCATGCTTCCCACTCAGCTCCTCAGGTCAGCAAGTCTACTTCTCTGCCTATTTTGTATACTCT
CTTTAATATGTGCCTAGCTTTGGAAAGTCTAGAATGGGTCCCTGGTGCCTTTTTACTTTGAAGAAATCAG
TTTCTGCCTCTTTTTGGAAAAGAAAACAAAGTGCAATTGTTTTTTACTGGAAAGTTACCCAATAGCATGA
GGTGAACAGGACGTAGTTAGGCCTTCCTGTAAACAGAAAATCATATCAAAACACTATCTTCCCATCTGTT
TCTCAATGCCTGCTACTTCTTGTAGATATTTCATTTCAGGAGAGCAGCAGTTAAACCCGTGGATTTTGTA
GTTAGGAACCTGGGTTCAAACCCTCTTCCACTAATTGGCTATGTCTCTGGACAAGTTTTTTTTTTTTTTT
TTTTTTAAACCCTTTCTGAACTTTCACTTTCTATGTCTACCTCAAAGAATTGTTGTGAGGCTTGAGATAA
TGCATTTGTAAAGGGTCTGCCAGATAGGAAGATGCTAGTTATGGATTTACAAGGTTGTTAAGGCTGTAAG
AGTCTAAAACCTACAGTGAATCACAATGCATTTACCCCCACTGACTTGGACATAAGTGAAAACTAGCCAG
AAGTCTCTTTTTCAAATTACTTACAGGTTATTCAATATAAAATTTTTGTAATGGATAATCTTATTTATCT
AAACTAAAGCTTCCTGTTTATACACACTCCTGTTATTCTGGGATAAGATAAATGACCACAGTACCTTAAT
TTCTAGGTGGGTGCCTGTGATGGTTCATTGTAGGTAAGGACATTTTCTCTTTTTCAGCAGCTGTGTAGGT
CCAGAGCCTCTGGGAGAGGAGGGGGGTAGCATGCACCCAGCAGGGGACTGAACTGGGAAACTCAAGGTTC
TTTTTACTGTGGGGTAGTGAGCTGCCTTTCTGTGATCGGTTTCCCTAGGGATGTTGCTGTTCCCCTCCTT
GCTATTCGCAGCTACATACAACGTGGCCAACCCCAGTAGGCTGATCCTATATATGATCAGTGCTGGTGCT
GACTCTCAATAGCCCCACCCAAGCTGGCTATAGGTTTACAGATACATTAATTAGGCAACCTAAAATATTG
ATGCTGGTGTTGGTGTGACATAATGCTATGGCCAGAACTGAAACTTAGAGTTATAATTCATGTATTAGGG
TTCTCCAGAGGGACAGAATTAGTAGGATATATGTATATATGAAAGGGAGGTTATTAGGGAGAACTGGCTC
CCACAGTTAGAAGGCGAAGTCGCACAATAGGCCGTCTGCAAGCTGGGTTAGAGAGAAGCCAGTAGTGGCT
CAGCCTGAGTTCAAAAACCTCAAAACTGGGGAAGCTGACAGTGCAGCCAGCCTTCAGTCTGTGGCCAAAG
GCCCAAGAGCCCCTGGCAACCAACCCACTGGTGCAAGTCCTAGATTCCAAAGGCTGAAGAACCTGGAGTC
TGATGTCCAAGAGCAGGAAGAGTGGAAGAAAGCCAGAAGACTCAGCAAACAAGGTAGACAGTGTCTACCA
CCATAGTGGCCATACCAAAGAGGCTACCGATTCCTTCCTGCTACCTGGATCCCTGAAGTTGCCCTGGTCT
CTGCACCTTCTAAACCTAGTTCTTAAGAGCTTTCCATTACATGAGCTGTCTCAAAGCCCTCCAATAAATT
CTCAGTGTAAGCTTCTGTTGCTTGTGGACAGAAAATTCTGACAGACCTACCCTATAAGTGTTACTGTCAG
GATAACATGAGAACGCACAACAGTAAGTGGTCACTAAGTGTTAGCTACGGTTATTTTGCCCAAGGTAGCA
TGGCTAGTTGATGCCGGTTGATGGGGCTTAAACCCAGCTCCCTCATCTTCCAGGCCTCTGTACTCCCTAT
TCCACTAAACTACCTCTCAGGTTTATTTTTTTAAATTCTTACTCTGCAAGTACATAGGACCACATTTACC
TGGGAAAACAAGAATAAAGGCTGCTCTGCATTTTTTAGAAACTTTTTTGAAAGGGAGATGGGAATGCCTG
CACCCCCAAGTCCAGACCAACACAATGGTTAATTGAGATGAATAATAAAGGAAAGACTGTTCTGGGCTTC
CCAGAATAGCTTGGTCCTTAAATTGTGGCACAAACAACCTCCTGTCAGAGCCAGCCTCCTGCCAGGAAGA
GGGGTAGGAGACTAGAGGCCGTGTGTGCAGCCTTGCCCTGAAGGCTAGGGTGACAATTTGGAGGCTGTCC
AAACACCCTGGCCTCTAGAGCTGGCCTGTCTATTTGAAATGCCGGCTCTGATGCTAATCGGCGACCCTCA
GGCAAGTTACTTAACCTTACATGCCTCAGTTTTCTCATCTGGAAAATGAGAACCCTAGGTTTAGGGTTGT
TAGAAAAGTTAAATGAGTTAAGACAAGTGCCTGGGACACAGTAGCCTCTTGTGTGTGTTTATCATTATGT
CCTCAGCAGGTCGTAGAAGCAGCTTCTCAGGTGTGAGGCTGGCGCGATTATCTGGAGTGGGTTGGGTTTT
CTAGGATGGACCCCCTGCTGCATTTTCCTCATTCATCCACCAGGGCTTAATGGGGAATCAAGGAATCCAT
GTGTAACTGTATAATAACTGTAGCCACACTCCAATGACCACCTACTAGTTGTCCCTGGCACTGCTTATAC
ATATGTCCATCAAATCAATCCTATGAAGTAGATACTGTCTTCATTTTATAGATCAGAGACAATTGGGGTT
CAGAGAGCTGATGTGATTTTCCCAGGGTCACAGAGAGTCCCAGATTCAGGCACAACTCTTGTATTCCAAG
ACACAACCACTACATGTCCAAAGGCTGCCCAGAGCCACCGGGCACGGCAAATTGTGACATATCCCTAAAG
AGGCTGAGCACCTGGTCAGGATCTGATGGCTGACAGTGTGTCCAGATGCAGAGCTGGAGTGGGGGAGGGG
AAGGGGGGCTCCTTGGGACAGAGAAGGCTTTCTGTGCTTTCTCTGAAGGGAGCAGTCTGAGGACCAAGGG
AACCCGGCAAACAGCACCTCAGGTACTCCAGGCCCTCCTGGCTGGAGAGGGCTGTGGCAATGGAAAATTA
GTGCCAACTGCAATGAGTCAGCCTCGGTTAAATAGAGAGTGAAGAATGCTGGACAGGAACCTCCACCCTC
ATGTCACATTTCTTCAGTGTGACCCTTCTGGCCCCTCTCCTCCTGACAGCGGAACAATGACTGCCCCGAT
AGGTGAGGCTGGAGGAAGAATCAGTCCTGTCCTTGGCAAGCTCTTCACTATGACAGTAAAGGCTCTCTGC
CTGCTGCCAAGGCCTGTGACTTTCTAACCTGGCCTCACGCTGGGTAAGCTTAAGGTAGAGGTGCAGGATT
AGCAAGCCCACCTGGCTACCAGGCCGACAGCTACATCTTTCAACTGACCCTGATCAACGAAGAGGGACTT
GTGTCTCTCAGTTGGTTCCAAATGAAACCAGGGAGCAGGGGCGTTAGGAAGCTCCAACAGGATGGTACTT
AATGGGGCATTTGAGTGGAGAGGTAGGTGACATAGTGCTTTGGAGCCCAGGGAGGGAAAGGTTCTGCTGA
AGTTGAATTCAAGACTGTTCTTTCATCACAAACTTGAGTTTCCTGGACATTTGTTTGCAGAAACAACCGT
AGGGTTTTGCCTTAACCTCGTGGGTTTATTATTACCTCATAGGGACTTTGCCTCCTGACAGCAGTTTATG
GGTGTTCATTGTGGCACTTGAGTTTTCTTGCATACTTGTTAGAGAAACCAAGTTTGTCATCAACTTCTTA
TTTAACCCCCTGGCTATAACTTCATGGATTATGTTATAATTAAGCCATCCAGAGTAAAATCTGTTTAGAT
TATCTTGGAGTAAGGGGGAAAAAATCTGTAATTTTTTCTCCTCAACTAGATATATACATAAAAAATGATT
GTATTGCTTCATTTAAAAAATATAACGCAAAATCTCTTTTCCTTCTAA (NM_203331.3 Homo
sapiens CD59 molecule (CD59 blood group) (CD59), transcript variant 4,
mRNA)

In some embodiments, the nucleotide sequence of an mRNA encoding CD59 is set forth in SEQ ID NO:5:

GGGGCCGGGGGGCGGAGCCTTGCGGGCTGGAGCGAAAGAATGCGGGGGCTGAGCGCAGAAGCGGCTCGAG
GCTGGAAGAGGATCTTGGGCGCCGCCAGTCTCTCTCTGTTGCCCAAGCTGGAGTGCAGTGGCACAGTCTT
GGCTCACTGCAACCTCCACCTCCTGGGTGCAAGCGATTCTCGTGTCTCAGCCTCTCAAGTAGCTGGGATT
ACAGTCTTTAGCACCAGTTGGTGTAGGAGTTGAGACCTACTTCACAGTAGTTCTGTGGACAATCACAATG
GGAATCCAAGGAGGGTCTGTCCTGTTCGGGCTGCTGCTCGTCCTGGCTGTCTTCTGCCATTCAGGTCATA
GCCTGCAGTGCTACAACTGTCCTAACCCAACTGCTGACTGCAAAACAGCCGTCAATTGTTCATCTGATTT
TGATGCGTGTCTCATTACCAAAGCTGGGTTACAAGTGTATAACAAGTGTTGGAAGTTTGAGCATTGCAAT
TTCAACGACGTCACAACCCGCTTGAGGGAAAATGAGCTAACGTACTACTGCTGCAAGAAGGACCTGTGTA
ACTTTAACGAACAGCTTGAAAATGGTGGGACATCCTTATCAGAGAAAACAGTTCTTCTGCTGGTGACTCC
ATTTCTGGCAGCAGCCTGGAGCCTTCATCCCTAAGTCAACACCAGGAGAGCTTCTCCCAAACTCCCCGTT
CCTGCGTAGTCCGCTTTCTCTTGCTGCCACATTCTAAAGGCTTGATATTTTCCAAATGGATCCTGTTGGG
AAAGAATAAAATTAGCTTGAGCAACCTGGCTAAGATAGAGGGGCTCTGGGAGACTTTGAAGACCAGTCCT
GTTTGCAGGGAAGCCCCACTTGAAGGAAGAAGTCTAAGAGTGAAGTAGGTGTGACTTGAACTAGATTGCA
TGCTTCCTCCTTTGCTCTTGGGAAGACCAGCTTTGCAGTGACAGCTTGAGTGGGTTCTCTGCAGCCCTCA
GATTATTTTTCCTCTGGCTCCTTGGATGTAGTCAGTTAGCATCATTAGTACATCTTTGGAGGGTGGGGCA
GGAGTATATGAGCATCCTCTCTCACATGGAACGCTTTCATAAACTTCAGGGATCCCGTGTTGCCATGGAG
GCATGCCAAATGTTCCATATGTGGGTGTCAGTCAGGGACAACAAGATCCTTAATGCAGAGCTAGAGGACT
TCTGGCAGGGAAGTGGGGAAGTGTTCCAGATAGCAGGGCATGAAAACTTAGAGAGGTACAAGTGGCTGAA
AATCGAGTTTTTCCTCTGTCTTTAAATTTTATATGGGCTTTGTTATCTTCCACTGGAAAAGTGTAATAGC
ATACATCAATGGTGTGTTAAAGCTATTTCCTTGCCTTTTTTTTATTGGAATGGTAGGATATCTTGGCTTT
GCCACACACAGTTACAGAGTGAACACTCTACTACATGTGACTGGCAGTATTAAGTGTGCTTATTTTAAAT
GTTACTGGTAGAAAGGCAGTTCAGGTATGTGTGTATATAGTATGAATGCAGTGGGGACACCCTTTGTGGT
TACAGTTTGAGACTTCCAAAGGTCATCCTTAATAACAACAGATCTGCAGGGGTATGTTTTACCATCTGCA
TCCAGCCTCCTGCTAACTCCTAGCTGACTCAGCATAGATTGTATAAAATACCTTTGTAACGGCTCTTAGC
ACACTCACAGATGTTTGAGGCTTTCAGAAGCTCTTCTAAAAAATGATACACACCTTTCACAAGGGCAAAC
TTTTTCCTTTTCCCTGTGTATTCTAGTGAATGAATCTCAAGATTCAGTAGACCTAATGACATTTGTATTT
TATGATCTTGGCTGTATTTAATGGCATAGGCTGACTTTTGCAGATGGAGGAATTTCTTGATTAATGTTGA
AAAAAAACCCTTGATTATACTCTGTTGGACAAACCGAGTGCAATGAATGATGCTTTTCTGAAAATGAAAT
ATAACAAGTGGGTGAATGTGGTTATGGCCGAAAAGGATATGCAGTATGCTTAATGGTAGCAACTGAAAGA
AGACATCCTGAGCAGTGCCAGCTTTCTTCTGTTGATGCCGTTCCCTGAACATAGGAAAATAGAAACTTGC
TTATCAAAACTTAGCATTACCTTGGTGCTCTGTGTTCTCTGTTAGCTCAGTGTCTTTCCTTACATCAATA
GGTTTTTTTTTTTTTTTTTGGCCTGAGGAAGTACTGACCATGCCCACAGCCACCGGCTGAGCAAAGAAGC
TCATTTCATGTGAGTTCTAAGGAATGAGAAACAATTTTGATGAATTTAAGCAGAAAATGAATTTCTGGGA
ACTTTTTTGGGGGGGGGGGGGTGGGGAATTCAGCCACACTCCAGAAAGCCAGGAGTCGACAGTTTTGGAA
GCCTCTCTCAGGATTGAGATTCTAGGATGAGATTGGCTTACTGCTATCTTGTGTCATGTACCCACTTTTT
GGCCAGACTACACTGGGAAGAAGGTAGTCCTCTAAAGCAAAATCTGAGTGCCACTAAATGGGGAGATGGG
GCTGTTAAGCTGTCCAAATCAACAAGGGTCATATAAATGGCCTTAAACTTTGGGGTTGCTTTCTGCAAAA
AGTTGCTGTGACTCATGCCATAGACAAGGTTGAGTGCCTGGACCCAAAGGCAATACTGTAATGTAAAGAC
ATTTATAGTACTAGGCAAACAGCACCCCAGGTACTCCAGGCCCTCCTGGCTGGAGAGGGCTGTGGCAATA
GAAAATTAGTGCCAACTGCAGTGAGTCAGCCTAGGTTAAATAGAGAGTGTAAGAGTGCTGGACAGGAACC
TCCACCCTCATGTCACATTTCTTCAATGTGACCCTTCTGGCCCCTCTCCTCCTGACAGCGGAACAATGAC
TGCCCCGATAGGTGAGGCTGGAGGAAGAATCAGTCCTGTCCTTGGCAAGCTCTTCACTATGACAGTAAAG
GCTCTCTGCCTGCTGCCAAGGCCTGTGACTTTCTAACCTGGCCTCACGCTGGGTAAGCTTAAGGTAGAGG
TGCAGGATTAGCAAGCCCACCTGGCTACCAGGCCGACAGCTACATCCTCCAACTGACCCTGATCAACGAA
GAGGGATTCATGTGTCTGTCTCAGTTGGTTCCAAATGAAACCAGGGAGCAGGGGAGTTAGGAATCGAACA
CCAGTCATGCCTACTGGCTCTCTGCTCGAGAGCCAATACCCTGTGCCCTCCACTCATCTGGATTTACAGG
AACTGTCATAGTGTTCAGTATTGGGTGGTGATAAGCCCATTGGATTGTCCCCTTGGGGGGATGAGCTAGG
GGTGCAAGGAACACCTGATGAGTAGATAAGTGGAGCTCATGGTATTTCCTGAAAGATGCTAATCTATTTG
CCAAACTTGGTCTTGAATGTACTGGGGGCTTCAAGGTATGGGTATATTTTTCTTGTGTCCTTGCAGTTAG
CCCCCATGTCTTATGTGTGTCCTGAAAAAATAAGAGCCTGCCCAAGACTTTGGGCCTCTTGACAGAATTA
ACCACTTTTATACATCTGAGTTCTCTTGGTAAGTTCTTTAGCAGTGTTCAAAGTCTACTAGCTCGCATTA
GTTTCTGTTGCTGCCAACAGATCTGAACTAATGCTAACAGATCCCCCTGAGGGATTCTTGATGGGCTGAG
CAGCTGGCTGGAGCTAGTACTGACTGACATTCATTGTGATGAGGGCAGCTTTCTGGTACAGGATTCTAAG
CTCTATGTTTTATATACATTTTCATCTGTACTTGCACCTCACTTTACACAAGAGGAAACTATGCAAAGTT
AGCTGGATCGCTCAAGGTCACTTAGGTAAGTTGGCAAGTCCATGCTTCCCACTCAGCTCCTCAGGTCAGC
AAGTCTACTTCTCTGCCTATTTTGTATACTCTCTTTAATATGTGCCTAGCTTTGGAAAGTCTAGAATGGG
TCCCTGGTGCCTTTTTACTTTGAAGAAATCAGTTTCTGCCTCTTTTTGGAAAAGAAAACAAAGTGCAATT
GTTTTTTACTGGAAAGTTACCCAATAGCATGAGGTGAACAGGACGTAGTTAGGCCTTCCTGTAAACAGAA
AATCATATCAAAACACTATCTTCCCATCTGTTTCTCAATGCCTGCTACTTCTTGTAGATATTTCATTTCA
GGAGAGCAGCAGTTAAACCCGTGGATTTTGTAGTTAGGAACCTGGGTTCAAACCCTCTTCCACTAATTGG
CTATGTCTCTGGACAAGTTTTTTTTTTTTTTTTTTTTTAAACCCTTTCTGAACTTTCACTTTCTATGTCT
ACCTCAAAGAATTGTTGTGAGGCTTGAGATAATGCATTTGTAAAGGGTCTGCCAGATAGGAAGATGCTAG
TTATGGATTTACAAGGTTGTTAAGGCTGTAAGAGTCTAAAACCTACAGTGAATCACAATGCATTTACCCC
CACTGACTTGGACATAAGTGAAAACTAGCCAGAAGTCTCTTTTTCAAATTACTTACAGGTTATTCAATAT
AAAATTTTTGTAATGGATAATCTTATTTATCTAAACTAAAGCTTCCTGTTTATACACACTCCTGTTATTC
TGGGATAAGATAAATGACCACAGTACCTTAATTTCTAGGTGGGTGCCTGTGATGGTTCATTGTAGGTAAG
GACATTTTCTCTTTTTCAGCAGCTGTGTAGGTCCAGAGCCTCTGGGAGAGGAGGGGGGTAGCATGCACCC
AGCAGGGGACTGAACTGGGAAACTCAAGGTTCTTTTTACTGTGGGGTAGTGAGCTGCCTTTCTGTGATCG
GTTTCCCTAGGGATGTTGCTGTTCCCCTCCTTGCTATTCGCAGCTACATACAACGTGGCCAACCCCAGTA
GGCTGATCCTATATATGATCAGTGCTGGTGCTGACTCTCAATAGCCCCACCCAAGCTGGCTATAGGTTTA
CAGATACATTAATTAGGCAACCTAAAATATTGATGCTGGTGTTGGTGTGACATAATGCTATGGCCAGAAC
TGAAACTTAGAGTTATAATTCATGTATTAGGGTTCTCCAGAGGGACAGAATTAGTAGGATATATGTATAT
ATGAAAGGGAGGTTATTAGGGAGAACTGGCTCCCACAGTTAGAAGGCGAAGTCGCACAATAGGCCGTCTG
CAAGCTGGGTTAGAGAGAAGCCAGTAGTGGCTCAGCCTGAGTTCAAAAACCTCAAAACTGGGGAAGCTGA
CAGTGCAGCCAGCCTTCAGTCTGTGGCCAAAGGCCCAAGAGCCCCTGGCAACCAACCCACTGGTGCAAGT
CCTAGATTCCAAAGGCTGAAGAACCTGGAGTCTGATGTCCAAGAGCAGGAAGAGTGGAAGAAAGCCAGAA
GACTCAGCAAACAAGGTAGACAGTGTCTACCACCATAGTGGCCATACCAAAGAGGCTACCGATTCCTTCC
TGCTACCTGGATCCCTGAAGTTGCCCTGGTCTCTGCACCTTCTAAACCTAGTTCTTAAGAGCTTTCCATT
ACATGAGCTGTCTCAAAGCCCTCCAATAAATTCTCAGTGTAAGCTTCTGTTGCTTGTGGACAGAAAATTC
TGACAGACCTACCCTATAAGTGTTACTGTCAGGATAACATGAGAACGCACAACAGTAAGTGGTCACTAAG
TGTTAGCTACGGTTATTTTGCCCAAGGTAGCATGGCTAGTTGATGCCGGTTGATGGGGCTTAAACCCAGC
TCCCTCATCTTCCAGGCCTCTGTACTCCCTATTCCACTAAACTACCTCTCAGGTTTATTTTTTTAAATTC
TTACTCTGCAAGTACATAGGACCACATTTACCTGGGAAAACAAGAATAAAGGCTGCTCTGCATTTTTTAG
AAACTTTTTTGAAAGGGAGATGGGAATGCCTGCACCCCCAAGTCCAGACCAACACAATGGTTAATTGAGA
TGAATAATAAAGGAAAGACTGTTCTGGGCTTCCCAGAATAGCTTGGTCCTTAAATTGTGGCACAAACAAC
CTCCTGTCAGAGCCAGCCTCCTGCCAGGAAGAGGGGTAGGAGACTAGAGGCCGTGTGTGCAGCCTTGCCC
TGAAGGCTAGGGTGACAATTTGGAGGCTGTCCAAACACCCTGGCCTCTAGAGCTGGCCTGTCTATTTGAA
ATGCCGGCTCTGATGCTAATCGGCGACCCTCAGGCAAGTTACTTAACCTTACATGCCTCAGTTTTCTCAT
CTGGAAAATGAGAACCCTAGGTTTAGGGTTGTTAGAAAAGTTAAATGAGTTAAGACAAGTGCCTGGGACA
CAGTAGCCTCTTGTGTGTGTTTATCATTATGTCCTCAGCAGGTCGTAGAAGCAGCTTCTCAGGTGTGAGG
CTGGCGCGATTATCTGGAGTGGGTTGGGTTTTCTAGGATGGACCCCCTGCTGCATTTTCCTCATTCATCC
ACCAGGGCTTAATGGGGAATCAAGGAATCCATGTGTAACTGTATAATAACTGTAGCCACACTCCAATGAC
CACCTACTAGTTGTCCCTGGCACTGCTTATACATATGTCCATCAAATCAATCCTATGAAGTAGATACTGT
CTTCATTTTATAGATCAGAGACAATTGGGGTTCAGAGAGCTGATGTGATTTTCCCAGGGTCACAGAGAGT
CCCAGATTCAGGCACAACTCTTGTATTCCAAGACACAACCACTACATGTCCAAAGGCTGCCCAGAGCCAC
CGGGCACGGCAAATTGTGACATATCCCTAAAGAGGCTGAGCACCTGGTCAGGATCTGATGGCTGACAGTG
TGTCCAGATGCAGAGCTGGAGTGGGGGAGGGGAAGGGGGGCTCCTTGGGACAGAGAAGGCTTTCTGTGCT
TTCTCTGAAGGGAGCAGTCTGAGGACCAAGGGAACCCGGCAAACAGCACCTCAGGTACTCCAGGCCCTCC
TGGCTGGAGAGGGCTGTGGCAATGGAAAATTAGTGCCAACTGCAATGAGTCAGCCTCGGTTAAATAGAGA
GTGAAGAATGCTGGACAGGAACCTCCACCCTCATGTCACATTTCTTCAGTGTGACCCTTCTGGCCCCTCT
CCTCCTGACAGCGGAACAATGACTGCCCCGATAGGTGAGGCTGGAGGAAGAATCAGTCCTGTCCTTGGCA
AGCTCTTCACTATGACAGTAAAGGCTCTCTGCCTGCTGCCAAGGCCTGTGACTTTCTAACCTGGCCTCAC
GCTGGGTAAGCTTAAGGTAGAGGTGCAGGATTAGCAAGCCCACCTGGCTACCAGGCCGACAGCTACATCT
TTCAACTGACCCTGATCAACGAAGAGGGACTTGTGTCTCTCAGTTGGTTCCAAATGAAACCAGGGAGCAG
GGGCGTTAGGAAGCTCCAACAGGATGGTACTTAATGGGGCATTTGAGTGGAGAGGTAGGTGACATAGTGC
TTTGGAGCCCAGGGAGGGAAAGGTTCTGCTGAAGTTGAATTCAAGACTGTTCTTTCATCACAAACTTGAG
TTTCCTGGACATTTGTTTGCAGAAACAACCGTAGGGTTTTGCCTTAACCTCGTGGGTTTATTATTACCTC
ATAGGGACTTTGCCTCCTGACAGCAGTTTATGGGTGTTCATTGTGGCACTTGAGTTTTCTTGCATACTTG
TTAGAGAAACCAAGTTTGTCATCAACTTCTTATTTAACCCCCTGGCTATAACTTCATGGATTATGTTATA
ATTAAGCCATCCAGAGTAAAATCTGTTTAGATTATCTTGGAGTAAGGGGGAAAAAATCTGTAATTTTTTC
TCCTCAACTAGATATATACATAAAAAATGATTGTATTGCTTCATTTAAAAAATATAACGCAAAATCTCTT
TTCCTTCTAAAAAAAAAAAAAAAAAA (NM_203330.2 Homo sapiens CD59 molecule
(CD59 blood group) (CD59), transcript variant 1, mRNA)

In some embodiments, the nucleotide sequence of an mRNA encoding CD59 is set forth in SEQ ID NO:6:

AGAAGCGGCTCGAGGCTGGAAGAGGATCTTGGGCGCCGCCAGTCTCTCTCTGTTGCCCAAGCTGGAGTGC
AGTGGCACAGTCTTGGCTCACTGCAACCTCCACCTCCTGGGTGCAAGCGATTCTCGTGTCTCAGCCTCTC
AAGTAGCTGGGATTACAGGTTCTGTGGACAATCACAATGGGAATCCAAGGAGGGTCTGTCCTGTTCGGGC
TGCTGCTCGTCCTGGCTGTCTTCTGCCATTCAGGTCATAGCCTGCAGTGCTACAACTGTCCTAACCCAAC
TGCTGACTGCAAAACAGCCGTCAATTGTTCATCTGATTTTGATGCGTGTCTCATTACCAAAGCTGGGTTA
CAAGTGTATAACAAGTGTTGGAAGTTTGAGCATTGCAATTTCAACGACGTCACAACCCGCTTGAGGGAAA
ATGAGCTAACGTACTACTGCTGCAAGAAGGACCTGTGTAACTTTAACGAACAGCTTGAAAATGGTGGGAC
ATCCTTATCAGAGAAAACAGTTCTTCTGCTGGTGACTCCATTTCTGGCAGCAGCCTGGAGCCTTCATCCC
TAAGTCAACACCAGGAGAGCTTCTCCCAAACTCCCCGTTCCTGCGTAGTCCGCTTTCTCTTGCTGCCACA
TTCTAAAGGCTTGATATTTTCCAAATGGATCCTGTTGGGAAAGAATAAAATTAGCTTGAGCAACCTGGCT
AAGATAGAGGGGCTCTGGGAGACTTTGAAGACCAGTCCTGTTTGCAGGGAAGCCCCACTTGAAGGAAGAA
GTCTAAGAGTGAAGTAGGTGTGACTTGAACTAGATTGCATGCTTCCTCCTTTGCTCTTGGGAAGACCAGC
TTTGCAGTGACAGCTTGAGTGGGTTCTCTGCAGCCCTCAGATTATTTTTCCTCTGGCTCCTTGGATGTAG
TCAGTTAGCATCATTAGTACATCTTTGGAGGGTGGGGCAGGAGTATATGAGCATCCTCTCTCACATGGAA
CGCTTTCATAAACTTCAGGGATCCCGTGTTGCCATGGAGGCATGCCAAATGTTCCATATGTGGGTGTCAG
TCAGGGACAACAAGATCCTTAATGCAGAGCTAGAGGACTTCTGGCAGGGAAGTGGGGAAGTGTTCCAGAT
AGCAGGGCATGAAAACTTAGAGAGGTACAAGTGGCTGAAAATCGAGTTTTTCCTCTGTCTTTAAATTTTA
TATGGGCTTTGTTATCTTCCACTGGAAAAGTGTAATAGCATACATCAATGGTGTGTTAAAGCTATTTCCT
TGCCTTTTTTTTATTGGAATGGTAGGATATCTTGGCTTTGCCACACACAGTTACAGAGTGAACACTCTAC
TACATGTGACTGGCAGTATTAAGTGTGCTTATTTTAAATGTTACTGGTAGAAAGGCAGTTCAGGTATGTG
TGTATATAGTATGAATGCAGTGGGGACACCCTTTGTGGTTACAGTTTGAGACTTCCAAAGGTCATCCTTA
ATAACAACAGATCTGCAGGGGTATGTTTTACCATCTGCATCCAGCCTCCTGCTAACTCCTAGCTGACTCA
GCATAGATTGTATAAAATACCTTTGTAACGGCTCTTAGCACACTCACAGATGTTTGAGGCTTTCAGAAGC
TCTTCTAAAAAATGATACACACCTTTCACAAGGGCAAACTTTTTCCTTTTCCCTGTGTATTCTAGTGAAT
GAATCTCAAGATTCAGTAGACCTAATGACATTTGTATTTTATGATCTTGGCTGTATTTAATGGCATAGGC
TGACTTTTGCAGATGGAGGAATTTCTTGATTAATGTTGAAAAAAAACCCTTGATTATACTCTGTTGGACA
AACCGAGTGCAATGAATGATGCTTTTCTGAAAATGAAATATAACAAGTGGGTGAATGTGGTTATGGCCGA
AAAGGATATGCAGTATGCTTAATGGTAGCAACTGAAAGAAGACATCCTGAGCAGTGCCAGCTTTCTTCTG
TTGATGCCGTTCCCTGAACATAGGAAAATAGAAACTTGCTTATCAAAACTTAGCATTACCTTGGTGCTCT
GTGTTCTCTGTTAGCTCAGTGTCTTTCCTTACATCAATAGGTTTTTTTTTTTTTTTTTGGCCTGAGGAAG
TACTGACCATGCCCACAGCCACCGGCTGAGCAAAGAAGCTCATTTCATGTGAGTTCTAAGGAATGAGAAA
CAATTTTGATGAATTTAAGCAGAAAATGAATTTCTGGGAACTTTTTTGGGGGGGGGGGGGTGGGGAATTC
AGCCACACTCCAGAAAGCCAGGAGTCGACAGTTTTGGAAGCCTCTCTCAGGATTGAGATTCTAGGATGAG
ATTGGCTTACTGCTATCTTGTGTCATGTACCCACTTTTTGGCCAGACTACACTGGGAAGAAGGTAGTCCT
CTAAAGCAAAATCTGAGTGCCACTAAATGGGGAGATGGGGCTGTTAAGCTGTCCAAATCAACAAGGGTCA
TATAAATGGCCTTAAACTTTGGGGTTGCTTTCTGCAAAAAGTTGCTGTGACTCATGCCATAGACAAGGTT
GAGTGCCTGGACCCAAAGGCAATACTGTAATGTAAAGACATTTATAGTACTAGGCAAACAGCACCCCAGG
TACTCCAGGCCCTCCTGGCTGGAGAGGGCTGTGGCAATAGAAAATTAGTGCCAACTGCAGTGAGTCAGCC
TAGGTTAAATAGAGAGTGTAAGAGTGCTGGACAGGAACCTCCACCCTCATGTCACATTTCTTCAATGTGA
CCCTTCTGGCCCCTCTCCTCCTGACAGCGGAACAATGACTGCCCCGATAGGTGAGGCTGGAGGAAGAATC
AGTCCTGTCCTTGGCAAGCTCTTCACTATGACAGTAAAGGCTCTCTGCCTGCTGCCAAGGCCTGTGACTT
TCTAACCTGGCCTCACGCTGGGTAAGCTTAAGGTAGAGGTGCAGGATTAGCAAGCCCACCTGGCTACCAG
GCCGACAGCTACATCCTCCAACTGACCCTGATCAACGAAGAGGGATTCATGTGTCTGTCTCAGTTGGTTC
CAAATGAAACCAGGGAGCAGGGGAGTTAGGAATCGAACACCAGTCATGCCTACTGGCTCTCTGCTCGAGA
GCCAATACCCTGTGCCCTCCACTCATCTGGATTTACAGGAACTGTCATAGTGTTCAGTATTGGGTGGTGA
TAAGCCCATTGGATTGTCCCCTTGGGGGGATGAGCTAGGGGTGCAAGGAACACCTGATGAGTAGATAAGT
GGAGCTCATGGTATTTCCTGAAAGATGCTAATCTATTTGCCAAACTTGGTCTTGAATGTACTGGGGGCTT
CAAGGTATGGGTATATTTTTCTTGTGTCCTTGCAGTTAGCCCCCATGTCTTATGTGTGTCCTGAAAAAAT
AAGAGCCTGCCCAAGACTTTGGGCCTCTTGACAGAATTAACCACTTTTATACATCTGAGTTCTCTTGGTA
AGTTCTTTAGCAGTGTTCAAAGTCTACTAGCTCGCATTAGTTTCTGTTGCTGCCAACAGATCTGAACTAA
TGCTAACAGATCCCCCTGAGGGATTCTTGATGGGCTGAGCAGCTGGCTGGAGCTAGTACTGACTGACATT
CATTGTGATGAGGGCAGCTTTCTGGTACAGGATTCTAAGCTCTATGTTTTATATACATTTTCATCTGTAC
TTGCACCTCACTTTACACAAGAGGAAACTATGCAAAGTTAGCTGGATCGCTCAAGGTCACTTAGGTAAGT
TGGCAAGTCCATGCTTCCCACTCAGCTCCTCAGGTCAGCAAGTCTACTTCTCTGCCTATTTTGTATACTC
TCTTTAATATGTGCCTAGCTTTGGAAAGTCTAGAATGGGTCCCTGGTGCCTTTTTACTTTGAAGAAATCA
GTTTCTGCCTCTTTTTGGAAAAGAAAACAAAGTGCAATTGTTTTTTACTGGAAAGTTACCCAATAGCATG
AGGTGAACAGGACGTAGTTAGGCCTTCCTGTAAACAGAAAATCATATCAAAACACTATCTTCCCATCTGT
TTCTCAATGCCTGCTACTTCTTGTAGATATTTCATTTCAGGAGAGCAGCAGTTAAACCCGTGGATTTTGT
AGTTAGGAACCTGGGTTCAAACCCTCTTCCACTAATTGGCTATGTCTCTGGACAAGTTTTTTTTTTTTTT
TTTTTTTAAACCCTTTCTGAACTTTCACTTTCTATGTCTACCTCAAAGAATTGTTGTGAGGCTTGAGATA
ATGCATTTGTAAAGGGTCTGCCAGATAGGAAGATGCTAGTTATGGATTTACAAGGTTGTTAAGGCTGTAA
GAGTCTAAAACCTACAGTGAATCACAATGCATTTACCCCCACTGACTTGGACATAAGTGAAAACTAGCCA
GAAGTCTCTTTTTCAAATTACTTACAGGTTATTCAATATAAAATTTTTGTAATGGATAATCTTATTTATC
TAAACTAAAGCTTCCTGTTTATACACACTCCTGTTATTCTGGGATAAGATAAATGACCACAGTACCTTAA
TTTCTAGGTGGGTGCCTGTGATGGTTCATTGTAGGTAAGGACATTTTCTCTTTTTCAGCAGCTGTGTAGG
TCCAGAGCCTCTGGGAGAGGAGGGGGGTAGCATGCACCCAGCAGGGGACTGAACTGGGAAACTCAAGGTT
CTTTTTACTGTGGGGTAGTGAGCTGCCTTTCTGTGATCGGTTTCCCTAGGGATGTTGCTGTTCCCCTCCT
TGCTATTCGCAGCTACATACAACGTGGCCAACCCCAGTAGGCTGATCCTATATATGATCAGTGCTGGTGC
TGACTCTCAATAGCCCCACCCAAGCTGGCTATAGGTTTACAGATACATTAATTAGGCAACCTAAAATATT
GATGCTGGTGTTGGTGTGACATAATGCTATGGCCAGAACTGAAACTTAGAGTTATAATTCATGTATTAGG
GTTCTCCAGAGGGACAGAATTAGTAGGATATATGTATATATGAAAGGGAGGTTATTAGGGAGAACTGGCT
CCCACAGTTAGAAGGCGAAGTCGCACAATAGGCCGTCTGCAAGCTGGGTTAGAGAGAAGCCAGTAGTGGC
TCAGCCTGAGTTCAAAAACCTCAAAACTGGGGAAGCTGACAGTGCAGCCAGCCTTCAGTCTGTGGCCAAA
GGCCCAAGAGCCCCTGGCAACCAACCCACTGGTGCAAGTCCTAGATTCCAAAGGCTGAAGAACCTGGAGT
CTGATGTCCAAGAGCAGGAAGAGTGGAAGAAAGCCAGAAGACTCAGCAAACAAGGTAGACAGTGTCTACC
ACCATAGTGGCCATACCAAAGAGGCTACCGATTCCTTCCTGCTACCTGGATCCCTGAAGTTGCCCTGGTC
TCTGCACCTTCTAAACCTAGTTCTTAAGAGCTTTCCATTACATGAGCTGTCTCAAAGCCCTCCAATAAAT
TCTCAGTGTAAGCTTCTGTTGCTTGTGGACAGAAAATTCTGACAGACCTACCCTATAAGTGTTACTGTCA
GGATAACATGAGAACGCACAACAGTAAGTGGTCACTAAGTGTTAGCTACGGTTATTTTGCCCAAGGTAGC
ATGGCTAGTTGATGCCGGTTGATGGGGCTTAAACCCAGCTCCCTCATCTTCCAGGCCTCTGTACTCCCTA
TTCCACTAAACTACCTCTCAGGTTTATTTTTTTAAATTCTTACTCTGCAAGTACATAGGACCACATTTAC
CTGGGAAAACAAGAATAAAGGCTGCTCTGCATTTTTTAGAAACTTTTTTGAAAGGGAGATGGGAATGCCT
GCACCCCCAAGTCCAGACCAACACAATGGTTAATTGAGATGAATAATAAAGGAAAGACTGTTCTGGGCTT
CCCAGAATAGCTTGGTCCTTAAATTGTGGCACAAACAACCTCCTGTCAGAGCCAGCCTCCTGCCAGGAAG
AGGGGTAGGAGACTAGAGGCCGTGTGTGCAGCCTTGCCCTGAAGGCTAGGGTGACAATTTGGAGGCTGTC
CAAACACCCTGGCCTCTAGAGCTGGCCTGTCTATTTGAAATGCCGGCTCTGATGCTAATCGGCGACCCTC
AGGCAAGTTACTTAACCTTACATGCCTCAGTTTTCTCATCTGGAAAATGAGAACCCTAGGTTTAGGGTTG
TTAGAAAAGTTAAATGAGTTAAGACAAGTGCCTGGGACACAGTAGCCTCTTGTGTGTGTTTATCATTATG
TCCTCAGCAGGTCGTAGAAGCAGCTTCTCAGGTGTGAGGCTGGCGCGATTATCTGGAGTGGGTTGGGTTT
TCTAGGATGGACCCCCTGCTGCATTTTCCTCATTCATCCACCAGGGCTTAATGGGGAATCAAGGAATCCA
TGTGTAACTGTATAATAACTGTAGCCACACTCCAATGACCACCTACTAGTTGTCCCTGGCACTGCTTATA
CATATGTCCATCAAATCAATCCTATGAAGTAGATACTGTCTTCATTTTATAGATCAGAGACAATTGGGGT
TCAGAGAGCTGATGTGATTTTCCCAGGGTCACAGAGAGTCCCAGATTCAGGCACAACTCTTGTATTCCAA
GACACAACCACTACATGTCCAAAGGCTGCCCAGAGCCACCGGGCACGGCAAATTGTGACATATCCCTAAA
GAGGCTGAGCACCTGGTCAGGATCTGATGGCTGACAGTGTGTCCAGATGCAGAGCTGGAGTGGGGGGGG
GAAGGGGGGCTCCTTGGGACAGAGAAGGCTTTCTGTGCTTTCTCTGAAGGGAGCAGTCTGAGGACCAAGG
GAACCCGGCAAACAGCACCTCAGGTACTCCAGGCCCTCCTGGCTGGAGAGGGCTGTGGCAATGGAAAATT
AGTGCCAACTGCAATGAGTCAGCCTCGGTTAAATAGAGAGTGAAGAATGCTGGACAGGAACCTCCACCCT
CATGTCACATTTCTTCAGTGTGACCCTTCTGGCCCCTCTCCTCCTGACAGCGGAACAATGACTGCCCCGA
TAGGTGAGGCTGGAGGAAGAATCAGTCCTGTCCTTGGCAAGCTCTTCACTATGACAGTAAAGGCTCTCTG
CCTGCTGCCAAGGCCTGTGACTTTCTAACCTGGCCTCACGCTGGGTAAGCTTAAGGTAGAGGTGCAGGAT
TAGCAAGCCCACCTGGCTACCAGGCCGACAGCTACATCTTTCAACTGACCCTGATCAACGAAGAGGGACT
TGTGTCTCTCAGTTGGTTCCAAATGAAACCAGGGAGCAGGGGCGTTAGGAAGCTCCAACAGGATGGTACT
TAATGGGGCATTTGAGTGGAGAGGTAGGTGACATAGTGCTTTGGAGCCCAGGGAGGGAAAGGTTCTGCTG
AAGTTGAATTCAAGACTGTTCTTTCATCACAAACTTGAGTTTCCTGGACATTTGTTTGCAGAAACAACCG
TAGGGTTTTGCCTTAACCTCGTGGGTTTATTATTACCTCATAGGGACTTTGCCTCCTGACAGCAGTTTAT
GGGTGTTCATTGTGGCACTTGAGTTTTCTTGCATACTTGTTAGAGAAACCAAGTTTGTCATCAACTTCTT
ATTTAACCCCCTGGCTATAACTTCATGGATTATGTTATAATTAAGCCATCCAGAGTAAAATCTGTTTAGA
TTATCTTGGAGTAAGGGGGAAAAAATCTGTAATTTTTTCTCCTCAACTAGATATATACATAAAAAATGAT
TGTATTGCTTCATTTAAAAAATATAACGCAAAATCTCTTTTCCTTCTAA (NM_203329.3 Homo
sapiens CD59 molecule (CD59 blood group) (CD59), transcript variant 3,
mRNA)

In some embodiments, the nucleotide sequence of an mRNA encoding CD59 is set forth in SEQ ID NO:7:

ATTTGACTCACTGACCCTGATGGGGAAGATAGTGTTATTCCCACAGTGTGGTAGTACACACTGGGGCTTA
TAGGGACTGAGCCTACTCAAGGGTATATGGTGCTGTGGGTCAGAGCTGGGGCATGGCCCAGGGATTCAGT
GTGCCTTGACTCCCCCTGTAAATGTTCCTCTCAGAAGCCTTCTTGGCCTTCCAGCCCTTGGTTTTTGAGA
CAACCAGCAGTCATTTGTTCGTTCCTGACATTCCTTCCTGTCCCTTCCTTCCAGGTTCTGTGGACAATCA
CAATGGGAATCCAAGGAGGGTCTGTCCTGTTCGGGCTGCTGCTCGTCCTGGCTGTCTTCTGCCATTCAGG
TCATAGCCTGCAGTGCTACAACTGTCCTAACCCAACTGCTGACTGCAAAACAGCCGTCAATTGTTCATCT
GATTTTGATGCGTGTCTCATTACCAAAGCTGGGTTACAAGTGTATAACAAGTGTTGGAAGTTTGAGCATT
GCAATTTCAACGACGTCACAACCCGCTTGAGGGAAAATGAGCTAACGTACTACTGCTGCAAGAAGGACCT
GTGTAACTTTAACGAACAGCTTGAAAATGGTGGGACATCCTTATCAGAGAAAACAGTTCTTCTGCTGGTG
ACTCCATTTCTGGCAGCAGCCTGGAGCCTTCATCCCTAAGTCAACACCAGGAGAGCTTCTCCCAAACTCC
CCGTTCCTGCGTAGTCCGCTTTCTCTTGCTGCCACATTCTAAAGGCTTGATATTTTCCAAATGGATCCTG
TTGGGAAAGAATAAAATTAGCTTGAGCAACCTGGCTAAGATAGAGGGGCTCTGGGAGACTTTGAAGACCA
GTCCTGTTTGCAGGGAAGCCCCACTTGAAGGAAGAAGTCTAAGAGTGAAGTAGGTGTGACTTGAACTAGA
TTGCATGCTTCCTCCTTTGCTCTTGGGAAGACCAGCTTTGCAGTGACAGCTTGAGTGGGTTCTCTGCAGC
CCTCAGATTATTTTTCCTCTGGCTCCTTGGATGTAGTCAGTTAGCATCATTAGTACATCTTTGGAGGGTG
GGGCAGGAGTATATGAGCATCCTCTCTCACATGGAACGCTTTCATAAACTTCAGGGATCCCGTGTTGCCA
TGGAGGCATGCCAAATGTTCCATATGTGGGTGTCAGTCAGGGACAACAAGATCCTTAATGCAGAGCTAGA
GGACTTCTGGCAGGGAAGTGGGGAAGTGTTCCAGATAGCAGGGCATGAAAACTTAGAGAGGTACAAGTGG
CTGAAAATCGAGTTTTTCCTCTGTCTTTAAATTTTATATGGGCTTTGTTATCTTCCACTGGAAAAGTGTA
ATAGCATACATCAATGGTGTGTTAAAGCTATTTCCTTGCCTTTTTTTTATTGGAATGGTAGGATATCTTG
GCTTTGCCACACACAGTTACAGAGTGAACACTCTACTACATGTGACTGGCAGTATTAAGTGTGCTTATTT
TAAATGTTACTGGTAGAAAGGCAGTTCAGGTATGTGTGTATATAGTATGAATGCAGTGGGGACACCCTTT
GTGGTTACAGTTTGAGACTTCCAAAGGTCATCCTTAATAACAACAGATCTGCAGGGGTATGTTTTACCAT
CTGCATCCAGCCTCCTGCTAACTCCTAGCTGACTCAGCATAGATTGTATAAAATACCTTTGTAACGGCTC
TTAGCACACTCACAGATGTTTGAGGCTTTCAGAAGCTCTTCTAAAAAATGATACACACCTTTCACAAGGG
CAAACTTTTTCCTTTTCCCTGTGTATTCTAGTGAATGAATCTCAAGATTCAGTAGACCTAATGACATTTG
TATTTTATGATCTTGGCTGTATTTAATGGCATAGGCTGACTTTTGCAGATGGAGGAATTTCTTGATTAAT
GTTGAAAAAAAACCCTTGATTATACTCTGTTGGACAAACCGAGTGCAATGAATGATGCTTTTCTGAAAAT
GAAATATAACAAGTGGGTGAATGTGGTTATGGCCGAAAAGGATATGCAGTATGCTTAATGGTAGCAACTG
AAAGAAGACATCCTGAGCAGTGCCAGCTTTCTTCTGTTGATGCCGTTCCCTGAACATAGGAAAATAGAAA
CTTGCTTATCAAAACTTAGCATTACCTTGGTGCTCTGTGTTCTCTGTTAGCTCAGTGTCTTTCCTTACAT
CAATAGGTTTTTTTTTTTTTTTTTGGCCTGAGGAAGTACTGACCATGCCCACAGCCACCGGCTGAGCAAA
GAAGCTCATTTCATGTGAGTTCTAAGGAATGAGAAACAATTTTGATGAATTTAAGCAGAAAATGAATTTC
TGGGAACTTTTTTGGGGGCGGGGGGGTGGGGAATTCAGCCACACTCCAGAAAGCCAGGAGTCGACAGTTT
TGGAAGCCTCTCTCAGGATTGAGATTCTAGGATGAGATTGGCTTACTGCTATCTTGTGTCATGTACCCAC
TTTTTGGCCAGACTACACTGGGAAGAAGGTAGTCCTCTAAAGCAAAATCTGAGTGCCACTAAATGGGGAG
ATGGGGCTGTTAAGCTGTCCAAATCAACAAGGGTCATATAAATGGCCTTAAACTTTGGGGTTGCTTTCTG
CAAAAAGTTGCTGTGACTCATGCCATAGACAAGGTTGAGTGCCTGGACCCAAAGGCAATACTGTAATGTA
AAGACATTTATAGTACTAGGCAAACAGCACCCCAGGTACTCCAGGCCCTCCTGGCTGGAGAGGGCTGTGG
CAATAGAAAATTAGTGCCAACTGCAGTGAGTCAGCCTAGGTTAAATAGAGAGTGTAAGAGTGCTGGACAG
GAACCTCCACCCTCATGTCACATTTCTTCAATGTGACCCTTCTGGCCCCTCTCCTCCTGACAGCGGAACA
ATGACTGCCCCGATAGGTGAGGCTGGAGGAAGAATCAGTCCTGTCCTTGGCAAGCTCTTCACTATGACAG
TAAAGGCTCTCTGCCTGCTGCCAAGGCCTGTGACTTTCTAACCTGGCCTCACGCTGGGTAAGCTTAAGGT
AGAGGTGCAGGATTAGCAAGCCCACCTGGCTACCAGGCCGACAGCTACATCCTCCAACTGACCCTGATCA
ACGAAGAGGGATTCATGTGTCTGTCTCAGTTGGTTCCAAATGAAACCAGGGAGCAGGGGAGTTAGGAATC
GAACACCAGTCATGCCTACTGGCTCTCTGCTCGAGAGCCAATACCCTGTGCCCTCCACTCATCTGGATTT
ACAGGAACTGTCATAGTGTTCAGTATTGGGTGGTGATAAGCCCATTGGATTGTCCCCTTGGGGGGATGAG
CTAGGGGTGCAAGGAACACCTGATGAGTAGATAAGTGGAGCTCATGGTATTTCCTGAAAGATGCTAATCT
ATTTGCCAAACTTGGTCTTGAATGTACTGGGGGCTTCAAGGTATGGGTATATTTTTCTTGTGTCCTTGCA
GTTAGCCCCCATGTCTTATGTGTGTCCTGAAAAAATAAGAGCCTGCCCAAGACTTTGGGCCTCTTGACAG
AATTAACCACTTTTATACATCTGAGTTCTCTTGGTAAGTTCTTTAGCAGTGTTCAAAGTCTACTAGCTCG
CATTAGTTTCTGTTGCTGCCAACAGATCTGAACTAATGCTAACAGATCCCCCTGAGGGATTCTTGATGGG
CTGAGCAGCTGGCTGGAGCTAGTACTGACTGACATTCATTGTGATGAGGGCAGCTTTCTGGTACAGGATT
CTAAGCTCTATGTTTTATATACATTTTCATCTGTACTTGCACCTCACTTTACACAAGAGGAAACTATGCA
AAGTTAGCTGGATCGCTCAAGGTCACTTAGGTAAGTTGGCAAGTCCATGCTTCCCACTCAGCTCCTCAGG
TCAGCAAGTCTACTTCTCTGCCTATTTTGTATACTCTCTTTAATATGTGCCTAGCTTTGGAAAGTCTAGA
ATGGGTCCCTGGTGCCTTTTTACTTTGAAGAAATCAGTTTCTGCCTCTTTTTGGAAAAGAAAACAAAGTG
CAATTGTTTTTTACTGGAAAGTTACCCAATAGCATGAGGTGAACAGGACGTAGTTAGGCCTTCCTGTAAA
CAGAAAATCATATCAAAACACTATCTTCCCATCTGTTTCTCAATGCCTGCTACTTCTTGTAGATATTTCA
TTTCAGGAGAGCAGCAGTTAAACCCGTGGATTTTGTAGTTAGGAACCTGGGTTCAAACCCTCTTCCACTA
ATTGGCTATGTCTCTGGACAAGTTTTTTTTTTTTTTTTTTTTTAAACCCTTTCTGAACTTTCACTTTCTA
TGTCTACCTCAAAGAATTGTTGTGAGGCTTGAGATAATGCATTTGTAAAGGGTCTGCCAGATAGGAAGAT
GCTAGTTATGGATTTACAAGGTTGTTAAGGCTGTAAGAGTCTAAAACCTACAGTGAATCACAATGCATTT
ACCCCCACTGACTTGGACATAAGTGAAAACTAGCCAGAAGTCTCTTTTTCAAATTACTTACAGGTTATTC
AATATAAAATTTTTGTAATGGATAATCTTATTTATCTAAACTAAAGCTTCCTGTTTATACACACTCCTGT
TATTCTGGGATAAGATAAATGACCACAGTACCTTAATTTCTAGGTGGGTGCCTGTGATGGTTCATTGTAG
GTAAGGACATTTTCTCTTTTTCAGCAGCTGTGTAGGTCCAGAGCCTCTGGGAGAGGAGGGGGGTAGCATG
CACCCAGCAGGGGACTGAACTGGGAAACTCAAGGTTCTTTTTACTGTGGGGTAGTGAGCTGCCTTTCTGT
GATCGGTTTCCCTAGGGATGTTGCTGTTCCCCTCCTTGCTATTCGCAGCTACATACAACGTGGCCAACCC
CAGTAGGCTGATCCTATATATGATCAGTGCTGGTGCTGACTCTCAATAGCCCCACCCAAGCTGGCTATAG
GTTTACAGATACATTAATTAGGCAACCTAAAATATTGATGCTGGTGTTGGTGTGACATAATGCTATGGCC
AGAACTGAAACTTAGAGTTATAATTCATGTATTAGGGTTCTCCAGAGGGACAGAATTAGTAGGATATATG
TATATATGAAAGGGAGGTTATTAGGGAGAACTGGCTCCCACAGTTAGAAGGCGAAGTCGCACAATAGGCC
GTCTGCAAGCTGGGTTAGAGAGAAGCCAGTAGTGGCTCAGCCTGAGTTCAAAAACCTCAAAACTGGGGAA
GCTGACAGTGCAGCCAGCCTTCAGTCTGTGGCCAAAGGCCCAAGAGCCCCTGGCAACCAACCCACTGGTG
CAAGTCCTAGATTCCAAAGGCTGAAGAACCTGGAGTCTGATGTCCAAGAGCAGGAAGAGTGGAAGAAAGC
CAGAAGACTCAGCAAACAAGGTAGACAGTGTCTACCACCATAGTGGCCATACCAAAGAGGCTACCGATTC
CTTCCTGCTACCTGGATCCCTGAAGTTGCCCTGGTCTCTGCACCTTCTAAACCTAGTTCTTAAGAGCTTT
CCATTACATGAGCTGTCTCAAAGCCCTCCAATAAATTCTCAGTGTAAGCTTCTGTTGCTTGTGGACAGAA
AATTCTGACAGACCTACCCTATAAGTGTTACTGTCAGGATAACATGAGAACGCACAACAGTAAGTGGTCA
CTAAGTGTTAGCTACGGTTATTTTGCCCAAGGTAGCATGGCTAGTTGATGCCGGTTGATGGGGCTTAAAC
CCAGCTCCCTCATCTTCCAGGCCTCTGTACTCCCTATTCCACTAAACTACCTCTCAGGTTTATTTTTTTA
AATTCTTACTCTGCAAGTACATAGGACCACATTTACCTGGGAAAACAAGAATAAAGGCTGCTCTGCATTT
TTTAGAAACTTTTTTGAAAGGGAGATGGGAATGCCTGCACCCCCAAGTCCAGACCAACACAATGGTTAAT
TGAGATGAATAATAAAGGAAAGACTGTTCTGGGCTTCCCAGAATAGCTTGGTCCTTAAATTGTGGCACAA
ACAACCTCCTGTCAGAGCCAGCCTCCTGCCAGGAAGAGGGGTAGGAGACTAGAGGCCGTGTGTGCAGCCT
TGCCCTGAAGGCTAGGGTGACAATTTGGAGGCTGTCCAAACACCCTGGCCTCTAGAGCTGGCCTGTCTAT
TTGAAATGCCGGCTCTGATGCTAATCGGCGACCCTCAGGCAAGTTACTTAACCTTACATGCCTCAGTTTT
CTCATCTGGAAAATGAGAACCCTAGGTTTAGGGTTGTTAGAAAAGTTAAATGAGTTAAGACAAGTGCCTG
GGACACAGTAGCCTCTTGTGTGTGTTTATCATTATGTCCTCAGCAGGTCGTAGAAGCAGCTTCTCAGGTG
TGAGGCTGGCGCGATTATCTGGAGTGGGTTGGGTTTTCTAGGATGGACCCCCTGCTGCATTTTCCTCATT
CATCCACCAGGGCTTAATGGGGAATCAAGGAATCCATGTGTAACTGTATAATAACTGTAGCCACACTCCA
ATGACCACCTACTAGTTGTCCCTGGCACTGCTTATACATATGTCCATCAAATCAATCCTATGAAGTAGAT
ACTGTCTTCATTTTATAGATCAGAGACAATTGGGGTTCAGAGAGCTGATGTGATTTTCCCAGGGTCACAG
AGAGTCCCAGATTCAGGCACAACTCTTGTATTCCAAGACACAACCACTACATGTCCAAAGGCTGCCCAGA
GCCACCGGGCACGGCAAATTGTGACATATCCCTAAAGAGGCTGAGCACCTGGTCAGGATCTGATGGCTGA
CAGTGTGTCCAGATGCAGAGCTGGAGTGGGGGAGGGGAAGGGGGGCTCCTTGGGACAGAGAAGGCTTTCT
GTGCTTTCTCTGAAGGGAGCAGTCTGAGGACCAAGGGAACCCGGCAAACAGCACCTCAGGTACTCCAGGC
CCTCCTGGCTGGAGAGGGCTGTGGCAATGGAAAATTAGTGCCAACTGCAATGAGTCAGCCTCGGTTAAAT
AGAGAGTGAAGAATGCTGGACAGGAACCTCCACCCTCATGTCACATTTCTTCAGTGTGACCCTTCTGGCC
CCTCTCCTCCTGACAGCGGAACAATGACTGCCCCGATAGGTGAGGCTGGAGGAAGAATCAGTCCTGTCCT
TGGCAAGCTCTTCACTATGACAGTAAAGGCTCTCTGCCTGCTGCCAAGGCCTGTGACTTTCTAACCTGGC
CTCACGCTGGGTAAGCTTAAGGTAGAGGTGCAGGATTAGCAAGCCCACCTGGCTACCAGGCCGACAGCTA
CATCTTTCAACTGACCCTGATCAACGAAGAGGGACTTGTGTCTCTCAGTTGGTTCCAAATGAAACCAGGG
AGCAGGGGCGTTAGGAAGCTCCAACAGGATGGTACTTAATGGGGCATTTGAGTGGAGAGGTAGGTGACAT
AGTGCTTTGGAGCCCAGGGAGGGAAAGGTTCTGCTGAAGTTGAATTCAAGACTGTTCTTTCATCACAAAC
TTGAGTTTCCTGGACATTTGTTTGCAGAAACAACCGTAGGGTTTTGCCTTAACCTCGTGGGTTTATTATT
ACCTCATAGGGACTTTGCCTCCTGACAGCAGTTTATGGGTGTTCATTGTGGCACTTGAGTTTTCTTGCAT
ACTTGTTAGAGAAACCAAGTTTGTCATCAACTTCTTATTTAACCCCCTGGCTATAACTTCATGGATTATG
TTATAATTAAGCCATCCAGAGTAAAATCTGTTTAGATTATCTTGGAGTAAGGGGGAAAAAATCTGTAATT
TTTTCTCCTCAACTAGATATATACATAAAAAATGATTGTATTGCTTCATTTAAAAAATATAACGCAAAAT
CTCTTTTCCTTCTAAAAAAAAAAAAAAAAAA (NM_001127223.1 Homo sapiens CD59
molecule (CD59 blood group) (CD59), transcript variant 5, mRNA)

In some embodiments, the nucleotide sequence of an mRNA encoding CD59 is set forth in SEQ ID NO:8:

ATTTGACTCACTGACCCTGATGGGGAAGATAGTGTTATTCCCACAGTGTGGTAGTACACACTGGGGCTTA
TAGGGACTGAGCCTACTCAAGGGTATATGGTGCTGTGGGTCAGAGCTGGGGCATGGCCCAGGGATTCAGT
GTGCCTTGACTCCCCCTGTTCTGTGGACAATCACAATGGGAATCCAAGGAGGGTCTGTCCTGTTCGGGCT
GCTGCTCGTCCTGGCTGTCTTCTGCCATTCAGGTCATAGCCTGCAGTGCTACAACTGTCCTAACCCAACT
GCTGACTGCAAAACAGCCGTCAATTGTTCATCTGATTTTGATGCGTGTCTCATTACCAAAGCTGGGTTAC
AAGTGTATAACAAGTGTTGGAAGTTTGAGCATTGCAATTTCAACGACGTCACAACCCGCTTGAGGGAAAA
TGAGCTAACGTACTACTGCTGCAAGAAGGACCTGTGTAACTTTAACGAACAGCTTGAAAATGGTGGGACA
TCCTTATCAGAGAAAACAGTTCTTCTGCTGGTGACTCCATTTCTGGCAGCAGCCTGGAGCCTTCATCCCT
AAGTCAACACCAGGAGAGCTTCTCCCAAACTCCCCGTTCCTGCGTAGTCCGCTTTCTCTTGCTGCCACAT
TCTAAAGGCTTGATATTTTCCAAATGGATCCTGTTGGGAAAGAATAAAATTAGCTTGAGCAACCTGGCTA
AGATAGAGGGGCTCTGGGAGACTTTGAAGACCAGTCCTGTTTGCAGGGAAGCCCCACTTGAAGGAAGAAG
TCTAAGAGTGAAGTAGGTGTGACTTGAACTAGATTGCATGCTTCCTCCTTTGCTCTTGGGAAGACCAGCT
TTGCAGTGACAGCTTGAGTGGGTTCTCTGCAGCCCTCAGATTATTTTTCCTCTGGCTCCTTGGATGTAGT
CAGTTAGCATCATTAGTACATCTTTGGAGGGTGGGGCAGGAGTATATGAGCATCCTCTCTCACATGGAAC
GCTTTCATAAACTTCAGGGATCCCGTGTTGCCATGGAGGCATGCCAAATGTTCCATATGTGGGTGTCAGT
CAGGGACAACAAGATCCTTAATGCAGAGCTAGAGGACTTCTGGCAGGGAAGTGGGGAAGTGTTCCAGATA
GCAGGGCATGAAAACTTAGAGAGGTACAAGTGGCTGAAAATCGAGTTTTTCCTCTGTCTTTAAATTTTAT
ATGGGCTTTGTTATCTTCCACTGGAAAAGTGTAATAGCATACATCAATGGTGTGTTAAAGCTATTTCCTT
GCCTTTTTTTTATTGGAATGGTAGGATATCTTGGCTTTGCCACACACAGTTACAGAGTGAACACTCTACT
ACATGTGACTGGCAGTATTAAGTGTGCTTATTTTAAATGTTACTGGTAGAAAGGCAGTTCAGGTATGTGT
GTATATAGTATGAATGCAGTGGGGACACCCTTTGTGGTTACAGTTTGAGACTTCCAAAGGTCATCCTTAA
TAACAACAGATCTGCAGGGGTATGTTTTACCATCTGCATCCAGCCTCCTGCTAACTCCTAGCTGACTCAG
CATAGATTGTATAAAATACCTTTGTAACGGCTCTTAGCACACTCACAGATGTTTGAGGCTTTCAGAAGCT
CTTCTAAAAAATGATACACACCTTTCACAAGGGCAAACTTTTTCCTTTTCCCTGTGTATTCTAGTGAATG
AATCTCAAGATTCAGTAGACCTAATGACATTTGTATTTTATGATCTTGGCTGTATTTAATGGCATAGGCT
GACTTTTGCAGATGGAGGAATTTCTTGATTAATGTTGAAAAAAAACCCTTGATTATACTCTGTTGGACAA
ACCGAGTGCAATGAATGATGCTTTTCTGAAAATGAAATATAACAAGTGGGTGAATGTGGTTATGGCCGAA
AAGGATATGCAGTATGCTTAATGGTAGCAACTGAAAGAAGACATCCTGAGCAGTGCCAGCTTTCTTCTGT
TGATGCCGTTCCCTGAACATAGGAAAATAGAAACTTGCTTATCAAAACTTAGCATTACCTTGGTGCTCTG
TGTTCTCTGTTAGCTCAGTGTCTTTCCTTACATCAATAGGTTTTTTTTTTTTTTTTTGGCCTGAGGAAGT
ACTGACCATGCCCACAGCCACCGGCTGAGCAAAGAAGCTCATTTCATGTGAGTTCTAAGGAATGAGAAAC
AATTTTGATGAATTTAAGCAGAAAATGAATTTCTGGGAACTTTTTTGGGGGGGGGGGGGTGGGGAATTCA
GCCACACTCCAGAAAGCCAGGAGTCGACAGTTTTGGAAGCCTCTCTCAGGATTGAGATTCTAGGATGAGA
TTGGCTTACTGCTATCTTGTGTCATGTACCCACTTTTTGGCCAGACTACACTGGGAAGAAGGTAGTCCTC
TAAAGCAAAATCTGAGTGCCACTAAATGGGGAGATGGGGCTGTTAAGCTGTCCAAATCAACAAGGGTCAT
ATAAATGGCCTTAAACTTTGGGGTTGCTTTCTGCAAAAAGTTGCTGTGACTCATGCCATAGACAAGGTTG
AGTGCCTGGACCCAAAGGCAATACTGTAATGTAAAGACATTTATAGTACTAGGCAAACAGCACCCCAGGT
ACTCCAGGCCCTCCTGGCTGGAGAGGGCTGTGGCAATAGAAAATTAGTGCCAACTGCAGTGAGTCAGCCT
AGGTTAAATAGAGAGTGTAAGAGTGCTGGACAGGAACCTCCACCCTCATGTCACATTTCTTCAATGTGAC
CCTTCTGGCCCCTCTCCTCCTGACAGCGGAACAATGACTGCCCCGATAGGTGAGGCTGGAGGAAGAATCA
GTCCTGTCCTTGGCAAGCTCTTCACTATGACAGTAAAGGCTCTCTGCCTGCTGCCAAGGCCTGTGACTTT
CTAACCTGGCCTCACGCTGGGTAAGCTTAAGGTAGAGGTGCAGGATTAGCAAGCCCACCTGGCTACCAGG
CCGACAGCTACATCCTCCAACTGACCCTGATCAACGAAGAGGGATTCATGTGTCTGTCTCAGTTGGTTCC
AAATGAAACCAGGGAGCAGGGGAGTTAGGAATCGAACACCAGTCATGCCTACTGGCTCTCTGCTCGAGAG
CCAATACCCTGTGCCCTCCACTCATCTGGATTTACAGGAACTGTCATAGTGTTCAGTATTGGGTGGTGAT
AAGCCCATTGGATTGTCCCCTTGGGGGGATGAGCTAGGGGTGCAAGGAACACCTGATGAGTAGATAAGTG
GAGCTCATGGTATTTCCTGAAAGATGCTAATCTATTTGCCAAACTTGGTCTTGAATGTACTGGGGGCTTC
AAGGTATGGGTATATTTTTCTTGTGTCCTTGCAGTTAGCCCCCATGTCTTATGTGTGTCCTGAAAAAATA
AGAGCCTGCCCAAGACTTTGGGCCTCTTGACAGAATTAACCACTTTTATACATCTGAGTTCTCTTGGTAA
GTTCTTTAGCAGTGTTCAAAGTCTACTAGCTCGCATTAGTTTCTGTTGCTGCCAACAGATCTGAACTAAT
GCTAACAGATCCCCCTGAGGGATTCTTGATGGGCTGAGCAGCTGGCTGGAGCTAGTACTGACTGACATTC
ATTGTGATGAGGGCAGCTTTCTGGTACAGGATTCTAAGCTCTATGTTTTATATACATTTTCATCTGTACT
TGCACCTCACTTTACACAAGAGGAAACTATGCAAAGTTAGCTGGATCGCTCAAGGTCACTTAGGTAAGTT
GGCAAGTCCATGCTTCCCACTCAGCTCCTCAGGTCAGCAAGTCTACTTCTCTGCCTATTTTGTATACTCT
CTTTAATATGTGCCTAGCTTTGGAAAGTCTAGAATGGGTCCCTGGTGCCTTTTTACTTTGAAGAAATCAG
TTTCTGCCTCTTTTTGGAAAAGAAAACAAAGTGCAATTGTTTTTTACTGGAAAGTTACCCAATAGCATGA
GGTGAACAGGACGTAGTTAGGCCTTCCTGTAAACAGAAAATCATATCAAAACACTATCTTCCCATCTGTT
TCTCAATGCCTGCTACTTCTTGTAGATATTTCATTTCAGGAGAGCAGCAGTTAAACCCGTGGATTTTGTA
GTTAGGAACCTGGGTTCAAACCCTCTTCCACTAATTGGCTATGTCTCTGGACAAGTTTTTTTTTTTTTTT
TTTTTTAAACCCTTTCTGAACTTTCACTTTCTATGTCTACCTCAAAGAATTGTTGTGAGGCTTGAGATAA
TGCATTTGTAAAGGGTCTGCCAGATAGGAAGATGCTAGTTATGGATTTACAAGGTTGTTAAGGCTGTAAG
AGTCTAAAACCTACAGTGAATCACAATGCATTTACCCCCACTGACTTGGACATAAGTGAAAACTAGCCAG
AAGTCTCTTTTTCAAATTACTTACAGGTTATTCAATATAAAATTTTTGTAATGGATAATCTTATTTATCT
AAACTAAAGCTTCCTGTTTATACACACTCCTGTTATTCTGGGATAAGATAAATGACCACAGTACCTTAAT
TTCTAGGTGGGTGCCTGTGATGGTTCATTGTAGGTAAGGACATTTTCTCTTTTTCAGCAGCTGTGTAGGT
CCAGAGCCTCTGGGAGAGGAGGGGGGTAGCATGCACCCAGCAGGGGACTGAACTGGGAAACTCAAGGTTC
TTTTTACTGTGGGGTAGTGAGCTGCCTTTCTGTGATCGGTTTCCCTAGGGATGTTGCTGTTCCCCTCCTT
GCTATTCGCAGCTACATACAACGTGGCCAACCCCAGTAGGCTGATCCTATATATGATCAGTGCTGGTGCT
GACTCTCAATAGCCCCACCCAAGCTGGCTATAGGTTTACAGATACATTAATTAGGCAACCTAAAATATTG
ATGCTGGTGTTGGTGTGACATAATGCTATGGCCAGAACTGAAACTTAGAGTTATAATTCATGTATTAGGG
TTCTCCAGAGGGACAGAATTAGTAGGATATATGTATATATGAAAGGGAGGTTATTAGGGAGAACTGGCTC
CCACAGTTAGAAGGCGAAGTCGCACAATAGGCCGTCTGCAAGCTGGGTTAGAGAGAAGCCAGTAGTGGCT
CAGCCTGAGTTCAAAAACCTCAAAACTGGGGAAGCTGACAGTGCAGCCAGCCTTCAGTCTGTGGCCAAAG
GCCCAAGAGCCCCTGGCAACCAACCCACTGGTGCAAGTCCTAGATTCCAAAGGCTGAAGAACCTGGAGTC
TGATGTCCAAGAGCAGGAAGAGTGGAAGAAAGCCAGAAGACTCAGCAAACAAGGTAGACAGTGTCTACCA
CCATAGTGGCCATACCAAAGAGGCTACCGATTCCTTCCTGCTACCTGGATCCCTGAAGTTGCCCTGGTCT
CTGCACCTTCTAAACCTAGTTCTTAAGAGCTTTCCATTACATGAGCTGTCTCAAAGCCCTCCAATAAATT
CTCAGTGTAAGCTTCTGTTGCTTGTGGACAGAAAATTCTGACAGACCTACCCTATAAGTGTTACTGTCAG
GATAACATGAGAACGCACAACAGTAAGTGGTCACTAAGTGTTAGCTACGGTTATTTTGCCCAAGGTAGCA
TGGCTAGTTGATGCCGGTTGATGGGGCTTAAACCCAGCTCCCTCATCTTCCAGGCCTCTGTACTCCCTAT
TCCACTAAACTACCTCTCAGGTTTATTTTTTTAAATTCTTACTCTGCAAGTACATAGGACCACATTTACC
TGGGAAAACAAGAATAAAGGCTGCTCTGCATTTTTTAGAAACTTTTTTGAAAGGGAGATGGGAATGCCTG
CACCCCCAAGTCCAGACCAACACAATGGTTAATTGAGATGAATAATAAAGGAAAGACTGTTCTGGGCTTC
CCAGAATAGCTTGGTCCTTAAATTGTGGCACAAACAACCTCCTGTCAGAGCCAGCCTCCTGCCAGGAAGA
GGGGTAGGAGACTAGAGGCCGTGTGTGCAGCCTTGCCCTGAAGGCTAGGGTGACAATTTGGAGGCTGTCC
AAACACCCTGGCCTCTAGAGCTGGCCTGTCTATTTGAAATGCCGGCTCTGATGCTAATCGGCGACCCTCA
GGCAAGTTACTTAACCTTACATGCCTCAGTTTTCTCATCTGGAAAATGAGAACCCTAGGTTTAGGGTTGT
TAGAAAAGTTAAATGAGTTAAGACAAGTGCCTGGGACACAGTAGCCTCTTGTGTGTGTTTATCATTATGT
CCTCAGCAGGTCGTAGAAGCAGCTTCTCAGGTGTGAGGCTGGCGCGATTATCTGGAGTGGGTTGGGTTTT
CTAGGATGGACCCCCTGCTGCATTTTCCTCATTCATCCACCAGGGCTTAATGGGGAATCAAGGAATCCAT
GTGTAACTGTATAATAACTGTAGCCACACTCCAATGACCACCTACTAGTTGTCCCTGGCACTGCTTATAC
ATATGTCCATCAAATCAATCCTATGAAGTAGATACTGTCTTCATTTTATAGATCAGAGACAATTGGGGTT
CAGAGAGCTGATGTGATTTTCCCAGGGTCACAGAGAGTCCCAGATTCAGGCACAACTCTTGTATTCCAAG
ACACAACCACTACATGTCCAAAGGCTGCCCAGAGCCACCGGGCACGGCAAATTGTGACATATCCCTAAAG
AGGCTGAGCACCTGGTCAGGATCTGATGGCTGACAGTGTGTCCAGATGCAGAGCTGGAGTGGGGGAGGGG
AAGGGGGGCTCCTTGGGACAGAGAAGGCTTTCTGTGCTTTCTCTGAAGGGAGCAGTCTGAGGACCAAGGG
AACCCGGCAAACAGCACCTCAGGTACTCCAGGCCCTCCTGGCTGGAGAGGGCTGTGGCAATGGAAAATTA
GTGCCAACTGCAATGAGTCAGCCTCGGTTAAATAGAGAGTGAAGAATGCTGGACAGGAACCTCCACCCTC
ATGTCACATTTCTTCAGTGTGACCCTTCTGGCCCCTCTCCTCCTGACAGCGGAACAATGACTGCCCCGAT
AGGTGAGGCTGGAGGAAGAATCAGTCCTGTCCTTGGCAAGCTCTTCACTATGACAGTAAAGGCTCTCTGC
CTGCTGCCAAGGCCTGTGACTTTCTAACCTGGCCTCACGCTGGGTAAGCTTAAGGTAGAGGTGCAGGATT
AGCAAGCCCACCTGGCTACCAGGCCGACAGCTACATCTTTCAACTGACCCTGATCAACGAAGAGGGACTT
GTGTCTCTCAGTTGGTTCCAAATGAAACCAGGGAGCAGGGGCGTTAGGAAGCTCCAACAGGATGGTACTT
AATGGGGCATTTGAGTGGAGAGGTAGGTGACATAGTGCTTTGGAGCCCAGGGAGGGAAAGGTTCTGCTGA
AGTTGAATTCAAGACTGTTCTTTCATCACAAACTTGAGTTTCCTGGACATTTGTTTGCAGAAACAACCGT
AGGGTTTTGCCTTAACCTCGTGGGTTTATTATTACCTCATAGGGACTTTGCCTCCTGACAGCAGTTTATG
GGTGTTCATTGTGGCACTTGAGTTTTCTTGCATACTTGTTAGAGAAACCAAGTTTGTCATCAACTTCTTA
TTTAACCCCCTGGCTATAACTTCATGGATTATGTTATAATTAAGCCATCCAGAGTAAAATCTGTTTAGAT
TATCTTGGAGTAAGGGGGAAAAAATCTGTAATTTTTTCTCCTCAACTAGATATATACATAAAAAATGATT
GTATTGCTTCATTTAAAAAATATAACGCAAAATCTCTTTTCCTTCTAA (NM_001127225.2 Homo
sapiens CD59 molecule (CD59 blood group) (CD59), transcript variant 6,
mRNA)

In some embodiments, the nucleotide sequence of an mRNA encoding CD59 is set forth in SEQ ID NO:9:

ATTTGACTCACTGACCCTGATGGGGAAGATAGTGTTATTCCCACAGTGTGGTAGTACACACTGGGGCTTA
TAGGGACTGAGCCTACTCAAGGGTTCTGTGGACAATCACAATGGGAATCCAAGGAGGGTCTGTCCTGTTC
GGGCTGCTGCTCGTCCTGGCTGTCTTCTGCCATTCAGGTCATAGCCTGCAGTGCTACAACTGTCCTAACC
CAACTGCTGACTGCAAAACAGCCGTCAATTGTTCATCTGATTTTGATGCGTGTCTCATTACCAAAGCTGG
GTTACAAGTGTATAACAAGTGTTGGAAGTTTGAGCATTGCAATTTCAACGACGTCACAACCCGCTTGAGG
GAAAATGAGCTAACGTACTACTGCTGCAAGAAGGACCTGTGTAACTTTAACGAACAGCTTGAAAATGGTG
GGACATCCTTATCAGAGAAAACAGTTCTTCTGCTGGTGACTCCATTTCTGGCAGCAGCCTGGAGCCTTCA
TCCCTAAGTCAACACCAGGAGAGCTTCTCCCAAACTCCCCGTTCCTGCGTAGTCCGCTTTCTCTTGCTGC
CACATTCTAAAGGCTTGATATTTTCCAAATGGATCCTGTTGGGAAAGAATAAAATTAGCTTGAGCAACCT
GGCTAAGATAGAGGGGCTCTGGGAGACTTTGAAGACCAGTCCTGTTTGCAGGGAAGCCCCACTTGAAGGA
AGAAGTCTAAGAGTGAAGTAGGTGTGACTTGAACTAGATTGCATGCTTCCTCCTTTGCTCTTGGGAAGAC
CAGCTTTGCAGTGACAGCTTGAGTGGGTTCTCTGCAGCCCTCAGATTATTTTTCCTCTGGCTCCTTGGAT
GTAGTCAGTTAGCATCATTAGTACATCTTTGGAGGGTGGGGCAGGAGTATATGAGCATCCTCTCTCACAT
GGAACGCTTTCATAAACTTCAGGGATCCCGTGTTGCCATGGAGGCATGCCAAATGTTCCATATGTGGGTG
TCAGTCAGGGACAACAAGATCCTTAATGCAGAGCTAGAGGACTTCTGGCAGGGAAGTGGGGAAGTGTTCC
AGATAGCAGGGCATGAAAACTTAGAGAGGTACAAGTGGCTGAAAATCGAGTTTTTCCTCTGTCTTTAAAT
TTTATATGGGCTTTGTTATCTTCCACTGGAAAAGTGTAATAGCATACATCAATGGTGTGTTAAAGCTATT
TCCTTGCCTTTTTTTTATTGGAATGGTAGGATATCTTGGCTTTGCCACACACAGTTACAGAGTGAACACT
CTACTACATGTGACTGGCAGTATTAAGTGTGCTTATTTTAAATGTTACTGGTAGAAAGGCAGTTCAGGTA
TGTGTGTATATAGTATGAATGCAGTGGGGACACCCTTTGTGGTTACAGTTTGAGACTTCCAAAGGTCATC
CTTAATAACAACAGATCTGCAGGGGTATGTTTTACCATCTGCATCCAGCCTCCTGCTAACTCCTAGCTGA
CTCAGCATAGATTGTATAAAATACCTTTGTAACGGCTCTTAGCACACTCACAGATGTTTGAGGCTTTCAG
AAGCTCTTCTAAAAAATGATACACACCTTTCACAAGGGCAAACTTTTTCCTTTTCCCTGTGTATTCTAGT
GAATGAATCTCAAGATTCAGTAGACCTAATGACATTTGTATTTTATGATCTTGGCTGTATTTAATGGCAT
AGGCTGACTTTTGCAGATGGAGGAATTTCTTGATTAATGTTGAAAAAAAACCCTTGATTATACTCTGTTG
GACAAACCGAGTGCAATGAATGATGCTTTTCTGAAAATGAAATATAACAAGTGGGTGAATGTGGTTATGG
CCGAAAAGGATATGCAGTATGCTTAATGGTAGCAACTGAAAGAAGACATCCTGAGCAGTGCCAGCTTTCT
TCTGTTGATGCCGTTCCCTGAACATAGGAAAATAGAAACTTGCTTATCAAAACTTAGCATTACCTTGGTG
CTCTGTGTTCTCTGTTAGCTCAGTGTCTTTCCTTACATCAATAGGTTTTTTTTTTTTTTTTTGGCCTGAG
GAAGTACTGACCATGCCCACAGCCACCGGCTGAGCAAAGAAGCTCATTTCATGTGAGTTCTAAGGAATGA
GAAACAATTTTGATGAATTTAAGCAGAAAATGAATTTCTGGGAACTTTTTTGGGGGGGGGGGGGTGGGGA
ATTCAGCCACACTCCAGAAAGCCAGGAGTCGACAGTTTTGGAAGCCTCTCTCAGGATTGAGATTCTAGGA
TGAGATTGGCTTACTGCTATCTTGTGTCATGTACCCACTTTTTGGCCAGACTACACTGGGAAGAAGGTAG
TCCTCTAAAGCAAAATCTGAGTGCCACTAAATGGGGAGATGGGGCTGTTAAGCTGTCCAAATCAACAAGG
GTCATATAAATGGCCTTAAACTTTGGGGTTGCTTTCTGCAAAAAGTTGCTGTGACTCATGCCATAGACAA
GGTTGAGTGCCTGGACCCAAAGGCAATACTGTAATGTAAAGACATTTATAGTACTAGGCAAACAGCACCC
CAGGTACTCCAGGCCCTCCTGGCTGGAGAGGGCTGTGGCAATAGAAAATTAGTGCCAACTGCAGTGAGTC
AGCCTAGGTTAAATAGAGAGTGTAAGAGTGCTGGACAGGAACCTCCACCCTCATGTCACATTTCTTCAAT
GTGACCCTTCTGGCCCCTCTCCTCCTGACAGCGGAACAATGACTGCCCCGATAGGTGAGGCTGGAGGAAG
AATCAGTCCTGTCCTTGGCAAGCTCTTCACTATGACAGTAAAGGCTCTCTGCCTGCTGCCAAGGCCTGTG
ACTTTCTAACCTGGCCTCACGCTGGGTAAGCTTAAGGTAGAGGTGCAGGATTAGCAAGCCCACCTGGCTA
CCAGGCCGACAGCTACATCCTCCAACTGACCCTGATCAACGAAGAGGGATTCATGTGTCTGTCTCAGTTG
GTTCCAAATGAAACCAGGGAGCAGGGGAGTTAGGAATCGAACACCAGTCATGCCTACTGGCTCTCTGCTC
GAGAGCCAATACCCTGTGCCCTCCACTCATCTGGATTTACAGGAACTGTCATAGTGTTCAGTATTGGGTG
GTGATAAGCCCATTGGATTGTCCCCTTGGGGGGATGAGCTAGGGGTGCAAGGAACACCTGATGAGTAGAT
AAGTGGAGCTCATGGTATTTCCTGAAAGATGCTAATCTATTTGCCAAACTTGGTCTTGAATGTACTGGGG
GCTTCAAGGTATGGGTATATTTTTCTTGTGTCCTTGCAGTTAGCCCCCATGTCTTATGTGTGTCCTGAAA
AAATAAGAGCCTGCCCAAGACTTTGGGCCTCTTGACAGAATTAACCACTTTTATACATCTGAGTTCTCTT
GGTAAGTTCTTTAGCAGTGTTCAAAGTCTACTAGCTCGCATTAGTTTCTGTTGCTGCCAACAGATCTGAA
CTAATGCTAACAGATCCCCCTGAGGGATTCTTGATGGGCTGAGCAGCTGGCTGGAGCTAGTACTGACTGA
CATTCATTGTGATGAGGGCAGCTTTCTGGTACAGGATTCTAAGCTCTATGTTTTATATACATTTTCATCT
GTACTTGCACCTCACTTTACACAAGAGGAAACTATGCAAAGTTAGCTGGATCGCTCAAGGTCACTTAGGT
AAGTTGGCAAGTCCATGCTTCCCACTCAGCTCCTCAGGTCAGCAAGTCTACTTCTCTGCCTATTTTGTAT
ACTCTCTTTAATATGTGCCTAGCTTTGGAAAGTCTAGAATGGGTCCCTGGTGCCTTTTTACTTTGAAGAA
ATCAGTTTCTGCCTCTTTTTGGAAAAGAAAACAAAGTGCAATTGTTTTTTACTGGAAAGTTACCCAATAG
CATGAGGTGAACAGGACGTAGTTAGGCCTTCCTGTAAACAGAAAATCATATCAAAACACTATCTTCCCAT
CTGTTTCTCAATGCCTGCTACTTCTTGTAGATATTTCATTTCAGGAGAGCAGCAGTTAAACCCGTGGATT
TTGTAGTTAGGAACCTGGGTTCAAACCCTCTTCCACTAATTGGCTATGTCTCTGGACAAGTTTTTTTTTT
TTTTTTTTTTTAAACCCTTTCTGAACTTTCACTTTCTATGTCTACCTCAAAGAATTGTTGTGAGGCTTGA
GATAATGCATTTGTAAAGGGTCTGCCAGATAGGAAGATGCTAGTTATGGATTTACAAGGTTGTTAAGGCT
GTAAGAGTCTAAAACCTACAGTGAATCACAATGCATTTACCCCCACTGACTTGGACATAAGTGAAAACTA
GCCAGAAGTCTCTTTTTCAAATTACTTACAGGTTATTCAATATAAAATTTTTGTAATGGATAATCTTATT
TATCTAAACTAAAGCTTCCTGTTTATACACACTCCTGTTATTCTGGGATAAGATAAATGACCACAGTACC
TTAATTTCTAGGTGGGTGCCTGTGATGGTTCATTGTAGGTAAGGACATTTTCTCTTTTTCAGCAGCTGTG
TAGGTCCAGAGCCTCTGGGAGAGGAGGGGGGTAGCATGCACCCAGCAGGGGACTGAACTGGGAAACTCAA
GGTTCTTTTTACTGTGGGGTAGTGAGCTGCCTTTCTGTGATCGGTTTCCCTAGGGATGTTGCTGTTCCCC
TCCTTGCTATTCGCAGCTACATACAACGTGGCCAACCCCAGTAGGCTGATCCTATATATGATCAGTGCTG
GTGCTGACTCTCAATAGCCCCACCCAAGCTGGCTATAGGTTTACAGATACATTAATTAGGCAACCTAAAA
TATTGATGCTGGTGTTGGTGTGACATAATGCTATGGCCAGAACTGAAACTTAGAGTTATAATTCATGTAT
TAGGGTTCTCCAGAGGGACAGAATTAGTAGGATATATGTATATATGAAAGGGAGGTTATTAGGGAGAACT
GGCTCCCACAGTTAGAAGGCGAAGTCGCACAATAGGCCGTCTGCAAGCTGGGTTAGAGAGAAGCCAGTAG
TGGCTCAGCCTGAGTTCAAAAACCTCAAAACTGGGGAAGCTGACAGTGCAGCCAGCCTTCAGTCTGTGGC
CAAAGGCCCAAGAGCCCCTGGCAACCAACCCACTGGTGCAAGTCCTAGATTCCAAAGGCTGAAGAACCTG
GAGTCTGATGTCCAAGAGCAGGAAGAGTGGAAGAAAGCCAGAAGACTCAGCAAACAAGGTAGACAGTGTC
TACCACCATAGTGGCCATACCAAAGAGGCTACCGATTCCTTCCTGCTACCTGGATCCCTGAAGTTGCCCT
GGTCTCTGCACCTTCTAAACCTAGTTCTTAAGAGCTTTCCATTACATGAGCTGTCTCAAAGCCCTCCAAT
AAATTCTCAGTGTAAGCTTCTGTTGCTTGTGGACAGAAAATTCTGACAGACCTACCCTATAAGTGTTACT
GTCAGGATAACATGAGAACGCACAACAGTAAGTGGTCACTAAGTGTTAGCTACGGTTATTTTGCCCAAGG
TAGCATGGCTAGTTGATGCCGGTTGATGGGGCTTAAACCCAGCTCCCTCATCTTCCAGGCCTCTGTACTC
CCTATTCCACTAAACTACCTCTCAGGTTTATTTTTTTAAATTCTTACTCTGCAAGTACATAGGACCACAT
TTACCTGGGAAAACAAGAATAAAGGCTGCTCTGCATTTTTTAGAAACTTTTTTGAAAGGGAGATGGGAAT
GCCTGCACCCCCAAGTCCAGACCAACACAATGGTTAATTGAGATGAATAATAAAGGAAAGACTGTTCTGG
GCTTCCCAGAATAGCTTGGTCCTTAAATTGTGGCACAAACAACCTCCTGTCAGAGCCAGCCTCCTGCCAG
GAAGAGGGGTAGGAGACTAGAGGCCGTGTGTGCAGCCTTGCCCTGAAGGCTAGGGTGACAATTTGGAGGC
TGTCCAAACACCCTGGCCTCTAGAGCTGGCCTGTCTATTTGAAATGCCGGCTCTGATGCTAATCGGCGAC
CCTCAGGCAAGTTACTTAACCTTACATGCCTCAGTTTTCTCATCTGGAAAATGAGAACCCTAGGTTTAGG
GTTGTTAGAAAAGTTAAATGAGTTAAGACAAGTGCCTGGGACACAGTAGCCTCTTGTGTGTGTTTATCAT
TATGTCCTCAGCAGGTCGTAGAAGCAGCTTCTCAGGTGTGAGGCTGGCGCGATTATCTGGAGTGGGTTGG
GTTTTCTAGGATGGACCCCCTGCTGCATTTTCCTCATTCATCCACCAGGGCTTAATGGGGAATCAAGGAA
TCCATGTGTAACTGTATAATAACTGTAGCCACACTCCAATGACCACCTACTAGTTGTCCCTGGCACTGCT
TATACATATGTCCATCAAATCAATCCTATGAAGTAGATACTGTCTTCATTTTATAGATCAGAGACAATTG
GGGTTCAGAGAGCTGATGTGATTTTCCCAGGGTCACAGAGAGTCCCAGATTCAGGCACAACTCTTGTATT
CCAAGACACAACCACTACATGTCCAAAGGCTGCCCAGAGCCACCGGGCACGGCAAATTGTGACATATCCC
TAAAGAGGCTGAGCACCTGGTCAGGATCTGATGGCTGACAGTGTGTCCAGATGCAGAGCTGGAGTGGGGG
AGGGGAAGGGGGGCTCCTTGGGACAGAGAAGGCTTTCTGTGCTTTCTCTGAAGGGAGCAGTCTGAGGACC
AAGGGAACCCGGCAAACAGCACCTCAGGTACTCCAGGCCCTCCTGGCTGGAGAGGGCTGTGGCAATGGAA
AATTAGTGCCAACTGCAATGAGTCAGCCTCGGTTAAATAGAGAGTGAAGAATGCTGGACAGGAACCTCCA
CCCTCATGTCACATTTCTTCAGTGTGACCCTTCTGGCCCCTCTCCTCCTGACAGCGGAACAATGACTGCC
CCGATAGGTGAGGCTGGAGGAAGAATCAGTCCTGTCCTTGGCAAGCTCTTCACTATGACAGTAAAGGCTC
TCTGCCTGCTGCCAAGGCCTGTGACTTTCTAACCTGGCCTCACGCTGGGTAAGCTTAAGGTAGAGGTGCA
GGATTAGCAAGCCCACCTGGCTACCAGGCCGACAGCTACATCTTTCAACTGACCCTGATCAACGAAGAGG
GACTTGTGTCTCTCAGTTGGTTCCAAATGAAACCAGGGAGCAGGGGCGTTAGGAAGCTCCAACAGGATGG
TACTTAATGGGGCATTTGAGTGGAGAGGTAGGTGACATAGTGCTTTGGAGCCCAGGGAGGGAAAGGTTCT
GCTGAAGTTGAATTCAAGACTGTTCTTTCATCACAAACTTGAGTTTCCTGGACATTTGTTTGCAGAAACA
ACCGTAGGGTTTTGCCTTAACCTCGTGGGTTTATTATTACCTCATAGGGACTTTGCCTCCTGACAGCAGT
TTATGGGTGTTCATTGTGGCACTTGAGTTTTCTTGCATACTTGTTAGAGAAACCAAGTTTGTCATCAACT
TCTTATTTAACCCCCTGGCTATAACTTCATGGATTATGTTATAATTAAGCCATCCAGAGTAAAATCTGTT
TAGATTATCTTGGAGTAAGGGGGAAAAAATCTGTAATTTTTTCTCCTCAACTAGATATATACATAAAAAA
TGATTGTATTGCTTCATTTAAAAAATATAACGCAAAATCTCTTTTCCTTCTAA (NM_001127226.2
Homo sapiens CD59 molecule (CD59 blood group) (CD59), transcript
variant 7, mRNA)

In some embodiments, the nucleotide sequence of an mRNA encoding CD59 is set forth in SEQ ID NO:10:

ATACTTCTTACAGTGAGGAGCCAGAGCTTCCAGGTTCTGTGGACAATCACAATGGGAATCCAAGGAGGGT
CTGTCCTGTTCGGGCTGCTGCTCGTCCTGGCTGTCTTCTGCCATTCAGGTCATAGCCTGCAGTGCTACAA
CTGTCCTAACCCAACTGCTGACTGCAAAACAGCCGTCAATTGTTCATCTGATTTTGATGCGTGTCTCATT
ACCAAAGCTGGGTTACAAGTGTATAACAAGTGTTGGAAGTTTGAGCATTGCAATTTCAACGACGTCACAA
CCCGCTTGAGGGAAAATGAGCTAACGTACTACTGCTGCAAGAAGGACCTGTGTAACTTTAACGAACAGCT
TGAAAATGGTGGGACATCCTTATCAGAGAAAACAGTTCTTCTGCTGGTGACTCCATTTCTGGCAGCAGCC
TGGAGCCTTCATCCCTAAGTCAACACCAGGAGAGCTTCTCCCAAACTCCCCGTTCCTGCGTAGTCCGCTT
TCTCTTGCTGCCACATTCTAAAGGCTTGATATTTTCCAAATGGATCCTGTTGGGAAAGAATAAAATTAGC
TTGAGCAACCTGGCTAAGATAGAGGGGCTCTGGGAGACTTTGAAGACCAGTCCTGTTTGCAGGGAAGCCC
CACTTGAAGGAAGAAGTCTAAGAGTGAAGTAGGTGTGACTTGAACTAGATTGCATGCTTCCTCCTTTGCT
CTTGGGAAGACCAGCTTTGCAGTGACAGCTTGAGTGGGTTCTCTGCAGCCCTCAGATTATTTTTCCTCTG
GCTCCTTGGATGTAGTCAGTTAGCATCATTAGTACATCTTTGGAGGGTGGGGCAGGAGTATATGAGCATC
CTCTCTCACATGGAACGCTTTCATAAACTTCAGGGATCCCGTGTTGCCATGGAGGCATGCCAAATGTTCC
ATATGTGGGTGTCAGTCAGGGACAACAAGATCCTTAATGCAGAGCTAGAGGACTTCTGGCAGGGAAGTGG
GGAAGTGTTCCAGATAGCAGGGCATGAAAACTTAGAGAGGTACAAGTGGCTGAAAATCGAGTTTTTCCTC
TGTCTTTAAATTTTATATGGGCTTTGTTATCTTCCACTGGAAAAGTGTAATAGCATACATCAATGGTGTG
TTAAAGCTATTTCCTTGCCTTTTTTTTATTGGAATGGTAGGATATCTTGGCTTTGCCACACACAGTTACA
GAGTGAACACTCTACTACATGTGACTGGCAGTATTAAGTGTGCTTATTTTAAATGTTACTGGTAGAAAGG
CAGTTCAGGTATGTGTGTATATAGTATGAATGCAGTGGGGACACCCTTTGTGGTTACAGTTTGAGACTTC
CAAAGGTCATCCTTAATAACAACAGATCTGCAGGGGTATGTTTTACCATCTGCATCCAGCCTCCTGCTAA
CTCCTAGCTGACTCAGCATAGATTGTATAAAATACCTTTGTAACGGCTCTTAGCACACTCACAGATGTTT
GAGGCTTTCAGAAGCTCTTCTAAAAAATGATACACACCTTTCACAAGGGCAAACTTTTTCCTTTTCCCTG
TGTATTCTAGTGAATGAATCTCAAGATTCAGTAGACCTAATGACATTTGTATTTTATGATCTTGGCTGTA
TTTAATGGCATAGGCTGACTTTTGCAGATGGAGGAATTTCTTGATTAATGTTGAAAAAAAACCCTTGATT
ATACTCTGTTGGACAAACCGAGTGCAATGAATGATGCTTTTCTGAAAATGAAATATAACAAGTGGGTGAA
TGTGGTTATGGCCGAAAAGGATATGCAGTATGCTTAATGGTAGCAACTGAAAGAAGACATCCTGAGCAGT
GCCAGCTTTCTTCTGTTGATGCCGTTCCCTGAACATAGGAAAATAGAAACTTGCTTATCAAAACTTAGCA
TTACCTTGGTGCTCTGTGTTCTCTGTTAGCTCAGTGTCTTTCCTTACATCAATAGGTTTTTTTTTTTTTT
TTTGGCCTGAGGAAGTACTGACCATGCCCACAGCCACCGGCTGAGCAAAGAAGCTCATTTCATGTGAGTT
CTAAGGAATGAGAAACAATTTTGATGAATTTAAGCAGAAAATGAATTTCTGGGAACTTTTTTGGGGGCGG
GGGGGTGGGGAATTCAGCCACACTCCAGAAAGCCAGGAGTCGACAGTTTTGGAAGCCTCTCTCAGGATTG
AGATTCTAGGATGAGATTGGCTTACTGCTATCTTGTGTCATGTACCCACTTTTTGGCCAGACTACACTGG
GAAGAAGGTAGTCCTCTAAAGCAAAATCTGAGTGCCACTAAATGGGGAGATGGGGCTGTTAAGCTGTCCA
AATCAACAAGGGTCATATAAATGGCCTTAAACTTTGGGGTTGCTTTCTGCAAAAAGTTGCTGTGACTCAT
GCCATAGACAAGGTTGAGTGCCTGGACCCAAAGGCAATACTGTAATGTAAAGACATTTATAGTACTAGGC
AAACAGCACCCCAGGTACTCCAGGCCCTCCTGGCTGGAGAGGGCTGTGGCAATAGAAAATTAGTGCCAAC
TGCAGTGAGTCAGCCTAGGTTAAATAGAGAGTGTAAGAGTGCTGGACAGGAACCTCCACCCTCATGTCAC
ATTTCTTCAATGTGACCCTTCTGGCCCCTCTCCTCCTGACAGCGGAACAATGACTGCCCCGATAGGTGAG
GCTGGAGGAAGAATCAGTCCTGTCCTTGGCAAGCTCTTCACTATGACAGTAAAGGCTCTCTGCCTGCTGC
CAAGGCCTGTGACTTTCTAACCTGGCCTCACGCTGGGTAAGCTTAAGGTAGAGGTGCAGGATTAGCAAGC
CCACCTGGCTACCAGGCCGACAGCTACATCCTCCAACTGACCCTGATCAACGAAGAGGGATTCATGTGTC
TGTCTCAGTTGGTTCCAAATGAAACCAGGGAGCAGGGGAGTTAGGAATCGAACACCAGTCATGCCTACTG
GCTCTCTGCTCGAGAGCCAATACCCTGTGCCCTCCACTCATCTGGATTTACAGGAACTGTCATAGTGTTC
AGTATTGGGTGGTGATAAGCCCATTGGATTGTCCCCTTGGGGGGATGAGCTAGGGGTGCAAGGAACACCT
GATGAGTAGATAAGTGGAGCTCATGGTATTTCCTGAAAGATGCTAATCTATTTGCCAAACTTGGTCTTGA
ATGTACTGGGGGCTTCAAGGTATGGGTATATTTTTCTTGTGTCCTTGCAGTTAGCCCCCATGTCTTATGT
GTGTCCTGAAAAAATAAGAGCCTGCCCAAGACTTTGGGCCTCTTGACAGAATTAACCACTTTTATACATC
TGAGTTCTCTTGGTAAGTTCTTTAGCAGTGTTCAAAGTCTACTAGCTCGCATTAGTTTCTGTTGCTGCCA
ACAGATCTGAACTAATGCTAACAGATCCCCCTGAGGGATTCTTGATGGGCTGAGCAGCTGGCTGGAGCTA
GTACTGACTGACATTCATTGTGATGAGGGCAGCTTTCTGGTACAGGATTCTAAGCTCTATGTTTTATATA
CATTTTCATCTGTACTTGCACCTCACTTTACACAAGAGGAAACTATGCAAAGTTAGCTGGATCGCTCAAG
GTCACTTAGGTAAGTTGGCAAGTCCATGCTTCCCACTCAGCTCCTCAGGTCAGCAAGTCTACTTCTCTGC
CTATTTTGTATACTCTCTTTAATATGTGCCTAGCTTTGGAAAGTCTAGAATGGGTCCCTGGTGCCTTTTT
ACTTTGAAGAAATCAGTTTCTGCCTCTTTTTGGAAAAGAAAACAAAGTGCAATTGTTTTTTACTGGAAAG
TTACCCAATAGCATGAGGTGAACAGGACGTAGTTAGGCCTTCCTGTAAACAGAAAATCATATCAAAACAC
TATCTTCCCATCTGTTTCTCAATGCCTGCTACTTCTTGTAGATATTTCATTTCAGGAGAGCAGCAGTTAA
ACCCGTGGATTTTGTAGTTAGGAACCTGGGTTCAAACCCTCTTCCACTAATTGGCTATGTCTCTGGACAA
GTTTTTTTTTTTTTTTTTTTTTAAACCCTTTCTGAACTTTCACTTTCTATGTCTACCTCAAAGAATTGTT
GTGAGGCTTGAGATAATGCATTTGTAAAGGGTCTGCCAGATAGGAAGATGCTAGTTATGGATTTACAAGG
TTGTTAAGGCTGTAAGAGTCTAAAACCTACAGTGAATCACAATGCATTTACCCCCACTGACTTGGACATA
AGTGAAAACTAGCCAGAAGTCTCTTTTTCAAATTACTTACAGGTTATTCAATATAAAATTTTTGTAATGG
ATAATCTTATTTATCTAAACTAAAGCTTCCTGTTTATACACACTCCTGTTATTCTGGGATAAGATAAATG
ACCACAGTACCTTAATTTCTAGGTGGGTGCCTGTGATGGTTCATTGTAGGTAAGGACATTTTCTCTTTTT
CAGCAGCTGTGTAGGTCCAGAGCCTCTGGGAGAGGAGGGGGGTAGCATGCACCCAGCAGGGGACTGAACT
GGGAAACTCAAGGTTCTTTTTACTGTGGGGTAGTGAGCTGCCTTTCTGTGATCGGTTTCCCTAGGGATGT
TGCTGTTCCCCTCCTTGCTATTCGCAGCTACATACAACGTGGCCAACCCCAGTAGGCTGATCCTATATAT
GATCAGTGCTGGTGCTGACTCTCAATAGCCCCACCCAAGCTGGCTATAGGTTTACAGATACATTAATTAG
GCAACCTAAAATATTGATGCTGGTGTTGGTGTGACATAATGCTATGGCCAGAACTGAAACTTAGAGTTAT
AATTCATGTATTAGGGTTCTCCAGAGGGACAGAATTAGTAGGATATATGTATATATGAAAGGGAGGTTAT
TAGGGAGAACTGGCTCCCACAGTTAGAAGGCGAAGTCGCACAATAGGCCGTCTGCAAGCTGGGTTAGAGA
GAAGCCAGTAGTGGCTCAGCCTGAGTTCAAAAACCTCAAAACTGGGGAAGCTGACAGTGCAGCCAGCCTT
CAGTCTGTGGCCAAAGGCCCAAGAGCCCCTGGCAACCAACCCACTGGTGCAAGTCCTAGATTCCAAAGGC
TGAAGAACCTGGAGTCTGATGTCCAAGAGCAGGAAGAGTGGAAGAAAGCCAGAAGACTCAGCAAACAAGG
TAGACAGTGTCTACCACCATAGTGGCCATACCAAAGAGGCTACCGATTCCTTCCTGCTACCTGGATCCCT
GAAGTTGCCCTGGTCTCTGCACCTTCTAAACCTAGTTCTTAAGAGCTTTCCATTACATGAGCTGTCTCAA
AGCCCTCCAATAAATTCTCAGTGTAAGCTTCTGTTGCTTGTGGACAGAAAATTCTGACAGACCTACCCTA
TAAGTGTTACTGTCAGGATAACATGAGAACGCACAACAGTAAGTGGTCACTAAGTGTTAGCTACGGTTAT
TTTGCCCAAGGTAGCATGGCTAGTTGATGCCGGTTGATGGGGCTTAAACCCAGCTCCCTCATCTTCCAGG
CCTCTGTACTCCCTATTCCACTAAACTACCTCTCAGGTTTATTTTTTTAAATTCTTACTCTGCAAGTACA
TAGGACCACATTTACCTGGGAAAACAAGAATAAAGGCTGCTCTGCATTTTTTAGAAACTTTTTTGAAAGG
GAGATGGGAATGCCTGCACCCCCAAGTCCAGACCAACACAATGGTTAATTGAGATGAATAATAAAGGAAA
GACTGTTCTGGGCTTCCCAGAATAGCTTGGTCCTTAAATTGTGGCACAAACAACCTCCTGTCAGAGCCAG
CCTCCTGCCAGGAAGAGGGGTAGGAGACTAGAGGCCGTGTGTGCAGCCTTGCCCTGAAGGCTAGGGTGAC
AATTTGGAGGCTGTCCAAACACCCTGGCCTCTAGAGCTGGCCTGTCTATTTGAAATGCCGGCTCTGATGC
TAATCGGCGACCCTCAGGCAAGTTACTTAACCTTACATGCCTCAGTTTTCTCATCTGGAAAATGAGAACC
CTAGGTTTAGGGTTGTTAGAAAAGTTAAATGAGTTAAGACAAGTGCCTGGGACACAGTAGCCTCTTGTGT
GTGTTTATCATTATGTCCTCAGCAGGTCGTAGAAGCAGCTTCTCAGGTGTGAGGCTGGCGCGATTATCTG
GAGTGGGTTGGGTTTTCTAGGATGGACCCCCTGCTGCATTTTCCTCATTCATCCACCAGGGCTTAATGGG
GAATCAAGGAATCCATGTGTAACTGTATAATAACTGTAGCCACACTCCAATGACCACCTACTAGTTGTCC
CTGGCACTGCTTATACATATGTCCATCAAATCAATCCTATGAAGTAGATACTGTCTTCATTTTATAGATC
AGAGACAATTGGGGTTCAGAGAGCTGATGTGATTTTCCCAGGGTCACAGAGAGTCCCAGATTCAGGCACA
ACTCTTGTATTCCAAGACACAACCACTACATGTCCAAAGGCTGCCCAGAGCCACCGGGCACGGCAAATTG
TGACATATCCCTAAAGAGGCTGAGCACCTGGTCAGGATCTGATGGCTGACAGTGTGTCCAGATGCAGAGC
TGGAGTGGGGGAGGGGAAGGGGGGCTCCTTGGGACAGAGAAGGCTTTCTGTGCTTTCTCTGAAGGGAGCA
GTCTGAGGACCAAGGGAACCCGGCAAACAGCACCTCAGGTACTCCAGGCCCTCCTGGCTGGAGAGGGCTG
TGGCAATGGAAAATTAGTGCCAACTGCAATGAGTCAGCCTCGGTTAAATAGAGAGTGAAGAATGCTGGAC
AGGAACCTCCACCCTCATGTCACATTTCTTCAGTGTGACCCTTCTGGCCCCTCTCCTCCTGACAGCGGAA
CAATGACTGCCCCGATAGGTGAGGCTGGAGGAAGAATCAGTCCTGTCCTTGGCAAGCTCTTCACTATGAC
AGTAAAGGCTCTCTGCCTGCTGCCAAGGCCTGTGACTTTCTAACCTGGCCTCACGCTGGGTAAGCTTAAG
GTAGAGGTGCAGGATTAGCAAGCCCACCTGGCTACCAGGCCGACAGCTACATCTTTCAACTGACCCTGAT
CAACGAAGAGGGACTTGTGTCTCTCAGTTGGTTCCAAATGAAACCAGGGAGCAGGGGCGTTAGGAAGCTC
CAACAGGATGGTACTTAATGGGGCATTTGAGTGGAGAGGTAGGTGACATAGTGCTTTGGAGCCCAGGGAG
GGAAAGGTTCTGCTGAAGTTGAATTCAAGACTGTTCTTTCATCACAAACTTGAGTTTCCTGGACATTTGT
TTGCAGAAACAACCGTAGGGTTTTGCCTTAACCTCGTGGGTTTATTATTACCTCATAGGGACTTTGCCTC
CTGACAGCAGTTTATGGGTGTTCATTGTGGCACTTGAGTTTTCTTGCATACTTGTTAGAGAAACCAAGTT
TGTCATCAACTTCTTATTTAACCCCCTGGCTATAACTTCATGGATTATGTTATAATTAAGCCATCCAGAG
TAAAATCTGTTTAGATTATCTTGGAGTAAGGGGGAAAAAATCTGTAATTTTTTCTCCTCAACTAGATATA
TACATAAAAAATGATTGTATTGCTTCATTTAAAAAATATAACGCAAAATCTCTTTTCCTTCTAA
(NM_001127227.2 Homo sapiens CD59 molecule (CD59 blood group) (CD59) ,
transcript variant 8, mRNA)

In some embodiments, the nucleotide sequence of the full open reading frame (ORF) of a cDNA clone for CD59 is set forth in SEQ ID NO:11:

ATGGGAATCCAAGGAGGGTCTGTCCTGTTCGGGCTGCTGCTCGTCCTGG
CTGTCTTCTGCCATTCAGGTCATAGCCTGCAGTGCTACAACTGTCCTAA
CCCAACTGCTGACTGCAAAACAGCCGTCAATTGTTCATCTGATTTTGAT
GCGTGTCTCATTACCAAAGCTGGGTTACAAGTGTATAACAAGTGTTGGA
AGTTTGAGCATTGCAATTTCAACGACGTCACAACCCGCTTGAGGGAAAA
TGAGCTAACGTACTACTGCTGCAAGAAGGACCTGTGTAACTTTAACGAA
CAGCTTGAAAATGGTGGGACATCCTTATCAGAGAAAACAGTTCTTCTGC
TGGTGACTCCATTTCTGGCAGCAGCCTGGAGCCTTCATCCCTAA
(CR541722.1 Homo sapiens full open reading frame
cDNA clone RZPDo834D0829D for gene CD59, CD59
antigen p18-20 (antigen identified by monoclonal
antibodies 16.3A5, EJ16, EJ30, EL32 and G344);
complete cds, incl. stop codon).

In some embodiments, the nucleotide sequence of the full open reading frame (ORF) of a cDNA clone for CD59 is set forth in SEQ ID NO:12:

ATAGCCTGCAGTGCTACAACTGTCCTAACCCAACTGCTGACTGCAAAAC
AGCCGTCAATTGTTCATCTGATTTTGATGCGTGTCTCATTACCAAAGCT
GGGTTACAAGTGTATAACAAGTGTTGGAAGTTTGAGCATTGCAATTTCA
ACGACGTCACAACCCGCTTGAGGGAAAATGAGCTAACGTACTACTGCTG
CAAGAAGGACCTGTGTAACTTTAACGAACAGCTTGAAAATGGTGGGACA
TCCTTATCAGAGAAAACAGTTCTTCTGCTGGTGACTCCATTTCTGGCAG
CAGCCTGGAGCCTTCATCCC
(CR407661.1 Homo sapiens full open reading frame
cDNA clone RZPDo834A033D for gene CD59, CD59
antigen p18-20 (antigen identified by monoclonal
antibodies 16.3A5, EJ16, EJ30, EL32 and G344)
complete cds, without stop codon).

### Definitions

“Antisense oligonucleotide” or “ASO” encompasses an oligonucleotide having a nucleobase sequence that is complementary to a target nucleic acid or region or segment thereof. An antisense oligonucleotide is specifically hybridizable to a target nucleic acid or region or segment thereof, the hybridization of which results in RNase H mediated cleavage of the target nucleic acid.

“Conjugate group” encompasses a group of atoms that is directly attached to a polynucleotide. In certain embodiments, conjugate groups include a conjugate moiety and a conjugate linker that attaches the conjugate moiety to the polynucleotide.

“Contiguous” in the context of an oligonucleotide encompasses nucleosides, nucleobases, sugar moieties, or internucleoside linkages that are immediately adjacent to each other. For example, “contiguous nucleobases” means nucleobases that are immediately adjacent to each other in a sequence.

“Gapmer” encompasses an antisense oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” In certain embodiments, an antisense oligonucleotide is a gapmer.

“Mismatch” or “non-complementary” encompasses a nucleobase of a first polynucleotide that is not complementary to the corresponding nucleobase of a second polynucleotide or target nucleic acid when the first and second polynucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first polynucleotide that is not capable of hybridizing to the corresponding nucleobase of a second polynucleotide or target nucleic acid when the first and second polynucleotides are aligned is a mismatch or non-complementary nucleobase.

“Overhanging nucleosides” encompasses unpaired nucleotides at either or both ends of a duplex formed by hybridization of an antisense RNAi oligonucleotide and a sense RNAi oligonucleotide.

The nucleobase may be naturally occurring or synthetic. The nucleobase and sugar base may each, independently, be modified or unmodified. “Modified nucleoside” means a nucleoside comprising a modified nucleobase and/or a modified sugar moiety. Modified nucleosides can include abasic nucleosides, which lack a nucleobase.

“Portion” encompasses a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Polynucleotide” encompasses a polymer of linked nucleosides each of which can be modified or unmodified, independent one from another. In some embodiments, polynucleotides comprise 8-80 linked nucleosides. “Modified polynucleotides” means a polynucleotide, wherein at least one sugar, nucleobase, or internucleoside linkage is modified. “Unmodified polynucleotides” means polynucleotides that do not comprise any sugar, nucleobase, or internucleoside modification.

A “gene” encompasses an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

In some embodiments, the nucleic acid molecule such as an RNA molecule described herein can hybridize to a sequence of interest, e.g., a DNA sequence or an RNA sequence. A nucleic acid molecule is “hybridizable” or “hybridized” to another nucleic acid molecule, such as a cDNA, genomic DNA, or RNA, when a single stranded form of the nucleic acid molecule can anneal to the other nucleic acid molecule under appropriate conditions of temperature and ionic strength solution. While not limited to a particular mechanism, the most common mechanism of hybridization involves hydrogen bonding, which may be Watson-Crick, Hoogsteen or reversed Hoogsteen hydrogen bonding, between complementary nucleobases. In some embodiments, complementary nucleic acid molecules include, but are not limited to, an antisense compound and a nucleic acid target. In some embodiments, complementary nucleic acid molecules include, but are not limited to, a polynucleotide and a target nucleic acid.

“Specifically hybridizable” refers to a polynucleotide having a sufficient degree of complementarity between the polynucleotide and a target nucleic acid to induce a desired effect, while exhibiting minimal or no effects on non-target nucleic acids. In certain embodiments, specific hybridization occurs under physiological conditions.

The term “complementary” is used to describe the relationship between nucleotide bases and/or polynucleotides that are capable of hybridizing to one another, e.g., the nucleotide sequence of such polynucleotides or one or more regions thereof matches the nucleotide sequence of another polynucleotide or one or more regions thereof when the two nucleotide sequences are aligned in opposing directions. Nucleobase matches or complementary nucleobases, as described herein, include the following pairs: adenine (A) with thymine (T), adenine (A) with uracil (U), cytosine (C) with guanine (G), and 5-methyl cytosine (^{m}C) with guanine (G). Complementary polynucleotides and/or nucleic acids need not have nucleobase complementarity at each nucleoside and may include one or more nucleobase mismatches. Accordingly, the present disclosure also includes isolated polynucleotides that are complementary to sequences as disclosed or used herein as well as those substantially similar nucleic acid sequences.

The degree to which two polynucleotides have matching nucleobases can be expressed in terms of “percent complementarity” or “percent complementary.” In some embodiments, a polynucleotide has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% complementarity with another polynucleotide or a target nucleic acid provided herein. In embodiments wherein two polynucleotides or a polynucleotide and a target nucleic acid are “fully complementary” or “100% complementary,” such polynucleotides have nucleobase matches at each nucleoside without any nucleobase mismatches. In one embodiment, percent complementarity is the percent of the nucleobases of the shorter sequence that are complementary to the longer sequence.

As used herein, the terms “sequence similarity” or “% similarity” may be used interchangeably with “sequence identity” or “% identity” having all the same meanings and qualities. In some embodiments, sequence similarity encompasses the degree of identity or correspondence between nucleic acid sequences or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, which do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

Sequence similarity can be determined by sequence alignment using methods known in the field, such as, for example, BLAST, MUSCLE, Clustal (including ClustalW and ClustalX), and T-Coffee (including variants such as, for example, M-Coffee, R-Coffee, and Expresso). In some embodiments, only specific portions of two or more polynucleotide or polypeptide sequences are aligned to determine sequence identity. In some embodiments, only specific domains of two or more sequences are aligned to determine sequence similarity. A comparison window can be a segment of at least 10 to over 1000 residues, at least 20 to about 1000 residues, or at least 50 to 500 residues in which the sequences can be aligned and compared. Methods of alignment for determination of sequence identity are well-known and can be performed using publicly available databases such as BLAST. For example, in some embodiments, “percent identity” of two nucleotide sequences is determined using the algorithm of Karlin and Altschul, Proc Nat Acad Sci USA 87:2264-2268 (1990), modified as in Karlin and Altschul, Proc Nat Acad Sci USA 90:5873-5877 (1993). Such algorithms are incorporated into BLAST programs, e.g., BLAST+ or the NBLAST and XBLAST programs described in Altschul et al., J Mol Biol, 215: 403-410 (1990). BLAST protein searches can be performed with programs such as, e.g., the XBLAST program, score=50, word length=3 to obtain amino acid sequences homologous to the protein molecules of the disclosure. Where gaps exist between two sequences, Gapped BLAST can be utilized as described in Altschul et al., Nucleic Acids Res 25(17): 3389-3402 (1997). When utilizing BLAST and Gapped BLAST programs, the default parameters of the respective programs (e.g., XBLAST and NBLAST) can be used.

A DNA “coding sequence” is one of the strands of a double-stranded DNA sequence that is transcribed and translated into a polypeptide in a cell in vitro or in vivo when placed under the control of suitable regulatory sequences. “Regulatory sequences” refer to non-coding polynucleotide sequences located upstream (i.e., 5′), within, or downstream (i.e., 3′) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, bacterial and archaeal polynucleotides, cDNA from mRNA, genomic DNA polynucleotides, and synthetic DNA polynucleotides. If the coding sequence is intended for expression in a eukaryotic cell, a polyadenylation signal and transcription termination sequence are typically located 3′ of the coding sequence.

In some embodiments, canonical functions of CD59 comprise those functions related to the role of CD59 as a protector from C9 deposition, wherein CD59 inhibits membrane attack complex (MAC) assembly.

In some embodiments, a non-canonical function of CD59 comprises a function not related to the role of CD59 as a protector from C9 deposition and inhibition of membrane attack complex (MAC) assembly. In some embodiments, a non-canonical function of CD59 may comprise regulation of insulin resistance.

### Compounds

In some embodiments, a compound for use in the methods disclosed herein comprises an antibody or a binding fragment thereof, a protein, a peptide, a small molecule, or an oligonucleotide. In some embodiments, the compound as disclosed herein is an antisense oligonucleotide. In certain embodiments, the compound is a ribozyme. In some other embodiments, the compound can be a peptide, an antibody, or a chemical compound. In some embodiments, an oligo nucleotide used in a method disclosed herein comprises an antisense RNA, a ribozyme, an interfering RNA (RNAi), a double stranded short-interfering RNA (siRNA), or a single stranded RNAi.

In certain embodiments, the compounds described herein are interfering RNA compounds (RNAi), which include double-stranded RNA compounds (also referred to as short-interfering RNA or siRNA) and single-stranded RNAi compounds (or ssRNA). Such compounds work at least in part through the RNA-induced silencing complex (RISC) pathway to degrade and/or sequester a target nucleic acid (thus, include microRNA/microRNA-mimic compounds). As used herein, the term “siRNA” is meant to be equivalent to other terms used to describe nucleic acid molecules that are capable of mediating sequence-specific RNAi, for example, short interfering RNA (siRNA), double-stranded RNA (dsRNA), micro-RNA (miRNA), short hairpin RNA (shRNA), short interfering polynucleotide, short interfering nucleic acid, short interfering modified polynucleotide, chemically modified siRNA, post-transcriptional gene silencing RNA (ptgsRNA), and others. In addition, as used herein, the term “RNAi” is meant to be equivalent to other terms used to describe sequence-specific RNA interference, such as post transcriptional gene silencing, translational inhibition, or epigenetics.

In some embodiments, the compound is a single-stranded oligonucleotide. In certain embodiments, the term “oligonucleotide” may be used interchangeably with the term “polynucleotide” having all the same qualities and meanings.

In some embodiments, a single-stranded polynucleotide is capable of binding to a complementary polynucleotide to form a double-stranded duplex. In some embodiments, the single-stranded polynucleotide comprises a self-complementary sequence. “Self-complementary” means that a polynucleotide can at least partially hybridize to itself. In some embodiments, the single-stranded polynucleotide comprises a RNA polynucleotide. In some embodiments, the single-stranded polynucleotide is an ssRNA, or an antisense oligonucleotide (ASO).

In some embodiments, the polynucleotide compound is double-stranded. In some embodiments, the double-stranded compounds comprise a first polynucleotide having a region complementary to a target nucleic acid (e.g., an antisense RNAi polynucleotide) and a second polynucleotide having a region complementary to the first polynucleotide (e.g., a sense RNAi polynucleotide). In some embodiments, the double-stranded compound comprises a DNA polynucleotide. In certain embodiments, the compound comprises an RNA polynucleotide. In such embodiments, the thymine nucleobases in the polynucleotides are replaced by uracil nucleobases. The polynucleotides of double-stranded compounds may include non-complementary overhanging nucleosides.

In certain embodiments, the polynucleotide compound comprises one or more modified nucleosides in which the 2′ position of the sugar contains a halogen (such as fluorine group; 2′-F) or contains an alkoxy group (such as a methoxy group; 2′-OMe). In certain embodiments, the polynucleotide comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the compound. In certain embodiments, the polynucleotide comprises one or more linkages between adjacent nucleosides other than a naturally occurring phosphodiester linkage. Examples of such linkages include phosphoramide, phosphorothioate, and phosphorodithioate linkages. The polynucleotide may also be chemically modified nucleic acid molecules as taught in U.S. Pat. No. 6,673,661. In other embodiments, the polynucleotide compound contains one or two capped strands, as disclosed, for example, by WO 00/63364, filed Apr. 19, 2000. An example of double-stranded compounds is siRNA.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in the gap of a gapmer is tolerated.

In certain antisense activities, compounds described herein, or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In some embodiments, a target nucleic acid comprises any one of the nucleic acid sequences set forth in SEQ ID NOs: 2-12. In some embodiments, a target nucleic acid comprises any one of the nucleic acid sequences set forth in SEQ ID NOs: 2-12 or a nucleic acid sequence that is at least 80% (e.g., at least 85%, 90%, 95%, 96%, 97%, 98%, or 99%) identical to the nucleic acid sequences set forth in any of SEQ ID NOs: 2-12, for example but not limited to identity as determined using Blastn software of the National Center of Biotechnology Information (NCBI) using default parameters.

A skilled artisan would appreciate that percent identity (% identity) provides a number that describes how similar the query sequence is to the target sequence (i.e., how many nucleic acids in each sequence are identical). The higher the percentage identity is, the more significant the match.

When used in relation to polynucleotide (or oligonucleotide) sequences, the term “identity” refers to the degree of identity between two or more polynucleotide (or oligonucleotide) sequences or fragments thereof. Typically, the degree of similarity between two or more polynucleotide (or oligonucleotide) sequences refers to the degree of similarity of the composition, order, or arrangement of two or more nucleotides of the two or more polynucleotide sequences (or oligonucleotide sequences).

### RNAi Compounds

In certain embodiments, RNAi compounds comprise an antisense RNAi polynucleotide and optionally a sense RNAi polynucleotide. In certain embodiments, RNAi compounds may also comprise terminal groups and/or conjugate groups which may be attached to the antisense RNAi polynucleotide or the sense RNAi polynucleotide (when present).

In certain embodiments, RNAi compounds comprising an antisense RNAi polynucleotide and a sense RNAi polynucleotide may form a duplex, because the sense RNAi polynucleotide comprises an antisense-hybridizing region that is complementary to the antisense RNAi polynucleotide. In certain embodiments, each nucleobase of the antisense RNAi polynucleotide and the sense RNAi polynucleotide are complementary to one another. In certain embodiments, the two RNAi polynucleotides have at least one mismatch relative to one another.

In certain embodiments, the antisense hybridizing region constitutes the entire length of the sense RNAi polynucleotide and the antisense RNAi polynucleotide. In certain embodiments, one or both of the antisense RNAi polynucleotide and the sense RNAi polynucleotide comprise additional nucleosides at one or both ends that do not hybridize (overhanging nucleosides). In certain embodiments, overhanging nucleosides are DNA. In certain embodiments, overhanging nucleosides are linked to each other (where there is more than one) and to the first non-overhanging nucleoside with phosphorothioate linkages.

In certain embodiments, polynucleotides comprise a stabilized phosphate group at the 5′-end. In certain such embodiments, the compound is a ssRNAi compound or the compound is a siRNA and the polynucleotide comprising a stabilized phosphate group is the antisense strand of the siRNA compound. In certain embodiments, the 5′-end phosphorus-containing group can be 5′-end phosphate (5′-P), 5′-end phosphorothioate (5′-PS), 5′-end phosphorodithioate (5′-PS2), 5′-end vinylphosphonate (5′-VP), 5′-end methylphosphonate (MePhos) or 5′-deoxy-5′-C-malonyl. When the 5′-end phosphorus-containing group is 5′-end vinylphosphonate, the 5′VP can be either 5′-E-VP isomer (i.e., trans-vinylphosphate), 5′-Z-VP isomer (i.e., cis-vinylphosphate), or mixtures thereof. Although such phosphate group can be attached to either the antisense RNAi polynucleotide or the antisense RNAi polynucleotide, it will typically be attached to the antisense RNAi polynucleotide as that has been shown to improve activity of certain RNAi compounds. See, e.g., Prakash et al., Nucleic Acids Res., 43(6):2993-3011, 2015; Elkayam, et al., Nucleic Acids Res., 45(6):3528-3536, 2017; Parmar, et al. ChemBioChem, 17(11)985-989; 2016; Harastzi, et al., Nucleic Acids Res., 45(13):7581-7592, 2017. In certain embodiments, the phosphate stabilizing group is 5′-cyclopropyl phosphonate. See e.g., WO/2018/027106.

In some embodiments, an siRNA compound used in a method disclosed herein comprises an siRNA for posttranscriptional gene knock down of CD59, as provided in Geis et al. (2010) Current Cancer Drug Targets. 10:922-931, which is incorporated herein in full. In some embodiments, an siRNA compound comprises the sequence set forth as SEQ ID NO: 13: ggaccuguguaacuuuaacuu, which is a sense strand. In some embodiments, an siRNA compound comprises the sequence set forth as SEQ ID NO: 14: 3′ uuccuggacacauugaaauug 5′, which is the antisense strand.

In certain embodiments, RNAi compounds comprise a sense RNAi polynucleotide. In such embodiments, sense RNAi polynucleotide comprises an antisense hybridizing region complementary to the antisense RNAi polynucleotide. In certain embodiments, the antisense hybridizing region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 contiguous nucleotides. In certain embodiments, the antisense hybridizing region constitutes 70%, 80%, 85%, 90%, 95% of the nucleosides of the sense RNAi polynucleotide. In certain embodiments, the antisense hybridizing region constitutes all of the nucleosides of the sense RNAi polynucleotide. In certain embodiments, the antisense hybridizing region of the sense RNAi polynucleotide is at least 99%, 95%, 90%, 85%, or 80% complementary to the antisense RNAi polynucleotide. In certain embodiments, the antisense hybridizing region of the sense RNAi oligonucleotide is 100% complementary to the antisense RNAi polynucleotide.

The hybridizing region of a sense RNAi polynucleotide hybridizes with the antisense RNAi polynucleotide to form a duplex region. In certain embodiments, such duplex region consists of 7 hybridized pairs of nucleosides (one of each pair being on the antisense RNAi polynucleotide and the other of each pair being on the sense RNAi polynucleotide). In certain embodiments, a duplex region comprises at least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 hybridized pairs. In certain embodiments, each nucleoside of antisense RNAi polynucleotide is paired in the duplex region (i.e., the antisense RNAi polynucleotide has no overhanging nucleosides). In certain embodiments, the antisense RNAi polynucleotide includes unpaired nucleosides at the 3′-end and/or the 5′end (overhanging nucleosides). In certain embodiments, each nucleoside of sense RNAi polynucleotide is paired in the duplex region (i.e., the sense RNAi polynucleotide has no overhanging nucleosides). In certain embodiments, the sense RNAi polynucleotide includes unpaired nucleosides at the 3′-end and/or the 5′end (overhanging nucleosides). In certain embodiments, duplexes formed by the antisense RNAi polynucleotide and the sense RNAi polynucleotide do not include any overhangs at one or both ends. Such ends without overhangs are referred to as blunt. In certain embodiments wherein the antisense RNAi polynucleotide has overhanging nucleosides, one or more of those overhanging nucleosides are complementary to the target nucleic acid. In certain embodiments wherein the antisense RNAi polynucleotide has overhanging nucleosides, one or more of those overhanging nucleosides are not complementary to the target nucleic acid.

In some embodiments, the compound of the present disclosure comprises a miRNA. A “microRNA” or “miRNA” is a single-stranded RNA polynucleotide of about 15 to about 30 nucleotides in length that functions in vivo in RNA silencing and post-transcriptional regulation of gene expression. miRNA functions via base-pairing with complementary sequences with mRNA. As a result of the miRNA base-pairing, the mRNA is “silenced” by one or more of the following processes: (1) cleavage of the mRNA strand into two pieces; (2) destabilization of the mRNA through shortening of its poly(A) tail; and (3) less efficient translation of the mRNA. miRNAs are similar to siRNAs described herein, except that miRNA generally derive from regions of RNA that fold back on themselves to form hairpin structures, whereas siRNA derive from longer regions of double-stranded RNA. See, e.g., Filipowicz et al., Curr Opin Struct Biol 15, 331-341, (2005), van Rooij et al., J Clin Invest 117, 2369-2376 (2007), and MacFarlane et al., Curr Genomics 11(7), 537-561 (2010).

In some embodiments, a RNAi used in a method disclosed herein comprises an oligonucleotide that disrupts a non-canonical function of CD59. In some embodiments, a RNAi used herein binds to a CD59 gene sequence, as set forth in SEQ ID NO: 2 or a gene homolog having at least 80% identity with SEQ ID NO: 2. In some embodiments, a RNAi used herein binds to a CD59 mRNA transcript sequence, as set forth in any of SEQ ID NOs: 3-10 or a mRNA homolog thereof having at least 80% identity with SEQ ID NOs: 3-10. In some embodiments, a RNAi used herein binds to a CD59 Open Reading Frame (ORF) sequence, as set forth in any of SEQ ID NOs: 11-12 or an ORF homolog thereof having at least 80% identity with SEQ ID NOs: 11-12.

In some embodiments, the RNAi disrupts a non-canonical function of CD59. In some embodiments, the RNAi disrupts a non-canonical function of CD59 in liver and or muscle tissue. In some embodiments, a RNAi used in a method disclosed herein disrupts the improper secretion of insulin from pancreatic beta-cells. In some embodiments, a RNAi disrupts a non-canonical function of CD59 but not a canonical function of CD59 in the complement cascade.

In some embodiments, the methods of use of an RNAi modulates pancreatic beta-cell insulin secretion. It should be understood that the term “modulating” as used herein generally refers to any change of an attribute. The methods described herein in some embodiments, prevent a subject from developing diabetes and decrease or eliminate weight gain in a subject that increases insulin resistance and therefore causes higher insulin secretion. Following treatments, the amount of insulin secreted is reduced. In some embodiments, modulation comprises a decrease of insulin secreted.

### Polynucleotides

In some embodiments, a compound comprises a polynucleotide.

In some embodiments, the present disclosure provides a polynucleotide comprising 8 to 80 linked nucleosides. In some embodiments, the polynucleotide comprises 8 to 50 linked nucleosides. In some embodiments, the polynucleotide comprises 10 to 30 linked nucleosides. In some embodiments, the polynucleotide comprises 12 to 30 linked nucleosides. In some embodiments, the polynucleotide comprises 12 to 22 linked nucleosides. In some embodiments, the polynucleotide comprises 14 to 30 linked nucleosides. In some embodiments, the polynucleotide comprises 15 to 30 linked nucleosides. In some embodiments, the polynucleotide comprises 16 to 30 linked nucleosides. In some embodiments, the polynucleotide comprises 17 to 30 linked nucleosides. In some embodiments, the polynucleotide comprises 12 to 20 linked nucleosides. In some embodiments, the polynucleotide comprises 15 to 20 linked nucleosides. In some embodiments, the polynucleotide comprises 16 to 20 linked nucleosides. In some embodiments, the polynucleotide comprises 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 5 57, 58, 59, 60, 61, 62, 63, 64, 65, 66, 67, 68, 69, 70, 71, 72, 73, 74, 75, 76, 77, 78, 79, or 80 linked nucleosides. In some embodiments, the polynucleotide comprises about 8, about 9, about 10, about 11, about 12, about 13, about 14, about 15, about 16, about 17, about 18, about 19, or about 20 linked nucleosides.

In some embodiments, the polynucleotide disclosed herein has a nucleobase sequence at least 50%, at least 55%, at least 60%, at least 65%, at least 70%, at least 75%, at least 80%, at least 85%, at least 90%, at least 95%, or about 100% complementary to an equal length portion of a nucleic acid encoding CD59 (e.g., any one of SEQ ID NOs:2-12).

In some embodiments, the present disclosure provides a polynucleotide comprising 8 to 50 linked nucleosides and having at least 90% sequence complementarity to an equal length portion of a nucleic acid encoding CD59. In some embodiments, the polynucleotide comprises 10 to 30 linked nucleosides and has at least 90% sequence complementarity an equal length portion of a nucleic acid encoding CD59. In some embodiments, the polynucleotide comprises 12 to 20 linked nucleosides and has at least 90% sequence complementarity to an equal length portion of a nucleic acid encoding CD59.

In some embodiments, the polynucleotide disclosed herein has a nucleobase sequence at least 50%, at least 55%, at least 60%, at least 65%, at least 70%, at least 75%, at least 80%, at least 85%, at least 90%, at least 95%, or about 100% complementary to an equal length portion of a transcription initiation site, a translation initiation site, 5′-untranslated sequence, 3′-untranslated sequence, coding sequence, a pre-mRNA sequence, and/or an intron/exon junction of an mRNA encoding the CD59 protein. In some embodiments, the polynucleotide has a nucleobase sequence at least 90%, at least 91%, at least 92%, at least 93%, at least 94%, at least 95%, at least 96%, at least 97%, at least 98%, at least 99%, or about 100% complementary to equal length portion of a transcription initiation site, a translation initiation site, 5′-untranslated sequence, 3′-untranslated sequence, coding sequence, a pre-mRNA sequence, and/or an intron/exon junction of an mRNA encoding the CD59 protein. In some embodiments, the polynucleotide has a nucleobase sequence capable of hybridizing with an equal length portion or all of transcription initiation site, a translation initiation site, 5′-untranslated sequence, 3′-untranslated sequence, coding sequence, a pre-mRNA sequence, and/or an intron/exon junction of an mRNA encoding the CD59 protein. In some embodiments, the polynucleotide has a nucleobase sequence at least 50%, at least 55%, at least 60%, at least 65%, at least 70%, at least 75%, at least 80%, at least 85%, at least 90%, at least 95%, or about 100% complementary to an equal length portion of any one of SEQ ID NOs:2-12. In some embodiments, the polynucleotide has a nucleobase sequence at least 90%, at least 91%, at least 92%, at least 93%, at least 94%, at least 95%, at least 96%, at least 97%, at least 98%, at least 99%, or about 100% complementary to an equal length portion of any one of SEQ ID NOs:2-12.

In some embodiments, the polynucleotide disclosed herein comprises at least one modification such as at least one modified internucleoside linkage, at least one modified sugar moiety, or at least one modified nucleobase.

In some embodiments, the polynucleotide disclosed herein comprises at least one modified internucleoside linkage. The naturally occurring internucleoside linkage of RNA and DNA is a 3′ to 5′ phosphodiester linkage. In some embodiments, the polynucleotides described herein having one or more modified, i.e., non-naturally occurring, internucleoside linkages are often selected over polynucleotides having naturally occurring internucleoside linkages because of desirable properties such as, for example, enhanced cellular uptake, enhanced affinity for target nucleic acids, and increased stability in the presence of nucleases. In some embodiments, nucleosides of modified polynucleotides may be linked together using any internucleoside linkage generally known in the art.

In some embodiments, a polynucleotide used in a method disclosed herein comprises an oligonucleotide that disrupts a non-canonical function of CD59. In some embodiments, a polynucleotide used herein binds to a CD59 gene sequence, as set forth in SEQ ID NO: 2 or a gene homolog having at least 80% identity with SEQ ID NO: 2. In some embodiments, a polynucleotide used herein binds to a CD59 mRNA transcript sequence, as set forth in any of SEQ ID NOs: 3-10 or a mRNA homolog thereof having at least 80% identity with SEQ ID NOs: 3-10. In some embodiments, a polynucleotide used herein binds to a CD59 Open Reading Frame (ORF) sequence, as set forth in any of SEQ ID NOs: 11-12 or an ORF homolog thereof having at least 80% identity with SEQ ID NOs: 11-12.

In some embodiments, the polynucleotide disrupts a non-canonical function of CD59. In some embodiments, the polynucleotide disrupts a non-canonical function of CD59 in liver and or muscle tissue. In some embodiments, a polynucleotide used in a method disclosed herein disrupts the improper secretion of insulin from pancreatic beta-cells. In some embodiments, a polynucleotide disrupts a non-canonical function of CD59 but not a canonical function of CD59 in the complement cascade.

In some embodiments, the methods of use of a polynucleotide modulates pancreatic beta-cell insulin secretion. It should be understood that the term “modulating” as used herein generally refers to any change of an attribute. The methods described herein in some embodiments, prevent a subject from developing diabetes and decrease or eliminate weight gain in a subject that increases insulin resistance and therefore causes higher insulin secretion. Following treatments, the amount of insulin secreted is reduced. In some embodiments, modulation comprises a decrease of insulin secreted.

### Conjugate Groups

In some embodiments, the polynucleotide disclosed herein comprises conjugate groups. In some embodiments, conjugate groups that are attached to either or both ends of a polynucleotide are terminal groups. In certain such embodiments, conjugate groups or terminal groups are attached at the 3′ and/or 5′-end of a polynucleotide. In certain such embodiments, conjugate groups (or terminal groups) are attached at the 3′-end of a polynucleotide. In some embodiments, conjugate groups are attached near the 3′-end of a polynucleotide. In some embodiments, conjugate groups (or terminal groups) are attached at the 5′-end of a polynucleotide. In some embodiments, conjugate groups are attached near the 5′-end of a polynucleotide.

In some embodiments, the conjugate/terminal group of a polynucleotide comprises a capping group, a phosphate moiety, a protecting group, or a modified or unmodified nucleoside. In some embodiments, the conjugate/terminal group includes one or more of an intercalator, a reporter, a polyamine, a polyamide, a peptide, a carbohydrate (e.g., GalNAc), a vitamin, a polyethylene glycol, a thioether, a polyether, a folate, a lipid, a phospholipid, biotin, phenazine, phenanthridine, anthraquinone, adamantane, acridine, fluorescein, rhodamine, coumarin, a fluorophore, and a dye.

In some embodiments, the conjugate/terminal group of a polynucleotide comprises a targeting moiety. In some embodiments, the targeting moiety is at the 5′ end of the polynucleotide. In some embodiments, the targeting moiety is at the 3′ end of the polynucleotide. In some embodiments, the targeting moiety targets the polynucleotide to a specific subcellular location and/or a specific cell or tissue type. In some embodiments, the targeting moiety comprises a ligand for a receptor. In some embodiments, the receptor is specific to a type of cell and/or tissue (e.g., liver or liver cells). In some embodiments, recognition of the targeting moiety (e.g., ligand) by the receptor mediates endocytosis of the polynucleotide conjugated to the targeting moiety.

In some embodiments, the targeting moiety targets a liver cell (also referred to herein as a hepatocyte). In some embodiments, the liver cell is a human liver cell. In some embodiments, the liver cell expresses an asialoglycoprotein receptor (ASGPr) on its cell surface. In some embodiments, the targeting moiety is a ligand for the ASGPr. In some embodiments, the targeting moiety comprises an N-acetylgalactosamine (GalNAc) moiety. In some embodiments, the targeting moiety comprises 1 to 5 GalNAc moieties. In some embodiments, the targeting moiety comprises 1, 2, 3, 4, or 5 GalNAc moieties. In some embodiments, the targeting moiety comprises 3 GalNAc moieties. In some embodiments, the targeting moiety comprises 3 GalNAc moieties in a triantennary arrangement (a triantennary GalNAc). In some embodiments, the polynucleotide comprises a triantennary GalNAc at the 5′ of the polynucleotide.

In certain embodiments, a polynucleotide is complementary to the target nucleic acid over the entire length of the polynucleotide. In certain embodiments, the polynucleotides are 99%, 95%, 90%, 85%, or 80% complementary to the target nucleic acid. In certain embodiments, polynucleotides are at least 80% complementary to the target nucleic acid over the entire length of the polynucleotide and comprise a region that is 100% or fully complementary to a target nucleic acid. In certain embodiments, the region of full complementarity is from 6 to 20, 10 to 18, or 18 to 20 nucleobases in length.

In certain embodiments, the polynucleotides disclosed herein comprise a targeting region complementary to the target nucleic acid. In certain embodiments, the targeting region comprises at least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 contiguous nucleotides. In certain embodiments, the targeting region constitutes 70%, 80%, 85%, 90%, 95% of the nucleosides of the polynucleotide. In certain embodiments, the targeting region constitutes all of the nucleosides of the polynucleotide. In certain embodiments, the targeting region of the polynucleotide is at least 99%, 95%, 90%, 85%, or 80% complementary to the target nucleic acid. In certain embodiments, the targeting region of the polynucleotide is 100% complementary to the target nucleic acid.

In some embodiments, the compound of the present disclosure for treating or preventing fatty liver disease in a subject in need thereof or for lowering of CD59 expression comprises a siRNA. A “short-interfering RNA,” “small-interfering RNA,” “silencing RNA,” or “siRNA,” is a class of compound comprising complementary RNA polynucleotides hybridized to one another, each comprising about 15 to about 30 linked nucleosides. siRNA operates in vivo within the RNA interference (RNAi) pathway and acts, at least in part, through RISC or Ago2 to interfere with expression of specific genes with complementary nucleotide sequences by degrading mRNA after transcription, thereby preventing translation. See, e.g., Dana et al., Int J Biomed Sci 13(2), 48-57 (2017), Whitehead et al., Ann Rev Chem Biomol Eng 2, 77-96 (2011), Filipowicz et al., Curr Opin Struct Biol 15, 331-341, (2005)

In some embodiments, the polynucleotides disclosed herein capable of hybridizing with a nucleic acid encoding the CD59 protein and capable of inhibiting expression of the CD59 protein is a siRNA or a “microRNA” or “miRNA”. In some embodiments, the siRNA or miRNA comprises a nucleotide sequence at least 50%, at least 55%, at least 60%, at least 65%, at least 70%, at least 75%, at least 80%, at least 85%, at least 90%, at least 95%, or about 100% complementary to an equal length portion of a nucleic acid sequence encoding the CD59 protein. In some embodiments, the siRNA or miRNA comprises a nucleotide sequence at least 90%, at least 91%, at least 92%, at least 93%, at least 94%, at least 95%, at least 96%, at least 97%, at least 98%, at least 99%, or about 100% complementary to an equal length portion of a sequence encoding the CD59 protein. In some embodiments, the siRNA or miRNA comprises a nucleotide sequence at least at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or about 100% complementary to an equal length portion of any one of SEQ ID NOs:2-12.

### Proteins and Peptides

In some embodiments, a protein comprises a polypeptide. In some embodiments a polypeptide comprises an antibody or a binding fragment thereof. As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. In some embodiments, a target antigen comprises CD59. In some embodiments, a target antigen comprises a cell surface epitope of CD59. In some embodiments, a target antigen comprises a GPI-linked cell surface CD59.

As used herein, the term “antibody” encompasses an antibody fragment or fragments that retain binding specificity including, but not limited to, IgG, heavy chain variable regions (VH), light chain variable regions (VL), Fab fragments, F(ab′)2 fragments, scFv fragments, Fv fragments, a nanobody, minibodies, diabodies, triabodies, tetrabodies, and single domain antibodies (see, e.g., Hudson and Souriau, Nature Med. 9: 129-134 (2003)). Also encompassed are humanized, primatized, and chimeric antibodies as these terms are generally understood in the art.

The term “antibody” as used herein further includes a peptide coding for one or more complementarity-determining regions (CDRs). In one embodiment, CDR peptides (“minimal recognition units”) can be obtained by constructing genes encoding the CDR of an antibody of interest.

By “specifically binding” is meant that the binding is selective for the antigen of interest and can be discriminated from unwanted or nonspecific interactions. For example, an antibody is said to specifically bind a CD59 epitope when the equilibrium dissociation constant is ≤10-5, 10-6, or 10-7 M. In some embodiments, the equilibrium dissociation constant may be ≤10-8 M or 10-9 M. In some further embodiments, the equilibrium dissociation constant may be ≤10-10 M, 10-11 M, or 10-12M. In some embodiments, the equilibrium dissociation constant may be in the range of ≤10-5 M to 10-12M.

Half maximal effective concentration (EC50) refers to the concentration of a drug, antibody or toxicant which induces a response halfway between the baseline and maximum responses after a specified exposure time. In some embodiments, the response comprises a binding affinity. In some embodiments, the response comprises a functional response for example a response showing regulation of pancreatic beta-cell insulin secretion. In some embodiments, the response comprises a functional response, for example prevention of a decline of insulin resistance, or prevention or treating of fatty liver disease, or treating glucose intolerance, or a combination thereof.

In some embodiments, glucose intolerance may encompass metabolic conditions, which result in higher than normal blood glucose levels—hyperglycemia. Glucose intolerance can be defined as dysglycemia that comprises both prediabetes and diabetes. Glucose intolerance may include the conditions of impaired fasting glucose (IFG) and impaired glucose tolerance (IGT) and diabetes mellitus (DM).

The methods described herein in some embodiments, prevent a subject from becoming diabetic and decrease or eliminate weight gain in a subject that increases insulin resistance and therefore causes higher insulin secretion. Following treatments, the amount of insulin secreted is reduced. In some embodiments, a response comprises a decrease of insulin secreted.

A skilled artisan would appreciate that as used herein in certain embodiments, the EC50 measurement of an anti-CD59 antibody disclosed herein provides a measure of a half-maximal binding of the anti-CD59 antibody to the cell surface CD59 antigen (EC50 binding). Measure of EC50 binding affinity comprises measuring the binding of an anti-CD59 antibody described herein to the CD59 antigen. The skilled artisan would appreciate that as used herein in certain embodiments, the EC50 measurement of an anti-CD59 antibody disclosed herein provides a measure of a half-maximal effective concentration of the anti-CD59 antibody to treat glucose intolerance, insulin resistance, and or fatty liver disease. Measure of EC50 functional activity comprises measuring the effects of the anti-CD59 on pancreatic beta-cell insulin secretion.

In some embodiments, a polypeptide used in a method disclosed herein comprises an antibody that disrupts a non-canonical function of CD59. In some embodiments, an antibody used herein binds to a CD59 cell surface epitope comprised within the sequence set forth in SEQ ID NO: 1 or an amino acid homolog thereof having at least 80% identity with the epitope comprised within SEQ ID NO: 1.

In some embodiments, the antibody disrupts a non-canonical function of CD59. In some embodiments, the antibody disrupts a non-canonical function of CD59 in liver and or muscle tissue. In some embodiments, an antibody used in a method disclosed herein disrupts the improper secretion of insulin from pancreatic beta-cells. In some embodiments, an antibody disrupts a non-canonical function of CD59 but not a canonical function of CD59 in the complement cascade.

In some embodiments, the methods of use of an anti-CD59 antibody modulates pancreatic beta-cell insulin secretion. The methods described herein in some embodiments, prevent a subject from becoming diabetic and decrease or eliminate weight gain in a subject that increases insulin resistance and therefore causes higher insulin secretion. Following treatments, the amount of insulin secreted is reduced. In some embodiments, modulation comprises a decrease of insulin secreted.

In some embodiments, a protein comprises a cyclic protein.

As used herein the term “peptide” includes native peptides (either degradation products, synthetically synthesized peptides or recombinant peptides) and peptidomimetics (typically, synthetically synthesized peptides, cyclic), such as peptoids and semipeptoids which are peptide analogs, which may have, for example, modifications rendering the peptides more stable while in a body. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH, —C(O)—O, —CO—(O)—, —CH(OH)—CH2-), or CF═CH, retro amide bond, backbone modifications, and residue modification.

As used herein the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

In some embodiments, the peptide specifically binds a CD59 epitope. In some embodiments, the peptide specifically binds a cell surface epitope of CD59. In some embodiments, the peptide specifically binds a GPI-linked cell surface CD59.

In some embodiments, a peptide used herein binds to a CD59 cell surface epitope. In some embodiments, a peptide used herein binds to a CD59 cell surface epitope comprised within the sequence set forth in SEQ ID NO: 1 or an amino acid homolog thereof having at least 80% identity with the epitope comprised within SEQ ID NO: 1. In some embodiments, a peptide used herein binds to a CD59 polynucleotide sequences. In some embodiments, a peptide used herein binds to a CD59 polynucleotide sequences comprised within any of the sequences set forth in SEQ ID NOs: 2-12 or an polynucleotide homolog thereof having at least 80% identity with a sequence set forth in any of SEQ ID NO: 2-12.

In some embodiments, a peptide used in a method disclosed herein disrupts a non-canonical function of CD59. In some embodiments, the peptide disrupts a non-canonical function of CD59 in liver and or muscle tissue. In some embodiments, a peptide used in a method disclosed herein disrupts the improper secretion of insulin from pancreatic beta-cells. In some embodiments, a peptide disrupts a non-canonical function of CD59 but not a canonical function of CD59 in the complement cascade.

In some embodiments, the methods of use of a CD59 polypeptide binding compound modulates pancreatic beta-cell insulin secretion. In some embodiments, the methods of use of a CD59 peptide binding compound modulates pancreatic beta-cell insulin secretion. The methods described herein in some embodiments, prevent a subject becoming diabetic and decrease or eliminate weight gain in a subject that increases insulin resistance and therefore causes higher insulin secretion. Following treatments, the amount of insulin secreted is reduced. In some embodiments, modulation comprises a decrease of insulin secreted.

In some embodiments, a small molecule used in the methods disclosed herein comprises a neutralizing minibody, for example but not limited to the anti-CD50 minibody MB-59, disclosed in United States Patent Application Publication No. 2009/0053225, which is incorporated herein in full.

In some embodiments, a small molecule used in the methods disclosed herein comprises a nuclear targeted REST peptide, which inhibits expression of CD59 at the mRNA and protein level, as disclosed in International Publication No. WO 2009/147384, which is incorporated herein in full.

In some embodiments, a small molecule used in the methods disclosed herein comprises a recombinant bacterial toxin intermedilysin fragment that binds CD59, as disclosed in Hu et al. (2010) J Immunol.; 184(1):359-68, which is incorporated herein in full.

### Small Molecules

In some embodiments, a “small molecule” may encompass a substantially non-peptidic, non-oligomeric organic molecule either prepared in the laboratory or found in nature. Small molecules, as used herein, may in certain embodiments encompass small molecules that are “natural product-like,” however, the term “small molecule” is not limited to “natural product-like” compounds. Rather, a small molecule is typically characterized in that it contains several carbon-carbon bonds and has a molecular weight of less than 1500 g/mol, less than 1250 g/mol, less than 1000 g/mol, less than 750 g/mol, less than 500 g/mol, or less than 250 g/mol, although this characterization is not intended to be limiting for the purposes of the small molecules disclosed herein.

In some embodiments, a small molecule specifically binds a ligand. In certain embodiments, a small molecule binds to a polypeptide ligand. In certain embodiments, the ligand comprises a CD59 polypeptide. In certain embodiments, the ligand comprises a cell-surface CD59 polypeptide. In certain embodiments, the ligand comprises a cell-surface GPI-bound CD59 polypeptide. In certain embodiments, the ligand comprises a CD59 polypeptide set forth in SEQ ID NO: 1.

In certain embodiments, a small molecule binds to a polynucleotide ligand. In certain embodiments, the ligand comprises a CD59 polynucleotide. In certain embodiments, the ligand comprises a gene or mRNA sequence encoding a CD59 polypeptide. In certain embodiments, the ligand comprises a CD59 polynucleotide set forth in any of SEQ ID NOs: 2-12.

It will be understood by those skilled in the art that the term “ligand” generally refers to a substance that forms a complex with another biomolecule.

In some embodiments, a small molecule used in a method disclosed herein disrupts a non-canonical function of CD59. In some embodiments, the small molecule disrupts a non-canonical function of CD59 in liver and or muscle tissue. In some embodiments, a small molecule used in a method disclosed herein disrupts the improper secretion of insulin from pancreatic beta-cells. In some embodiments, a small molecule disrupts a non-canonical function of CD59 but not a canonical function of CD59 in the complement cascade.

In some embodiments, the methods of use of a small molecule modulates pancreatic beta-cell insulin secretion. In some embodiments, the methods of use of a small molecule modulates pancreatic beta-cell insulin secretion.

### Targeted Delivery to Liver, Muscle, and Adipose Tissue

The liver is the largest single internal organ in mammals and is involved in metabolism, detoxification, synthesis of proteins and lipids, secretion of cytokines and growth factors and immune/inflammatory responses. Safe and efficient delivery of therapeutic molecules (e.g., compounds including drugs, small molecules, oligonucleotides, peptides, or proteins) into the liver is very important to increase the clinical efficacy of these molecules and to reduce their side effects in other organs. Several liver cell-targeted delivery systems have been developed and well-known in the art. For example, various strategies have been proposed to improve the delivery of different drugs to liver and hepatocytes which includes passive accumulation of nanoparticle therapeutics and active targeting by surface modifications of nanoparticles with specific ligands such as carbohydrates, peptides, proteins and antibodies (see e.g. Gorad et al., IJPSR, 2013, 4:4145-4157; Mahdinloo et al., Acta Pharmaceutica Sinica B, 2020, 10:1279-1293).

In general, liver targeting systems employ passive trapping of microparticles by reticuloendothelium or active targeting based on recognition between hepatic receptor and ligand-bearing particulates. For example, following systemic administration of nanoparticles, the defining size properties (typically <200 nm in diameter) of nanoparticle therapeutics greatly facilitates passive liver targeting in the absence of significant self-aggregation or aggregation with serum proteins. This effectively builds up a high local concentration of nanoparticle therapeutics in the space of Disse, where diffusion to the various liver cell types can occur. With regard to active targeting to liver cells, targeting of therapeutics by ligand-mediated approaches to hepatic stellate cells and hepatocytes are well-known in the art. Liposomes, nanoparticles, and polymeric micelles have also been widely used as drug carriers for liver targeting.

Targeted delivery of oligonucleotides to liver hepatocytes using N-acetylgalactosamine (GalNAc) conjugates that bind to the asialoglycoprotein receptor has become a breakthrough approach in the therapeutic oligonucleotide field. The use of this delivery system for small interfering RNAs (siRNAs) and antisense molecules has led to downregulation of target mRNA and protein. This delivery approach can also be used with anti-microRNAs and small activating RNAs (see Debacker et al., Mol. Ther. 2020, 28:1759-1771).

In some embodiments, a therapeutic compound, as disclosed here is targeted to the liver. In some embodiments, an oligonucleotide disclosed here is targeted to the liver. In some embodiments, a polynucleotide disclosed here is targeted to the liver. In some embodiments, a polypeptide disclosed here is targeted to the liver. In some embodiments, a peptide disclosed here is targeted to the liver. In some embodiments, an antibody disclosed here is targeted to the liver. In some embodiments, a small molecule disclosed here is targeted to the liver.

Skeletal muscle is essential for metabolism, both for its role in glucose uptake and its importance in exercise and metabolic disease. During a meal, insulin stimulates glucose storage by the liver as glycogen. The insulin released from the liver acts on adipose and muscle tissue to stimulate glucose uptake. Further, skeletal muscle is a regulator of glucose homeostasis, responsible for 80% of postprandial glucose uptake (the glucose concentration in your bloodstream in the period up to four hours after eating a meal) from the circulation. Lean muscle helps the pancreas as the organ has to produce less insulin to regulate the body. Adipose tissue also plays a primary metabolic role. In the feeding state, insulin-dependent glucose transport 4 (GLUT 4) allows the uptake of glucose from the bloodstream to adipocytes.

One skilled in the art would appreciate that adipose tissue may encompass visceral and epicardial fat.

Safe and efficient delivery of therapeutic molecules (e.g., compounds disclosed herein including small molecules, oligonucleotides/polynucleotides, or proteins/peptides/antibodies) comprising administration by intravenous (IV), intramuscular (IM), intra-adipose, intra-hepatic, or oral routes, is very important to increase the clinical efficacy of these molecules and to reduce their side effects in other organs. In some embodiments, administration is by IV. In some embodiments, administration is by intramuscular administration. In some embodiments, administration is by intra-adipose administration. In some embodiments, administration is by intra-hepatic administration. In some embodiments, administration is by oral administration. In some embodiments, administration is by IV, IM, intra-adipose, intra-hepatic, or oral administration.

With regard to active targeting to muscle and or adipose tissue cells, targeting of therapeutics by ligand-mediated approaches are well-known in the art. Liposomes, nanoparticles, and polymeric micelles have also been widely used as drug carriers for skeletal muscle and or adipose tissue targeting.

For targeting to skeletal muscle tissue methods may include but are not limited to use of carnitine conjugates for improving muscle update uptake via OCTN2 transport. Binding to muscle surface recognition elements followed by endocytosis may in some embodiments, allow even large molecules such as antibodies to enter muscle cells. Hybrid adeno-associated viral vectors have shown promise for high skeletal muscle selectivity in gene transfer applications. Delivery technology methods, including electroporation of DNA plasmids, have also been investigated for selective muscle uptake. (See for example Nicholson et al., (2023) Pharmaceutics, 15(1), 237; Winkler et al., (2023) Orv Hetil; 164(1):3-10; Ebner et al. (2015) Curr Pharm Des; 21(10):1327-36.) Further methods may include targeting oligonucleotides conjugated to cholesterol or octa-guanidine dendrimer (also known as vivo-morpholino structures), which have demonstrated efficacy to suppress proteins in skeletal muscle and increase muscle size following IV administration in mice (See for example Kang et al., (2011) Mol. Ther.; 19:159-164; Khan et al., (2016) Mol. Ther. Nucleic Acids.; 5:e342.) Another approach involved a nanoparticle complex containing targeted siRNA and atelocollagen, which was administered by a single IM injection. Lastly, intramuscular administration of targeted siRNA in combination with muscle-specific microRNAs was superior compared to either agent alone for promoting a muscle effect.

In some embodiments, a compound disclosed here is targeted to skeletal muscle tissue. In some embodiments, an oligonucleotide disclosed here is targeted to skeletal muscle tissue. In some embodiments, a polynucleotide disclosed here is targeted to the skeletal muscle tissue. In some embodiments, a polypeptide disclosed here is targeted to the skeletal muscle tissue. In some embodiments, a peptide disclosed here is targeted to the skeletal muscle tissue. In some embodiments, an antibody disclosed here is targeted to the skeletal muscle tissue. In some embodiments, a small molecule disclosed here is targeted to the skeletal muscle tissue.

In some embodiments, a compound described herein disclosed here is targeted to adipose tissue. In some embodiments, an oligonucleotide described herein disclosed here is targeted to adipose tissue. In some embodiments, a polynucleotide disclosed here is targeted to the adipose tissue. In some embodiments, a polypeptide disclosed here is targeted to the adipose tissue. In some embodiments, a peptide disclosed here is targeted to the adipose tissue. In some embodiments, an antibody disclosed here is targeted to the adipose tissue. In some embodiments, a small molecule disclosed here is targeted to the adipose tissue.

In some embodiments, a compound described herein disclosed here is targeted to liver, skeletal muscle, or adipose tissue, or any combination thereof. In some embodiments, an oligonucleotide described herein disclosed here is targeted to liver, skeletal muscle, or adipose tissue, or any combination thereof. In some embodiments, a polynucleotide disclosed here is targeted to liver, skeletal muscle, or adipose tissue, or any combination thereof. In some embodiments, a polypeptide disclosed here is targeted to liver, skeletal muscle, or adipose tissue, or any combination thereof. In some embodiments, a peptide disclosed here is targeted to liver, skeletal muscle, or adipose tissue, or any combination thereof. In some embodiments, an antibody disclosed here is targeted to liver, skeletal muscle, or adipose tissue, or any combination thereof. In some embodiments, a small molecule disclosed here is targeted to liver, skeletal muscle, or adipose tissue, or any combination thereof.

### CRISPR/Cas

In some embodiments, the present disclosure provides methods of preventing or treating liver diseases or conditions (e.g., nonalcoholic fatty liver disease or nonalcoholic steatohepatitis, etc.) in a subject comprising silencing or down-regulating CD59 in hepatocytes of the subject. In some embodiments, the present disclosure provides methods of preventing or treating liver diseases or conditions (e.g., nonalcoholic fatty liver disease or nonalcoholic steatohepatitis, etc.) in a subject comprising silencing or down-regulating CD59 in a liver cell of the subject. In some embodiments, the present disclosure provides methods of preventing or treating liver diseases or conditions (e.g., nonalcoholic fatty liver disease or nonalcoholic steatohepatitis, etc.) in a subject comprising silencing or down-regulating CD59 in striated muscle cells of the subject. This may be achieved using genome editing technology to silence or reduce CD59 expression. In certain embodiments, CD59 is silenced or downregulated using a RNA-guided nuclease. The RNA-guided nuclease is a CRISPR-Cas9 combination comprising a Cas9 enzyme and a guide RNA (gRNA) that specifically binds to, recognizes, or hybridizes to a CD59 transcript. In one embodiment, the gRNA specifically binds to, recognizes, or hybridizes to the CD59 gene or fragment thereof. In another embodiment, the gRNA specifically binds to, recognizes, or hybridizes a nucleic acid encoding the CD59 protein. In one embodiment, the guide RNA (gRNA) specifically binds to, recognizes, or hybridizes to a part of the nucleic acid sequence of CD59 transcript having the sequence of any one of SEQ ID NOs:2-12. In one embodiment, the gRNA binds to, recognizes, or hybridizes to a part of the nucleic acid sequence encoding a CD59 having the amino acid sequence of SEQ ID NO:1.

The CRISPR/Cas system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/CAS system can thereby be engineered to cleave virtually any DNA sequence by redesigning the gRNA in cell lines or primary cells. The CRISPR/CAS system can simultaneously target multiple genomic loci by co-expressing a single CAS9 protein with two or more gRNAs, making this system uniquely suited for multiple gene editing or synergistic activation of target genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In one embodiment, CD59 can be downregulated by introducing a Cas expression vector and a guide nucleic acid sequence specific for the CD59 gene into a target cell. In one embodiment, the CRISPR system comprises an expression vector, such as, but not limited to, a pAd5F35-CRISPR vector. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

The guide nucleic acid sequence is specific for a gene and targets that gene for Cas endonuclease-induced double strand breaks. The sequence of the guide nucleic acid sequence may be within a loci of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. In one embodiment, the guide RNA (gRNA) specifically binds to, recognizes, or hybridizes to the CD59 gene, a fragment thereof, or a nucleic acid encoding the CD59 protein.

One example of a CRISPR/Cas system used to inhibit gene expression, CRISPRi, is described in U.S. Publication No.: 2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

### Introduction of Nucleic Acids

In certain embodiments, the methods disclosed herein rely on known methods of introducing nucleic acids to administer the siRNA and/or Cas9 enzyme and a guide RNA (gRNA) that specifically binds to, recognizes, or hybridizes to CD59 target sequences.

Methods of introducing nucleic acids into a cell include physical, biological, and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II, Amaxa Biosystems, Cologne, Germany; ECM 830 (BTX), Harvard Instruments, Boston, Mass.) or the Gene Pulser II (BioRad, Denver, Colo.), or Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12:861-70 (2001)).

Biological methods for introducing a polynucleotide of interest into a host cell include the use of DNA or RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to an inhibitor, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots).

The present disclosure also provides for vectors containing the siRNA or gRNA of the disclosure. The vector can have a nucleic acid sequence containing an origin of replication. The vector can be a plasmid, bacteriophage, bacterial artificial chromosome, or yeast artificial chromosome. The vector can be either a self-replicating extrachromosomal vector or a vector which integrates into a host genome.

### Diseases and Conditions

In some embodiments, disclosed herein are methods of treating a liver disease or condition. In some embodiments, a subject suffering from a liver disease or condition, may also be suffering from diabetes. In some embodiments, diabetes is diabetes associated with insulin resistance. In some embodiments, diabetes is diabetes associated with weight gain. In some embodiments, diabetes is Type II diabetes. In some embodiments, diabetes is Type I diabetes. In some embodiments, diabetes is prediabetes. In some embodiments, a subject suffering from a liver disease or condition, may also be obese. In some embodiments, a subject suffering from a liver disease or condition, may also be suffering from diabetes and obesity.

In some embodiments a liver disease comprises a fatty liver disease. In some embodiments, a liver disease or condition comprises a disease or condition associated with liver diseases or conditions. In some embodiments, the condition associated with a liver disease or condition comprises glucose intolerance or insulin resistance. In some embodiments, insulin resistance comprises peripheral insulin resistance.

In some embodiments, the present disclosure provides methods of treating subject at risk of, or has, fatty liver disease or non-alcoholic fatty liver disease (NAFLD). NAFLD is defined as fat accumulation in the liver exceeding 5% by weight, in the absence of significant alcohol consumption, steatogenic medication, or hereditary disorders (Kotronen et al, Arterioscler Thromb. Vasc. Biol. 2008, 28: 27-38). NAFLD covers a spectrum of liver disease from steatosis to non-alcoholic steatohepatitis (NASH) and cirrhosis. Non-alcoholic steatohepatitis (NASH) is NAFLD with signs of inflammation and hepatic injury. NASH is defined histologically by macrovesicular steatosis, hepatocellular ballooning, and lobular inflammatory infiltrates (Sanyal, Hepatol. Res. 2011. 41: 670-4). NASH is estimated to affect 2-3% of the general population. In the presence of other pathologies, such as obesity or diabetes, the estimated prevalence increases to 7% and 62% respectively (Hashimoto et al, J. Gastroenterol. 2011. 46(1): 63-69).

Fatty liver disease can include an increase in one or more of intracellular fat content, liver weight, liver triglyceride content, plasma circulating alanine aminotransferase (ALT), and lipid content. Treatment of fatty liver disease may be further complicated due to fatty liver disease drugs, e.g., anti-NASH drugs, in clinical development causing increase in cholesterol, in particular LDL cholesterol which is a known risk factor for cardiovascular disease.

In some embodiments, disclosed herein are methods of treating glucose intolerance. In certain embodiments, glucose intolerance comprises metabolic conditions that result in higher than normal blood glucose levels—hyperglycemia. In some embodiments, glucose intolerance comprises dysglycemia. A skilled artisan would appreciate that dysglycemia comprises both prediabetes and diabetes. In some embodiments, dysglyemia includes the conditions of impaired fasting glucose (IFG), impaired glucose tolerance (IGT), and diabetes mellitus (DM). In some embodiments, DM comprises type 2 DM.

The World Health Organization definition for IFG and IGT, glucose intolerance is defined as: A fasting blood glucose level of above 6.0 mmol/L or a blood glucose level of over 7.8 mmol/L 2 hours after consuming 75 g of glucose.

In some embodiments, disclosed herein are methods of reducing insulin resistance. Insulin resistance occurs when cells become insensitive to insulin, resulting in increased glucose in the blood stream.

In some embodiments, insulin resistance comprises a resistance to the hormone insulin, resulting in increasing blood sugar. In some embodiments, insulin resistance is observed when cells in your muscles, fat, and liver don't respond well to insulin and can't use glucose from your blood for energy. To make up for it, the pancreas makes more insulin. Therefore, over time, your blood sugar levels go up. In some embodiments, the term “insulin resistance” may be used interchangeably with the term “insulin sensitivity” having the same meanings and qualities.

In some embodiments, insulin resistance is expressed in the periphery, i.e., organ peripheral to the pancreas. In some embodiments, insulin resistance is expressed in the liver, in skeletal muscle, or in adipose tissue, or in a combination thereof. Weight loss and or exercise may decrease insulin resistance in combination with methods of administering a compound, as disclosed herein. In some embodiments, methods of administering a compound disclosed herein, leads to better glucose use by an organ in the periphery such as but not limited to the liver, skeletal muscle, and or adipose tissue.

In some embodiments, insulin resistance comprises peripheral insulin resistance. In some embodiments, peripheral insulin resistance comprises a failure of a target tissue to increase glucose disposal in response to insulin. In some embodiments, peripheral insulin resistance comprises impaired glucose uptake by liver. In some embodiments, peripheral insulin resistance comprises impaired glucose uptake by skeletal muscle or adipose tissue. In some embodiments, peripheral insulin resistance comprises impaired glucose uptake by skeletal muscle. In some embodiments, peripheral insulin resistance comprises impaired glucose uptake by adipose tissue.

In some embodiments, insulin resistance syndrome comprises diseases and conditions such as obesity, high blood pressure, high cholesterol, and type 2 DM. In some embodiments, insulin resistance is acute. In some embodiments, insulin resistance is chronic.

In some embodiments, a subject administered an effective compound described herein, suffers from diabetes. In some embodiments, a subject administered an effective compound described herein, suffers from diabetes associated with insulin resistance. In some embodiments, a subject administered an effective compound described herein, suffers from diabetes or a related condition. In some embodiments, a subject administered an effective compound described herein, does not suffer from diabetes.

One skilled in the art would appreciate that a subject may suffer from diabetes and further suffer from other liver diseases as are disclosed herein. Moreover, the skilled artisan would appreciate that the term “diabetes” may encompass type 1 and type 2 diabetes and complications due to diabetes, for example retinopathy, nephropathy, and neurosis developed with vascular disorders. Diabetes is classified into insulin-dependent diabetes (IDDM; type 1 diabetes) and non-insulin-dependent diabetes (NIDDM; type 2 diabetes) according to the type of disease a subject is suffering from. In some embodiments, diabetes and related conditions comprise type 2 diabetes, type 1 diabetes, diabetes associated with weight gain, diabetes associated with insulin resistance, or prediabetes. In some embodiments, a subject treated by a method disclosed herein suffers from type 2 diabetes. In some embodiments, a subject treated by a method disclosed herein suffers from type 1 diabetes. In some embodiments, a subject treated by a method disclosed herein suffers from pre-diabetes. In some embodiments, a subject treated by a method disclosed herein suffers from diabetes associated with weight gain. In some embodiments, a subject treated by a method disclosed herein suffers from diabetes associated with insulin resistance.

As known in the art, a high fat diet (HFD) can induce diabetes mellitus that is not seen subjects on a low fat diet (LD), as shown in the Examples below for the wild-type mice. In some embodiments, a HFD comprises a pro-diabetic condition. In diabetes (type 2 for example but not limited to) there is an insulin resistance in the periphery, i.e., in organs such as but not limited to muscles, liver, and or adipose tissue that are peripheral to the pancreas.

In some embodiments, a subject treated using the methods and compounds described herein is suffering from type 1 insulin-dependent diabetes. In some embodiments, a subject treated using the methods and compounds described herein is suffering from type 2 insulin-independent diabetes. In some embodiments, a subject treated using the methods and compounds described herein is pre-diabetic.

In some embodiments, a subject administered an effective compound described herein, suffers from obesity. In some embodiments, a subject administered an effective compound described herein, does not suffer from obesity. In some embodiments, a subject suffering from a liver disease also suffers from obesity. For example, non-alcoholic fatty liver disease (NAFLD) includes a range of conditions caused by a build-up of fat in the liver. It's usually seen in people who are overweight or obese. Early-stage NAFLD does not usually cause any harm, but it can lead to serious liver damage.

In some embodiments, disclosed herein are methods of modulating weight loss. In some embodiments of a method of modulating weight loss, the subject suffers from diabetes, from a liver disease or condition, is glucose intolerant, or is insulin resistant.

### Methods of Treatment

In some embodiments, disclosed herein are methods of treating a liver disease or condition comprising administering to a subject in need a compound effective in reducing GPI-anchored CD59 expression or activity in the liver, thereby treating a liver disease. In some embodiments, disclosed herein are methods of treating a liver disease or condition comprising administering to a subject in need a compound effective in reducing GPI-anchored CD59 expression or activity in skeletal muscle, thereby treating a liver disease. In some embodiments, disclosed herein are methods of treating a liver disease or condition comprising administering to a subject in need a compound effective in reducing GPI-anchored CD59 expression or activity in adipose tissue, thereby treating a liver disease. In some embodiments, disclosed herein are methods of treating a liver disease or condition comprising administering to a subject in need a compound effective in reducing GPI-anchored CD59 expression or activity in the liver, in skeletal muscle, or in adipose tissue, or in any combination thereof, thereby treating a liver disease.

In some embodiments, disclosed herein are methods of modulating weight loss in a subject in need, said method comprising administering to the subject a compound effective in reducing GPI-anchored CD59 expression or activity, said subject suffering from a liver disease or condition, said modulating weight loss comprising maintaining weight, reducing weight, or reducing increased weight gain compared with a subject not administered said compounds. In some embodiments, a compound disclosed herein has therapeutic effectiveness modulating weight loss and reducing GPI-anchored CD59 expression or activity in the liver. In some embodiments, a compound disclosed herein has therapeutic effectiveness modulating weight loss and reducing GPI-anchored CD59 expression or activity in skeletal muscle. In some embodiments, a compound disclosed herein has therapeutic effectiveness modulating weight loss and reducing GPI-anchored CD59 expression or activity in adipose tissue. In some embodiments, a compound disclosed herein has therapeutic effectiveness modulating weight loss and reducing GPI-anchored CD59 expression or activity in the liver, in skeletal muscle, or in adipose tissue, or in any combination thereof.

In some embodiments, disclosed herein are methods of modulating weight loss in a subject in need, said method comprising administering to the subject a compound effective in reducing GPI-anchored CD59 expression or activity, said modulating weight loss comprising maintaining weight, reducing weight, or reducing increased weight gain compared with a subject not administered said compounds. In some embodiments, a compound disclosed herein has therapeutic effectiveness modulating weight loss and reducing GPI-anchored CD59 expression or activity in the liver. In some embodiments, a compound disclosed herein has therapeutic effectiveness modulating weight loss and reducing GPI-anchored CD59 expression or activity in skeletal muscle. In some embodiments, a compound disclosed herein has therapeutic effectiveness modulating weight loss and reducing GPI-anchored CD59 expression or activity in adipose tissue. In some embodiments, a compound disclosed herein has therapeutic effectiveness modulating weight loss and reducing GPI-anchored CD59 expression or activity in the liver, in skeletal muscle, or in adipose tissue, or in any combination thereof.

In some embodiments, disclosed herein are methods of treating diabetes in a subject in need, said method comprising administering to the subject a compound effective in reducing GPI-anchored CD59 expression or activity, said treating diabetes reduces the severity of the diabetes, reduces insulin resistance, leads to weight loss, reduces glucose intolerance, compared with a subject not administered said compounds. In some embodiments, a compound disclosed herein has therapeutic effectiveness treating diabetes, and reducing GPI-anchored CD59 expression or activity in the liver. In some embodiments, a compound disclosed herein has therapeutic effectiveness treating diabetes, and reducing GPI-anchored CD59 expression or activity in skeletal muscle. In some embodiments, a compound disclosed herein has therapeutic effectiveness treating diabetes, and reducing GPI-anchored CD59 expression or activity in adipose tissue. In some embodiments, a compound disclosed herein has therapeutic effectiveness treating diabetes, and reducing GPI-anchored CD59 expression or activity in the liver, in skeletal muscle, or in adipose tissue, or in any combination thereof.

In some embodiments, disclosed herein are methods of reducing insulin resistance in a subject in need, said method comprising administering to the subject a compound effective in reducing GPI-anchored CD59 expression or activity, wherein said insulin resistance is reduced compared with a subject not administered said compounds. In some embodiments, said subject further suffers from diabetes, a liver disease or condition, or obesity, or any combination thereof. In some embodiments, a compound disclosed herein has therapeutic effectiveness reducing insulin resistance, and reducing GPI-anchored CD59 expression or activity in the liver. In some embodiments, a compound disclosed herein has therapeutic effectiveness reducing insulin resistance, and reducing GPI-anchored CD59 expression or activity in skeletal muscle. In some embodiments, a compound disclosed herein has therapeutic effectiveness reducing insulin resistance, and reducing GPI-anchored CD59 expression or activity in adipose tissue. In some embodiments, a compound disclosed herein has therapeutic effectiveness reducing insulin resistance, and reducing GPI-anchored CD59 expression or activity in the liver, in skeletal muscle, or in adipose tissue, or in any combination thereof.

In some embodiments, disclosed herein are methods of treating a liver disease or condition, of modulating weight loss in a subject suffering from a liver disease or condition, of modulating weight loss in a subject in need, of treating diabetes or a related condition in a subject in need, or of reducing insulin resistance in a subject in need, or any combination thereof, said method comprising administering to the subject a compound effective in reducing GPI-anchored CD59 expression or activity, wherein said insulin resistance is reduced compared with a subject not administered said compounds. In some embodiments, said subject further suffers from diabetes, a liver disease or condition, or obesity, or any combination thereof. In some embodiments, a compound disclosed herein has therapeutic effectiveness reducing insulin resistance, and reducing GPI-anchored CD59 expression or activity in the liver. In some embodiments, a compound disclosed herein has therapeutic effectiveness reducing insulin resistance, and reducing GPI-anchored CD59 expression or activity in skeletal muscle. In some embodiments, a compound disclosed herein has therapeutic effectiveness reducing insulin resistance, and reducing GPI-anchored CD59 expression or activity in adipose tissue. In some embodiments, a compound disclosed herein has therapeutic effectiveness reducing insulin resistance, and reducing GPI-anchored CD59 expression or activity in the liver, in skeletal muscle, or in adipose tissue, or in any combination thereof.

In some embodiments, methods of use of a therapeutic compound, described herein, reduce expression of CD59 in the liver; reduces the quantity of GPI-anchored CD59 in the liver; or inhibits functional activities of GPI-anchored CD59 in the liver; or a combination thereof. In some embodiments, methods of use of a therapeutic compound, described herein, reduce expression of CD59 in skeletal muscle; reduces the quantity of GPI-anchored CD59 in skeletal muscle; or inhibits functional activities of GPI-anchored CD59 in skeletal muscle; or a combination thereof. In some embodiments, methods of use of a therapeutic compound, described herein, reduce expression of CD59 in adipose tissue; reduces the quantity of GPI-anchored CD59 in adipose tissue; or inhibits functional activities of GPI-anchored CD59 in adipose tissue; or a combination thereof. In some embodiments, methods of use of a therapeutic compound, described herein, reduce expression of CD59 in the liver, skeletal muscle, or adipose tissue, or any combination thereof; reduces the quantity of GPI-anchored CD59 in the liver, skeletal muscle, or adipose tissue, or any combination thereof; or inhibits functional activities of GPI-anchored CD59 in the liver, skeletal muscle, or adipose tissue, or any combination thereof; or a combination thereof.

In some embodiments, a compound comprises a polynucleotide, a oligonucleotide, a protein, a peptide, an antibody or a small molecule disclosed herein. In some embodiments, a liver disease or condition comprises a fatty liver disease, insulin resistance, peripheral insulin resistance, or glucose intolerance. In some embodiments, the subject is further suffering from diabetes or obesity or a combination thereof.

In some embodiments, the present disclosure provides a method of treating or preventing fatty liver disease in a subject in need thereof, the method comprising administering a compound (e.g., a polynucleotide, a protein, a peptide, an antibody, or a small molecule provided herein) effective for lowering the expression of CD59 in the subject, thereby treating or preventing fatty liver disease in the subject. In some embodiments, the present disclosure provides a method of treating or preventing fatty liver disease in a subject in need thereof, the method comprising administering a compound (e.g., a polynucleotide, a protein, a peptide, an antibody, or a small molecule provided herein) effective for lowering the activity of CD59 in the subject, thereby treating or preventing fatty liver disease in the subject. It is contemplated that downregulation of CD59 in a subject with liver disease would confer the same protection as the CD59 loss-of-function mutant. Thus, in some embodiments, the present disclosure provides a method of lowering CD59 expression in a cell of a subject, the method comprising administering a compound comprising a polynucleotide disclosed herein effective for lowering the expression of CD59 in the subject.

In some embodiments, the present disclosure provides a method of treating or preventing insulin resistance in a subject in need thereof, the method comprising administering a compound (e.g., a polynucleotide, a protein, a peptide, an antibody, or a small molecule provided herein) effective for lowering the expression of CD59 in the subject, thereby treating or preventing insulin resistance in the subject. In some embodiments, the present disclosure provides a method of treating or preventing insulin resistance in a subject in need thereof, the method comprising administering a compound (e.g., a polynucleotide, a protein, a peptide, an antibody, or a small molecule provided herein) effective for lowering the activity of CD59 in the subject, thereby treating or preventing insulin resistance in the subject. In some embodiments, insulin resistance comprises peripheral insulin resistance.

In some embodiments, the present disclosure provides a method of treating or preventing glucose intolerance in a subject in need thereof, the method comprising administering a compound (e.g., a polynucleotide, a protein, a peptide, an antibody, or a small molecule provided herein) effective for lowering the expression of CD59 in the subject, thereby treating or preventing glucose intolerance in the subject. In some embodiments, the present disclosure provides a method of treating or preventing glucose intolerance in a subject in need thereof, the method comprising administering a compound (e.g., a polynucleotide, a protein, a peptide, an antibody, or a small molecule provided herein) effective for lowering the activity of CD59 in the subject, thereby treating or preventing glucose intolerance in the subject.

In some embodiments, the present disclosure provides a method of treating diabetes in a subject in need thereof, the method comprising administering a compound (e.g., a polynucleotide, a protein, a peptide, an antibody, or a small molecule provided herein) effective for lowering the expression of CD59 in the subject, thereby treating diabetes in the subject. It is contemplated that downregulation of CD59 in a subject suffering from diabetes would confer the same protection as the CD59 loss-of-function mutant. Thus, in some embodiments, the present disclosure provides a method of lowering CD59 expression in a cell of a subject, the method comprising administering a compound comprising a polynucleotide disclosed herein effective for lowering the expression of CD59 in the subject.

In some embodiments, the present disclosure provides a method of modulating weight loss in a subject in need thereof, the method comprising administering a compound (e.g., a polynucleotide, a protein, a peptide, an antibody, or a small molecule provided herein) effective for lowering the expression of CD59 in the subject, thereby modulating weight loss in the subject. It is contemplated that downregulation of CD59 in an obese subject or a subject with diabetes associated with weight gain or in a subject needed to reduce their weight for medical reasons, would confer the same protection as the CD59 loss-of-function mutant. Thus, in some embodiments, the present disclosure provides a method of lowering CD59 expression in a cell of a subject, the method comprising administering a compound comprising a polynucleotide disclosed herein effective for lowering the expression of CD59 in the subject.

In some embodiments, methods disclosed herein reduce GPI-anchored CD59 expression or activity. In certain embodiments, reduce GPI-anchored CD59 expression or activity comprises a reduced quantity of GPI-anchored CD59 or inhibiting functional activities of GPI-anchored CD59. In some embodiments, reduce GPI-anchored CD59 expression or activity comprises a reduced quantity of GPI-anchored CD59. In some embodiments, reduce GPI-anchored CD59 expression or activity comprises inhibiting functional activities of GPI-anchored CD59.

In some embodiments of a method treating or preventing fatty liver disease, the method also modulates weight loss in said subject, wherein said modulating weight loss comprises maintaining weight, reducing weight, or reducing increased weight gain compared with a subject not administered said compound.

In some embodiments, the present disclosure provides a method for modulating weight loss in a subject in need, the method comprising administering a compound (e.g., a polynucleotide, a protein, a peptide, an antibody, or a small molecule provided herein) effective for lowering the expression of CD59 in the subject, thereby modulating weight loss in a subject suffering from a liver disease or condition. In some embodiments, the present disclosure provides a method for modulating weight loss in a subject in need, the method comprising administering a compound (e.g., a polynucleotide, a protein, a peptide, an antibody, or a small molecule provided herein) effective for lowering the activity of CD59 in the subject, thereby modulating weight loss in a subject suffering from a liver disease or condition. It is contemplated that modulating weight loss with the downregulation of CD59 in a subject suffering from a liver disease or condition, would confer the same protection as the CD59 loss-of-function mutant. Thus, in some embodiments, the present disclosure provides a method of lowering CD59 expression in a cell of a subject, the method comprising administering a compound comprising a polynucleotide disclosed herein effective for lowering the expression of CD59 in the subject.

In some embodiments, a compound used in the methods disclosed herein comprises an oligonucleotide, an antibody or binding fragment thereof, a protein, a peptide, or a small molecule. In some embodiments, a compound used in the methods disclosed herein comprises a polynucleotide, an oligonucleotide, an antibody or binding fragment thereof, a protein, a peptide, or a small molecule. In some embodiments, a compound used in the methods disclosed herein comprises a polynucleotide. In some embodiments, a compound used in the methods disclosed herein comprises an oligonucleotide. In some embodiments, a compound used in the methods disclosed herein comprises an antibody or binding fragment thereof. In some embodiments, a compound used in the methods disclosed herein comprises a protein. In some embodiments, a compound used in the methods disclosed herein comprises a peptide. In some embodiments, a compound used in the methods disclosed herein comprises a small molecule.

In some embodiments, methods of treating a liver disease or condition comprising administering to a subject in need an oligo nucleotide effective in reducing GPI-anchored CD59 expression or activity in the liver, thereby treating a liver disease. In some embodiments, methods of treating a liver disease or condition comprising administering to a subject in need an oligo nucleotide effective in reducing GPI-anchored CD59 expression or activity in skeletal muscle, thereby treating a liver disease. In some embodiments, methods of treating a liver disease or condition comprising administering to a subject in need an oligo nucleotide effective in reducing GPI-anchored CD59 expression or activity in adipose tissue, thereby treating a liver disease. In some embodiments, methods of treating a liver disease or condition comprising administering to a subject in need an oligo nucleotide effective in reducing GPI-anchored CD59 expression or activity in the liver, skeletal muscle, or adipose tissue, or any combination thereof, thereby treating a liver disease. In some embodiments, method of treating a liver disease or condition comprising administering to a subject in need an oligonucleotide comprising an antisense oligonucleotide, an interfering RNA compound, a siRNA, a miRNA, or a guide RNA.

In some embodiments, methods for modulating weight loss in a subject suffering from a liver disease or condition comprise administering to the subject an oligo nucleotide effective in reducing GPI-anchored CD59 expression or activity in the liver, thereby modulating weight loss. In some embodiments, methods for modulating weight loss in a subject suffering from a liver disease or condition comprise administering to the subject an oligo nucleotide effective in reducing GPI-anchored CD59 expression or activity in skeletal muscle, thereby modulating weight loss. In some embodiments, methods for modulating weight loss in a subject suffering from a liver disease or condition comprise administering to the subject an oligo nucleotide effective in reducing GPI-anchored CD59 expression or activity in adipose tissue, thereby modulating weight loss. In some embodiments, methods for modulating weight loss in a subject suffering from a liver disease or condition comprise administering to the subject an oligo nucleotide effective in reducing GPI-anchored CD59 expression or activity in the liver, skeletal muscle, adipose tissue, or any combination thereof, thereby modulating weight loss. In some embodiments, method for modulating weight loss in a subject suffering from a liver disease or condition comprises administering to a subject in need an oligonucleotide comprising an antisense oligonucleotide, an interfering RNA compound, a siRNA, a miRNA, or a guide RNA.

In some embodiments, a method of use herein discloses use of an oligonucleotide comprising a conjugate group attached at the 5′ or 3′ end of the oligonucleotide. In some embodiments, methods of use comprise administration of an oligonucleotide comprising a conjugate comprising at least one GalNac moiety.

In some embodiments, methods of use comprise administration of an oligonucleotide comprising a contiguous nucleotide sequence at least 80%, 85%, 90%, 95%, or 100% complementary to an equivalent length of a target CD59 mRNA transcript or CD59 mRNA precursor. In some embodiments, the target CD59 mRNA comprises the nucleotide sequence set forth in any of SEQ ID NOs: 3-12.

In some embodiments, a method of use comprises use of an oligonucleotide comprising a guide RNA, wherein said method further comprises administering a polynucleotide encoding a CRISPR-Cas9 endonuclease operatively linked to a liver promoter, wherein (b) the guide RNA comprises a contiguous nucleotide sequence complementary to an equal length portion of a target nucleic acid sequence encoding the CD59 protein, said target nucleic acid sequence comprises the sequence of one of SEQ ID NOs:3-12, or is complementary to an equal length portion of a nucleic acid sequence upstream or downstream of the sequence encoding the CD59 protein.

In certain embodiments, the compounds comprise a polynucleotide or an oligonucleotide comprising a region that is complementary to a target nucleic acid, wherein the target nucleic acid is CD59 RNA. In each of the embodiments described herein, the compound may target CD59 RNA. In certain embodiments, CD59 RNA has the sequence set forth in any one of SEQ ID NOs:3-12. In certain embodiments, contacting a cell with a compound comprising a polynucleotide complementary to an equal length portion in any one of SEQ ID NOs:3-12 reduces the amount of CD59 RNA, and in certain embodiments reduces the amount of CD59 protein. In certain embodiments, the compound comprises a modified oligonucleotide. In certain embodiments, the cell is in a subject in need thereof. In certain embodiments, administering the subject with a compound comprising a polynucleotide complementary to an equal length portion in any one of SEQ ID NOs:3-12 results in reduced liver damage, steatosis, liver fibrosis, liver inflammation, liver scarring or cirrhosis, or liver failure in the subject. In certain embodiments, administering the subject with a compound comprising a polynucleotide complementary to an equal length portion in any one of SEQ ID NOs:3-12 results in modulated weight loss compare with a subject not administered the compound. In certain embodiments, the subject is human. In certain embodiments, the compound comprises a modified oligonucleotide and a conjugate group. In certain embodiments, the compound is a RNAi compound.

In some embodiments, a method of use comprises use of a compound comprising an anti-CD59 antibody or binding fragment thereof, wherein said use reduces GPI-anchored CD59 expression or activity in the liver, thereby treating a liver disease or condition in the subject. In some embodiments, a method of use comprises use of a compound comprising an anti-CD59 antibody or binding fragment thereof, wherein said use reduces GPI-anchored CD59 expression or activity in the liver, thereby modulating weight loss in a subject suffering from a liver disease or condition. In some embodiments, a method of use comprises use of a compound comprising an anti-CD59 antibody or binding fragment thereof, wherein said use reduces GPI-anchored CD59 expression or activity in skeletal muscle, thereby treating a liver disease or condition in the subject. In some embodiments, a method of use comprises use of a compound comprising an anti-CD59 antibody or binding fragment thereof, wherein said use reduces GPI-anchored CD59 expression or activity in skeletal muscle, thereby modulating weight loss in a subject suffering from a liver disease or condition. In some embodiments, a method of use comprises use of a compound comprising an anti-CD59 antibody or binding fragment thereof, wherein said use reduces GPI-anchored CD59 expression or activity in adipose tissue, thereby treating a liver disease or condition in the subject. In some embodiments, a method of use comprises use of a compound comprising an anti-CD59 antibody or binding fragment thereof, wherein said use reduces GPI-anchored CD59 expression or activity in adipose tissue, thereby modulating weight loss in a subject suffering from a liver disease or condition. In some embodiments, a method of use comprises use of a compound comprising an anti-CD59 antibody or binding fragment thereof, wherein said use reduces GPI-anchored CD59 expression or activity in the liver, skeletal muscle, or adipose tissue, or any combination thereof, thereby treating a liver disease or condition in the subject. In some embodiments, a method of use comprises use of a compound comprising an anti-CD59 antibody or binding fragment thereof, wherein said use reduces GPI-anchored CD59 expression or activity in the liver, skeletal muscle, or adipose tissue, or any combination thereof, thereby modulating weight loss in a subject suffering from a liver disease or condition.

In some embodiments, the anti-CD59 antibody or fragment thereof binds to a cell surface epitope of CD59. In some embodiments, method of use of an anti-CD59 antibody or fragment thereof reduces CD59 expression, reduces the quantity of GPI-anchored CD59, or inhibits the functional activities of GPI-anchored CD59.

In some embodiments, in method of use herein, the anti-CD59 antibody or fragment of use thereof binds to an epitope comprised within SEQ ID NO: 1. In some embodiments, in method of use herein, the anti-CD59 antibody or fragment of use thereof binds to an epitope comprised within an amino acid sequence at least 80%, 85%, 90%, 95%, or 100% identical with the amino acid sequence set forth in SEQ ID NO: 1.

In some embodiments, a method of use comprises use of a compound comprising a small molecule, wherein said small molecule administration reduces GPI-anchored CD59 expression or activity in the liver, thereby treating a liver disease or condition in the subject. In some embodiments, a method of use comprises use of a compound comprising a small molecule, wherein said small molecule administration reduces GPI-anchored CD59 expression or activity in skeletal muscle, thereby treating a liver disease or condition in the subject. In some embodiments, a method of use comprises use of a compound comprising a small molecule, wherein said small molecule administration reduces GPI-anchored CD59 expression or activity in adipose tissue, thereby treating a liver disease or condition in the subject. In some embodiments, a method of use comprises use of a compound comprising a small molecule, wherein said small molecule administration reduces GPI-anchored CD59 expression or activity in the liver, skeletal muscle, or adipose tissue, or any combination thereof, thereby treating a liver disease or condition in the subject. In some embodiments, the small molecule binds to a cell surface epitope of CD59. In some embodiments, a method of use of small molecule reduces CD59 expression, reduces the quantity of GPI-anchored CD59, or inhibits the functional activities of GPI-anchored CD59.

In some embodiments, in a method of use herein, the small molecule binds to a ligand binding region comprised within SEQ ID NO: 1. In some embodiments, in method of use herein, the small molecule use thereof binds to a ligand binding region comprised within an amino acid sequence at least 80%, 85%, 90%, 95%, or 100% identical with the amino acid sequence set forth in SEQ ID NO: 1.

In some embodiments, methods of use comprise administration of a small molecule that binds to a contiguous nucleotide sequence at least 80%, 85%, 90%, 95%, or 100% complementary to an equivalent length of a target CD59 mRNA transcript or CD59 mRNA precursor. In some embodiments, the target CD59 mRNA comprises the nucleotide sequence set forth in any of SEQ ID NOs: 3-12.

In some embodiments, the subject of the present disclosure in need of treatment or prevention of fatty liver disease has one or more of non-alcoholic fatty liver disease (NAFLD), non-alcoholic steatohepatitis (NASH) (cirrhotic or non-cirrhotic NASH), hepatocellular carcinoma (HCC) and/or liver fibrosis. In some embodiments, the subject of the present disclosure in need of treatment or prevention of fatty liver disease has alcoholic fatty liver disease (AFLD) or alcoholic steatohepatitis (ASH) (cirrhotic or non-cirrhotic ASH). In some embodiments, the subject of the present disclosure in need of treatment or prevention of fatty liver disease has one or more of liver damage, steatosis, liver fibrosis, liver inflammation, liver scarring or cirrhosis, and liver failure.

In some embodiments, the subject of the present disclosure in need of treatment or prevention of fatty liver disease has one or more of non-alcoholic fatty liver disease (NAFLD), non-alcoholic steatohepatitis (NASH) (cirrhotic or non-cirrhotic NASH), hepatocellular carcinoma (HCC), liver fibrosis, diabetes, and/or is suffering from obesity. In some embodiments, the subject of the present disclosure in need of treatment or prevention of fatty liver disease has alcoholic fatty liver disease (AFLD) or alcoholic steatohepatitis (ASH) (cirrhotic or non-cirrhotic ASH) or diabetes or is suffering from obesity or any combination thereof. In some embodiments, the subject of the present disclosure in need of treatment or prevention of fatty liver disease has one or more of liver damage, steatosis, liver fibrosis, liver inflammation, liver scarring or cirrhosis, liver failure, diabetes, and obesity.

In some embodiments, the subject of the present disclosure suffering from a liver disease or condition and in need of modulated weight loss has one or more of non-alcoholic fatty liver disease (NAFLD), non-alcoholic steatohepatitis (NASH) (cirrhotic or non-cirrhotic NASH), hepatocellular carcinoma (HCC), liver fibrosis, diabetes, and/or is suffering from obesity. In some embodiments, the subject of the present disclosure suffering from a liver disease or condition and in need of modulated weight loss has alcoholic fatty liver disease (AFLD) or alcoholic steatohepatitis (ASH) (cirrhotic or non-cirrhotic ASH) or diabetes or is suffering from obesity or any combination thereof. In some embodiments, the subject of the present disclosure suffering from a liver disease or condition and in need of modulated weight loss has one or more of liver damage, steatosis, liver fibrosis, liver inflammation, liver scarring or cirrhosis, liver failure, diabetes, and obesity.

In some embodiments, the method decreases one or more of intracellular fat content, liver weight, liver triglyceride content, and lipid content in the subject. In some embodiments, the amount of cholesterol and/or LDL of the subject decreases following administration of the compound disclosed herein. In some embodiments, the subject of the present disclosure in need of treatment or prevention of fatty liver disease or suffering from a liver disease and in need of weight loss modulation, has a cardiovascular disease such as dyslipidemia. In certain embodiments, the disease is mixed dyslipidemia. In certain embodiments, the disease is hypercholesterolemia. In certain embodiments, the disease is familial hypercholesterolemia.

In some embodiments, the present disclosure provides a method of lowering intracellular fat content in a liver cell in a subject, the method comprising administering a compound effective for lowering the expression of CD59 in the subject. In some embodiments, the compound is a polynucleotide provided herein.

In some embodiments, the present disclosure provides a method of lowering cholesterol in a subject, the method comprising administering a compound effective for lowering the expression of CD59 in the subject. In some embodiments, the compound is a polynucleotide provided herein.

In some embodiments, a liver disease or condition comprises fatty liver disease, NASH, or peripheral insulin resistance. In some embodiments, a fatty liver disease comprises non-alcoholic fatty liver disease (NAFLD), alcoholic fatty liver disease (AFLD), alcoholic steatohepatitis (ASH) (cirrhotic or non-cirrhotic ASH), non-alcoholic steatohepatitis (NASH) (cirrhotic or non-cirrhotic NASH), hepatocellular carcinoma (HCC), or liver fibrosis, or any combination thereof.

In some embodiments, a liver disease or condition comprises glucose intolerance or insulin resistance or a combination thereof. In some embodiments of methods disclosed herein, insulin resistance comprises peripheral insulin resistance.

In some embodiments, a subject administered a compound disclosed herein suffers from the fatty liver disease and has liver damage, steatosis, liver inflammation, liver scarring or cirrhosis, liver failure, or peripheral insulin resistance, or any combination thereof. In some embodiments, a subject administered a compound disclosed herein suffers from liver damage, steatosis, liver inflammation, liver scarring or cirrhosis, liver failure, or peripheral insulin resistance, or any combination thereof. In some embodiments, a subject administered a compound disclosed herein suffers from the fatty liver disease and has liver damage, steatosis, liver inflammation, liver scarring or cirrhosis, liver failure, or peripheral insulin resistance, or any combination thereof, in further combination with diabetes or obesity or a combination thereof. In some embodiments, a subject administered a compound disclosed herein suffers from liver damage, steatosis, liver inflammation, liver scarring or cirrhosis, liver failure, or peripheral insulin resistance, or any combination thereof, in further combination with diabetes or obesity or a combination thereof.

In some embodiments, methods of use administering a compound reduces expression of CD59 in the liver, reduces the quantity of GPI-anchored CD59, or inhibits functional activities of GPI-anchored CD59, or a combination thereof. In some embodiments, methods of use administering a compound reduces expression of CD59 in skeletal muscle, reduces the quantity of GPI-anchored CD59, or inhibits functional activities of GPI-anchored CD59, or a combination thereof. In some embodiments, methods of use administering a compound reduces expression of CD59 in adipose tissue, reduces the quantity of GPI-anchored CD59, or inhibits functional activities of GPI-anchored CD59, or a combination thereof. In some embodiments, methods of use administering a compound reduces expression of CD59 in the liver, skeletal muscle, or adipose tissue, or any combination thereof, reduces the quantity of GPI-anchored CD59, or inhibits functional activities of GPI-anchored CD59, or a combination thereof.

In some embodiments, methods of use administration of a polynucleotide, an oligonucleotide, an antibody or binding fragment thereof, or a small molecule reduces expression of CD59 in the liver, reduces the quantity of GPI-anchored CD59, or inhibits functional activities of GPI-anchored CD59, or a combination thereof. In some embodiments, methods of use administration of a polynucleotide, an oligonucleotide, an antibody or binding fragment thereof, or a small molecule reduces expression of CD59 in skeletal muscle, reduces the quantity of GPI-anchored CD59, or inhibits functional activities of GPI-anchored CD59, or a combination thereof. In some embodiments, methods of use administration of a polynucleotide, an oligonucleotide, an antibody or binding fragment thereof, or a small molecule reduces expression of CD59 in adipose tissue, reduces the quantity of GPI-anchored CD59, or inhibits functional activities of GPI-anchored CD59, or a combination thereof. In some embodiments, methods of use administration of a polynucleotide, an oligonucleotide, an antibody or binding fragment thereof, or a small molecule reduces expression of CD59 in the liver, skeletal muscle, or adipose tissue, or any combination thereof, reduces the quantity of GPI-anchored CD59, or inhibits functional activities of GPI-anchored CD59, or a combination thereof.

In some embodiments, a method of use disclosed herein targets a noncanonical function of CD59, wherein said method provides a protective role treating the development of glucose intolerance, insulin resistance, and or fatty liver, or a combination thereof in a subject in need. As shown in Example 2, CD59 knockout (KO) by itself did not reveal clear abrogation in insulin secretion or glucose intolerance; however, with special conditions such as a high fat diet (HFD), glucose intolerance was exposed in wild-type (WT) animals and surprisingly prevented in CD59 KO animals.

All references cited herein, including patents, patent applications, papers, textbooks and the like, and the references cited therein, to the extent that they are not already, are hereby incorporated herein by reference in their entirety.

### EXAMPLES

### Example 1: Materials and Methods

Metabolic cages will be used to verify food consumption and expenditure. Intraperitoneal glucose tolerance test (IPGTT), Insulin Tolerance Test (ITT), and glucose tolerance test (GTT) will be studied along with glucose, insulin, and glucagon levels. Histology and immune histology will be performed in muscle and three lobes of liver. Tissue studies will include MRI and PET-CT. Insulin signaling will be studied downstream and analysis of cell metabolism using LC-MS and isotope tracers will be performed.

Immunofluorescence labelling and pathology. Adult mice (WT and KO CD59) will be anesthetized, and different tissues will be excised and fixed for 30 minutes in 4% paraformaldehyde. The CD59 knocked out is the GPI-surface membrane bound form of CD59. Briefly, samples will be permeabilized by ethanol and blocked for 1 hour with 0.5% Triton X-100 PBS, incubated overnight at 40° C. with a variety of primary antibodies diluted in blocking solution, washed, and incubated again for 45 min at room temperature with secondary antibodies. Finally, slides will be mounted with Elvanol™ and analyzed. For mouse pancreas sections, rabbit polyclonal antibody against mouse CD59 will be used and other antibodies including guinea pig anti-insulin, and mouse anti-glucagon. Fluorophore-coupled secondary antibodies will include 488-coupled anti-rabbit IgG, Cy3-coupled anti-mouse IgG, Cy5-coupled anti-chicken IgG.24 Livers will be harvested from mice, placed into cassettes, and submerged in 10% formalin for 24 hrs. The cassettes will be moved into 70% ethanol for 24 hrs prior to creating the paraffin blocks. For mouse liver sections, rat antibody against mouse CD8, anti-CD4, anti-CD3, and anti-F4/80 will be used. Paraffin-embedded liver sections will be stained with hematoxylin and eosin (H&E) Fluorescence images will be obtained with a Zeiss confocal microscope fitted with a Hamamatsu ORCA-ER CCD camera (Hamamatsu City, Shizuoka Pref, Japan). Images will be acquired and processed using the Zen2012 (Carl Zeiss, Oberkochen, Germany) and Photoshop software (Adobe, San Jose, CA, USA).

Monitoring body weight, glucose level and food intake measurements during a lean diet (LD) and HFD. Mice will fast for 2 hours before performing body weight, glucose levels, and food intake measurements. Tail vein blood will be collected to monitor glucose levels using a Glucometer ACCU CHECK PERFORMA (DYN Diagnostics, Israel).

A HFD, Intraperitoneal Injection Glucose Tolerance Test (IPGTT), and Insulin Tolerance Test (ITT). The study population will include male C57BL/6 mice aged 2 months. Mice will be given unrestricted access to a pelleted HFD (45% kcal fat diet, 21% MF, 2% SBO) (TD.08811, Envigo Teklad Diets, Indianapolis, IN, USA) for 17 weeks. Glucose tolerance will be examined after HFD feeding for 4, 8, 12, and 16 weeks. The mice will be fasted for 16 hours (overnight fasting) prior to the GTTs by transferring them to clean cages with access to drinking water but with no food in the hopper or cage bottom. Mice will be weighed prior to the experiment. The volume of 30% glucose solution will be calculated (1.5 g of glucose/kg body mass) for IP injection. The glucose stock solution will be prepared by dissolving 3 g of D-glucose (Sigma-Aldrich), in 10 mL of sterile saline solution. The concentration of insulin solution will be 1 U/kg (10 μl of NovoRapid Insulin as part in 5 ml sterile saline). The solution will be sterilized by passing it through a 0.2-μm filter into a sterile 15-mL Falcon tube. Blood glucose of the mice will be measured according to the manufacturer's instructions (Accu-Chek Performa, Roche, Basel, Switzerland) in 5-μL samples collected from a small incision made at the tip of the tail immediately before treatment and at 0, 15, 30, 60, and 120 min after intraperitoneal injection of glucose.

The results will be presented as a time course of absolute blood glucose measurements (mg/dL) and as the area under the curve (AUC). The statistical comparison of glucose levels will be processed through an analysis of variance by Prism 6.0 (GraphPad Software, San Diego, CA) with data expressed as mean±SEM for the indicated number of observations. Data will be assessed for statistical significance using a two-tailed unpaired Student's t-test. A P value <0.05 will be considered statistically significant.

Insulin and Glucagon ELISA. Mice will fast for 4 hours before collecting blood samples. Insulin concentration will be determined using a commercial Insulin Elisa Kit (Crystal Chem, 90095). Glucagon concentration will be determined using a commercial Glucagon Elisa Kit (Mercodia, 10-1271-01).

Western blot analysis and sample preparations. Prior to animal scarification at week 16 of the high fat diet experiment, mice will be intraperitoneally injected with insulin solution (2.5 U/kg). 5 minutes post injection, Mice liver tissues will be harvested and immediately snapped frozen in liquid nitrogen and stored at −80° C. until further use. After adding the RIPA buffer, the frozen tissues will be sonicated for several seconds. To perform WB, the samples will be diluted 1:30 by ×3 sample buffer (0.5M Tris HCl 6.8 pH, SDS, Glycerol and Bromophenolblue) and heated at 95° C. for 5 min. 10% polyacrylamide gels will be used, then transferred to nitrocellulose/PVDF membranes. 5% BSA solution will be used as a blocking solution for 45 min. Membranes will be incubated overnight at 4° C. with the first antibody (1:1000) followed by extensive washes and incubation with HRP-conjugated secondary antibody (1:10,000) for 45 min. For insulin pathway signaling antibodies from Cell Signaling (Danvers, MA, USA): AKT (catalog #0.4691), pAKT (catalog #0.4060), pIRS (catalog #0.2381), pAMPK (catalog #0.2535), P110 (catalog #. C73F8), and P56(catalog #. D68F8) will be used. ECL reaction imaging will be done using the ChemiDoc XRP+ system (Bio-Rad, Hercules, CA, USA).

Metabolic Phenotyping Measurement (Promethion System, metabolic cages). The metabolic rate of the various mouse strains on a LD or HFD will be measured by using the Promethion Metabolic Phenotyping System by Sable Systems International (Las Vegas, NV, USA). By incorporating subsystems for open-circuit indirect calorimetry, feeding, water intake, activity, running wheel, BMI, and core temperature measurements in conventional live-in home cages that minimize stress (see configuration at: https://www.sablesys.com/products/promethion-line/promethion-high-definition-continuous-respirometry-system-for-mice/), this fully automated system is the “one-test” solution for simultaneous multi-parameter assessment for metabolic, behavioral and physiological research. While in the metabolic chambers, the mice will have free access to food and water. 8 individually caged mice will be analyzed at the same time. At the end of the experiments, mice will be returned to the home cage. Mice will be housed in these cages for 3 days. These are conventional live-in home cages with regular bedding.

In vivo micro-PET & MRI scanning. Experiments will be performed at the Wohl Institute for Translational Medicine at Hadassah Hebrew University Medical Center. PET-MRI images will be acquired on a 7T 24 cm bore cryogen-free MR scanner based on proprietary dry magnet technology (MR Solutions, Guildford, UK) with a 3-ring PET insert that uses the latest silicon photomultiplier (SiPM) technology. The PET subsystem contains 24 detector heads arranged in three octagons of 116 mm diameter. For MRI acquisition, a mouse quadrature RF volume coil will be used. Mice will be anesthetized with isoflurane vaporized with 02. Isoflurane will be used at 3.0% for induction and at 1.0-2.0% for maintenance. The mice will be positioned on a heated bed, which allows for continuous anesthesia and breathing rate monitoring. To determine the distribution of [18F]-FDG in mice, the tracer will be injected into the tail vein (230±30 mCi in 200 mL). Images will be analyzed using VivoQuant preclinical image postprocessing software (Invicro, Needham, MA, USA). PET-MRI raw data will be processed using the standard software provided by the manufacturers. Manual tissue segmentation of kidneys, liver, muscle, heart, brain and bladder will be carried out on coregistered 3D MR images. Regions-of-interest will be used to calculate tissue radiotracer uptake from the reconstructed PET images.

Analysis of Cell Metabolism Using LC-MS and Isotope Tracers. Prof. Gottleib from the Technion (please see a letter of collaboration) has established a relatively simple, quick method for sample extraction.38 His team has identified over 300 metabolites in various samples on their LC-MS platform by matching mass and retention time with commercial standard compounds. Metabolites that include amino acids, organic acids, sugars, phosphates (glycolysis and pentose phosphate pathways), nucleotides, and cofactors (such as CoA, NADH) will be studied. Stable isotope tracers and isotopologues of many metabolites found in their compound databases will be used for examining intracellular kinetics. We will initially study plasma and peripheral tissues, including muscle and liver (divided to three lobes). Sample preparation for proteomics. For tissue proteomics, mice will be sacrificed by cervical dislocation, and liver and muscle tissues will be harvested and snap-frozen immediately using liquid nitrogen. Plasma blood samples will be collected from the submandibular vein. All blood samples will be centrifuged at 2000 g for 15 min at 4° C. Representative samples will be excised on dry ice, added to extraction solution, and homogenized under dry ice vapor using a Precellys24 bead-based homogenizer (Bertin Instruments, Montigny-le-Bretonneux, France) for 3×20 s at 5000 rpm. All samples will be centrifuged at 16,000 g for 5 min at 4° C. Supernatant will be transferred to new tubes and incubated in a tabletop shaker in the dark at 1400 rpm for 1 h at room temperature. Protein concentration will be determined by BCA assay and samples stored at −80° C. until further processing.

RNA extraction. Total RNA from liver tissues of mice will be isolated using the RNeasy Mini kit (QIAGEN, GmbH, Hilden, Germany) according to the manufacturer's protocol. Complementary DNA (cDNA) will be synthesized with iScript cDNA Synthesis Kit (Bio-Rad Laboratories), according to the manufacturer's instructions.

### Example 2: Analysis of High Fat Diet (HFD) in CD59 Deficient Mice

Objective: To explore the role of CD59 in diabetes and the development of insulin resistance and fatty liver. This includes (1) exploring the effect of a high fat diet (HFD) on wild-type (WT) and CD59 deficient mice by performing Intraperitoneal Glucose Tolerance Test (IPGTT) and Insulin Tolerance Test (ITT) experiments; (2) understanding the consequences of a HFD on insulin signaling pathway in WT and CD59 deficient mice; (3) evaluating insulin and glucagon blood levels and secretion thereof in WT and CD59 deficient mice; (4) and explore the metabolic effects and differences between WT and CD59 deficient mice after consuming high fat diet in metabolic cages and by MRI/PET imaging.

### Methods:

Intraperitoneal Injection Glucose Tolerance Test (IPGTT). The study population comprised male C57BL/6 mice, all at age 2 months or 12 months old. The mice were fasted for 16 hours (overnight fasting) prior to the Glucose Tolerance Tests (GTTs) by transferring mice to clean cages with no food in the hopper or bottom of the cage, and with access to drinking water at all times. Mice were weighed before the experiment. The volume of 30% glucose solution was calculated (1.5 g of glucose/kg body mass) for intraperitoneal (IP) injection. The glucose stock solution was prepared by dissolving 3 g of D-glucose (Sigma-Aldrich), in 10 mL of sterile saline solution. The solution was then sterilized by passing it through a 0.2-μm filter into a sterile 15-mL Falcon tube. Blood glucose of mice was measured according to the manufacturer's instructions (Accu-Chek Performa, Roche, Basel, Switzerland) in 5-μL samples collected from a small incision made at the tip of the tail immediately before treatment and at 0, 15, 30, 60, and 120 min after IP injection of glucose.

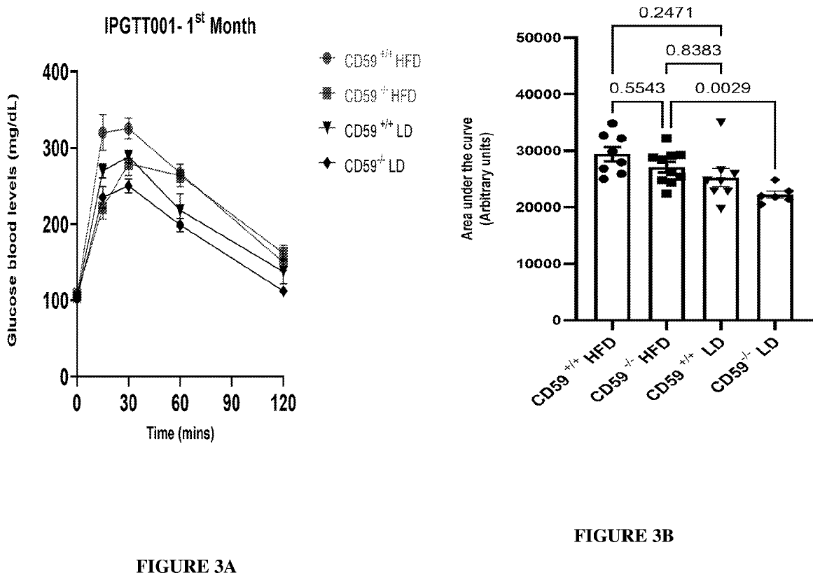

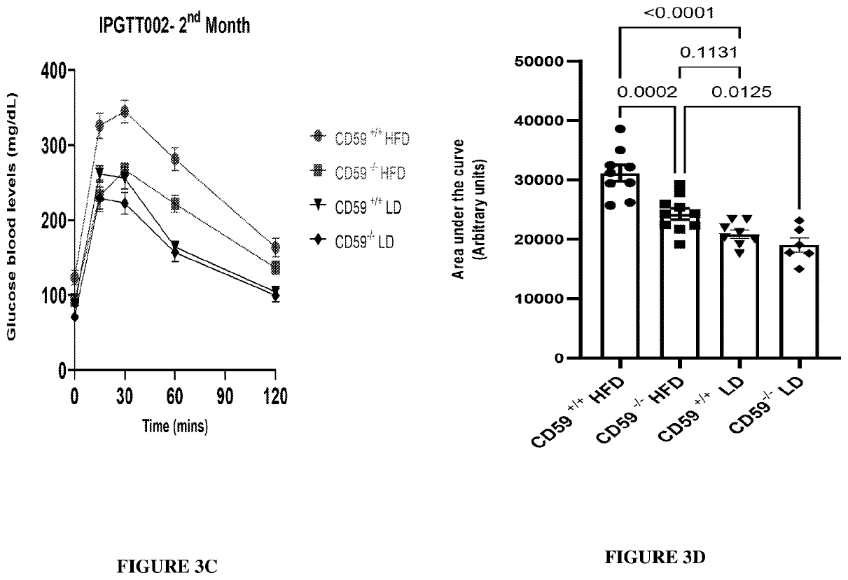

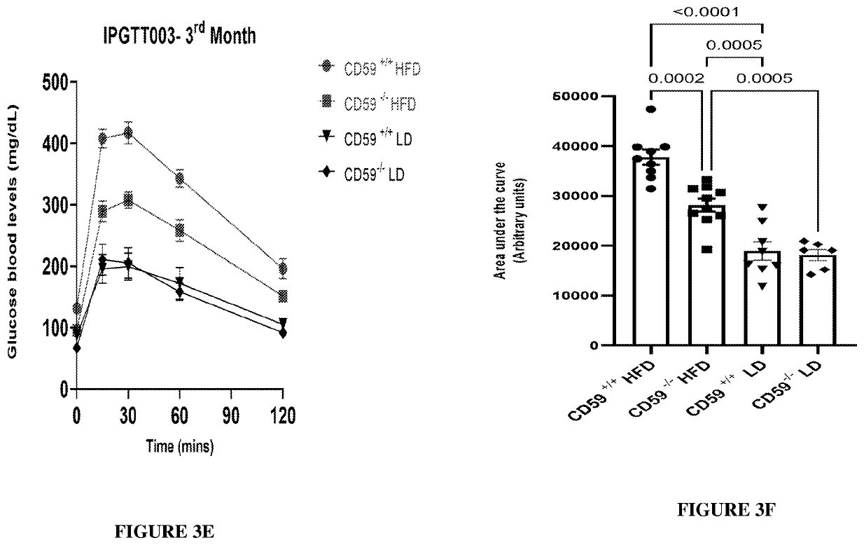

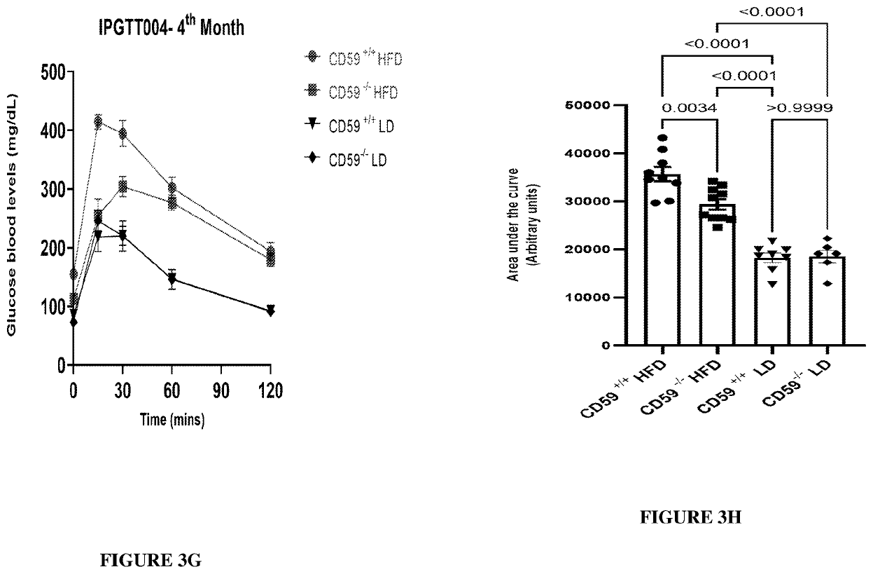

The results are presented herein as a time course of absolute blood glucose measurements (mg/dL) and as the area under the curve (AUC) (FIGS. 3A-3H). The statistical comparison of glucose levels was processed through an analysis of variance by Prism 6.0 (GraphPad Software, San Diego, CA). Data are expressed as mean±SEM for the indicated number of observations. Data were assessed for statistical significance by using a two-tailed unpaired Student's t-test. A P value of less than 0.05 was considered statistically significant.

High fat diet & Intraperitoneal Injection Glucose Tolerance Test (IPGTT). The study population comprised male C57BL/6 mice, all at age 2 months old. Mice were given unrestricted access to a pelleted high-fat diet [45% kcal Fat Diet (21% MF, 2% SBO) TD.08811, Envigo Teklad Diets, Indianapolis, Indiana, United States] for 17 weeks. The glucose tolerance of the mice was examined after high fat diet (HFD) feeding for 4, 8, 12 and 16 weeks. The mice were fasted for 16 hours (overnight fasting) prior to the GTTs by transferring mice to clean cages with no food in the hopper or the bottom of cage, and with access to drinking water at all times. Mice were weighed before the experiment. The volume of 30% glucose solution was calculated (1.5 g of glucose/kg body mass) for IP injection. The glucose stock solution was prepared by dissolving 3 g of D-glucose (Sigma-Aldrich), in 10 mL of sterile saline solution. The solution then was sterilized by passing it through a 0.2-μm filter into a sterile 15-mL Falcon tube. Blood glucose of mice was measured according to the manufacturer's instructions (Accu-Chek Performa, Roche, Basel, Switzerland) in 5-μL samples collected from a small incision made at the tip of the tail immediately before treatment and at 0, 15, 30, 60, and 120 min after intraperitoneal injection of glucose.

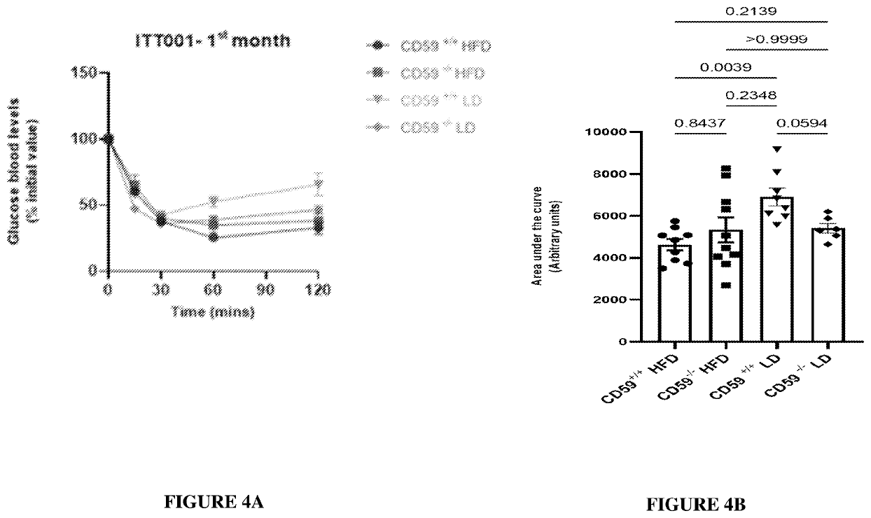

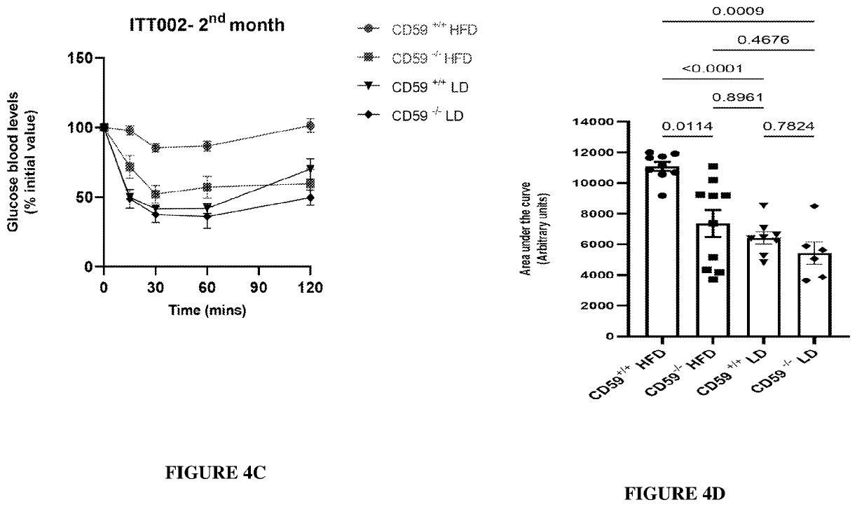

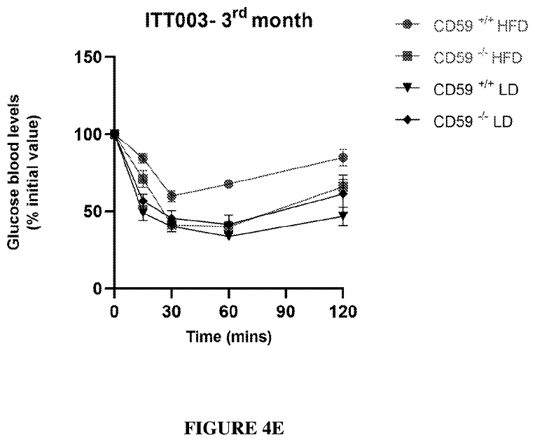

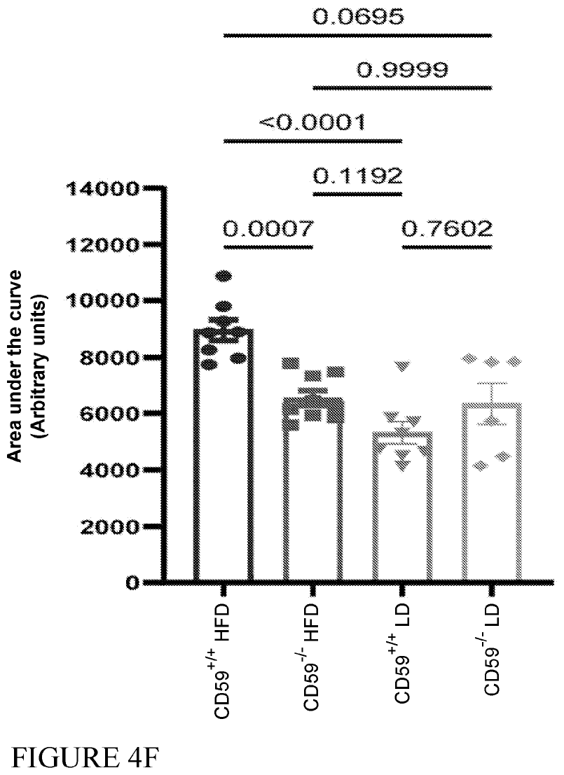

The results are presented herein as a time course of absolute blood glucose measurements (mg/dL) and as the area under the curve (AUC) (FIGS. 4A-4F). The statistical comparison of glucose levels was processed through an analysis of variance by Prism 6.0 (GraphPad Software, San Diego, CA). Data are expressed as mean±SEM for the indicated number of observations. Data were assessed for statistical significance by using a two-tailed unpaired Student's t-test. A P value of less than 0.05 was considered statistically significant.

Additional methods are provided above in Example 1.

### Results:

The results presented here focus on the possible peripheral effects of CD59. To address that effect, weekly changes in body weight of WT and CD59 KO on a LD or a HFD were measured.

The preliminary study presented here provided unexpected results and a role for CD59, or the absence thereof, in the treatment of fatty liver disease and peripheral insulin resistance not previously predicted by knowledge in the art.

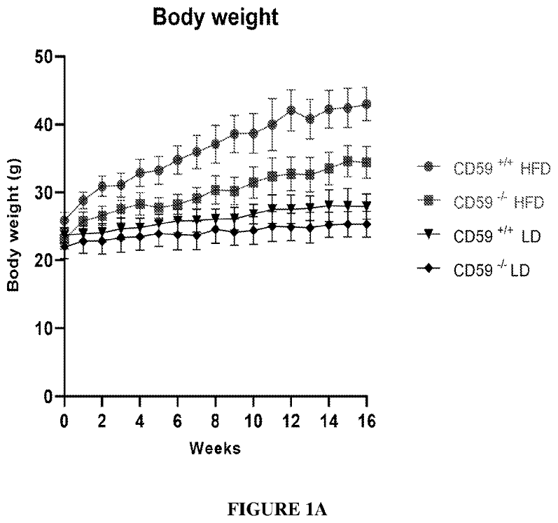

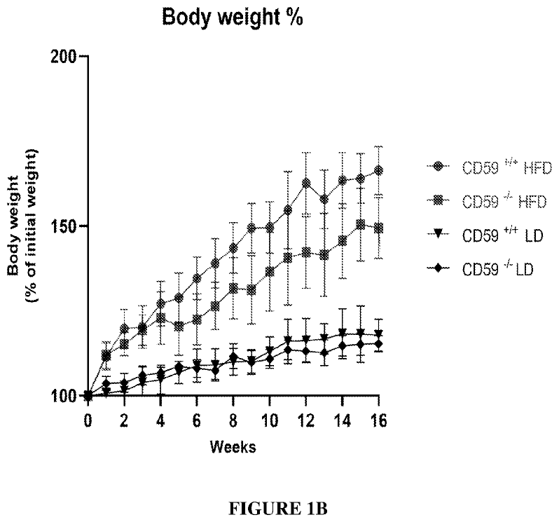

Surprisingly, CD59 wild-type (WT) mice gained significantly more weight compared to knockout (KO; knockout is for GPI-linked CD59) mice when on a HFD, but on lean diets both WT and CD59 KO mice had similar body weights (FIGS. 1A-1B). It was also observed that there was no significant difference in food intake (data not shown).

As a further step in characterization, CD59 protein will be stained by immunofluorescence staining in paraffin and frozen sections of CD59 WT mice. Immunofluorescence labeling of WT murine pancreas will be performed to localize CD59 in pancreas islets. Based on previous studies and research, it is expected to find CD59 on the plasma membrane, but intracellular j-cell CD59 and co-localization with insulin will be confirmed in addition to membrane staining.

Glucose blood levels of CD59 WT HFD mice were higher compared to KO HFD mice and other control groups on standard diet, i.e., a LD. Glucose blood levels of CD59 WT mice (WT mice on an LD) were higher compared to KO STD mice.

In order to verify that the animals consume calories similarly, metabolic cages were used.

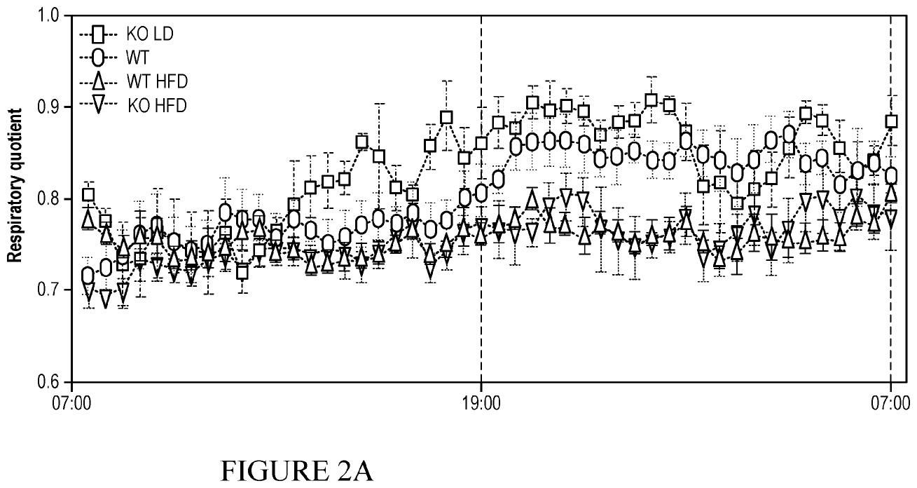

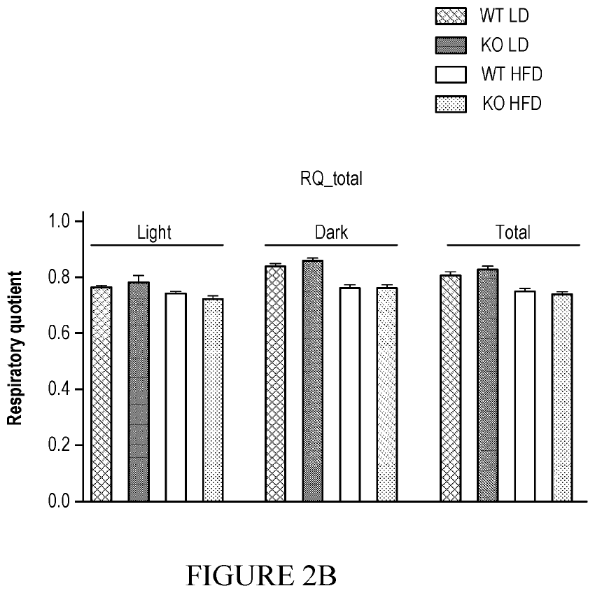

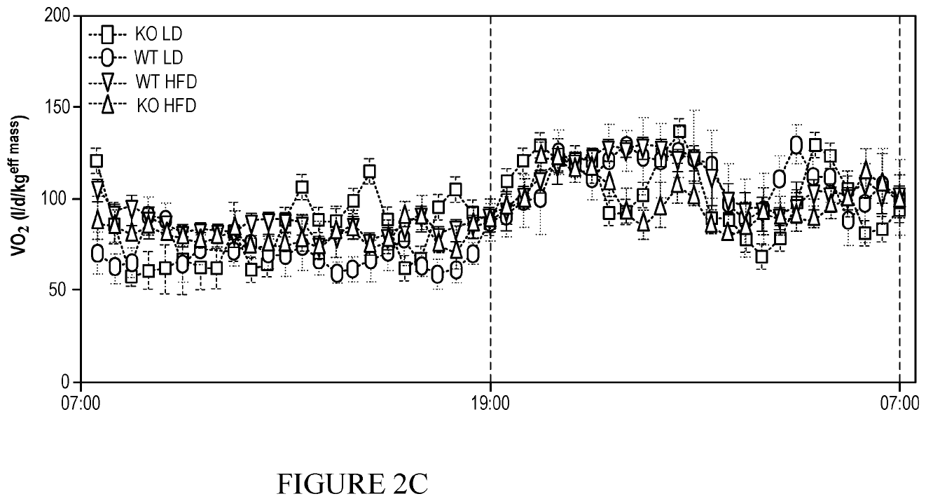

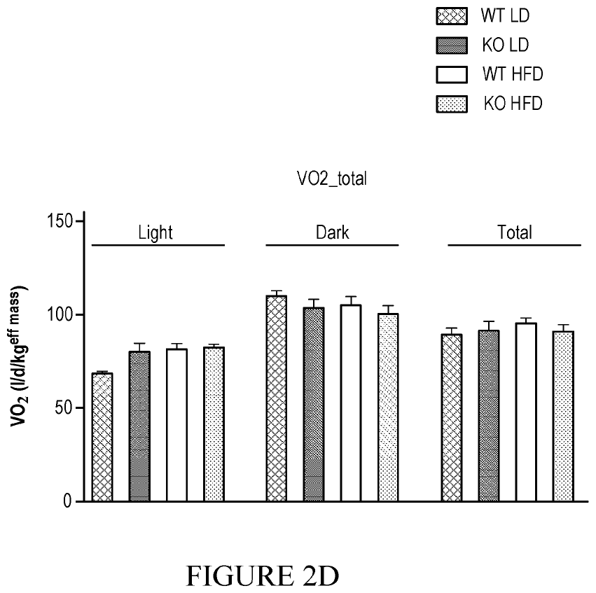

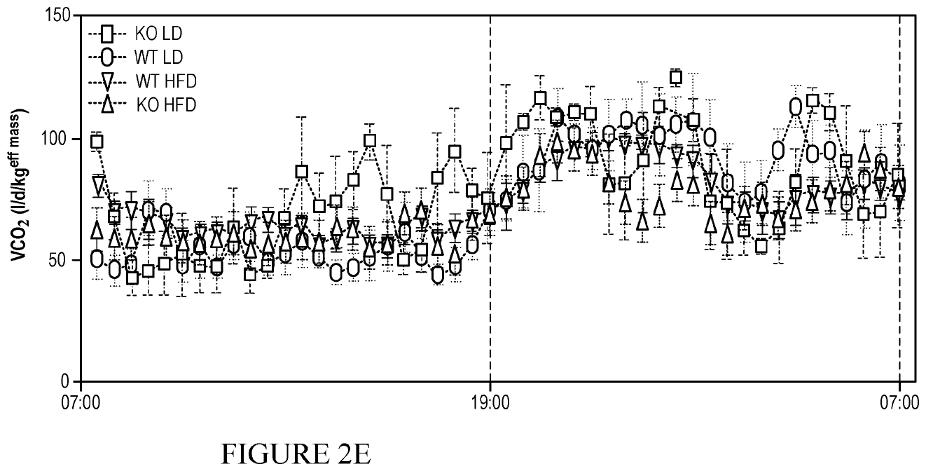

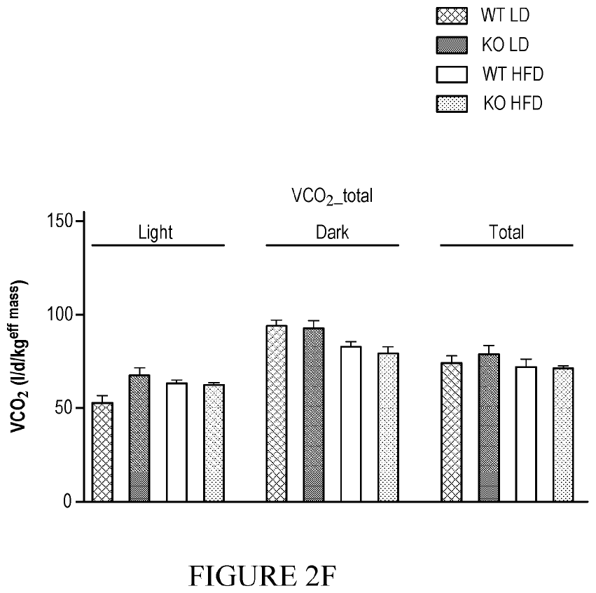

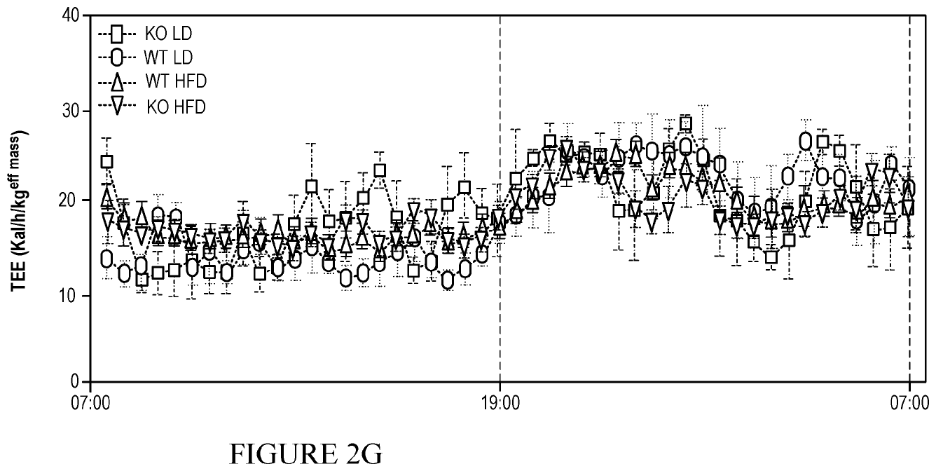

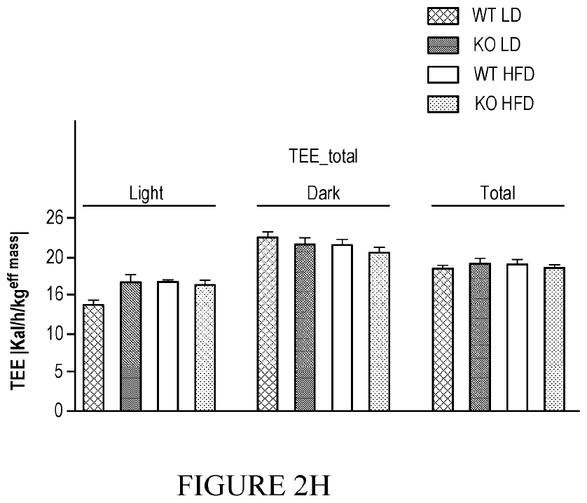

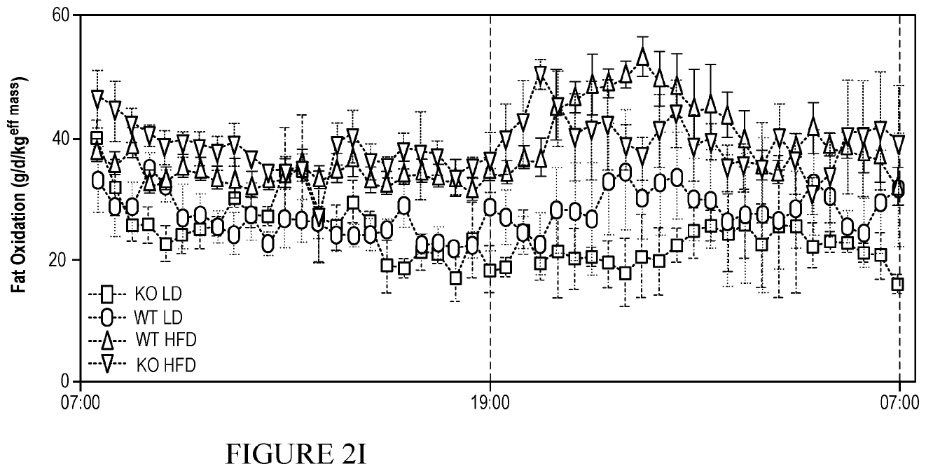

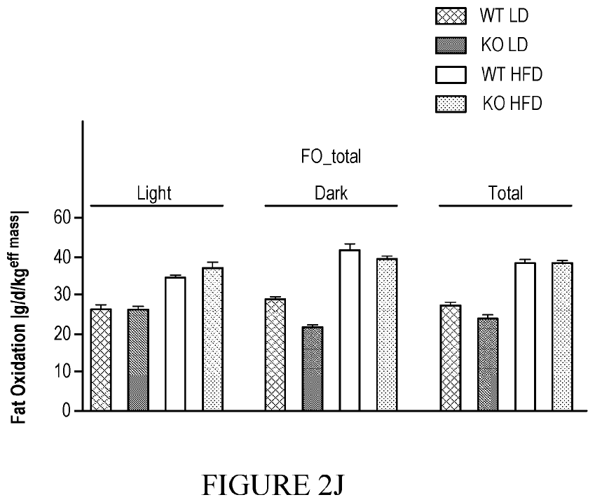

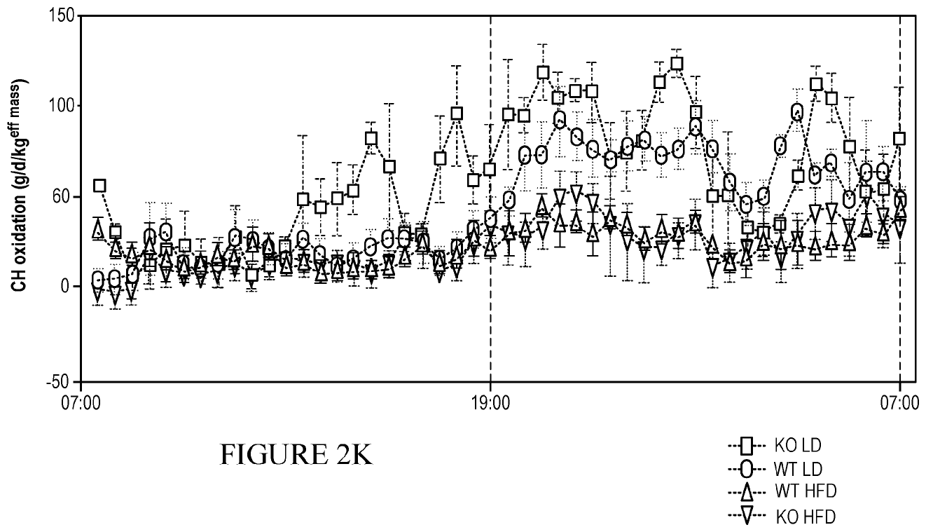

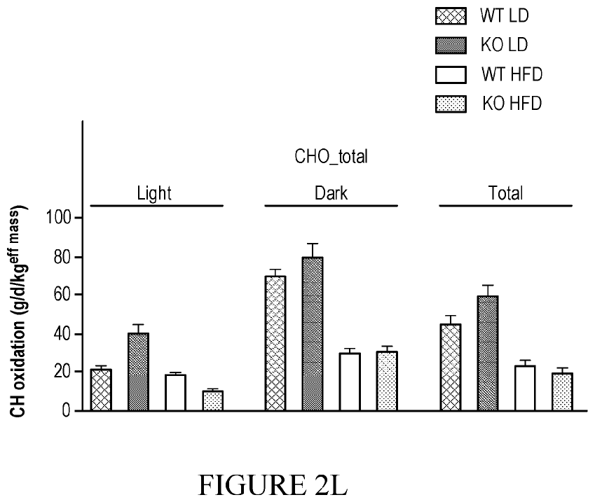

As shown in FIGS. 2A-2L, the animals consumed food similarly in regard to calories.

As shown in FIGS. 1A and 1B, there was an expected increase in body weight on a LD that did not differ much in WT or CD59 KO mice; however, there was a significant change in body weight increase in WT compared to a reduced increase in CD59 KO mice on the HFD. food consumption and activity were verified and were similar using metabolic cages. Preliminary results showed no difference in food intake and energy expenditure (FIGS. 2A-2L). Findings are representative of 3 experiments. Some expected differences were noted between the LD and HFD, but no significant differences were noted between WT and CD59 on HFD in food intake and energy expenditure.

Surprisingly because no one could predict such a role for CD59, but still confirming the observation presented in FIGS. 1A and 1B, IPGTT experiments showed an increased glucose intolerance in CD59 WT mice during the experiment compared to the other groups. (FIGS. 3A-3H) IPGTT experiments showed increased glucose intolerance in CD59 WT mice during the experiment compared to the other groups. These results were more significant during HFD time progression, as shown in area under curve (AUC) statistics. These results were more significant every month as shown in the statistics of Area Under Curve (AUC)

To directly assess insulin sensitivity, mice were subjected to an ITT at different stages of high-fat feeding. CD59 KO mice on HFD exhibited significantly greater reduction in blood glucose after insulin injection compared with CD59 WT mice on HFD. (FIGS. 4A-4F) No significant differences were observed in the clearance of glucose from the blood of the control groups, WT and KO mice on lean diet (LD).

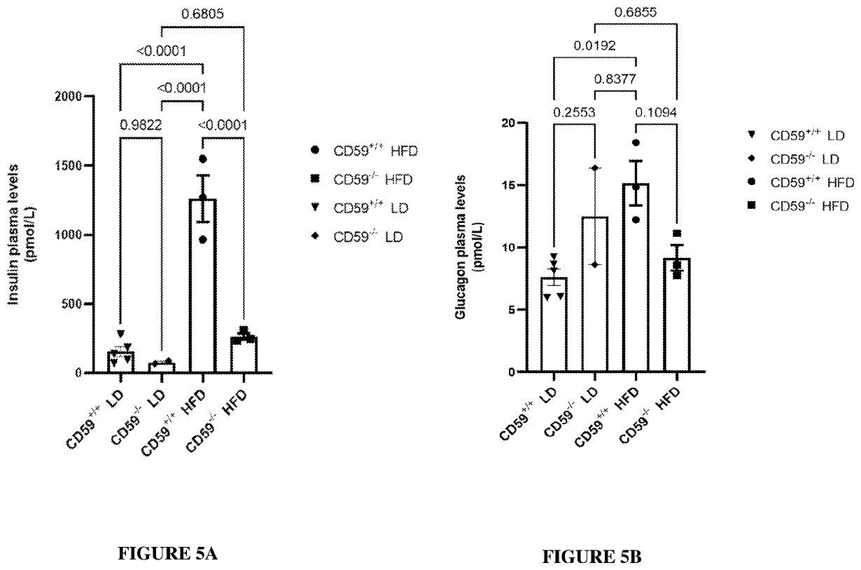

For further investigations, both insulin and glucagon plasma levels of fasted mice were measured by ELISA (FIGS. 5A-5B). As shown, significantly higher levels of insulin in CD59 WT on HFD group were observed compared to all other groups. High levels of insulin in HFD of WT in comparison to CD59 KO were observed (FIGS. 5A and 5B). To a lesser extent, a similar pattern was observed for glucagon in HFD WT and KO mice. These results are in agreement that insulin resistance was developed and that 3-cells of CD59 WT on HFD are in a compensation mode that resulted in hyperinsulinemia. Glucagon levels were significantly higher in CD59 KO mice on a HFD mice compared to a LD but did not reach significance when compared to CD59 KO levels.

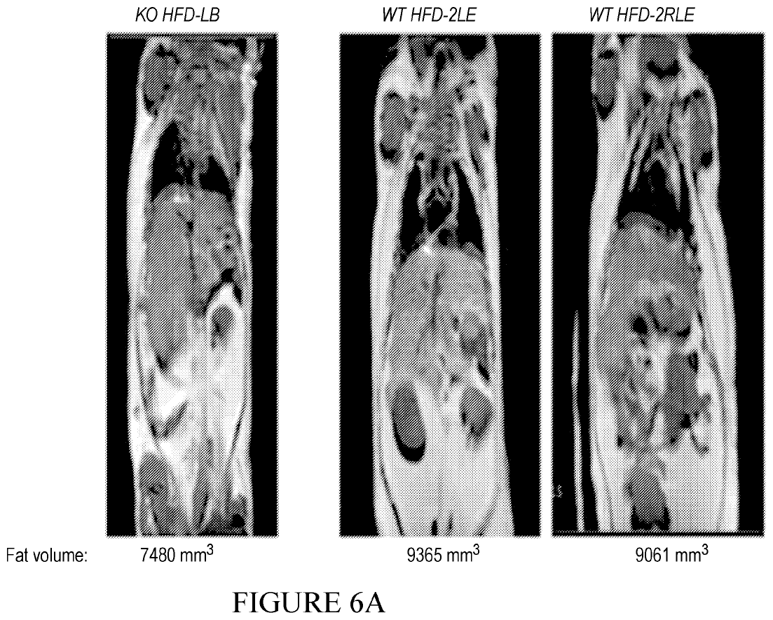

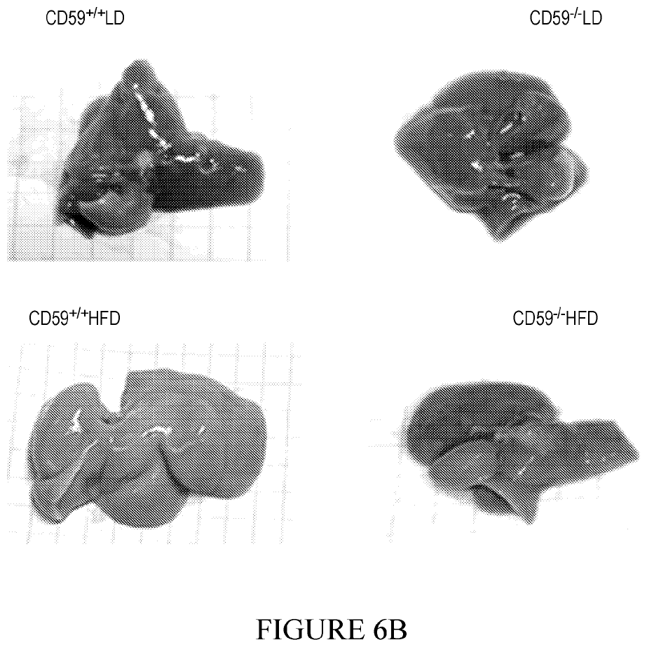

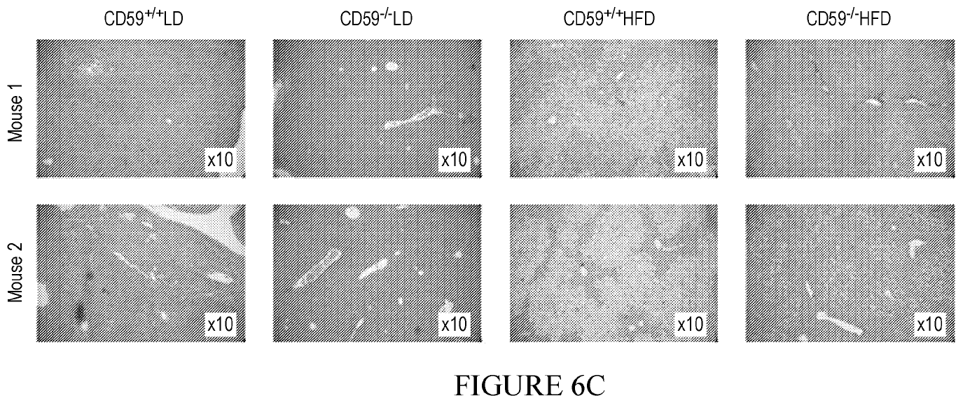

An MRI of the 4 groups of animals were then performed. FIG. 6A shows a representative MRI of 2 WT animals and one CD59 KO mouse, all on a HFD. As shown, fat volume was much more pronounced in WT mice and reached an average of 23% higher than in CD59 KO mice. This was also seen in liver macroscopic appearance (FIG. 6B) and microscopic appearance (FIG. 6C).

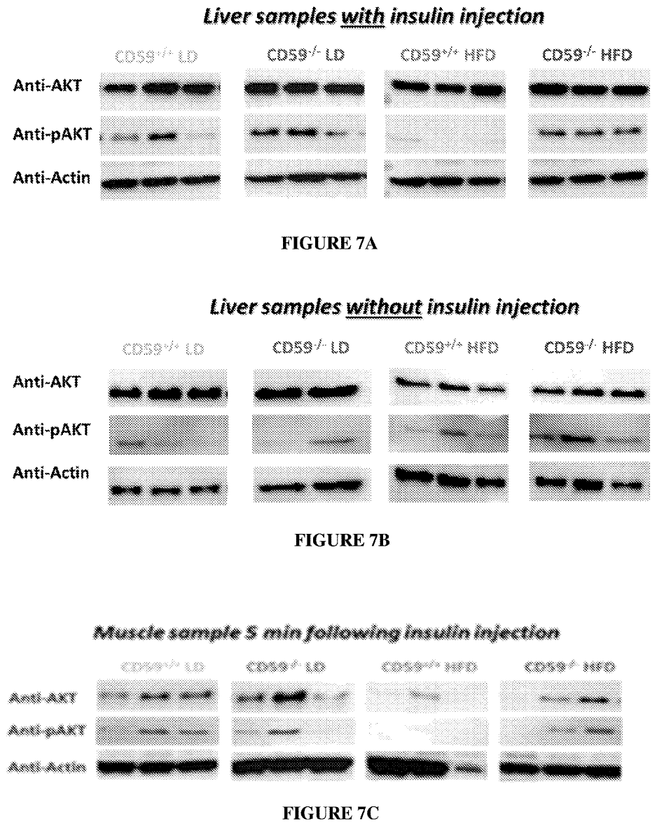

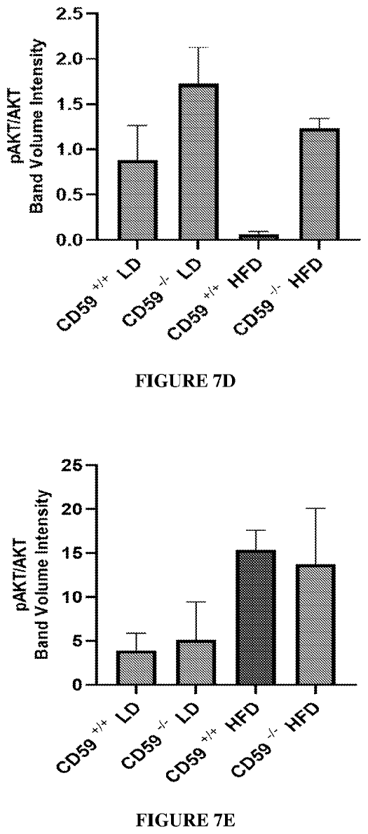

In order to further investigate the insulin resistance that was suggested by the previous observations, insulin signaling was studied. Released insulin participates in various metabolic pathways in cells, such as glycogen deposition in liver and skeletal muscles, downregulation of gluconeogenesis in liver, and a stimulation of lipogenesis and inhibition of lipolysis, but most importantly in increased glucose uptake through insulin receptor signaling pathway. One of its signaling pathways is the phosphatidylinositol 3 kinase (PI3K) pathway, which elicits AKT/PKB kinase phosphorylation. The pathway is activated when insulin attaches to insulin dimer receptors and triggers intracellular autophosphorylation of their tyrosine residues, which constitute an attachment for IRS proteins. This pathway is responsible for GLUT4 translocation to the cell membrane and glucose inflow. Insulin resistance has been previously proved in both in human and animal work and linked with defects to both upstream and downstream targets of Akt/PKB. Thus, in order to evaluate insulin resistance in each group of mice liver and muscle tissues were extracted post insulin injection and Western blots performed using AKT, pAKT, p110 and pS6 antibodies (FIGS. 7A-7E and 10). CD59 WT mice on a HFD developed insulin resistance where CD59 KO mice on a HFD remained normally sensitive to insulin. Phosphorylation of AKT, PI3K (110), and pS6 in CD59 WT on HFD in their liver and muscle samples was significantly decreased, and normal phosphorylation of AKT was found in CD59 KO mice on a HFD compared to those on LD.

The results presented in FIGS. 7A-7E show higher insulin resistance in CD59 WT on HFD in their liver and normal phosphorylation of AKT in CD59 KO mice on HFD compared to mice on standard diet.

As shown in FIGS. 7A-7E, 5 min following insulin injection, liver tissue showed a pAKT appearance in mice on a LD that was completely abrogated in WT on a HFD but not in CD59 KO mice. Muscle results were similar but need to be repeated due to Western blot quality.

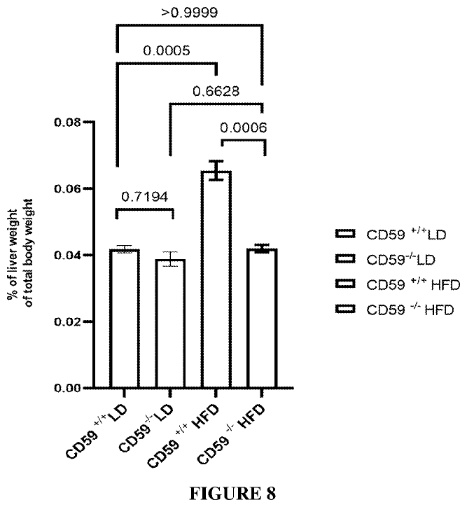

Higher liver weight in WT compared to CD59 KO was observed in mice on both the HFD and LD (FIG. 8). The color of the liver was whiter in the WT, wherein white identifies areas of fat accumulation.

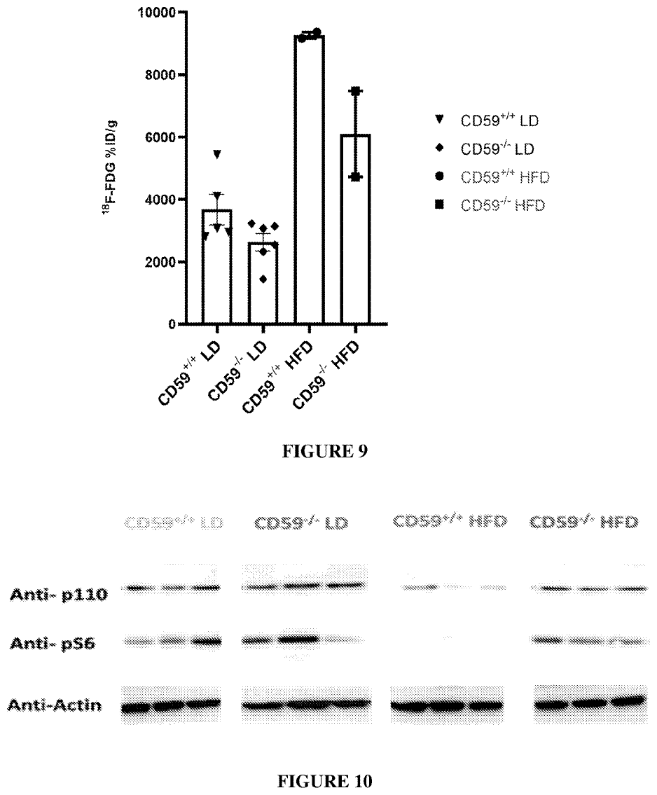

The observations based on FIGS. 7A-7E were confirmed using MRI (FIG. 9).

FIG. 8 taken together with Table 1 below, show higher liver weight of liver in WT compared to CD59 deficient. The color of the liver was whiter in the WT.

TABLE 1
Liver Weight/Total Body Weight

| | | | | Liver/Body |
|---|---|---|---|---|
| Mice | Label | Body Weight | Liver weight | weight |
| WT HFD | R3 + R3 | 45.8 | 3.52 | 8% |
| WT HFD | R1 + L3 | 43.6 | 2.44 | 6% |
| WT HFD | L3 | 47.6 | 3.166 | 7% |
| WT HFD | L1 | 41.4 | 2.52 | 6% |
| WT HFD | L1 + L3 + R | 38.2 | 2.5 | 7% |
| WT HFD | R3 | 38.2 | 2.55 | 7% |
| KO HFD | R1 | 28.2 | 1.13 | 4% |
| KO HFD | R + L | 33.7 | 1.27 | 4% |
| KO HFD | L1 + L3 | 33.4 | 1.304 | 4% |
| KO HFD | R3 + L3 | 34.4 | 1.5 | 4% |
| KO HFD | R3 + L1 | 42.5 | 1.9 | 4% |
| KO HFD | R1 + L1 | 36.5 | 1.65 | 5% |
| KO HFD | R1 + L3 | 39 | 1.7 | 4% |
| WT LD | R3 | 22.6 | 0.93 | 4% |
| WT LD | R1 | 27 | 0.997 | 4% |
| WT LD | R3 + R1 | 25 | 0.94 | 4% |
| WT LD | W/O | 28 | 1.17 | 4% |
| WT LD | L1 | 28.5 | 1.236 | 4% |
| WT LD | R1 | 35 | 1.53 | 4% |
| WT LD | L3 | 30.2 | 1.29 | 4% |
| WT LD | L1 | 28.2 | 1.319 | 5% |
| KO LD | W/O | 22.3 | 0.95 | 4% |
| KO LD | L1 | 28.2 | 1.15 | 4% |
| KO LD | R1 | 25.2 | 0.97 | 4% |
| KO LD | L1 + L3 | 27.8 | 0.85 | 3% |
| KO LD | R3 | 21.5 | 0.89 | 4% |

FIG. 10 shows p110 and pS6 signaling in the liver following injection of insulin. Weak-to-no appearance is seen with a HFD but not in CD59 KO mice.

Taken together, these results suggest that CD59 KO mice are protected from the development of insulin resistance, glucose intolerance, and fatty liver. These experiments will be repeated and the metabolomic effect on muscle and liver studied as well as full insulin signaling. Further exploring of insulin signaling together with RNA seq and LC-MS metabolomics investigation will be performed.

### Summary:

CD59 is known to inhibit the final step of membrane attack complex (MAC) formation to protect host cells from MAC-mediated injury.

Intracellular 3-cell (beta cell) CD59 was described as having a role in insulin secretion. In the current examples the role of CD59 was tested in the periphery in pro-diabetes conditions using HFD. The wild-type mice on HFD developed diabetes and liver disease and surprisingly, the CD59 KO mice were protected and did not develop diabetes or liver disease. Surprisingly and unexpectedly, CD59 deficient mice developed glucose intolerance when fed a high fat diet and gained significantly less weight than WT mice similarly maintained on a high fat diet. Moreover, the CD59 deficient mice did not develop fatty liver as expected. It is expected that treatment of wild-type mice with anti-CD59 compound that inhibits non-canonical functions of CD59 will protect the wild-type mice of HFD.

To summarize, it is surprising to find that silencing CD59 protected mice from developing fatty liver and diabetes that is expressed clearly in high fat diet model. Avoiding development of insulin resistance is an important aspect of preventing diabetes and or preventing the development of a fatty liver. The results shown here suggest that silencing CD59 in the liver will provide protection from the development of fatty liver and perhaps even will reverse the existence of fatty liver.

Because CD59 plays other roles in the body, an effective method of reducing or eliminating CD59 should be restricted to reducing or eliminating CD59 in certain organs, for example the liver, possibly striated muscles, and other organs. General silencing may be dangerous with regard to provoking a condition similar to Paroxysmal nocturnal hemoglobinuria (PNH) (if red blood cells (RBC) are silenced) and similarly in the peripheral nervous system. However, if CD59 reduction or elimination is restricted to liver, striated muscles, or adipose tissue, the negative effects of lack of CD59 expression and/or function in RBC could be avoided. Furthermore, it is expected that reduction or elimination of CD59 expression/function in the liver would treat fatty liver.

In conclusion, the results presented herein demonstrate that reduced or eliminated CD59 expression/function would protect against insulin resistance and fatty liver.

### Example 3: Analysis of High Fat Diet (HFD) in CD59 Deficient Mice

Objective: To study the noncanonical function of CD59.

Specific Aim. To evaluate the insulin signaling pathway and metabolic effects seen in CD59 KO. The insulin signaling and metabolomic effect of insulin and glucose in a CD59 KO model will be studied using intact liver and muscle samples and samples stimulated, in vivo, by insulin, followed by detection of the insulin signaling pathway using Western blots. First, metabolic analysis using antibodies against natural and phosphorylated AKT, p110, pS6, and pACC will be performed and according to the results obtained, further study of the insulin signaling pathway will occur. Example 2 above shows preliminary experiments using anti-AKT and pAKT with clear evidence of abrogated insulin signaling with a HFD for WT but not CD59 KO mice.

The metabolic effects and differences between WT and CD59 KO mice after consuming the HFD will be observed by evaluation of RNA Seq, LC-MS,38 and isotope tracers in plasma (only LC-MS), liver (divided to three lobes), and muscle, with a HFD and upon stimulation with insulin.

Specific Aim. To study the effect of CD59 on the generation of fatty liver with a HFD: pathophysiology and treatment. The results of Example 2 indicate that CD59 KO mice do not develop fatty liver. The effect of CD59 on the generation of fatty liver will be studied using immuno-histochemistry, trying to evaluate the fat and the inflammatory composition of liver inflammation and the activation of complement and using data obtained from Specific aims 1 and 2 above.

A preliminary method of treatment for WT fatty liver will be developed using cyclic proteins or siRNA. It may be that complement has both canonic and noncanonical functions. An important noncanonical role in insulin secretion was shown for CD59 in pancreatic β-cells. Example 2 demonstrated a surprising protective role for CD59 in the periphery, with protection from the development of glucose intolerance insulin resistance and fatty liver. This protective role will be studied and characterized in the periphery using CD59 KO mice, a HFD, insulin signaling, immune-histology, RNA-Seq, and an LC-MS metabolomics screen.

Hepatic steatosis is defined as intrahepatic fat comprising at least 5% of liver weight. Simple accumulation of triacylglycerols (TAG) in the liver could be hepatoprotective; however, prolonged hepatic lipid storage may lead to liver metabolic dysfunction, inflammation, and advanced forms of nonalcoholic fatty liver disease (NAFLD). Nonalcoholic hepatic steatosis is associated with obesity, type 2 diabetes, and dyslipidemia. Several mechanisms are involved in the accumulation of intrahepatic fat, including increased flux of fatty acids to the liver, increased de novo lipogenesis, and/or reduced clearance through β-oxidation or very-low-density lipoprotein secretion. NAFLD is a spectrum of liver disorders that encompasses the presence of simple hepatic steatosis and hepatic steatohepatitis, with or without fibrosis.

Additionally, a therapeutic intervention in WT HFD development of fatty liver will be designed. Both siRNA and cyclized peptide libraries will be developed. Protein-protein interactions (PPIs) are particularly important for controlling both physiologic and pathologic biological processes but are difficult to target due to their large and/or shallow interaction surfaces, which are unsuitable for small molecules. Linear peptides found in nature interact with some PPIs, and protein active regions can be used to design synthetic peptide compounds for inhibition of PPIs. However, linear peptides are limited therapeutically by poor metabolic and conformational stability, which can compromise their bioactivity and half-life. Cyclic peptidomimetics (modified peptides) can be used to overcome these challenges because they are more resistant to metabolic degradation and can be engineered to adopt desired conformations. Backbone cyclization is a strategy that has been developed to improve drug-like properties of linear peptide leads without jeopardizing the integrity of functionally relevant sidechains. An approach for developing backbone cyclized peptide compounds will be provided, based upon two straightforward ‘ABC’ and ‘DEF’ processes that represent a method for revealing active regions important for PPIs and identifying critical pharmacophores, as well as developing backbone cyclized peptide libraries and screening them using cycloscan.

While certain features of the methods of treating liver disease have been illustrated and described herein, many modifications, substitutions, changes, and equivalents will now occur to those of ordinary skill in the art. It is, therefore, to be understood that the appended claims are intended to cover all such modifications and changes as fall within the true spirit of these methods.

What is claimed is:

1. A compound effective in reducing GPI-anchored CD59 expression or activity in liver, skeletal muscle, or adipose tissue, or any combination thereof, for use in the treatment of a liver disease or condition in a subject in need thereof.

2. The compound for use of claim 1, wherein reduction of GPI-anchored CD59 expression or activity comprises reducing CD59 expression, reducing the quantity of GPI-anchored CD59, or inhibiting functional activities of GPI-anchored CD59.

3. The compound for use of claim 1 or claim 2, wherein the compound comprises an oligonucleotide, antibody or a binding fragment thereof, a polypeptide, a peptide, or small molecule.

4. The compound for use of claim 3, wherein said oligonucleotide comprises an antisense oligonucleotide, interfering RNA compounds (RNAi), a siRNA, a miRNA, or guide RNA.

5. The compound for use of claim 3 or claim 4, wherein said oligonucleotide comprises a conjugate group attached at the 5′ or 3′ end of the oligonucleotide.

6. The compound for use of claim 5, wherein said conjugate group comprises at least one GalNAc moiety.

7. The compound for use of claim 4, wherein said antisense oligonucleotide comprises a contiguous nucleotide sequence at least 80%, 85%, 90%, 95%, or 100% complementary to an equal length portion of a target CD59 mRNA transcript or CD59 mRNA precursor.

8. The compound for use of claim 7, wherein said target CD59 mRNA comprises the nucleotide sequence set forth in any one of SEQ ID NOs:3-10.

9. The compound for use of claim 4, wherein said siRNA or miRNA comprises a contiguous nucleotide sequence at least 80%, 85%, 90%, 95%, or 100% complementary to an equal length portion of a target nucleic acid sequence encoding the CD59 protein, said target nucleic acid sequence comprises the sequence of one of SEQ ID NOs:3-12, or is at least 80%, 85%, 90%, 95%, or 100% complementary to an equal length portion of a nucleic acid sequence upstream or downstream of the sequence encoding the CD59 protein.

10. The compound for use of claim 4, wherein when said oligonucleotide comprises a guide RNA,
(a) said use further comprises a polynucleotide encoding a CRISPR-Cas9 endonuclease operatively linked to a liver promoter; and
(b) the guide RNA comprises a contiguous nucleotide sequence complementary to an equal length portion of a target nucleic acid sequence encoding the CD59 protein, wherein said target nucleic acid sequence comprises the sequence of one of SEQ ID NOs:3-12, or is complementary to an equal length portion of a nucleic acid sequence upstream or downstream of the sequence encoding the CD59 protein.

11. The compound for use of any of claims 1-10, wherein said use reduces expression of CD59 in the liver, skeletal muscle, or adipose tissue, or any combination thereof; reduces the quantity of GPI-anchored CD59 in the liver, skeletal muscle, or adipose tissue, or any combination thereof; or reduces or inhibits functional activities of GPI-anchored CD59 in the liver, skeletal muscle, or adipose tissue, or any combination thereof; or a combination thereof.

12. The compound for use of any of claims 1-11, wherein said liver disease or condition comprises fatty liver disease, NASH, or peripheral insulin resistance, and optionally, wherein said subject is further suffering from diabetes or obesity, or a combination thereof.

13. The compound for use of claim 12, wherein said fatty liver disease comprises non-alcoholic fatty liver disease (NAFLD), alcoholic fatty liver disease (AFLD), alcoholic steatohepatitis (ASH) (cirrhotic or non-cirrhotic ASH), non-alcoholic steatohepatitis (NASH) (cirrhotic or non-cirrhotic NASH), hepatocellular carcinoma (HCC), or liver fibrosis, or any combination thereof.

14. The compound for use of claim 12 or claim 13, wherein said subject suffering from the fatty liver disease has liver damage, steatosis, liver inflammation, liver scarring or cirrhosis, liver failure, or peripheral insulin resistance, or any combination thereof.

15. The compound for use of any of claims 1-14, wherein said subject is suffering from diabetes or obesity, or a combination thereof.

16. The compound for use of any of claims 1-15, wherein said treatment further treats glucose intolerance, insulin resistance associated with said liver disease or condition, diabetes, or diabetes associated with weight gain, diabetes associated with insulin resistance, or a combination thereof.

17. The compound for use of any of claims 1-16, wherein said treatment further modulates weight loss in said subject, said modulating weight loss comprising maintaining weight, reducing weight, or reducing increased weight gain compared with a subject not administered said compound.

18. A compound effective in reducing GPI-anchored CD59 expression or activity for use in the modulation of weight loss in a subject in need, said modulation of weight loss comprising maintaining weight, reducing weight, or reducing increased weight gain compared with a subject not administered said compounds.

19. The compound for use of claim 18, wherein reduction of GPI-anchored CD59 expression or activity comprises reducing CD59 expression, reducing the quantity of GPI-anchored CD59, or inhibiting functional activities of GPI-anchored CD59.

20. The compound for use of claim 18 or claim 19, wherein the compound comprises an oligonucleotide, antibody or a binding fragment thereof, a polypeptide, a peptide, or small molecule.

21. The compound for use of claim 20, wherein said oligonucleotide comprises an antisense oligonucleotide, interfering RNA compounds (RNAi), a siRNA, a miRNA, or guide RNA.

22. The compound for use of claim 20 or claim 21, wherein said oligonucleotide comprises a conjugate group attached at the 5′ or 3′ end of the oligonucleotide.

23. The compound for use of claim 22, wherein said conjugate group comprises at least one GalNAc moiety.

24. The compound for use of claim 21, wherein said antisense oligonucleotide comprises a contiguous nucleotide sequence at least 80%, 85%, 90%, 95%, or 100% complementary to an equal length portion of a target CD59 mRNA transcript or CD59 mRNA precursor.

25. The compound for use of claim 24, wherein said target CD59 mRNA comprises the nucleotide sequence set forth in any one of SEQ ID NOs:3-10.

26. The compound for use of claim 21, wherein said siRNA or miRNA comprises a contiguous nucleotide sequence at least 80%, 85%, 90%, 95%, or 100% complementary to an equal length portion of a target nucleic acid sequence encoding the CD59 protein, said target nucleic acid sequence comprises the sequence of one of SEQ ID NOs:3-12, or is at least 80%, 85%, 90%, 95%, or 100% complementary to an equal length portion of a nucleic acid sequence upstream or downstream of the sequence encoding the CD59 protein.

27. The compound for use of claim 21, wherein when said oligonucleotide comprises a guide RNA,
(a) said use further comprises a polynucleotide encoding a CRISPR-Cas9 endonuclease operatively linked to a liver promoter; and
(b) the guide RNA comprises a contiguous nucleotide sequence complementary to an equal length portion of a target nucleic acid sequence encoding the CD59 protein, wherein said target nucleic acid sequence comprises the sequence of one of SEQ ID NOs:3-12, or is complementary to an equal length portion of a nucleic acid sequence upstream or downstream of the sequence encoding the CD59 protein.

28. The compound for use of any one of claims 18-27, wherein said use reduces expression of CD59 in the liver, skeletal muscle, or adipose tissue, or any combination thereof; reduces the quantity of GPI-anchored CD59 in the liver, skeletal muscle, or adipose tissue, or any combination thereof; or inhibits functional activities of GPI-anchored CD59 in the liver, skeletal muscle, or adipose tissue, or any combination thereof; or a combination thereof.

29. The compound for use of any of claims 18-28, wherein said subject is suffering from a liver disease or condition, from diabetes, from peripheral insulin resistance, or is obese, or any combination thereof.

30. The compound for use of claim 29, wherein said liver disease or condition comprises fatty liver disease or NASH, or a combination thereof.

31. The compound for use of claim 30, wherein said fatty liver disease comprises non-alcoholic fatty liver disease (NAFLD), alcoholic fatty liver disease (AFLD), alcoholic steatohepatitis (ASH) (cirrhotic or non-cirrhotic ASH), non-alcoholic steatohepatitis (NASH) (cirrhotic or non-cirrhotic NASH), hepatocellular carcinoma (HCC), or liver fibrosis, or any combination thereof.

32. The compound for use of claim 30 or claim 31, wherein said subject suffering from the fatty liver disease has liver damage, steatosis, liver inflammation, liver scarring or cirrhosis, liver failure, or peripheral insulin resistance, or any combination thereof.

33. The compound for use of any of claims 1-17, wherein said treating further treats glucose intolerance or insulin resistance associated with said liver disease or condition, or a combination thereof.

34. A compound effective in reducing GPI-anchored CD59 expression or activity in the liver, skeletal muscle, or adipose tissue, or any combination thereof for use in the treatment of diabetes or a related condition in a subject in need thereof.

35. The compound for use of claim 34, wherein reduction of GPI-anchored CD59 expression or activity comprises reducing CD59 expression, reducing the quantity of GPI-anchored CD59, or inhibiting functional activities of GPI-anchored CD59.

36. The compound for use of claim 34 or claim 35, wherein the compound comprises an oligonucleotide, antibody or a binding fragment thereof, a polypeptide, a peptide, or small molecule.

37. The compound for use of claim 36, wherein said oligonucleotide comprises an antisense oligonucleotide, interfering RNA compounds (RNAi), a siRNA, a miRNA, or guide RNA.

38. The compound for use of claim 36 or claim 37, wherein said oligonucleotide comprises a conjugate group attached at the 5′ or 3′ end of the oligonucleotide.

39. The compound for use of claim 38, wherein said conjugate group comprises at least one GalNAc moiety.

40. The compound for use of claim 37, wherein said antisense oligonucleotide comprises a contiguous nucleotide sequence at least 80%, 85%, 90%, 95%, or 100% complementary to an equal length portion of a target CD59 mRNA transcript or CD59 mRNA precursor.

41. The compound for use of claim 40, wherein said target CD59 mRNA comprises the nucleotide sequence set forth in any one of SEQ ID NOs:3-10.

42. The compound for use of claim 37, wherein said siRNA or miRNA comprises a contiguous nucleotide sequence at least 80%, 85%, 90%, 95%, or 100% complementary to an equal length portion of a target nucleic acid sequence encoding the CD59 protein, said target nucleic acid sequence comprises the sequence of one of SEQ ID NOs:3-12, or is at least 80%, 85%, 90%, 95%, or 100% complementary to an equal length portion of a nucleic acid sequence upstream or downstream of the sequence encoding the CD59 protein.

43. The compound for use of claim 37, wherein when said oligonucleotide comprises a guide RNA,
(a) said use further comprises a polynucleotide encoding a CRISPR-Cas9 endonuclease operatively linked to a liver promoter; and
(b) the guide RNA comprises a contiguous nucleotide sequence complementary to an equal length portion of a target nucleic acid sequence encoding the CD59 protein, wherein said target nucleic acid sequence comprises the sequence of one of SEQ ID NOs:3-12, or is complementary to an equal length portion of a nucleic acid sequence upstream or downstream of the sequence encoding the CD59 protein.

44. The compound for use of any of claims 34-43, wherein said use reduces expression of CD59 in the liver, skeletal muscle, or adipose tissue, or any combination thereof; reduces the quantity of GPI-anchored CD59 in the liver, skeletal muscle, or adipose tissue, or any combination thereof; or reduces or inhibits functional activities of GPI-anchored CD59 in the liver, skeletal muscle, or adipose tissue, or any combination thereof; or a combination thereof.

45. The compound for use of any of claims 34-44, wherein said diabetes or related condition comprises diabetes type II, diabetes type I, diabetes associated with weight gain, diabetes associated with insulin resistance, or prediabetes, or a combination thereof.

46. The compound for use of claim 45, wherein said subject is further suffering from a liver disease or obesity or a combination thereof, said liver disease selected from a fatty liver disease comprising non-alcoholic fatty liver disease (NAFLD), alcoholic fatty liver disease (AFLD), alcoholic steatohepatitis (ASH) (cirrhotic or non-cirrhotic ASH), non-alcoholic steatohepatitis (NASH) (cirrhotic or non-cirrhotic NASH), hepatocellular carcinoma (HCC), or liver fibrosis, or any combination thereof, and wherein optionally, said subject suffering from the fatty liver disease has liver damage, steatosis, liver inflammation, liver scarring or cirrhosis, liver failure, or peripheral insulin resistance, or any combination thereof.

47. The compound for use of any of claims 34-46, wherein said treating further treats glucose intolerance or peripheral insulin resistance, or any combination thereof.

48. The compound for use of any of claims 34-47, wherein said treatment further modulates weight loss in said subject, said modulating weight loss comprising maintaining weight, reducing weight, or reducing increased weight gain compared with a subject not administered said compound.

49. A compound effective in reducing GPI-anchored CD59 expression or activity in the liver, skeletal muscle, or adipose tissue, or any combination thereof for use in the reduction of insulin resistance in a subject in need thereof.

50. The compound for use of claim 49, wherein reduction of GPI-anchored CD59 expression or activity comprises reducing CD59 expression, reducing the quantity of GPI-anchored CD59, or inhibiting functional activities of GPI-anchored CD59.

51. The compound for use of claim 49 or claim 50, wherein the compound comprises an oligonucleotide, antibody or a binding fragment thereof, a polypeptide, a peptide, or small molecule.

52. The compound for use of claim 51, wherein said oligonucleotide comprises an antisense oligonucleotide, interfering RNA compounds (RNAi), a siRNA, a miRNA, or guide RNA.

53. The compound for use of claim 51 or claim 52, wherein said oligonucleotide comprises a conjugate group attached at the 5′ or 3′ end of the oligonucleotide.

54. The compound for use of claim 53, wherein said conjugate group comprises at least one GalNAc moiety.

55. The compound for use of claim 52, wherein said antisense oligonucleotide comprises a contiguous nucleotide sequence at least 80%, 85%, 90%, 95%, or 100% complementary to an equal length portion of a target CD59 mRNA transcript or CD59 mRNA precursor.

56. The compound for use of claim 55, wherein said target CD59 mRNA comprises the nucleotide sequence set forth in any one of SEQ ID NOs:3-10.

57. The compound for use of claim 52, wherein said siRNA or miRNA comprises a contiguous nucleotide sequence at least 80%, 85%, 90%, 95%, or 100% complementary to an equal length portion of a target nucleic acid sequence encoding the CD59 protein, said target nucleic acid sequence comprises the sequence of one of SEQ ID NOs:3-12, or is at least 80%, 85%, 90%, 95%, or 100% complementary to an equal length portion of a nucleic acid sequence upstream or downstream of the sequence encoding the CD59 protein.

58. The compound for use of claim 52, wherein when said oligonucleotide comprises a guide RNA,
(a) said use further comprises administering a polynucleotide encoding a CRISPR-Cas9 endonuclease operatively linked to a liver promoter; and
(b) the guide RNA comprises a contiguous nucleotide sequence complementary to an equal length portion of a target nucleic acid sequence encoding the CD59 protein, wherein said target nucleic acid sequence comprises the sequence of one of SEQ ID NOs:3-12, or is complementary to an equal length portion of a nucleic acid sequence upstream or downstream of the sequence encoding the CD59 protein.

59. The compound for use of any of claims 49-58, wherein said use reduces expression of CD59 in the liver, skeletal muscle, or adipose tissue, or any combination thereof; reduces the quantity of GPI-anchored CD59 in the liver, skeletal muscle, or adipose tissue, or any combination thereof; or reduces or inhibits functional activities of GPI-anchored CD59 in the liver, skeletal muscle, or adipose tissue, or any combination thereof; or a combination thereof.

60. The compound for use of any of claims 49-59, wherein insulin resistance is reduced in, liver, muscle or adipose tissue, or any combination thereof.

61. The compound for use of any of claims 49-60, wherein said subject further suffers from diabetes or a related condition, from a liver disease, or from obesity,
said diabetes comprising diabetes type II, diabetes type I, diabetes associated with weight gain, diabetes associated with insulin resistance, or prediabetes, or a combination thereof; and
said liver disease comprising from a fatty liver disease comprising non-alcoholic fatty liver disease (NAFLD), alcoholic fatty liver disease (AFLD), alcoholic steatohepatitis (ASH) (cirrhotic or non-cirrhotic ASH), non-alcoholic steatohepatitis (NASH) (cirrhotic or non-cirrhotic NASH), hepatocellular carcinoma (HCC), or liver fibrosis, or any combination thereof, and wherein optionally, said subject suffering from the fatty liver disease has liver damage, steatosis, liver inflammation, liver scarring or cirrhosis, liver failure, or peripheral insulin resistance, or any combination thereof.

62. The compound for use of any of claims 49-61, wherein said treatment further treats glucose intolerance or reduces or inhibits the occurrence of diabetes in said subject, or any combination thereof.

63. The compound for use of any of claims 49-62, wherein said treating further modulates weight loss in said subject, said modulating weight loss comprising maintaining weight, reducing weight, or reducing increased weight gain compared with a subject not administered said compound.
